(19) United States
(12) Patent Application Publication
(10) Pub. No.: US 2012/0165331 A1
(43) Pub. Date: Jun. 28, 2012

(54) Di/tri-aza-spiro-C9-C11alkanes

(76) Inventors: Sangamesh BADIGER, Bangalore (IN); Dirk BEHNKE, Grenzach-Wyhlen (DE); Claudia BETSCHART, Basel (CH); Vinod CHAUDHARI, Bangalore (IN); Simona COTESTA, Basel (CH); Samuel HINTERMANN, Basel (CH); Andreas LERCHNER, Binningen (CH); Fatma LIMAM, Lingoisheim (FR); Silvio OFNER, Muenchenstein (CH); Chetan PANDIT, Bangalore (IN); Jürgen WAGNER, Bottmingen (CH)

(21) Appl. No.: 13/329,868

(22) Filed: Dec. 19, 2011

(30) Foreign Application Priority Data
Dec. 22, 2010 (IN) 3075/DEL/2010
Jun. 8, 2011 (IN) 1621/DEL/2011
Nov. 14, 2011 (IN) 3233/DEL/2011

(51) Int. Cl. A61K 31/5386; A61K 31/498; A61K 31/501; A61K 31/499; A61K 31/497; A61K 31/517; A61K 31/506; A61K 31/438; A61K 31/427; A61K 31/423; C07D 487/10; C07D 498/04; C07D 471/10; A61P 25/28; C07D 498/10; A61K 31/502

(52) U.S. Cl. 514/235.2; 514/248; 514/249; 514/252.06; 514/252.15; 514/255.05; 514/266.2; 514/275; 514/278; 514/370; 514/375; 544/71; 544/230; 544/231; 546/15; 546/16; 548/147; 548/216

(57) ABSTRACT

The invention relates to compounds of the formula I

A-D-C(R₁)₂—B (I),

in which the substituents are as defined in the specification; in free form or in salt form; to its preparation, to its use as medicament and to medicaments comprising it.

### FIELD OF THE INVENTION

The invention relates to di/tri-aza-spiro-C9-C11alkanes, to their preparation, to their use as medicaments and to medicaments comprising them.

### BACKGROUND

Orexins (orexin A/OX-A and orexin B/OX-B), which are also known as hypocretins, are neuropeptides. Orexin A is a 33 amino acid peptide and orexin B is a 28 amino acid peptide (Sakurai T. et al., Cell, 1998, 92, 573-585). Orexins are produced in discrete neurons of the lateral hypothalamus and bind to G-protein-coupled receptors, the orexin receptors (also known as hypocretin receptors): known are the orexin-1 receptor (OXR1) and the orexin-2 receptor (OXR2). The orexin-1 receptor has some selectivity for OX-A, whereas the orexin-2 receptor binds OX-A and OX-B with similar affinity. Orexins regulate states of sleep and wakefulness, opening potentially novel therapeutic approaches for narcolepsy as well as insomnia and other sleep disorders (Chemelli R. M. et al., Cell, 1999, 98, 437-45 1). Furthermore, orexins were found to stimulate food consumption in rats suggesting a physiological role for these peptides as mediators in the central feedback mechanism that regulates feeding behavior (Sakurai T. et al., Cell, 1998, 92, 573-585). Still furthermore, orexins were shown to play a role in brain reward function/motivation suggesting usefulness to treat substance-related disorders (Harris A. C. et al, Nature, 2005, 437, 556-559). Still furthermore, it has been shown that amyloid beta levels inversely correlate with orexin levels in rodents and humans (brain and/or CSF), and that an orexin receptor antagonist reduces both amyloid beta levels and amyloid plaque load in Alzheimer's transgenic mice, thus suggesting usefulness in the treatment of Alzheimers disease (Kang J. E. et al, Science 2009, 326, 1005-1007).

Orexin receptors may have numerous implications in disorders such as

i) sleep disorders, e.g. sleep apnea, narcolepsy, insomnia, parasomnia, jet lag syndrome, disturbed biological and circadian rhythms; sleep disturbances associated with diseases such as neurological disorders, neuropathic pain and restless leg syndrome;

ii) eating disorders, e.g. appetite and taste disorders;

iii) substance-related disorders, e.g. substance abuse, substance dependence and substance withdrawal disorders, such as nicotine withdrawal or narcotics withdrawal;

iv) Alzheimers disease;

v) psychiatric, neurological and neurodegenerative disorders, e.g. depression; anxiety; addictions, obsessive compulsive disorder; affective neurosis; depressive neurosis; anxiety neurosis; dysthymic disorder; mood disorder; sexual dysfunction; psychosexual dysfunction; sex disorder; schizophrenia; manic depression; delirium; dementia; severe mental retardation and dyskinesias such as Huntington's disease and Tourette syndrome; Parkinson's disease; ischemic or hemorrhagic stroke; migraine; and neurodegenerative disorders including nosological entities such as disinhibition-dementia-parkinsonism-amyotrophy complex; pallido-ponto-nigral degeneration epilepsy; seizure disorders;

vi) cardiovascular diseases, diabetes; asthma; Cushing's syndrome/disease; basophile adenoma; prolactinoma; hyperprolactinemia; hypopituitarism; hypophysis tumor/adenoma; hypothalamic diseases; Froehlich's syndrome; hypophysis diseases, hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; heart and lung diseases, acute and congestive heart failure; hypotension; hypertension; urinary retention; osteoporosis; angina pectoris; myocardial infarction; subarachnoid hemorrhage; ulcers; allergies; benign prostatic hypertrophy; chronic renal failure; renal disease; impaired glucose tolerance; vomiting and nausea; inflammatory bowel disease; gastric dyskinesia; gastric ulcers; urinary bladder incontinence e.g. urge incontinence; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; conditions associated with visceral pain such as irritable bowel syndrome, migraine and angina; and

vii) other diseases related to general orexin system dysfunction.

Orexin receptor antagonists are considered to be useful in the treatment of a wide range of disorders, in particular sleep disorders, eating disorders and substance-related disorders.

Therefore, there is a need to provide new orexin receptor antagonists that are good drug candidates. In particular, preferred compounds should bind potently to the orexin receptors (either as OXR1 or OXR2 subtype selective antagonists or as dual OXR1/OXR2 antagonists) whilst showing little affinity for other receptors. They should be well absorbed from the gastrointestinal tract, be sufficiently metabolically stable and possess favorable pharmacokinetic properties. When targeted against receptors in the central nervous system they should cross the blood brain barrier freely and when targeted selectively against receptors in the peripheral nervous system they should not cross the blood brain barrier. They should be non-toxic and demonstrate few side-effects. Furthermore, the ideal drug candidate will be able to exist in a physical form that is stable, non-hygroscopic and easily formulated.

The compounds of the invention are orexin receptor antagonists and are therefore potentially useful in the treatment of a wide range of disorders, particularly sleep disorders, eating disorders, substance-related disorders and Alzheimers disease.

### DETAILED DESCRIPTION OF THE INVENTION

In a first aspect, the invention relates to

a compound of the formula I

A-D-C(R₁)₂—B (I),

wherein

each R₁ independently is hydrogen, C₁₋₆alkyl, C₁₋₆halogenalkyl, C₃₋₇cycloalkyl or C₃₋₇cycloalkyl(C₁₋₄alkyl), or two R₁ together with the carbon atom to which they are bound form a C₃₋₄cycloalkyl;

A is a five- to six-membered monocyclic aromatic ring system which may contain from 1 to 4 hetero atoms selected from nitrogen, oxygen and sulfur, and which is substituted once or more than once by R₂;

or A is an eight- to ten-membered fused bicyclic aromatic ring system which may contain from 1 to 4 hetero atoms selected from nitrogen, oxygen and sulfur, and which may be substituted once or more than once by R₂;

each R₂ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; or a three- to seven-membered monocyclic ring system which may be aromatic, saturated or unsaturated non-aromatic, which may contain from 1 to 4 hetero atoms selected from nitrogen, oxygen and sulfur, and which may be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; or two R₂ at adjacent ring atoms form together with said ring atoms a fused five- to seven-membered unsaturated non-aromatic ring system which may contain from 1 to 4 hetero atoms selected from nitrogen, oxygen and sulfur, and which may be substituted once or more than once by R₃;

each R₃ independently is halogen, C₁₋₆alkyl or C₁₋₆alkoxy, or two R₃ at the same ring atom together are oxo;

B is a five- to six-membered monocyclic aromatic ring system which may contain from 1 to 4 hetero atoms selected from nitrogen, oxygen and sulfur, and which is substituted once or more than once by R₄;

or B is a eight- to ten-membered fused bicyclic aromatic ring system which may contain from 1 to 4 hetero atoms selected from nitrogen, oxygen and sulfur, and which may be substituted once or more than once by R₄;

each R₄ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; B1; or two R₄ at adjacent ring atoms form together with said ring atoms a fused five- to seven-membered unsaturated non-aromatic ring system which may contain from 1 to 4 hetero atoms selected from nitrogen, oxygen and sulfur, and which may in turn be substituted once or more than once by R₅;

B1 is a three- to seven-membered monocyclic ring system which may be aromatic, saturated or unsaturated non-aromatic, which may contain from 1 to 4 hetero atoms selected from nitrogen, oxygen and sulfur, and which may in turn be substituted once or more than once by C₁₋₆alkyl, C₁₋₆halogenalkyl, C₁₋₄alkoxy-C₁₋₆alkyl, C₁₋₆alkoxy, C₃₋₇cycloalkoxy, C₁₋₆halogenalkoxy, C₃₋₇cycloalkylC₁₋₄alkoxy, C₁₋₄alkoxy-C₁₋₄alkoxy, C₁₋₄alkylcarbonyl, N—C₁₋₄alkylaminocarbonyl, C₁₋₄alkylamino, di(C₁₋₄alkyl)amino, halogen, cyano, a 6-membered saturated heterocycle containing 1 to 4 hetero atoms selected from nitrogen, oxygen and sulfur, a 5-membered aromatic ring containing 1 to 4 hetero atoms selected from nitrogen, oxygen and sulfur which may be substituted once or more than once by C₁₋₄alkyl;

or two substituents at adjacent ring atoms of B1 form together with said ring atoms a fused five- to seven-membered unsaturated non-aromatic ring system which may contain from 1 to 4 hetero atoms selected from nitrogen, oxygen and sulfur;

each R₅ independently is halogen, C₁₋₆alkyl or C₁₋₆alkoxy, or two R₅ at the same ring atom together are oxo;

D is selected from the group consisting of

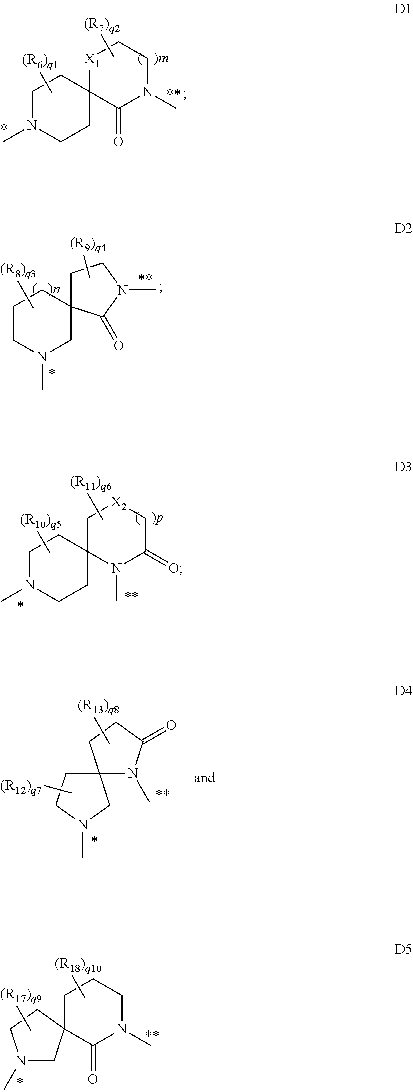

wherein the bond marked with one asterisk is attached to A and the bond marked with two asterisks is attached to C(R₁)₂—B;

wherein when D is D5, B is a five- to six-membered monocyclic aromatic ring system which may contain from 1 to 4 hetero atoms selected from nitrogen, oxygen and sulfur, which is substituted once by B1; or B is a eight- to ten-membered fused bicyclic aromatic ring system which may contain from 1 to 4 hetero atoms selected from nitrogen, oxygen and sulfur, and which may be substituted once or more than once by R₄;

X₁ is —C(R₁₄)₂— or —N(R₁₅)— and m is 0;

or X₁ is —O— or —N(R₁₅)— and m is 1;

each R₆ or R₇ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₆ at the same carbon atom form together with said carbon atom C₃₋₇cycloalkyl, or two R₇ at the same carbon atom form together with said carbon atom C₃₋₇cycloalkyl;

each R₁₄ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

R₁₅ is hydrogen, C₁₋₆alkyl, C₃₋₇cycloalkyl or C₃₋₇cycloalkyl(C₁₋₄alkyl);

q1 is 0, 1, 2, 3, 4, 5 or 6;

q2 is 0, 1, 2, 3 or 4;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6;

q4 is 0, 1, 2, 3 or 4;

X₂ is —C(R₁₆)₂— and p is 0;

or X₂ is —O— and p is 0 or 1;

each R₁₀ or R₁₁ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₀ at the same carbon atom form together with said carbon atom C₃₋₇cycloalkyl, or two R₁₁ at the same carbon atom form together with said carbon atom C₃₋₇cycloalkyl;

each R₁₆ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

q5 is 0, 1, 2, 3, 4, 5 or 6;

q6 is 0, 1, 2, 3 or 4;

each R₁₂ or R₁₃ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₂ at the same carbon atom form together with said carbon atom C₃₋₇cycloalkyl, or two R₁₃ at the same carbon atom form together with said carbon atom C₃₋₇cycloalkyl;

q7 is 0, 1, 2, 3 or 4; and

q8 is 0, 1, 2, 3 or 4;

each R₁₇ or R₁₈ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₇ at the same carbon atom form together with said carbon atom C₃₋₇cycloalkyl, or two R₁₈ at the same carbon atom form together with said carbon atom C₃₋₇cycloalkyl;

q9 is 0, 1, 2, 3 or 4; and

q10 is 0, 1, 2, 3, 4, 5 or 6;

in free form or in salt form or in pharmaceutically acceptable salt form.

In a second aspect, the invention relates to a compound of the formula I

A-D-C(R₁)₂—B (I),

wherein

each R₁ independently is hydrogen, C₁₋₆alkyl, C₁₋₆halogenalkyl, C₃₋₇cycloalkyl or C₃₋₇cycloalkyl(C₁₋₄alkyl), or two R₁ together with the carbon atom to which they are bound form a C₃₋₄cycloalkyl;

A is a five- to six-membered monocyclic aromatic ring system which may contain from 1 to 4 hetero atoms selected from nitrogen, oxygen and sulfur, and which is substituted once or more than once by R₂;

or A is an eight- to ten-membered fused bicyclic aromatic ring system which may contain from 1 to 4 hetero atoms selected from nitrogen, oxygen and sulfur, and which may be substituted once or more than once by R₂;

each R₂ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; or a three- to seven-membered monocyclic ring system which may be aromatic, saturated or unsaturated non-aromatic, which may contain from 1 to 4 hetero atoms selected from nitrogen, oxygen and sulfur, and which may be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; or two R₂ at adjacent ring atoms form together with said ring atoms a fused five- to seven-membered unsaturated non-aromatic ring system which may contain from 1 to 4 hetero atoms selected from nitrogen, oxygen and sulfur, and which may be substituted once or more than once by R₃;

each R₃ independently is halogen, C₁₋₆alkyl or C₁₋₆alkoxy, or two R₃ at the same ring atom together are oxo;

B is a five- to six-membered monocyclic aromatic ring system which may contain from 1 to 4 hetero atoms selected from nitrogen, oxygen and sulfur, and which is substituted once or more than once by R₄;

or B is a eight- to ten-membered fused bicyclic aromatic ring system which may contain from 1 to 4 hetero atoms selected from nitrogen, oxygen and sulfur, and which may be substituted once or more than once by R₄;

each R₄ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; or a three- to seven-membered monocyclic ring system which may be aromatic, saturated or unsaturated non-aromatic, which may contain from 1 to 4 hetero atoms selected from nitrogen, oxygen and sulfur, and which may in turn be substituted once or more than once by C₁₋₆alkyl, C₁₋₆halogenalkyl, C₁₋₄alkoxy-C₁₋₆alkyl, C₁₋₆alkoxy, C₃₋₇cycloalkoxy, C₁₋₆halogenalkoxy, C₃₋₇cycloalkylC₁₋₄alkoxy, C₁₋₄alkylcarbonyl, N—C₁₋₄alkylaminocarbonyl, C₁₋₄alkylamino, di(C₁₋₄alkyl)amino, a 5-membered aromatic ring containing 1 to 4 hetero atoms selected from nitrogen, oxygen and sulfur which may be substituted once or more than once by C₁₋₄alkyl, halogen, cyano or two substituents at adjacent ring atoms form together with said ring atoms a fused five- to seven-membered unsaturated non-aromatic ring system which may contain from 1 to 4 hetero atoms selected from nitrogen, oxygen and sulfur; or two R₄ at adjacent ring atoms form together with said ring atoms a fused five- to seven-membered unsaturated non-aromatic ring system which may contain from 1 to 4 hetero atoms selected from nitrogen, oxygen and sulfur, and which may in turn be substituted once or more than once by R₅;

each R₅ independently is halogen, C₁₋₆alkyl or C₁₋₆alkoxy, or two R₅ at the same ring atom together are oxo;

D is selected from the group consisting of

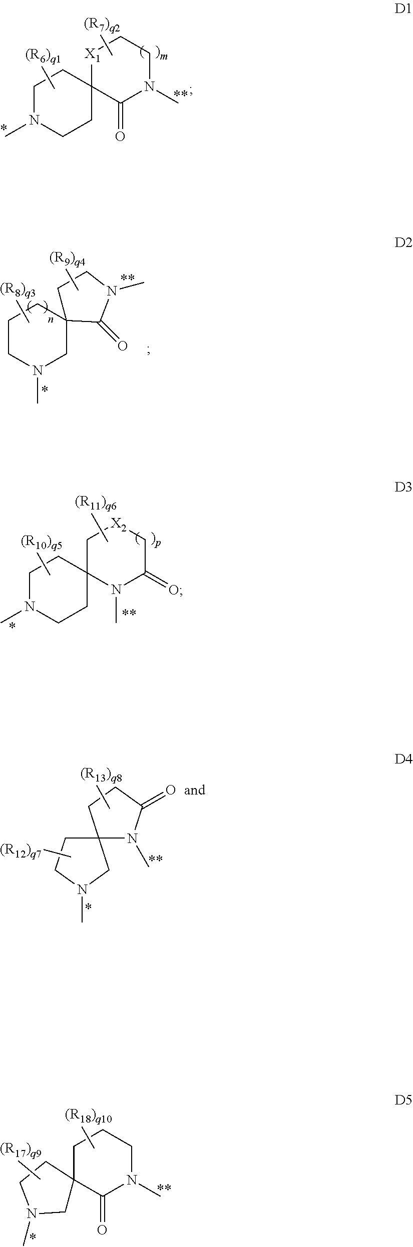

wherein the bond marked with one asterisk is attached to A and the bond marked with two asterisks is attached to C(R₁)₂—B;

wherein when D is D5, B is a five- to six-membered monocyclic aromatic ring system which may contain from 1 to 4 hetero atoms selected from nitrogen, oxygen and sulfur, which is substituted once by B1; or B is a eight- to ten-membered fused bicyclic aromatic ring system which may contain from 1 to 4 hetero atoms selected from nitrogen, oxygen and sulfur, and which may be substituted once or more than once by R₄;

X₁ is —C(R₁₄)₂— or —N(R₁₅)— and m is 0;

or X₁ is —O— or —N(R₁₅)— and m is 1;

each R₆ or R₇ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₆ at the same carbon atom form together with said carbon atom C₃₋₇cycloalkyl, or two R₇ at the same carbon atom form together with said carbon atom C₃₋₇cycloalkyl;

each R₁₄ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

R₁₅ is hydrogen, C₁₋₆alkyl, C₃₋₇cycloalkyl or C₃₋₇cycloalkyl(C₁₋₄alkyl);

q1 is 0, 1, 2, 3, 4, 5 or 6;

q2 is 0, 1, 2, 3 or 4;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6;

q4 is 0, 1, 2, 3 or 4;

X₂ is —C(R₁₆)₂— and p is 0;

or X₂ is —O— and p is 0 or 1;

each R₁₀ or R₁₁ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₀ at the same carbon atom form together with said carbon atom C₃₋₇cycloalkyl, or two R₁₁ at the same carbon atom form together with said carbon atom C₃₋₇cycloalkyl;

each R₁₆ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

q5 is 0, 1, 2, 3, 4, 5 or 6;

q6 is 0, 1, 2, 3 or 4;

each R₁₂ or R₁₃ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₂ at the same carbon atom form together with said carbon atom C₃₋₇cycloalkyl, or two R₁₃ at the same carbon atom form together with said carbon atom C₃₋₇cycloalkyl;

q7 is 0, 1, 2, 3 or 4; and

q8 is 0, 1, 2, 3 or 4;

each R₁₇ or R₁₈ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₇ at the same carbon atom form together with said carbon atom C₃₋₇cycloalkyl, or two R₁₈ at the same carbon atom form together with said carbon atom C₃₋₇cycloalkyl;

q9 is 0, 1, 2, 3 or 4; and

q10 is 0, 1, 2, 3, 4, 5 or 6;

in free form or in salt form or in pharmaceutically acceptable salt form.

In a third aspect, the invention relates to a compound of formula (I)

A-D-C(R₁)₂—B (I),

wherein

each R₁ independently is hydrogen, C₁₋₆alkyl, C₁₋₆halogenalkyl, C₃₋₇cycloalkyl or C₃₋₇cycloalkyl(C₁₋₄alkyl), or two R₁ together with the carbon atom to which they are bound form a C₃₋₄cycloalkyl;

A is a five- to six-membered monocyclic aromatic ring system which may contain from 1 to 4 hetero atoms selected from nitrogen, oxygen and sulfur, and which is substituted once or more than once by R₂;

or A is an eight- to ten-membered fused bicyclic aromatic ring system which may contain from 1 to 4 hetero atoms selected from nitrogen, oxygen and sulfur, and which may be substituted once or more than once by R₂;

each R₂ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; or a three- to seven-membered monocyclic ring system which may be aromatic, saturated or unsaturated non-aromatic, which may contain from 1 to 4 hetero atoms selected from nitrogen, oxygen and sulfur, and which may in turn be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; or two R₂ at adjacent ring atoms form together with said ring atoms a fused five- to seven-membered unsaturated non-aromatic ring system which may contain from 1 to 4 hetero atoms selected from nitrogen, oxygen and sulfur, and which may in turn be substituted once or more than once by R₃;

each R₃ independently is halogen, C₁₋₆alkyl or C₁₋₆alkoxy, or two R₃ at the same ring atom together are oxo;

B is a five- to six-membered monocyclic aromatic ring system which may contain from 1 to 4 hetero atoms selected from nitrogen, oxygen and sulfur, and which is substituted once or more than once by R₄;

or B is a eight- to ten-membered fused bicyclic aromatic ring system which may contain from 1 to 4 hetero atoms selected from nitrogen, oxygen and sulfur, and which may be substituted once or more than once by R₄;

each R₄ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; or a three- to seven-membered monocyclic ring system which may be aromatic, saturated or unsaturated non-aromatic, which may contain from 1 to 4 hetero atoms selected from nitrogen, oxygen and sulfur, and which may in turn be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; or two R₄ at adjacent ring atoms form together with said ring atoms a fused five- to seven-membered unsaturated non-aromatic ring system which may contain from 1 to 4 hetero atoms selected from nitrogen, oxygen and sulfur, and which may in turn be substituted once or more than once by R₅;

each R₅ independently is halogen, C₁₋₆alkyl or C₁₋₆alkoxy, or two R₅ at the same ring atom together are oxo;

D is selected from the group consisting of

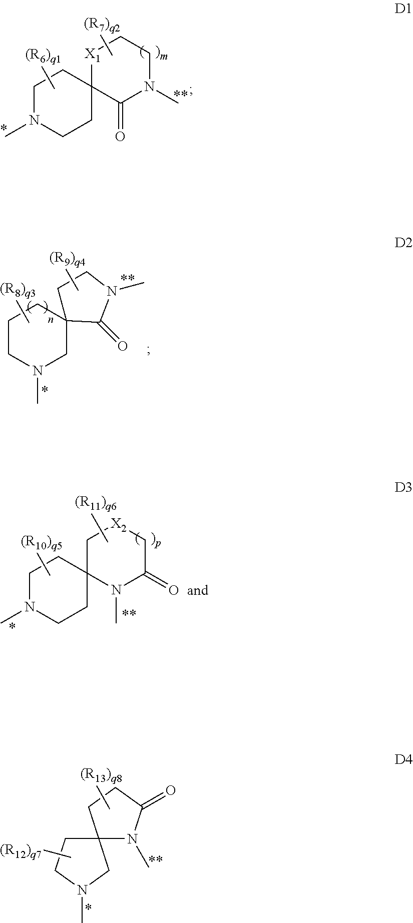

wherein the bond marked with one asterisk is attached to A and the bond marked with two asterisks is attached to C(R₁)₂—B;

X₁ is —C(R₁₄)₂— and m is 0;

or X₁ is —O— or —N(R₁₅)— and m is 1;

each R₆ or R₇ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₆ at the same carbon atom form together with said carbon atom C₃₋₇cycloalkyl, or two R₇ at the same carbon atom form together with said carbon atom C₃₋₇cycloalkyl;

each R₁₄ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

R₁₅ is hydrogen, C₁₋₆alkyl, C₃₋₇cycloalkyl or C₃₋₇cycloalkyl(C₁₋₄alkyl);

q1 is 0, 1, 2, 3, 4, 5 or 6;

q2 is 0, 1, 2, 3 or 4;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6;

q4 is 0, 1, 2, 3 or 4;

X₂ is —C(R₁₆)₂— and p is 0;

or X₂ is —O— and p is 0 or 1;

each R₁₀ or R₁₁ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₀ at the same carbon atom form together with said carbon atom C₃₋₇cycloalkyl, or two R₁₁ at the same carbon atom form together with said carbon atom C₃₋₇cycloalkyl;

each R₁₆ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

q5 is 0, 1, 2, 3, 4, 5 or 6;

q6 is 0, 1, 2, 3 or 4;

each R₁₂ or R₁₃ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₂ at the same carbon atom form together with said carbon atom C₃₋₇cycloalkyl, or two R₁₃ at the same carbon atom form together with said carbon atom C₃₋₇cycloalkyl;

q7 is 0, 1, 2, 3 or 4; and

q8 is 0, 1, 2, 3 or 4;

in free form or in salt form.

In one embodiment of the invention, there is provided a compound of formula (I-a) in free form or in pharmaceutically acceptable salt form,

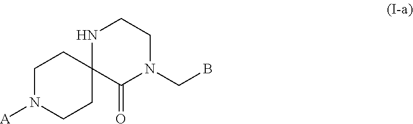

wherein A and B are as defined hereinabove in relation to a compound of formula (I).

In one embodiment of the invention, there is provided a compound of formula (I-b) in free form or in pharmaceutically acceptable salt form,

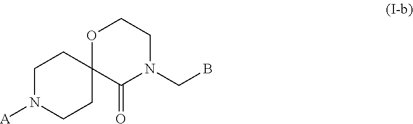

wherein A and B are as defined hereinabove in relation to a compound of formula (I).

In one aspect of the invention, there is provided a compound of formula (I-c) in free form or in pharmaceutically acceptable salt form,

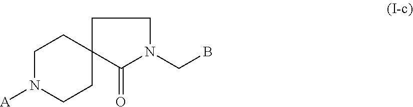

wherein A and B are as defined hereinabove in relation to a compound of formula (I).

In one embodiment of the invention, there is provided a compound of formula (I-d) in free form or in pharmaceutically acceptable salt form,

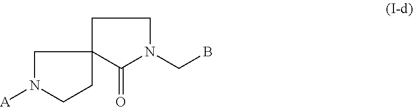

wherein A and B are as defined hereinabove in relation to a compound of formula (I).

In one embodiment of the invention, there is provided a compound of formula (I-e) in free form or in pharmaceutically acceptable salt form,

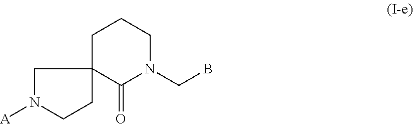

wherein A is as defined hereinabove in relation to a compound of formula (I) and wherein B is a five- to six-membered monocyclic aromatic ring system which may contain from 1 to 4 hetero atoms selected from nitrogen, oxygen and sulfur, which is substituted once by B1; or B is a eight- to ten-membered fused bicyclic aromatic ring system which may contain from 1 to 4 hetero atoms selected from nitrogen, oxygen and sulfur, and which may be substituted once or more than once by R₄.

In one embodiment of the invention, there is provided a compound of formula (I-f) in free form or in pharmaceutically acceptable salt form,

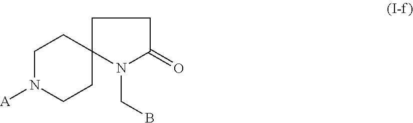

wherein A and B are as defined hereinabove in relation to a compound of formula (I).

In one embodiment of the invention, there is provided a compound of formula (I-g) in free form or in pharmaceutically acceptable salt form,

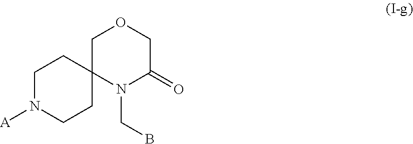

wherein A and B are as defined hereinabove in relation to a compound of formula (I).

In one embodiment of the invention, there is provided a compound of formula (I-h) in free form or in pharmaceutically acceptable salt form,

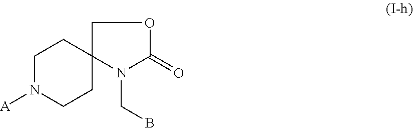

wherein A and B are as defined hereinabove in relation to a compound of formula (I).

Unless specified otherwise, the term “compounds of the present invention” refers to compounds of Formula (I), (I-a), (I-b), (I-c), (I-d), (I-e), (I-f), (I-g), (I-h) prodrugs thereof, salts of the compound and/or prodrugs, hydrates or solvates of the compounds, salts and/or prodrugs, as well as all stereoisomers (including diastereoisomers and enantiomers), tautomers and isotopically labeled compounds (including deuterium substitutions), as well as inherently formed moieties (e.g., polymorphs, solvates and/or hydrates).

Unless indicated otherwise, the expressions used in this invention have the following meaning:

“Alkyl” represents a straight-chain or branched-chain alkyl group, for example, methyl, ethyl, n- or iso-propyl or tert-butyl; in one embodiment, C₁₋₆alkyl represents a straight-chain or branched-chain C₁₋₄alkyl, e.g. methyl, ethyl, n-propyl, iso-propyl and tert-butyl.

Each alkyl part of “alkoxy”, “halogenalkyl” and so on shall have the same meaning as described in the above-mentioned definition of “alkyl”, especially regarding linearity and size.

“C₃₋₇cycloalkyl” represents a saturated alicyclic moiety having from three to seven carbon atoms. This term refers to groups such as cyclopropyl, cyclobutyl, cyclopentyl and cyclohexyl.

A substituent being substituted “once or more than once”, for example as defined for ring system A, is typically substituted by one to three substituents; e.g. the ring system A may be substituted three times by R₂, wherein one R₂ is halogen and two R₂ at adjacent ring atoms form together with said ring atoms a fused five-membered unsaturated non-aromatic ring system.

Halogen is generally fluorine, chlorine, bromine or iodine; e.g. fluorine, chlorine or bromine. In one embodiment, halogenalkyl groups have a chain length of 1 to 4 carbon atoms and are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,2-trichloroethyl, 1,1,2,2-tetrafluoroethyl, 2,2,3,3-tetrafluoropropyl, 2,2,3,3,3-pentafluoropropyl or 2,2,3,4,4,4-hexafluorobutyl; typically fluoromethyl, difluoromethyl or trifluoromethyl.

In the context of the invention, the definitions of A and B as “five- to six-membered monocyclic or eight- to ten-membered fused bicyclic aromatic ring system” encompasses a C₆- or C₁₀-aromatic hydrocarbon group or a five-, six-, eight-, nine- or ten-membered heterocyclic aromatic ring system.

The term “fused bicyclic aromatic ring system” refers to an aromatic substituent which consists of two aromatic rings that are fused together.

In the context of the invention, the definition of e.g. R₂ as a “three- to seven-membered monocyclic ring system” encompasses a C₆-aromatic hydrocarbon group, a five- to six-membered heterocyclic aromatic ring system and a three- to seven-membered monocyclic aliphatic or heterocyclic ring system.

In the context of the invention, the definition of e.g. two R₂ as a “fused five- to seven-membered unsaturated non-aromatic ring system” encompasses five- to seven-membered hydrocarbon and heterocyclic groups which comprise at least one double-bond, which is shared with the aromatic ring system they are fused to.

In the context of the invention, the definition of e.g. R₄ as a “three- to seven-membered monocyclic ring system which may be aromatic, saturated or unsaturated non-aromatic, which may contain from 1 to 4 hetero atoms selected from nitrogen, oxygen and sulfur encompasses a C₆-aromatic hydrocarbon group, a five to six-membered heterocyclic aromatic ring system and a three- to seven-membered monocyclic aliphatic or heterocyclic ring system.

A C₆- or C₁₀-aromatic hydrocarbon group is typically phenyl or naphthyl respectively. A C₆-aromatic hydrocarbon group is especially phenyl.

Typically, but also depending on further substituent definition, “five- to six-membered monocyclic or eight- to ten-membered fused bicyclic aromatic ring systems which contain from 1 to 4 hetero atoms” consist of 5-6 or 8-10 ring atoms of which 1-4 ring atoms are hetero atoms. Such heterocyclic aromatic ring systems may be present as a single aromatic ring system or as multiple, e.g. two, fused aromatic ring systems; typically such rings systems are single ring systems or benz-annelated ring systems.

Examples of heterocyclic ring systems are: imidazo[2,1-b]thiazole, pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazolidine, isothiazole, isothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pteridine, and the corresponding benz-annelated heterocycles, e.g. indole, isoindole, coumarin, isoquinoline, quinoline, quinoxaline and the like. Further examples of heterocycles are: quinoxaline, indole, pyridine, 1H-benzo[d]imidazole, quinoline, pyrimidine, 1,3,4-oxadiazole, isoxazole, pyrrole or benzo[d]isoxazole.

Typical examples of five-membered heterocyclic aromatic ring systems include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl.

Typical examples of six-membered heterocyclic aromatic ring systems include 2-, 3-, or 4-pyridyl, 3- or 4-pyridazinyl, 3-, 4-, or 5-pyrazinyl, 2-pyrazinyl, and 2-, 4-, or 5-pyrimidinyl.

Unless otherwise indicated herein, the compounds of Formula (I), (I-a), (I-b), (I-c), (I-d), (I-e), (I-f), (I-g), (I-h) may exist in optically active form or in form of mixtures of optical isomers, e.g. in form of racemic mixtures or diastereomeric mixtures. In particular, asymmetrical carbon atom(s) may be present in the compounds of Formula (I), (I-a), (I-b), (I-c), (I-d), (I-e), (I-f), (I-g), (I-h) and their salts. Unless otherwise indicated herein, all optical isomers and their mixtures, including the racemic mixtures, are embraced by the invention.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the invention and includes geometric isomers. It is understood that a substituent may be attached at a chiral center of a carbon atom. Therefore, unless otherwise indicated, the invention includes enantiomers, diastereomers or racemates of the compound.

“Enantiomers” are a pair of stereoisomers that are non-superimposable mirror images of each other.

A 1:1 mixture of a pair of enantiomers is a “racemic” mixture. The term is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. The compounds described herein may contain one or more asymmetric centers and may thus give rise to enantiomers, diastereomers, and other stereoisomeric forms that may be defined, in terms of absolute stereochemistry, as (R)- or (S)-. Unless otherwise indicated, the invention is meant to include all such possible isomers, including racemic mixtures, optically pure forms or intermediate mixtures. Optically active (R)- and (S)-isomers may be prepared using chiral synthons or chiral reagents, or resolved using conventional techniques.

If the compound contains a double bond, unless otherwise indicated herein, the substituent may be E or Z configuration.

If the compound contains a disubstituted cycloalkyl, unless otherwise indicated herein, the cycloalkyl substituent may have a cis- or trans-configuration.

Unless otherwise indicated herein, any asymmetric atom (e.g. carbon or the like) of the compound(s) of the invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein and unless otherwise indicated herein, a compound of the invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Depending on substituent definition, compounds of Formula (I), (I-a), (I-b), (I-c), (I-d), (I-e), (I-f), (I-g), (I-h) may occur in various tautomeric forms. All tautomeric forms of the compounds of Formula (I), (I-a), (I-b), (I-c), (I-d), (I-e), (I-f), (I-g), (I-h) are embraced by the invention.

Compounds of Formula (I), (I-a), (I-b), (I-c), (I-d), (I-e), (I-f), (I-g), (I-h) may exist in free form or as a salt. In this specification, unless otherwise indicated, language such as “compound of Formula (I), (I-a), (I-b), (I-c), (I-d), (I-e), (I-f), (I-g), (I-h)” is to be understood as embracing the compounds in any form, for example free or acid addition salt form. Salts which are unsuitable for pharmaceutical uses but which can be employed, for example, for the isolation or purification of free compounds of Formula (I), (I-a), (I-b), (I-c), (I-d), (I-e), (I-f), (I-g), (I-h), such as picrates or perchlorates, are also included. For therapeutic use, only pharmaceutically acceptable salts or free compounds are employed (where applicable in the form of pharmaceutical preparations), and are therefore preferred. Salts are preferably physiologically acceptable salts, formed by the addition of an acid.

As used herein, the term “pharmaceutically acceptable salts” refers to salts that retain the biological effectiveness and properties of the compounds of this invention and, which typically are not biologically or otherwise undesirable. The compounds of the invention may be capable of forming acid salts by virtue of the presence of suitable groups, such as amino groups.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

The pharmaceutically acceptable salts of the invention can be synthesized from a parent compound by conventional chemical methods. Generally, such salts can be prepared by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The invention includes all pharmaceutically acceptable isotopically-labeled compounds of the invention, i.e. compounds of Formula (I), (I-a), (I-b), (I-c), (I-d), (I-e), (I-f), (I-g), (I-h) wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio.

Examples of isotopes suitable for inclusion in the compounds of the invention comprises isotopes of hydrogen, such as ²H and ³H, carbon, such as ¹¹C_{,} ¹³C and ¹⁴C, chlorine, such as ³⁶Cl, fluorine, such as ¹⁸F, iodine, such as ¹²³I and ¹²³I, nitrogen, such as ¹³N and ¹⁵N, oxygen, such as ¹⁶O, ¹⁷O and ¹⁸O, phosphorus, such as ³²P, and sulfur, such as ³³S.

Certain isotopically-labeled compounds of Formula (I), (I-a), (I-b), (I-c), (I-d), (I-e), (I-f), (I-g), (I-h) for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements or an improvement in therapeutic index, and hence may be preferred in some circumstances. It is understood that deuterium in this context is regarded as a substituent of a compound of Formula (I), (I-a), (I-b), (I-c), (I-d), (I-e), (I-f), (I-g), (I-h). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Substitution with positron emitting isotopes, such as ¹¹C, ¹⁸F, ¹⁵O and ¹³N, can be useful in Positron Emission Tomography (PET) studies for examining substrate receptor occupancy.

Isotopically-labeled compounds of Formula (I), (I-a), (I-b), (I-c), (I-d), (I-e), (I-f), (I-g), (I-h) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of Formula (I), (I-a), (I-b), (I-c), (I-d), (I-e), (I-f), (I-g), (I-h) that contain groups capable of acting as donors and/or acceptors for hydrogen bonds may be capable of forming co-crystals with suitable co-crystal formers. These co-crystals may be prepared from compounds of Formula (I), (I-a), (I-b), (I-c), (I-d), (I-e), (I-f), (I-g), (I-h) by known co-crystal forming procedures. Such procedures include grinding, heating, co-subliming, co-melting, or contacting in solution compounds of formula I with the co-crystal former under crystallization conditions and isolating co-crystals thereby formed. Suitable co-crystal formers include those described in WO 2004/078163. Hence the invention further provides co-crystals comprising a compound of Formula (I), (I-a), (I-b), (I-c), (I-d), (I-e), (I-f), (I-g), (I-h).

Compounds of the invention are either obtained in the free form, as a salt thereof, or as prodrug derivatives thereof.

The invention also envisages the use of pro-drugs of the compounds of the invention that convert in vivo to the compounds of the invention. Typically, a pro-drug is an inactive compound that is modified chemically through in vivo physiological action, such as hydrolysis, metabolism and the like, into a compound of the invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001).

Furthermore, the compounds of the invention, including their salts, can also be obtained in the form of their hydrates, or include other solvents used for their crystallization.

Preferred substituents, preferred ranges of numerical values or preferred ranges of the radicals present in compounds of Formula (I), (I-a), (I-b), (I-c), (I-d), (I-e), (I-f), (I-g), (I-h) and the corresponding intermediate compounds are defined below. The definition of the substituents applies to the end-products as well as to the corresponding intermediates. The definitions of the substituents may be combined at will, e.g. preferred substituents R₁ and particularly preferred substituents R₂.

In especially preferred embodiments, the invention relates to one or more than one of the compounds of Formula (I), (I-a), (I-b), (I-c), (I-d), (I-e), (I-f), (I-g), (I-h) mentioned in the Examples hereinafter, in free form or in salt form.

In one class of compounds of formula (I) of the invention, R₁ is hydrogen, C₁₋₆alkyl or C₁₋ₛhalogenalkyl.

In one class of compounds of formula (I) of the invention, R₁ is hydrogen.

In one class of compounds of the invention, A is a five- to six-membered monocyclic aromatic ring system which contains from 1 to 4 hetero atoms selected from nitrogen, oxygen and sulfur, and which is substituted once or more than once by R₂. In one embodiment of said class, each R₂ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, A is a six-membered aromatic ring system which contains 1 or 2 nitrogen atoms, and which is substituted once or more than once by R₂. In one embodiment of said class, each R₂ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, A is selected from pyrimidin-2-yl, pyridin-2-yl, phenyl, pyrazin-2-yl and pyrimidin-4-yl, all of which are substituted once or more than once by R₂. In one embodiment of said class, each R₂ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, A is pyrid-2-yl being substituted in the 4-position and/or in the 6-position by R₂. In one embodiment of said class, each R₂ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, A is pyrimidin-2-yl being substituted in the 4-position or in the 4-position and in the 6-position by R₂. In one embodiment of said class, each R₂ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, A is an eight- to ten-membered fused bicyclic aromatic ring system which may contain from 1 to 4 hetero atoms selected from nitrogen, oxygen and sulfur, and which may be substituted once or more than once by R₂. In one embodiment of said class, each R₂ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, A is selected from quinoxalin-2-yl, naphthalen-1-yl, 1H-benzo[d]imidazol-2-yl and benzo[d]oxazol-2-yl, all of which may be substituted once or more than once by R₂. In one embodiment of said class, each R₂ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, A is quinoxalin-2-yl.

In one class of compounds of the invention, B is an eight- to ten-membered fused bicyclic aromatic ring system which may contain from 1 to 4 hetero atoms selected from nitrogen, oxygen and sulfur, and which may be substituted once or more than once by R₄. In one embodiment of said class, each R₄ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is selected from 1H-indol-3-yl, 1H-benzo[d]imidazol-2-yl, naphthalen-1-yl and 1H-indol-4-yl, all of which may be substituted once or more than once by R₄. In one embodiment of said class, each R₄ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is a nine-membered fused bicyclic aromatic ring system which contains from 1 to 4 hetero atoms selected from nitrogen, oxygen and sulfur, and which may be substituted once or more than once by R₄. In one embodiment of said class, each R₄ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is indolyl which may be substituted once or more than once by R₄. In one embodiment of said class, each R₄ independently is halogen, cyano, C₁₋₆alkyl, C₁₋₆halogenalkyl, C₁₋₄alkoxy-C₁₋₆alkyl or C₁₋₆alkoxy. In one further embodiment of said class, R₄ is a five-membered heterocyclic aromatic ring system.

In one class of compounds of the invention, B is indol-3-yl which may be substituted once or more than once by R₄. In one embodiment of said class, each R₄ independently is halogen, cyano, C₁₋₆alkyl, C₁₋₆halogenalkyl, C₁₋₄alkoxy-C₁₋₆alkyl or C₁₋₆alkoxy. In one further embodiment of said class, R₄ is a five-membered heterocyclic aromatic ring system.

In one class of compounds of the invention, B is indol-4-yl which may be substituted once or more than once by R₄. In one embodiment of said class, each R₄ independently is halogen, cyano, C₁₋₆alkyl, C₁₋₆halogenalkyl, C₁₋₄alkoxy-C₁₋₆alkyl or C₁₋₆alkoxy. In one further embodiment of said class, R₄ is a five-membered heterocyclic aromatic ring system.

In one class of compounds of the invention, B is a five- to six-membered monocyclic aromatic ring system which may contain from 1 to 4 hetero atoms selected from nitrogen, oxygen and sulfur, and which is substituted once or more than once by R₄. In one embodiment of said class, each R₄ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is phenyl which is substituted once or more than once by R₄; each R₄ independently is C₁₋₆alkyl, C₁₋₆halogenalkyl, C₁₋₄alkoxy-C₁₋₆alkyl, C₁₋₆alkoxy, C₁₋₆halogenalkoxy, halogen, cyano.

In one class of compounds of the invention, B is a six-membered monocyclic aromatic ring system which may contain 1 to 2 nitrogen atoms, and which is substituted once by R₄ₐ, and which may be further substituted once or more than once by R_{4b};

R₄ₐ is a five- to six-membered monocyclic aromatic ring system, which may contain from 1 to 4 hetero atoms selected from nitrogen, oxygen and sulfur, and which may in turn be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; and

each R_{4b} independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is a six-membered monocyclic aromatic ring system which may contain 1 to 2 nitrogen atoms, and which is substituted once by R₄ₐ, and which may be further substituted once or more than once by R_{4b};

R₄ₐ is a five-membered monocyclic aromatic ring system, which contains from 1 to 4 hetero atoms selected from nitrogen, oxygen and sulfur, and which may in turn be substituted once or more than once by C₁₋₆alkyl, C₁₋₆halogenalkyl, C₁₋₆alkoxy, C₁₋₄alkoxy-C₁₋₆alkyl, C₁₋₆halogenalkoxy, halogen or cyano; and

each R_{4b} independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is phenyl which is substituted once by R₄ₐ, and which may be further substituted once or more than once by R_{4b};

R₄ₐ is a five-membered monocyclic aromatic ring system, which contains from 1 to 4 hetero atoms selected from nitrogen, oxygen and sulfur, and which may in turn be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; and

each R_{4b} independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is a five-membered monocyclic aromatic ring system which contains from 1 to 4 hetero atoms selected from nitrogen, oxygen and sulfur, and which is substituted once or more than once by R₄. In one embodiment of said class, each R₄ independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is a five-membered monocyclic aromatic ring system which contains from 1 to 4 hetero atoms selected from nitrogen, oxygen and sulfur, and which is substituted once by R₄ₐ, and which may be further substituted once or more than once by R_{4b};

R₄ₐ is a six-membered monocyclic aromatic ring system, which may contain from 1 to 2 nitrogen atoms, and which may in turn be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; and

each R_{4b} independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is a five-membered monocyclic aromatic ring system which contains from 1 to 4 hetero atoms selected from nitrogen, oxygen and sulfur, and which is substituted once by R₄ₐ, and which may be further substituted once or more than once by R_{4b};

R₄ₐ is phenyl, which may be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; and

each R_{4b} independently is halogen, cyano, C₁₋₆alkyl, C₁₋₆halogenalkyl, C₁₋₄alkoxy-C₁₋₆alkyl or C₁₋₆alkoxy.

In one class of compounds of the invention, B is a five-membered monocyclic aromatic ring system which contains from 1 to 4 hetero atoms selected from nitrogen, oxygen and sulfur, and which is substituted once by phenyl, which may in turn be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano.

In one class of compounds of formula (I) of the invention, D is D1.

In one class of compounds of formula (I) of the invention, D is D1; X₁ is —C(R₁₄)₂— and m is 0.

In one embodiment of said class, R₁₄ is hydrogen and q1 and q2 are both 0.

In one class of compounds of formula (I) of the invention, D is D1; X₁ is —N(R₁₅)— and m is 0.

In one embodiment of said class, R₁₅ is hydrogen and q1 and q2 are both 0.

In one class of compounds of formula (I) of the invention, D is D1; X₁ is —O— or —N(R₁₅)— and m is 1. In one embodiment of said class, R₁₅ is hydrogen and q1 and q2 are both 0.

In one class of compounds of formula (I) of the invention, D is D1; X₁ is —O— and m is 1. In one embodiment of said class, q1 and q2 are both 0.

In one class of compounds of formula (I) of the invention, D is D1; X₁ is —N(R₁₅)— and m is 1.

In one embodiment of said class, R₁₅ is hydrogen and q1 and q2 are both 0.

In one class of compounds of formula (I) of the invention, D is D2.

In one class of compounds of formula (I) of the invention, D is D2; and n, q3 and q4 are all 0.

In one class of compounds of formula (I) of the invention, D is D2; n is 1 and q3 and q4 are both 0.

In one class of compounds of formula (I) the invention, D is D2a

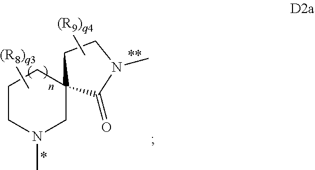

wherein the bond marked with one asterisk is attached to A and the bond marked with two asterisks is attached to C(R₁)₂—B;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6; and

q4 is 0, 1, 2, 3 or 4.

In one class of compounds of formula (I) of the invention, D is D2a; and n, q3 and q4 are all 0.

In one class of compounds of formula (I) of the invention, D is D2a; n is 1 and q3 and q4 are both 0.

In one class of compounds of formula (I) of the invention, D is D2b

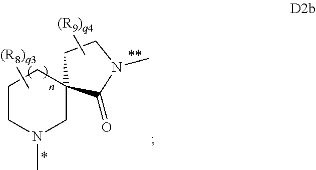

wherein the bond marked with one asterisk is attached to A and the bond marked with two asterisks is attached to C(R₁)₂—B;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6; and

q4 is 0, 1, 2, 3 or 4.

In one class of compounds of formula (I) of the invention, D is D2b; and n, q3 and q4 are all 0.

In one class of compounds of formula (I) of the invention, D is D2b; n is 1 and q3 and q4 are both 0.

In one class of compounds of formula (I) of the invention, D is D3.

In one class of compounds of formula (I) of the invention, D is D3; X₂ is —C(R₁₆)₂—; p is 0; R₁₆ is hydrogen; and q5 and q6 are both 0.

In one class of compounds of formula (I) of the invention, D is D3; X₂ is —O— and p is 0 or 1.

In one class of compounds of formula (I) of the invention, D is D3; X₂ is —O— and p is 0. In one embodiment of said class, q5 and q6 are both 0.

In one class of compounds of formula (I) of the invention, D is D3; X₂ is —O— and p is 1. In one embodiment of said class, q5 and q6 are both 0.

In one class of compounds of formula (I) of the invention, D is D4.

In one class of compounds of formula (I) of the invention, D is D4; q7 and q8 are both 0.

In one class of compounds of formula (I) of the invention, D is D4a

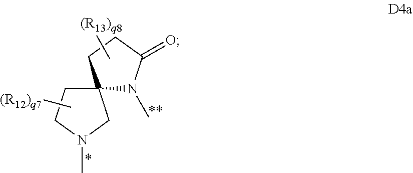

wherein the bond marked with one asterisk is attached to A and the bond marked with two asterisks is attached to C(R₁)₂—B;

each R₁₂ or R₁₃ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₂ at the same carbon atom form together with said carbon atom C₃₋₇cycloalkyl, or two R₁₃ at the same carbon atom form together with said carbon atom C₃₋₇cycloalkyl;

q7 is 0, 1, 2, 3 or 4; and

q8 is 0, 1, 2, 3 or 4.

In one class of compounds of formula (I) of the invention, D is D4a; and q7 and q8 are all 0.

In one class of compounds of formula (I) of the invention, D is D4b

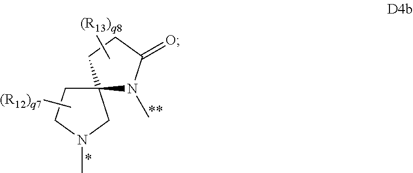

wherein the bond marked with one asterisk is attached to A and the bond marked with two asterisks is attached to C(R₁)₂—B;

each R₁₂ or R₁₃ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₂ at the same carbon atom form together with said carbon atom C₃₋₇cycloalkyl, or two R₁₃ at the same carbon atom form together with said carbon atom C₃₋₇cycloalkyl;

q7 is 0, 1, 2, 3 or 4; and

q8 is 0, 1, 2, 3 or 4.

In one class of compounds of formula (I) of the invention, D is D4b; and q7 and q8 are all 0.

In one class of compounds of formula (I) of the invention, D is D5.

In one class of compounds of formula (I) of the invention, D is D5; and q9 and q10 are all 0.

In one class of compounds of formula (I) of the invention, D is D5a

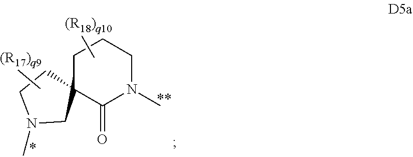

wherein the bond marked with one asterisk is attached to A and the bond marked with two asterisks is attached to C(R₁)₂—B;

each R₁₇ or R₁₈ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₇ at the same carbon atom form together with said carbon atom C₃₋₇cycloalkyl, or two R₁₈ at the same carbon atom form together with said carbon atom C₃₋₇cycloalkyl;

q7 is 0, 1, 2, 3 or 4; and

q8 is 0, 1, 2, 3 or 4.

In one class of compounds of formula (I) of the invention, D is D5a; and q9 and q10 are all 0.

In one class of compounds of formula (I) of the invention, D is D5b

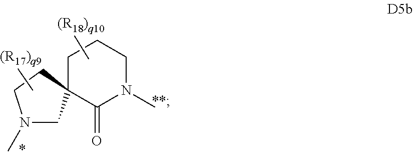

wherein the bond marked with one asterisk is attached to A and the bond marked with two asterisks is attached to C(R₁)₂—B;

each R₁₇ or R₁₈ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₇ at the same carbon atom form together with said carbon atom C₃₋₇cycloalkyl, or two R₁₈ at the same carbon atom form together with said carbon atom C₃₋₇cycloalkyl;

q7 is 0, 1, 2, 3 or 4; and

q8 is 0, 1, 2, 3 or 4.

In one class of compounds of formula (I) of the invention, D is D5b; and q9 and q10 are all 0.

In one embodiment, the invention provides a compound selected from
4-((1H-Indo)-3-yl)methyl)-9-(4,6-dimethylpyrimidin-2-yl)-1,4,9-triazaspiro[5.5]undecan-5-one;
4-((1H-Indo)-3-yl)methyl)-9-(4,6-dimethylpyrimidin-2-yl)-1-methyl-1,4,9-triazaspiro[5.5]undecan-5-one;
9-(4,6-Dimethyl-pyrimidin-2-yl)-4-(I-methyl-4-phenyl-1H-pyrazol-3-ylmethyl)-1,4,9-triaza-spiro[5.5]undecan-5-on;
9-(4,6-Dimethylpyrimidin-2-yl)-4-((5-fluoro-1H-indol-3-yl)methyl)-1,4,9-triazaspiro[5.5]undecan-5-one;
9-(4,6-Dimethylpyrimidin-2-yl)-4-((5-(3-methoxyphenyl)-2-methyl-2H-1,2,3-triazol-4-yl)methyl)-1,4,9-triazaspiro[5.5]undecan-5-one;
4-((1H-Indo)-3-yl)methyl)-9-(4-methylpyrimidin-2-yl)-1,4,9-triazaspiro[5.5]undecan-5-one;
4-((1H-Indo)-3-yl)methyl)-9-(4-methoxypyrimidin-2-yl)-1,4,9-triazaspiro[5.5]undecan-5-one;
4-((1H-Indo)-3-yl)methyl)-9-(4-methoxy-6-methylpyrimidin-2-yl)-1,4,9-triazaspiro[5.5]undecan-5-one;
9-(4,6-Dimethylpyrimidin-2-yl)-4-((5-(3-methoxyphenyl)oxazol-4-yl)methyl)-1,4,9-triazaspiro[5.5]undecan-5-one;
4-((5-(3-Methoxyphenyl)oxazol-4-yl)methyl)-9-(4-methylpyrimidin-2-yl)-1,4,9-triazaspiro[5.5]undecan-5-one;
2-(4-((5-(3-Methoxyphenyl)oxazol-4-yl)methyl)-5-oxo-1,4,9-triazaspiro[5.5]undecan-9-yl)-6-methylpyrimidine-4-carbonitrile;
4-((5-(3-Methoxyphenyl)oxazol-4-yl)methyl)-9-(4-methoxypyrimidin-2-yl)-1,4,9-triazaspiro[5.5]undecan-5-one;
9-(4-Methoxy-6-methylpyrimidin-2-yl)-4-((5-(3-methoxyphenyl)oxazol-4-yl)methyl)-1,4,9-triazaspiro[5.5]undecan-5-one;
9-(4,6-Dimethylpyrimidin-2-yl)-4-((5-(3-methoxyphenyl)-2-methyloxazol-4-yl)methyl)-1,4,9-triazaspiro[5.5]undecan-5-one;
4-((5-(3-Methoxyphenyl)-2-methyloxazol-4-yl)methyl)-9-(4-methylpyrimidin-2-yl)-1,4,9-triazaspiro[5.5]undecan-5-one;
2-(4-((5-(3-Methoxyphenyl)-2-methyloxazol-4-yl)methyl)-5-oxo-1,4,9-triazaspiro[5.5]undecan-9-yl)-6-methylpyrimidine-4-carbonitrile;
4-((5-(3-Methoxyphenyl)-2-methyloxazol-4-yl)methyl)-9-(4-methoxypyrimidin-2-yl)-1,4,9-triazaspiro[5.5]undecan-5-one;
9-(4-Methoxy-6-methylpyrimidin-2-yl)-4-((5-(3-methoxyphenyl)-2-methyloxazol-4-yl)methyl)-1,4,9-triazaspiro[5.5]undecan-5-one;
9-(4,6-Dimethylpyrimidin-2-yl)-4-((2-methyl-5-phenyloxazol-4-yl)methyl)-1,4,9-triazaspiro[5.5]undecan-5-one;
9-(4-Methylpyrimidin-2-yl)-4-((2-methyl-5-phenyloxazol-4-yl)methyl)-1,4,9-triazaspiro[5.5]undecan-5-one;
6-Methyl-2-(4-((2-methyl-5-phenyloxazol-4-yl)methyl)-5-oxo-1,4,9-triazaspiro[5.5]undecan-9-yl)pyrimidine-4-carbonitrile;
9-(4-Methoxypyrimidin-2-yl)-4-((2-methyl-5-phenyloxazol-4-yl)methyl)-1,4,9-triazaspiro[5.5]undecan-5-one;
9-(4-Methoxy-6-methylpyrimidin-2-yl)-4-((2-methyl-5-phenyloxazol-4-yl)methyl)-1,4,9-triazaspiro[5.5]undecan-5-one;
9-(4,6-Dimethylpyrimidin-2-yl)-4-((5-(3-methoxyphenyl)-2-methylthiazol-4-yl)methyl)-1,4,9-triazaspiro[5.5]undecan-5-one;
2-(4-((5-(3-Methoxyphenyl)-2-methylthiazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-methoxyphenyl)-2-methylthiazol-4-yl)methyl)-1,4,9-triazaspiro[5.5]undecan-5-one;
9-(4,6-Dimethylpyrimidin-2-yl)-4-((5-(3-(methoxymethyl)phenyl)oxazol-4-yl)methyl)-1,4,9-triazaspiro[5.5]undecan-5-one;
2-(4-((5-(3-(Methoxymethyl)phenyl)oxazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-(methoxymethyl)phenyl)oxazol-4-yl)methyl)-1,4,9-triazaspiro[5.5]undecan-5-one;
9-(4,6-Dimethylpyrimidin-2-yl)-4-((5-(3-(methoxymethyl)phenyl)-2-methyloxazol-4-yl)methyl)-1,4,9-triazaspiro[5.5]undecan-5-one;
2-(4-((5-(3-(Methoxymethyl)phenyl)-2-methyloxazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-(methoxymethyl)phenyl)-2-methyloxazol-4-yl)methyl)-1,4,9-triazaspiro[5.5]undecan-5-one;
9-(4,6-Dimethylpyrimidin-2-yl)-4-((4-(3-methoxyphenyl)-1-methyl-1H-pyrazol-3-yl)methyl)-1,4,9-triazaspiro[5.5]undecan-5-one;
9-(4,6-Dimethylpyrimidin-2-yl)-4-((5-methoxy-1H-indol-3-yl)methyl)-1,4,9-triazaspiro[5.5]undecan-5-one;
4-((4-(3,4-Dimethoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(4-methoxyphenyl)-1-methyl-1H-pyrazol-3-yl)methyl)-1,4,9-triazaspiro[5.5]undecan-5-one;
4-((4-(3,5-Dimethoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((1-methyl-4-(m-tolyl)-1H-pyrazol-3-yl)methyl)-1,4,9-triazaspiro[5.5]undecan-5-one;
9-(4,6-Dimethylpyrimidin-2-yl)-4-((5-(3-methoxyphenyl)-2H-1,2,3-triazol-4-yl)methyl)-1,4,9-triazaspiro[5.5]undecan-5-one;
2-(4-((1H-Indo)-3-yl)methyl)-5-oxo-1,4,9-triazaspiro[5.5]undecan-9-yl)-6-methylpyrimidine-4-carbonitrile;
9-(4,6-Dimethylpyrimidin-2-yl)-4-((1-methyl-4-(3-(trifluoromethyl)phenyl)-1H-pyrazol-3-yl)methyl)-1,4,9-triazaspiro[5.5]undecan-5-one;
4-((4-(3-Chlorophenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(3-fluorophenyl)-1-methyl-1H-pyrazol-3-yl)methyl)-1,4,9-triazaspiro[5.5]undecan-5-one;
3-(3-((9-(4,6-Dimethylpyrimidin-2-yl)-5-oxo-1,4,9-triazaspiro[5.5]undecan-4-yl)methyl)-1-methyl-1H-pyrazol-4-yl)benzonitrile;
4-(3-((9-(4,6-Dimethylpyrimidin-2-yl)-5-oxo-1,4,9-triazaspiro[5.5]undecan-4-yl)methyl)-1-methyl-1H-pyrazol-4-yl)benzonitrile;
9-(4,6-Dimethylpyrimidin-2-yl)-4-((1-methyl-4-(3-(trifluoromethoxy)phenyl)-1H-pyrazol-3-yl)methyl)-1,4,9-triazaspiro[5.5]undecan-5-one;
9-(4,6-Dimethylpyrimidin-2-yl)-4-((4-(3-isopropoxyphenyl)-1-methyl-1H-pyrazol-3-yl)methyl)-1,4,9-triazaspiro[5.5]undecan-5-one;
4-((4-(3-Acetylphenyl)-1-methyl-1H-pyrazol-3-yl)methyl)-9-(4,6-dimethylpyrimidin-2-yl)-1,4,9-triazaspiro[5.5]undecan-5-one;
3-(3-((9-(4,6-Dimethylpyrimidin-2-yl)-5-oxo-1,4,9-triazaspiro[5.5]undecan-4-yl)methyl)-1-methyl-1H-pyrazol-4-yl)-N-methylbenzamide;
4-((1H-Indazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(4-(methoxymethyl)phenyl)-1-methyl-1H-pyrazol-3-yl)methyl)-1,4,9-triazaspiro[5.5]undecan-5-one;
4-((4-(3-(Cyclopropylmethoxy)phenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(3-isobutoxyphenyl)-1-methyl-1H-pyrazol-3-yl)methyl)-1,4,9-triazaspiro[5.5]undecan-5-one;
9-(4,6-Dimethylpyrimidin-2-yl)-4-((4-(6-methoxypyridin-2-yl)-1-methyl-1H-pyrazol-3-yl)methyl)-1,4,9-triazaspiro[5.5]undecan-5-one;
9-(4,6-Dimethylpyrimidin-2-yl)-4-((4-(5-methoxypyridin-3-yl)-1-methyl-1H-pyrazol-3-yl)methyl)-1,4,9-triazaspiro[5.5]undecan-5-one;
9-(4,6-Dimethylpyrimidin-2-yl)-4-((5-methoxy-1H-indazol-3-yl)methyl)-1,4,9-triazaspiro[5.5]undecan-5-one;
4-((4-(3-(1H-Pyrazol-1-yl)phenyl)-1-methyl-1H-pyrazol-3-yl)methyl)-9-(4,6-dimethylpyrimidin-2-yl)-1,4,9-triazaspiro[5.5]undecan-5-one;
4-((4-(3-(Dimethylamino)phenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((1-methyl-4-(3-(5-methyl-1,3,4-oxadiazol-2-yl)phenyl)-1H-pyrazol-3-yl)methyl)-1,4,9-triazaspiro[5.5]undecan-5-one;
4-((4-(3-(3,5-Dimethyl-1H-pyrazol-1-yl)phenyl)-1-methyl-1H-pyrazol-3-yl)methyl)-9-(4,6-dimethylpyrimidin-2-yl)-1,4,9-triazaspiro[5.5]undecan-5-one;
4-((4-(2,3-Dihydrobenzo[b][1,4]dioxin-6-yl)-1-methyl-1H-pyrazol-3-yl)methyl)-9-(4,6-dimethylpyrimidin-2-yl)-1,4,9-triazaspiro[5.5]undecan-5-one;
4-((4-(3-Cyclopropoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-dimethylpyrimidin-2-yl)-4-(2-(3-(methoxymethyl)-1,2,4-oxadiazol-5-yl)benzyl)-1,4,9-triazaspiro[5.5]undecan-5-one;
9-(4,6-dimethylpyrimidin-2-yl)-4-(2-(5-methyloxazol-2-yl)benzyl)-1,4,9-triazaspiro[5.5]undecan-5-one;
4-((1H-indazol-3-yl)methyl)-9-(4-methoxy-6-methylpyrimidin-2-yl)-1,4,9-triazaspiro[5.5]undecan-5-one;
4-((1H-indol-3-yl)methyl)-9-(2-methoxy-6-methylpyrimidin-4-yl)-1,4,9-triazaspiro[5.5]undecan-5-one;
9-(4,6-dimethylpyrimidin-2-yl)-4-((4-(3-ethylphenyl)-1-methyl-1H-pyrazol-3-yl)methyl)-1,4,9-triazaspiro[5.5]undecan-5-one;
9-(4,6-dimethylpyrimidin-2-yl)-4-((4-(3-isopropylphenyl)-1-methyl-1H-pyrazol-3-yl)methyl)-1,4,9-triazaspiro[5.5]undecan-5-one;
9-(4,6-dimethylpyrimidin-2-yl)-4-(2-(3-methyl-1,2,4-oxadiazol-5-yl)benzyl)-1,4,9-triazaspiro[5.5]undecan-5-one;
9-(4,6-dimethylpyrimidin-2-yl)-4-((1-methyl-4-(3-propionylphenyl)-1H-pyrazol-3-yl)methyl)-1,4,9-triazaspiro[5.5]undecan-5-one;
4-((4-(3-(difluoromethyl)phenyl)-1-methyl-1H-pyrazol-3-yl)methyl)-9-(4,6-dimethylpyrimidin-2-yl)-1,4,9-triazaspiro[5.5]undecan-5-one;
9-(4,6-dimethylpyrimidin-2-yl)-4-((4-(3-(2-isopropoxyethoxy)phenyl)-1-methyl-1H-pyrazol-3-yl)methyl)-1,4,9-triazaspiro[5.5]undecan-5-one;
9-(4,6-dimethylpyrimidin-2-yl)-4-((1-methyl-4-(3-morpholinophenyl)-1H-pyrazol-3-yl)methyl)-1,4,9-triazaspiro[5.5]undecan-5-one;
9-(4,6-dimethylpyrimidin-2-yl)-4-((5-(2-methoxypyridin-4-yl)-2-methyl-2H-1,2,3-triazol-4-yl)methyl)-1,4,9-triazaspiro[5.5]undecan-5-one;
9-(4,6-dimethylpyrimidin-2-yl)-4-((5-(2-ethylpyridin-4-yl)-2-methyl-2H-1,2,3-triazol-4-yl)methyl)-1,4,9-triazaspiro[5.5]undecan-5-one;
9-(4,6-dimethylpyrimidin-2-yl)-4-((5-(3-(methoxymethyl)phenyl)-2-methyl-2H-1,2,3-triazol-4-yl)methyl)-1,4,9-triazaspiro[5.5]undecan-5-one;
9-(4,6-dimethylpyrimidin-2-yl)-4-((2-methyl-5-(3-morpholinophenyl)-2H-1,2,3-triazol-4-yl)methyl)-1,4,9-triazaspiro[5.5]undecan-5-one;
9-(4,6-dimethylpyrimidin-2-yl)-4-((5-(3-(2-methoxyethoxy)phenyl)-2-methyl-2H-1,2,3-triazol-4-yl)methyl)-1,4,9-triazaspiro[5.5]undecan-5-one;
9-(4,6-dimethylpyrimidin-2-yl)-4-((5-(3-isopropoxyphenyl)-2-methyl-2H-1,2,3-triazol-4-yl)methyl)-1,4,9-triazaspiro[5.5]undecan-5-one;
4-((5-(2,3-dihydrobenzo[b][1,4]dioxin-6-yl)-2-methyl-2H-1,2,3-triazol-4-yl)methyl)-9-(4,6-dimethylpyrimidin-2-yl)-1,4,9-triazaspiro[5.5]undecan-5-one;
9-(4,6-dimethylpyrimidin-2-yl)-4-((4-phenylisoxazol-3-yl)methyl)-1,4,9-triazaspiro[5.5]undecan-5-one;
9-(4,6-dimethylpyrimidin-2-yl)-4-((3-phenylisoxazol-4-yl)methyl)-1,4,9-triazaspiro[5.5]undecan-5-one;
9-(4,6-dimethylpyrimidin-2-yl)-4-((2-methyl-5-phenyl-2H-1,2,3-triazol-4-yl)methyl)-1,4,9-triazaspiro[5.5]undecan-5-one;
4-((1H-Indo)-3-yl)methyl)-9-(4,6-dimethylpyrimidin-2-yl)-1-oxa-4,9-diazaspiro[5.5]undecan-5-one;
9-(4,6-Dimethyl-pyrimidin-2-yl)-4-(1H-indazol-3-ylmethyl)-1-oxa-4,9-diaza-spiro[5.5]undecan-5-one;
9-(4,6-Dimethyl-pyrimidin-2-yl)-4-(2-furan-2-yl-benzyl)-1-oxa-4,9-diaza-spiro[5.5]undecan-5-one;
4-((1H-indazol-3-yl)methyl)-9-(4-methoxy-6-methylpyrimidin-2-yl)-1-oxa-4,9-diazaspiro[5.5]undecan-5-one;
4-((1H-Indol-3-yl)methyl)-9-(4-methoxypyrimidin-2-yl)-1-oxa-4,9-diazaspiro[5.5]undecan-5-one;
9-(4,6-Dimethylpyrimidin-2-yl)-4-((5-fluoro-1H-indol-3-yl)methyl)-1-oxa-4,9-diazaspiro[5.5]undecan-5-one;
9-(4,6-Dimethylpyrimidin-2-yl)-4-((5-methoxy-1H-indol-3-yl)methyl)-1-oxa-4,9-diazaspiro[5.5]undecan-5-one;
4-((1H-Indo)-3-yl)methyl)-9-(4-methylpyrimidin-2-yl)-1-oxa-4,9-diazaspiro[5.5]undecan-5-one;
9-(4,6-Dimethylpyrimidin-2-yl)-4-(2-(oxazol-2-yl)benzyl)-1-oxa-4,9-diazaspiro[5.5]undecan-5-one;
9-(4,6-Dimethylpyrimidin-2-yl)-4-(2-(3-methyl-1,2,4-oxadiazol-5-yl)benzyl)-1-oxa-4,9-diazaspiro[5.5]undecan-5-one;
9-(4,6-Dimethylpyrimidin-2-yl)-4-((1-methyl-4-phenyl-1H-pyrazol-3-yl)methyl)-1-oxa-4,9-diazaspiro[5.5]undecan-5-one;
9-(4,6-Dimethylpyrimidin-2-yl)-4-((2-methyl-5-phenyl-2H-1,2,3-triazol-4-yl)methyl)-1-oxa-4,9-diazaspiro[5.5]undecan-5-one;
9-(4,6-Dimethylpyrimidin-2-yl)-4-((4-phenyl-1H-pyrazol-3-yl)methyl)-1-oxa-4,9-diazaspiro[5.5]undecan-5-one;
9-(4,6-Dimethylpyrimidin-2-yl)-4-((5-phenyl-2H-1,2,3-triazol-4-yl)methyl)-1-oxa-4,9-diazaspiro[5.5]undecan-5-one;
9-(4,6-Dimethylpyrimidin-2-yl)-4-((1-methyl-4-phenyl-1H-1,2,3-triazol-5-yl)methyl)-1-oxa-4,9-diazaspiro[5.5]undecan-5-one;
9-(4,6-Dimethylpyrimidin-2-yl)-4-((1-methyl-5-phenyl-1H-1,2,3-triazol-4-yl)methyl)-1-oxa-4,9-diazaspiro[5.5]undecan-5-one;
9-(4,6-Dimethylpyrimidin-2-yl)-4-((5-(3-methoxyphenyl)-2H-1,2,3-triazol-4-yl)methyl)-1-oxa-4,9-diazaspiro[5.5]undecan-5-one;
4-((1H-Indol-3-yl)methyl)-9-(4-methoxy-6-methylpyrimidin-2-yl)-1-oxa-4,9-diazaspiro[5.5]undecan-5-one;
4-((1H-Pyrrolo[2,3-b]pyridin-3-yl)methyl)-9-(4-methoxy-6-methylpyrimidin-2-yl)-1-oxa-4,9-diazaspiro[5.5]undecan-5-one;
9-(4-Methoxy-6-methylpyrimidin-2-yl)-4-((5-(3-methoxyphenyl)oxazol-4-yl)methyl)-1-oxa-4,9-diazaspiro[5.5]undecan-5-one;
4-((1H-Indazol-3-yl)methyl)-9-(4-methylpyrimidin-2-yl)-1-oxa-4,9-diazaspiro[5.5]undecan-5-one;
4-((1H-indol-3-yl)methyl)-9-(4-ethylpyrimidin-2-yl)-1-oxa-4,9-diazaspiro[5.5]undecan-5-one;
4-((1H-indol-3-yl)methyl)-9-(4-ethyl-6-methylpyrimidin-2-yl)-1-oxa-4,9-diazaspiro[5.5]undecan-5-one;
4-((1H-indol-3-yl)methyl)-9-(4,5-dimethylpyrimidin-2-yl)-1-oxa-4,9-diazaspiro[5.5]undecan-5-one;
9-(4,6-dimethylpyrimidin-2-yl)-4-((4-phenylisoxazol-3-yl)methyl)-1-oxa-4,9-diazaspiro[5.5]undecan-5-one;
9-(4,6-dimethylpyrimidin-2-yl)-4-((3-phenylisoxazol-4-yl)methyl)-1-oxa-4,9-diazaspiro[5.5]undecan-5-one;
2-((1H-Indo)-3-yl)methyl)-8-(4,6-dimethylpyrimidin-2-yl)-2,8-diazaspiro[4.5]decan-1-one;
2-((1H-Indo)-3-yl)methyl)-8-(quinoxalin-2-yl)-2,8-diazaspiro[4.5]decan-1-one;
2-(2-((1H-Indol-3-yl)methyl)-1-oxo-2,8-diazaspiro[4.5]decan-8-yl)isonicotinonitrile;
2-((1H-Indo)-3-yl)methyl)-8-(6-methoxypyridin-2-yl)-2,8-diazaspiro[4.5]decan-1-one;
2-((1H-Indo)-3-yl)methyl)-8-(4-methylpyrimidin-2-yl)-2,8-diazaspiro[4.5]decan-1-one;
2-(Biphenyl-2-ylmethyl)-8-(quinoxalin-2-yl)-2,8-diazaspiro[4.5]decan-1-one;
8-(1H-Benzo[d]imidazol-2-yl)-2-(naphthalen-1-ylmethyl)-2,8-diazaspiro[4.5]decan-1-one;
2-(Biphenyl-2-ylmethyl)-8-(1-methyl-1H-benzo[d]imidazol-2-yl)-2,8-diazaspiro[4.5]decan-1-one;
8-(1H-Benzo[d]imidazol-2-yl)-2-(biphenyl-2-ylmethyl)-2,8-diazaspiro[4.5]decan-1-one;
8-(Benzo[d]thiazol-2-yl)-2-(biphenyl-2-ylmethyl)-2,8-diazaspiro[4.5]decan-1-one;
2-(Naphthalen-1-ylmethyl)-8-(quinoxalin-2-yl)-2,8-diazaspiro[4.5]decan-1-one;
8-(1H-Benzoimidazol-2-yl)-2-(1H-indol-3-ylmethyl)-2,8-diaza-spiro[4.5]decan-1-one;
8-Benzooxazol-2-yl-2-(1H-indol-3-ylmethyl)-2,8-diaza-spiro[4.5]decan-1-one;
2-(1H-Indo)-3-ylmethyl)-8-(4-methoxy-pyrimidin-2-yl)-2,8-diaza-spiro[4.5]decan-1-one;
8-(4-Methoxy-pyrimidin-2-yl)-2-(1H-pyrrolo[2,3-b]pyridin-3-ylmethyl)-2,8-diaza-spiro[4.5]decan-1-one;
2-(1H-Indazol-3-ylmethyl)-8-(4-methoxy-pyrimidin-2-yl)-2,8-diaza-spiro[4.5]decan-1-one;
8-(4-Methoxy-pyrimidin-2-yl)-2-[2-(3-methyl-[1,2,4]oxadiazol-5-yl)-benzyl]-2,8-diaza-spiro[4.5]decan-1-one;
2-(1H-Indo)-3-ylmethyl)-8-(4-methoxy-6-methyl-pyrimidin-2-yl)-2,8-diaza-spiro[4.5]decan-1-one;
8-(4,6-Dimethyl-pyrimidin-2-yl)-2-[2-(3-methyl-[1,2,4]oxadiazol-5-yl)-benzyl]-2,8-diaza-spiro[4.5]decan-1-one;
8-(4-Methoxy-6-methyl-pyrimidin-2-yl)-2-[2-(3-methyl-[1,2,4]oxadiazol-5-yl)-benzyl]-2,8-diaza-spiro[4.5]decan-1-one;
2-((1H-indazol-3-yl)methyl)-8-(4-methoxy-6-methylpyrimidin-2-yl)-2,8-diazaspiro[4.5]decan-1-one;
2-((1H-pyrrolo[2,3-b]pyridin-3-yl)methyl)-8-(4-methoxy-6-methylpyrimidin-2-yl)-2,8-diazaspiro[4.5]decan-1-one;
2-((1H-indol-5-yl)methyl)-8-(4,6-dimethylpyrimidin-2-yl)-2,8-diazaspiro[4.5]decan-1-one;
8-(4,6-dimethylpyrimidin-2-yl)-2-((2-methyl-5-phenyl-2H-1,2,3-triazol-4-yl)methyl)-2,8-diazaspiro[4.5]decan-1-one;
3-((1H-indol-3-yl)methyl)-8-(4,6-dimethylpyrimidin-2-yl)-1,3,8-triazaspiro[4.5]decan-4-one;
2-((1H-Indo)-3-yl)methyl)-7-(4-methoxypyrimidin-2-yl)-2,7-diazaspiro[4.4]nonan-1-one;
2-((1H-indol-3-yl)methyl)-7-(4-methoxypyrimidin-2-yl)-2,7-diazaspiro[4.4]nonan-1-one;
2-((1H-Indol-3-yl)methyl)-7-(4-methoxy-6-methylpyrimidin-2-yl)-2,7-diazaspiro[4.4]nonan-1-one;
2-((1H-Indo)-3-yl)methyl)-7-(quinoxalin-2-yl)-2,7-diazaspiro[4.4]nonan-1-one;
2-((1H-Indo)-3-yl)methyl)-7-(4-methylpyrimidin-2-yl)-2,7-diazaspiro[4.4]nonan-1-one;
2-((1H-Indo)-3-yl)methyl)-7-(4,6-dimethylpyrimidin-2-yl)-2,7-diazaspiro[4.4]nonan-1-one;
2-((1H-Indol-3-yl)methyl)-7-(benzo[d]oxazol-2-yl)-2,7-diazaspiro[4.4]nonan-1-one;
2-((1H-Indo)-4-yl)methyl)-7-(4,6-dimethylpyrimidin-2-yl)-2,7-diazaspiro[4.4]nonan-1-one;
2-((1H-Indol-4-yl)methyl)-7-(4-methoxy-6-methylpyrimidin-2-yl)-2,7-diazaspiro[4.4]nonan-1-one;
2-((1H-Pyrrolo[2,3-b]pyridin-3-yl)methyl)-7-(4-methoxypyrimidin-2-yl)-2,7-diazaspiro[4.4]nonan-1-one;
2-((1H-Indazol-3-yl)methyl)-7-(4-methoxypyrimidin-2-yl)-2,7-diazaspiro[4.4]nonan-1-one;
7-(4-Methoxypyrimidin-2-yl)-2-(2-(3-methyl-1,2,4-oxadiazol-5-yl)benzyl)-2,7-diazaspiro[4.4]nonan-1-one;
2-((5-(3-Methoxyphenyl)-2H-1,2,3-triazol-4-yl)methyl)-7-(4-methoxypyrimidin-2-yl)-2,7-diazaspiro[4.4]nonan-1-one;
2-((1H-Indo)-3-yl)methyl)-7-(5-chlorobenzo[d]oxazol-2-yl)-2,7-diazaspiro[4.4]nonan-1-one;
2-((1H-Indo)-3-yl)methyl)-7-(6-fluoroquinazolin-2-yl)-2,7-diazaspiro[4.4]nonan-1-one;
2-((1H-indol-3-yl)methyl)-7-(6-(trifluoromethyl)pyrimidin-4-yl)-2,7-diazaspiro[4.4]nonan-1-one;
7-(4-(1H-imidazol-1-yl)pyrimidin-2-yl)-2-((1H-indol-3-yl)methyl)-2,7-diazaspiro[4.4]nonan-1-one;
7-(2-(1H-imidazol-1-yl)pyrimidin-4-yl)-2-((1H-indol-3-yl)methyl)-2,7-diazaspiro[4.4]nonan-1-one;
7-(6-(1H-imidazol-1-yl)pyridazin-3-yl)-2-((1H-indol-3-yl)methyl)-2,7-diazaspiro[4.4]nonan-1-one;
2-((1H-indol-3-yl)methyl)-7-(2-cyclopropyl-6-methoxypyrimidin-4-yl)-2,7-diazaspiro[4.4]nonan-1-one;
2-((1H-indol-3-yl)methyl)-7-(4-(1-methyl-1H-imidazol-2-yl)pyrimidin-2-yl)-2,7-diazaspiro[4.4]nonan-1-one;
2-((1H-indol-3-yl)methyl)-7-(5-methylthiazol-2-yl)-2,7-diazaspiro[4.4]nonan-1-one;
2-((1H-indol-3-yl)methyl)-7-(7,8-dihydro-5H-pyrano[4,3-c]pyridazin-3-yl)-2,7-diazaspiro[4.4]nonan-1-one;
2-((1H-indol-3-yl)methyl)-7-(4-methyl-6-morpholinopyrimidin-2-yl)-2,7-diazaspiro[4.4]nonan-1-one;
2-((1H-indol-3-yl)methyl)-7-(2-methyl-6-morpholinopyrimidin-4-yl)-2,7-diazaspiro[4.4]nonan-1-one;
2-((1H-indol-3-yl)methyl)-7-(6-methylpyridin-2-yl)-2,7-diazaspiro[4.4]nonan-1-one;
2-((1H-indol-3-yl)methyl)-7-(4-methylpyridin-2-yl)-2,7-diazaspiro[4.4]nonan-1-one;
2-((1H-indol-3-yl)methyl)-7-(4,6-dimethylpyridin-2-yl)-2,7-diazaspiro[4.4]nonan-1-one;
2-((1H-indol-3-yl)methyl)-7-(4-methoxypyridin-2-yl)-2,7-diazaspiro[4.4]nonan-1-one;
2-((1H-indol-3-yl)methyl)-7-(2,6-dimethylpyrimidin-4-yl)-2,7-diazaspiro[4.4]nonan-1-one;
2-((1H-indol-3-yl)methyl)-7-(1-methyl-1H-pyrrolo[2,3-c]pyridin-7-yl)-2,7-diazaspiro[4.4]nonan-1-one;
2-((1H-indol-3-yl)methyl)-7-(7-methoxypyrazolo[1,5-a]pyrimidin-5-yl)-2,7-diazaspiro[4.4]nonan-1-one;
2-((1H-indol-3-yl)methyl)-7-(4-(3-methyl-1,2,4-oxadiazol-5-yl)pyridin-2-yl)-2,7-diazaspiro[4.4]nonan-1-one;
2-((1H-indol-3-yl)methyl)-7-(6-(pyrrolidin-1-yl)pyrimidin-4-yl)-2,7-diazaspiro[4.4]nonan-1-one;
2-((1H-indol-3-yl)methyl)-7-(2-methoxy-6-methylpyrimidin-4-yl)-2,7-diazaspiro[4.4]nonan-1-one;
2-((1H-indol-3-yl)methyl)-7-(4-methylthiazol-2-yl)-2,7-diazaspiro[4.4]nonan-1-one;
2-((1H-indol-3-yl)methyl)-7-(5-methoxypyrazolo[1,5-a]pyrimidin-7-yl)-2,7-diazaspiro[4.4]nonan-1-one;
2-((1H-indol-3-yl)methyl)-7-(4-isopropylpyrimidin-2-yl)-2,7-diazaspiro[4.4]nonan-1-one;
2-((1H-indol-3-yl)methyl)-7-(2-methylimidazo[1,2-a]pyrazin-8-yl)-2,7-diazaspiro[4.4]nonan-1-one;
2-((1H-indol-3-yl)methyl)-7-(4-(trifluoromethyl)pyrimidin-2-yl)-2,7-diazaspiro[4.4]nonan-1-one;
7-(4-methoxy-6-methylpyrimidin-2-yl)-2-(2-(3-methyl-1,2,4-oxadiazol-5-yl)benzyl)-2,7-diazaspiro[4.4]nonan-1-one;
7-(4-methoxy-6-methylpyrimidin-2-yl)-2-((2-methyl-5-phenyl-2H-1,2,3-triazol-4-yl)methyl)-2,7-diazaspiro[4.4]nonan-1-one;
7-(4-methoxy-6-methylpyrimidin-2-yl)-2-((2-methyl-5-(m-tolyl)-2H-1,2,3-triazol-4-yl)methyl)-2,7-diazaspiro[4.4]nonan-1-one;
7-(4-methoxy-6-methylpyrimidin-2-yl)-2-((5-(3-methoxyphenyl)-2-methyl-2H-1,2,3-triazol-4-yl)methyl)-2,7-diazaspiro[4.4]nonan-1-one;
2-((5-bromo-2-methyl-2H-1,2,3-triazol-4-yl)methyl)-7-(4-methoxy-6-methylpyrimidin-2-yl)-2,7-diazaspiro[4.4]nonan-1-one;
2-((1H-indol-3-yl)methyl)-7-(4-(dimethylamino)-6-(trifluoromethyl)pyrimidin-2-yl)-2,7-diazaspiro[4.4]nonan-1-one;
2-((1H-Indo)-3-yl)methyl)-7-(quinoxalin-2-yl)-2,7-diazaspiro[4.5]decan-1-one;
7-((1H-Indol-3-yl)methyl)-2-(4-methoxy-6-methylpyrimidin-2-yl)-2,7-diazaspiro[4.5]decan-6-one;
7-((1H-Pyrrolo[2,3-b]pyridin-3-yl)methyl)-2-(4-methoxypyrimidin-2-yl)-2,7-diazaspiro[4.5]decan-6-one;
7-((1H-Indazol-3-yl)methyl)-2-(4-methoxypyrimidin-2-yl)-2,7-diazaspiro[4.5]decan-6-one;
7-((1H-Indo)-3-yl)methyl)-2-(4-methoxypyrimidin-2-yl)-2,7-diazaspiro[4.5]decan-6-one;
2-(4-Methoxypyrimidin-2-yl)-7-(2-(3-methyl-1,2,4-oxadiazol-5-yl)benzyl)-2,7-diazaspiro[4.5]decan-6-one;
7-((5-(3-Methoxyphenyl)-2H-1,2,3-triazol-4-yl)methyl)-2-(4-methoxypyrimidin-2-yl)-2,7-diazaspiro[4.5]decan-6-one;
7-((5-(3-Methoxyphenyl)-2-methyl-2H-1,2,3-triazol-4-yl)methyl)-2-(4-methoxypyrimidin-2-yl)-2,7-diazaspiro[4.5]decan-6-one;
7-((1H-Indol-3-yl)methyl)-2-(benzo[d]oxazol-2-yl)-2,7-diazaspiro[4.5]decan-6-one;
7-((1H-indol-3-yl)methyl)-2-(4-methylpyrimidin-2-yl)-2,7-diazaspiro[4.5]decan-6-one;
7-((1H-indazol-3-yl)methyl)-2-(4-methoxy-6-methylpyrimidin-2-yl)-2,7-diazaspiro[4.5]decan-6-one;
7-((1H-pyrrolo[2,3-b]pyridin-3-yl)methyl)-2-(4-methoxy-6-methylpyrimidin-2-yl)-2,7-diazaspiro[4.5]decan-6-one;
2-(4-methoxy-6-methylpyrimidin-2-yl)-7-(2-(3-methyl-1,2,4-oxadiazol-5-yl)benzyl)-2,7-diazaspiro[4.5]decan-6-one;
7-((1H-indol-3-yl)methyl)-2-(1H-benzo[d]imidazol-2-yl)-2,7-diazaspiro[4.5]decan-6-one;
1-((1H-Indo)-4-yl)methyl)-8-(quinoxalin-2-yl)-1,8-diazaspiro[4.5]decan-2-one;
1-((1H-Indo)-4-yl)methyl)-8-(6-methylpyrazin-2-yl)-1,8-diazaspiro[4.5]decan-2-one;
1-((1H-Indo)-4-yl)methyl)-8-(4,6-dimethylpyrimidin-2-yl)-1,8-diazaspiro[4.5]decan-2-one;
2-(1-((1H-Indol-4-yl)methyl)-2-oxo-1,8-diazaspiro[4.5]decan-8-yl)isonicotinonitrile;
1-(2,5-Dimethylbenzyl)-8-(quinoxalin-2-yl)-1,8-diazaspiro[4.5]decan-2-one;
1-(2,5-Dimethylbenzyl)-8-(4,6-dimethylpyrimidin-2-yl)-1,8-diazaspiro[4.5]decan-2-one;
1-(2,5-Dimethylbenzyl)-9-(quinoxalin-2-yl)-4-oxa-1,9-diazaspiro[5.5]undecan-2-one;
1-((1H-Indo)-3-yl)methyl)-9-(quinoxalin-2-yl)-4-oxa-1,9-diazaspiro[5.5]undecan-2-one;
1-((1H-Indo)-4-yl)methyl)-9-(quinoxalin-2-yl)-4-oxa-1,9-diazaspiro[5.5]undecan-2-one;
1-((1-Methyl-1H-indol-4-yl)methyl)-9-(quinoxalin-2-yl)-4-oxa-1,9-diazaspiro[5.5]undecan-2-one;
1-(3-(Pyridin-2-yl)benzyl)-9-(quinoxalin-2-yl)-4-oxa-1,9-diazaspiro[5.5]undecan-2-one;
1-(3-(Pyridin-3-yl)benzyl)-9-(quinoxalin-2-yl)-4-oxa-1,9-diazaspiro[5.5]undecan-2-one;
1-(2,5-Dimethylbenzyl)-9-(6-methylpyrazin-2-yl)-4-oxa-1,9-diazaspiro[5.5]undecan-2-one;
1-((1H-Indo)-4-yl)methyl)-9-(6-methylpyrazin-2-yl)-4-oxa-1,9-diazaspiro[5.5]undecan-2-one;
1-(2,5-Dimethylbenzyl)-9-(4,6-dimethylpyrimidin-2-yl)-4-oxa-1,9-diazaspiro[5.5]undecan-2-one;
2-(1-((1H-Indol-4-yl)methyl)-2-oxo-4-oxa-1,9-diazaspiro[5.5]undecan-9-yl)isonicotinonitrile;
6-(1-((1H-Indol-4-yl)methyl)-2-oxo-4-oxa-1,9-diazaspiro[5.5]undecan-9-yl)picolinonitrile;
2-(1-((1H-Indol-4-yl)methyl)-2-oxo-4-oxa-1,9-diazaspiro[5.5]undecan-9-yl)pyrimidine-4-carbonitrile;
1-((1H-Indo)-4-yl)methyl)-9-(4-methylpyrimidin-2-yl)-4-oxa-1,9-diazaspiro[5.5]undecan-2-one;
1-(2,5-Dimethylbenzyl)-8-(quinoxalin-2-yl)-3-oxa-1,8-diazaspiro[4.5]decan-2-one;
1-((1H-Indo)-3-yl)methyl)-8-(quinoxalin-2-yl)-3-oxa-1,8-diazaspiro[4.5]decan-2-one;
1-((1H-Indo)-4-yl)methyl)-8-(quinoxalin-2-yl)-3-oxa-1,8-diazaspiro[4.5]decan-2-one;
1-((1H-Indo)-3-yl)methyl)-8-(6-methylpyrazin-2-yl)-3-oxa-1,8-diazaspiro[4.5]decan-2-one;
1-((1H-Indo)-3-yl)methyl)-8-(4,6-dimethylpyrimidin-2-yl)-3-oxa-1,8-diazaspiro[4.5]decan-2-one;
1-((1H-Indo)-4-yl)methyl)-8-(6-methylpyrazin-2-yl)-3-oxa-1,8-diazaspiro[4.5]decan-2-one, and
1-((1H-Indo)-4-yl)methyl)-7-(quinoxalin-2-yl)-1,7-diazaspiro[4.4]nonan-2-one;

and wherein said compound is in free form or in salt form.

In one embodiment, the invention provides a compound selected from
(R)-2-((1H-indol-3-yl)methyl)-7-(4-methoxypyrimidin-2-yl)-2,7-diazaspiro[4.4]nonan-1-one; and
(S)-2-((1H-indol-3-yl)methyl)-7-(4-methoxypyrimidin-2-yl)-2,7-diazaspiro[4.4]nonan-1-one; in free form or in salt form.

In one embodiment, the invention provides a compound selected from
(R)-7-((1H-indol-3-yl)methyl)-2-(4-methoxy-6-methylpyrimidin-2-yl)-2,7-diazaspiro[4.5]decan-6-one; and
(S)-7-((1H-indol-3-yl)methyl)-2-(4-methoxy-6-methylpyrimidin-2-yl)-2,7-diazaspiro[4.5]decan-6-one; in free form or in salt form.

In a further aspect, the invention provides a process for the production of a compound of the formula I, or a salt thereof,

comprising

(a) reacting a compound of formula II-1

A-D-H (II-1),

wherein A is as defined under formula I, and wherein—if present—a N—H bond in group A is optionally protected by an amino protecting group, such as tosyl, and wherein D is selected from

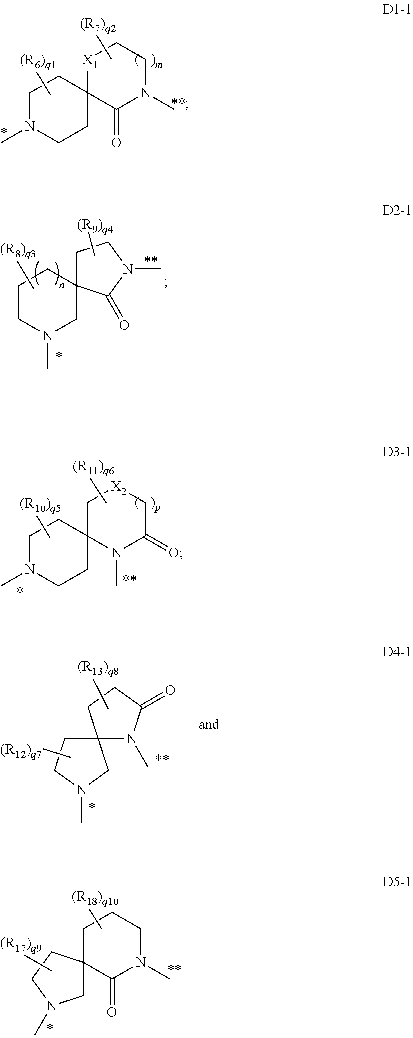

wherein the bond marked with one asterisk is attached to A and the bond marked with two asterisks is attached to the hydrogen atom of formula II-1, and wherein R₆, R₇, R₈, R₉, R₁₀, R₁₁, R₁₂, R₁₃, R₁₇, R₁₈, q1, q2, q3, q4, q5, q6, q7, q8, q9, q10, X₂, n and p are as defined under formula I, and wherein X₁ is —C(R₁₄)₂— or —N(Rₘₐ)— and m is 0 or X₁ is —O— or —N(R₁₅ₐ)— and m is 1, and wherein R₁₄ is as defined under formula I and R₁₅ₐ is hydrogen, C₁₋₆alkyl, C₃₋₇cycloalkyl, C₃₋₇cycloalkyl(C₁₋₄alkyl) or an amino protecting group, such as C₁₋₆alkoxycarbonyl, or a salt thereof,

with a compound of the formula III-1

B—C(R₁)₂—Rₐ (III-1),

wherein B and R₁ are as defined under formula I, and Rₐ is a leaving group, such as chloro, bromo or mesyl, and wherein—if present—a N—H bond in group B is optionally protected by an amino protecting group, such as tosyl, or a salt thereof,

in the presence of a base, such as a strong base, such as sodium hydride, in the presence of a suitable solvent, and optionally in the presence of a suitable catalyst, such as tetrakis(triphenylphosphine)palladium(0);

(b) optionally cleaving any amino protecting group(s) if present in compounds of formula II-1 or their salts and/or in compounds of formula III-1 or their salts; and

(c) optionally converting the compound of formula Ito a salt thereof.

In a further aspect, the invention provides a process for the production of a compound of the formula I, or a salt thereof,

comprising

(a) reacting a compound of formula II-2

H-D-C(R₁)₂—B (II-2),

wherein B and R₁ are as defined under formula I, and wherein D is selected from

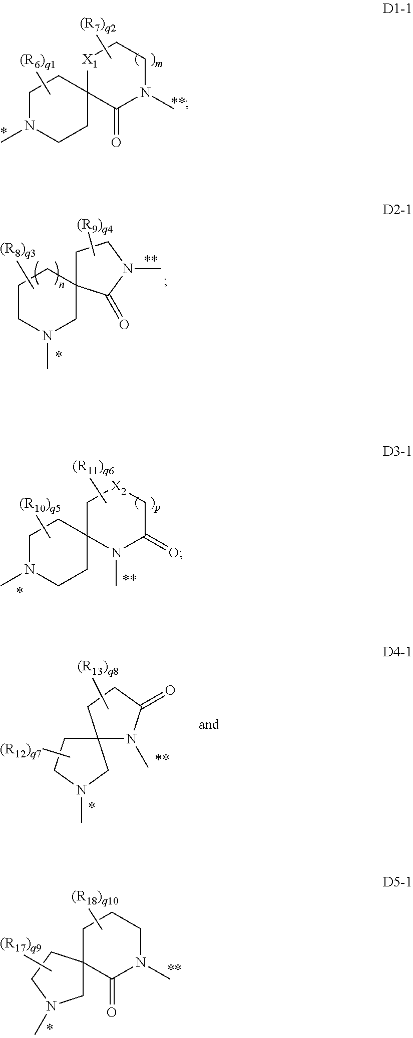

wherein the bond marked with one asterisk is attached to the hydrogen atom of formula II-2 and the bond marked with two asterisks is attached to C(R₁)₂—B, and wherein R₆, R₇, R₈, R₉, R₁₀, R₁₁, R₁₂, R₁₃, R₁₇, R₁₈, q1, q2, q3, q4, q5, q6, q7, q8, q9, q10, X₂, n and p are as defined under formula I, and wherein X₁ is —C(R₁₄)₂— or —N(R₁₅ₐ)— and m is 0 or X₁ is —O— or —N(R₁₅ₐ)— and m is 1, and wherein R₁₄ is as defined under formula I and R₁₅ₐ is hydrogen, C₁₋₆alkyl, C₃₋₇cycloalkyl, C₃₋₇cycloalkyl(C₁₋₄alkyl) or an amino protecting group, such as C₁₋₆alkoxycarbonyl, and wherein—if present—a N—H bond in group B is optionally protected by an amino protecting group, such as tosyl, or a salt thereof,

with a compound of the formula III-2

A-R_{b} (III-2),

wherein A is as defined under formula I, and R_{b} is a leaving group, such as chloro, bromo or mesyl, and wherein—if present—a N—H bond in group A is optionally protected by an amino protecting group, such as tosyl, or a salt thereof,

in the presence of a base, such as a strong base, such as sodium hydride, in the presence of a suitable solvent, and optionally in the presence of a suitable catalyst, such as tetrakis(triphenylphosphine)palladium(0);

(b) optionally cleaving any amino protecting group(s) if present in compounds of formula II-2 or their salts and/or in compounds of formula III-2 or their salts; and

(c) optionally converting the compound of formula Ito a salt thereof.

Further compounds of Formula (I), (I-a), (I-b), (I-c), (I-d), (I-e), (14), (I-g), (I-h) may be obtainable from compounds of Formula (I), (I-a), (I-b), (I-c), (I-d), (I-e), (14), (I-g), (I-h) prepared as described above—by reduction, oxidation and/or other functionalization of resulting compounds and/or by cleavage of any protecting group(s) optionally introduced, and of recovering the so obtainable compounds of Formula (I), (I-a), (I-b), (I-c), (I-d), (I-e), (I-f), (I-g), (I-h).

The reactions can be effected according to conventional methods, for example as described in the Examples.

The work-up of the reaction mixtures and the purification of the compounds thus obtainable may be carried out in accordance with known procedures.

Acid addition salts may be produced from the free bases in known manner, and vice-versa.

Compounds of Formula (I), (I-a), (I-b), (I-c), (I-d), (I-e), (14), (I-g), (I-h) can also be prepared by further conventional processes, e.g. as described in the Examples, which processes are further aspects of the invention.

The starting materials of the formulae II-1, II-2, III-1 and III-2 are known or may be prepared according to conventional procedures starting from known compounds, for example as described in the Examples. These may be used in free form or in salt form.

In another aspect, the invention provides a pharmaceutical composition comprising a compound of the invention and a pharmaceutically acceptable carrier. The pharmaceutical composition can be formulated for particular routes of administration such as oral administration, parenteral administration, and rectal administration, etc. In addition, the pharmaceutical compositions of the invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers etc.

Typically, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with carrier. Carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The invention further provides anhydrous pharmaceutical compositions and dosage forms comprising the compounds of the invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the invention as an active ingredient will decompose. Such agents, which are referred to herein as “stabilizers,” include, but are not limited to, antioxidants such as ascorbic acid, pH buffers, or salt buffers, etc.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of Formula (I), (I-a), (I-b), (I-c), (I-d), (I-e), (I-f), (I-g), (I-h) in free form or in pharmaceutically acceptable salt form, exhibit valuable pharmacological properties, e.g. orexin receptor modulating properties, e.g. as indicated in in-vitro and in-vivo tests as provided in the next sections and are therefore indicated for therapy.

Furthermore, compounds of formula I may be useful for research on orexin receptors, e.g. as tool compounds.

Preferred compounds of Formula (I), (I-a), (I-b), (I-c), (I-d), (I-e), (I-f), (I-g), (I-h) show an inhibition of calcium accumulation in recombinant cells expressing at least one of hOx1R or hOx2R at 10 μM of test compound of at least 10%.

Further preferred compounds of Formula (I), (I-a), (I-b), (I-c), (I-d), (I-e), (I-f), (I-g), (I-h) show a Ki value for said calcium accumulation in recombinant cells expressing at least one of hOx1R or hOx2R of at least 1 μM.

Further preferred compounds of Formula (I), (I-a), (I-b), (I-c), (I-d), (I-e), (I-f), (I-g), (I-h) show a Ki value for said calcium accumulation in recombinant cells expressing at least one of hOx1R or hOx2R of at least 500 nM.

Further preferred compounds of Formula (I), (I-a), (I-b), (I-c), (I-d), (I-e), (I-f), (I-g), (I-h) show a Ki value for said calcium accumulation in recombinant cells expressing at least one of hOx1R or hOx2R of at least 100 nM.

Further preferred compounds of Formula (I), (I-a), (I-b), (I-c), (I-d), (I-e), (I-f), (I-g), (I-h) show a Ki value for said calcium accumulation in recombinant cells expressing at least one of hOx1R or hOx2R of at least 50 nM.

Compounds of the invention may be useful in the treatment of an indication selected from:

i) sleep disorders;

ii) eating disorders;

iii) substance-related disorders;

iv) Alzheimers disease;

v) psychiatric, neurological and neurodegenerative disorders, such as depression; anxiety; addictions, obsessive compulsive disorder; affective neurosis; depressive neurosis; anxiety neurosis; dysthymic disorder; mood disorder; sexual dysfunction; psychosexual dysfunction; sex disorder; schizophrenia; manic depression; delirium; dementia; severe mental retardation and dyskinesias such as Huntington's disease and Tourette syndrome; Parkinson's disease; ischemic or haemorrhagic stroke; migraine; and neurodegenerative disorder including nosological entities such as disinhibition-dementia-parkinsonism-amyotrophy complex; pallido-ponto-nigral degeneration epilepsy; seizure disorders;

vi) cardiovascular diseases, diabetes; asthma; Cushing's syndrome/disease; basophil adenoma; prolactinoma; hyperprolactinemia; hypopituitarism; hypophysis tumor/adenoma; hypothalamic diseases; Froehlich's syndrome; hypophysis diseases, hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; heart and lung diseases, acute and congestive heart failure; hypotension; hypertension; urinary retention; osteoporosis; angina pectoris; myocardial infarction; subarachnoid haemorrhage; ulcers; allergies; benign prostatic hypertrophy; chronic renal failure; renal disease; impaired glucose tolerance; vomiting and nausea; inflammatory bowel disease; gastric dyskinesia; gastric ulcers; urinary bladder incontinence e.g. urge incontinence; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; conditions associated with visceral pain such as irritable bowel syndrome, migraine and angina; and

vii) other diseases related to general orexin system dysfunction.

Compounds of the invention may be especially useful in the treatment of an indication selected from: sleep disorders, eating disorders, substance-related disorders and Alzheimers disease.

“Eating disorders” may be defined as comprising metabolic dysfunction; dysregulated appetite control; compulsive obesities; emeto-bulimia or anorexia nervosa. This pathologically modified food intake may result from disturbed appetite (attraction or aversion for food); altered energy balance (intake vs expenditure); disturbed perception of food quality (high fat or carbohydrates, high palatability); disturbed food availability (unrestricted diet or deprivation) or disrupted water balance.

“Sleep disorders” include insomnias, narcolepsy and other disorders of excessive sleepiness, sleep-related dystonias; restless leg syndrome; sleep apneas; jet-lag syndrome; shift-work syndrome, delayed or advanced sleep phase syndrome. Insomnias are defined as comprising sleep disorders associated with aging; intermittent treatment of chronic insomnia; situational transient insomnia (new environment, noise) or short-term insomnia due to stress; grief; pain or illness.

“Substance-related disorders” include substance abuse, substance dependence and substance withdrawal disorders, e.g. nicotine withdrawal or narcotics withdrawal.

Thus, as a further embodiment, the invention provides the use of a compound of Formula (I), (I-a), (I-b), (I-c), (I-d), (I-e), (I-f), (I-g), (I-h) in free form or in pharmaceutically acceptable salt form as a medicament.

As a further embodiment, the invention provides the use of a compound of Formula (I), (I-a), (I-b), (I-c), (I-d), (I-e), (I-f), (I-g), (I-h) in free form or in pharmaceutically acceptable salt form in therapy.

In a further embodiment, the therapy is selected from a disease which is ameliorated by modulation, preferably antagonism, of orexin receptors. In another embodiment, the disease is selected from the afore-mentioned list, suitably sleep disorders, eating disorders, substance-related disorders or Alzheimers disease.

In another embodiment, the invention provides a method of treating a disease which is ameliorated by modulation, preferably antagonism, of orexin receptors comprising administration of a therapeutically acceptable amount of a compound of Formula (I), (I-a), (I-b), (I-c), (I-d), (I-e), (I-f), (I-g), (I-h) in free form or in pharmaceutically acceptable salt form. In a further embodiment, the disease is selected from the afore-mentioned list, suitably sleep disorders, eating disorders or Alzheimers disease.

In one embodiment, the invention provides a method of inhibiting orexin receptor activity in a subject, wherein the method comprises administering to the subject a therapeutically effective amount of a compound of Formula (I), (I-a), (I-b), (I-c), (I-d), (I-e), (I-f), (I-g), (I-h).

In a further embodiment, the invention provides a method of treating a disorder or a disease in a subject mediated by orexin receptors, wherein the method comprises administering to the subject a therapeutically effective amount of a compound of Formula (I), (I-a), (I-b), (I-c), (I-d), (I-e), (I-f), (I-g), (I-h). Preferably said disorder or said disease is selected from sleep disorders, eating disorders, substance-related disorders, mental health disorders or Alzheimer's disease.

In yet a further embodiment, the invention provides the use of a compound of Formula (I), (I-a), (I-b), (I-c), (I-d), (I-e), (I-f), (I-g), (I-h), for the treatment of a disorder or disease in a subject mediated by orexin receptors.

In yet a further embodiment, the invention provides the use of a compound of Formula (I), (I-a), (I-b), (I-c), (I-d), (I-e), (I-f), (I-g), (I-h), for the treatment of a disorder or disease in a subject characterized by an abnormal activity of orexin receptors. Preferably said disorder or said disease is selected from sleep disorders, eating disorders, substance-related disorders, mental health disorders or Alzheimer's disease.

The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to (1) at least partially alleviating, inhibiting, preventing and/or ameliorating a condition, or a disorder or a disease (i) mediated by orexin receptors, or (ii) associated with orexin receptor activity, or (iii) characterized by abnormal activity of orexin receptors; or (2) reducing or inhibiting the activity of orexin receptors; or (3) reducing or inhibiting the expression of orexin receptors. In another non-limiting embodiment, the term “a therapeutically effective amount” refers to the amount of the compound of the invention that, when administered to a cell, or a tissue, or a non-cellular biological material, or a medium, is effective to at least partially reducing or inhibiting the activity of orexin receptors; or at least partially reducing or inhibiting the expression of orexin receptors.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The pharmaceutical composition or combination of the invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound according to the invention can be assessed by in vitro & in vivo methods described herein.

The compound of the invention may be administered either simultaneously with, or before or after, at least one other therapeutic agent. The compound of the invention may be administered separately, by the same or different route of administration, or together in the same pharmaceutical composition.

The following Examples illustrate the invention, but do not limit it.

Abbreviations:
DBU 1,8-diazabicyclo[5.4.0]undec-7-en
DCE 1,2-dichloroethane
DCM dichloromethane
DIPEA diisopropylethylamine (Hunig's base)
DMAP 4-dimethylaminopyridine
DMSO dimethylsulfoxide
dppf 1,1′-bis(diphenylphosphino)ferrocene
EtOAc ethyl acetate
h hour(s)
HATU 2-(7-aza-1H-benzotriazole-1-yl)-1,1,3,3-tetramethyluronium hexafluorophosphate
Hex hexane
HPLC high pressure liquid chromatography
LDA lithium diisopropylamide
LCMS liquid chromatography mass spectroscopy
MW microwave
min minute(s)
NMP N-methyl-2-pyrrolidone
NMR nuclear magnetic resonance spectrometry
quant. quantitative
Rt retention time
rt room temperature
SEMCl 2-(trimethylsilyl)-ethoxymethyl chloride
TBAI tetrabutyl ammonium iodide
TBME tert-butyl methyl ether
THF tetrahydrofuran
TFA trifluoroacetic acid
TMS trimethylsilyl
Tos toluene-4-sulfonyl
XPhos 2-dicyclohexylphosphino-2′,4′,6′-triisopropylbiphenyl

LCMS Conditions (%=percent by volume):

Method A (Rt_{A}=retention time A)

Agilent 1100 series, LC-MSD; column Zorbax SB-C18 1.8 μm; 3×30 mm; gradient: A water+0.05% TFA/B acetonitrile+0.05% TFA; 0-3.25 min 100A:0B-0A:100B; 3.25-4.0 min 0A:100B; 4.0-4.25 min 0A:100B-100A:0 B; flow 0.7 ml/min; column temperature 35° C.

Method B (Rt_{B}=retention time B)

Agilent 1100 series, LC-MSD; column Zorbax SB-C18 1.8 μm; 3×30 mm; gradient: A water+0.05% TFA/B acetonitrile+0.05% TFA; 0-3.25 min 90A:10B-0A:100B; 3.25-4.0 min 0A:100B; 4.0-4.25 min 0A:100B-90A:10 B; flow 0.7 ml/min; column temperature 35° C.

Method C (Rt_{C}=retention time C)

Agilent 1100 series, LC-MSD; column Zorbax SB-C18 1.8 μm; 3×30 mm; gradient: A water+0.05% TFA/B acetonitrile+0.05% TFA; 0-3.25 min 70A:30B-0A:100B; 3.25-4.0 min 0A:100B; 4.0-4.25 min 0A:100B-70A:30 B; flow 0.7 ml/min; column temperature 35° C.

Method D (Rt_{D}=retention time D)

Waters UPLC Acquity-SQD: column Acquity HSS T3 1.8 μm 2.1×50 mm; A 0.05% formic acid+0.05% ammonium acetate in water/B 0.04% formic acid in acetonitrile; 0-1.40 min 98A:2B-2A:98B, 1.40-2.15 min 2A:98B, 2.15-2.20 min 98A:2B; flow 1.2 ml/min; column temperature 50° C.

Method E (Rt_{E}=retention time E)

Agilent 1100 series, LC-MSD; column Ascentis Express C18 2.7 μm; 2.1×30 mm; gradient: A water+0.05% formic acid+0.05% ammonium acetate/B acetonitrile+0.04% formic acid; 0-1.40 min 98A:2B-2A:98B; 1.40-2.15 min 2A:98B; 2.15-2.19 min 2A:98B-98A: 2B; 2.19-2.20 min 98A:2B; flow 1.2 ml/min; column temperature 50° C.

Method F (Rt_{F}=retention time F)

Agilent 1100 series, LC-MSD; column Zorbax SB-C18 1.8 μm; 3×30 mm; gradient: A water+0.05% TFA/B acetonitrile+0.05% TFA; 0-3.25 min 50A:50B-0A:100B; 3.25-4.0 min 0A:100B; 4.0-4.25 min 0A:100B-50A:50 B; flow 0.7 ml/min; column temperature 35° C.

Method G (Rt_{G}=retention time G)

Thar/Waters SFC-100 MS; column Chiralpak AS-H, 4.6×250 mm; mobile phase scCO₂/IPA 7/3 isocratic; flow 3 ml/min; 150 bar.

Method H (Rt_{H}=retention time H)

Agilent 1200 Chemstation; column Chiralpak AD-H 5 μM; 4.6×250 mm; mobile phase heptane/2-propanol 80:20; flow 1 ml/min.

Method I (Rt_{I}=retention time I)

Preparative Waters chromatography system with Micromass ZQ MS detection; column Waters X Bridge C18-ODB 5 μM; 30×150 mm; gradient: A water+0.79 g/l ammoniumcarbonate/B acetonitrile; 0-12 min 95A:5B to 25A:75B; flow 50 ml/min.

Method J (Rt_{J}=retention time J)

Agilent 1100 series, LC-MSD; column Zorbax SB-C18 1.8 μm; 3×30 mm; gradient: A water+0.05% TFA/B acetonitrile+0.05% TFA; 0-3.25 min 60A:40B-0A:100B; 3.25-4.0 min 0A:100B; 4.0-4.25 min 0A:100B-60A:40 B; flow 0.7 ml/min; column temperature 35° C.

¹H-NMR instruments: Bruker BioSpin (600 MHz), Bruker (400 MHz), Varian (400 MHz), Bruker Advance (600 MHz).

### EXAMPLES

### Building Block A1: tert-Butyl 5-oxo-1,4,9-triazaspiro[5.5]undecane-1-carboxylate

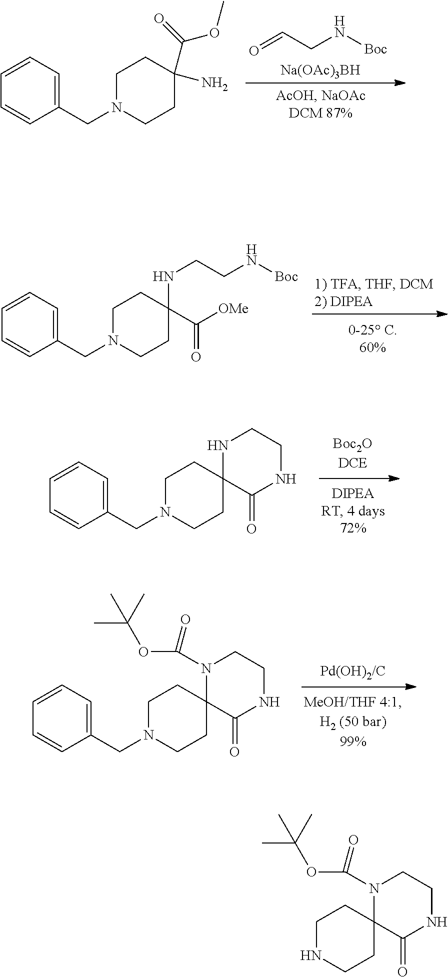

### a) Methyl 1-benzyl-4-((2-((tert-butoxycarbonynamino)ethyl)amino)piperidine-4-carboxylate

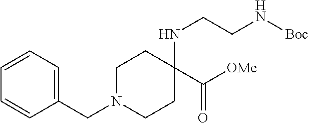

To a solution of methyl 4-amino-1-benzylpiperidine-4-carboxylate (2.0 g, 8.05 mmol) in DCM (100 ml) was added sodium acetate (4.0 g, 48 mmol), tert-butyl (2-oxoethyl)carbamate (3.8 g, 24 mmol), acetic acid (2.8 ml, 48 mmol) and sodium triacetoxyborohydride (10 g, 48 mmol) and the mixture was stirred for 40 min under argon at room temperature.

A saturated solution of NaHCO₃ was added to the reaction mixture at 0° C. and stirred for 5 min. The aqueous layer was extracted with DCM. The organic layers were washed with brine, dried over anhydrous sodium sulfate, filtered and concentrated under reduced pressure to afford a brown oil. The crude product was purified by flash column chromatography (gradient: 0-5% methanol in DCM) to yield the title compound as a pale yellow oil (2.75 g, 87%). [LCMS Rt_{A}=2.79 min, [M+H]⁺=392.2]

### b) 9-Benzyl-1,4,9-triazaspiro[5.5]undecan-5-one

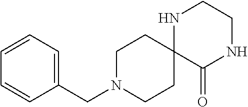

To a solution of methyl 1-benzyl-4-((2-((tert-butoxycarbonyl)amino)ethyl)amino)piperidine-4-carboxylate (0.5 g, 0.77 mmol) in dry DCM (3.3 ml) was added TFA (0.9 ml, 12 mmol) at rt under Argon. The orange reaction mixture was stirred for 1.5 h. TFA and DCM were evaporated to dryness and THF (6 ml) was added to the resulting black oil. The reaction mixture was cooled to 0° C. and Et₃N (1 ml, 7.2 mmol) was added dropwise. The reaction mixture was refluxed for 22 h (not finished after 4 h30). According to LC-MS 2, the reaction was finished, the expected product was formed.

TFA and DCM were evaporated to dryness and THF was added to the resulting black oil. The reaction mixture was cooled down to 0° C. and Et3N was added dropwise. The reaction mixture (black solution) was refluxed for 22 h. After cooling to rt ethyl acetate was added and the mixture washed twice with brine. The aqueous layers were extracted with DCM, the combined organic layers dried over anhydrous sodium sulfate, filtered and concentrated under reduced pressure to afford a brown solid. The crude product was purified by flash column chromatography (gradient: 0-50% ethanol in DCM) to yield the title compound (0.12 g, 60%). [¹H NMR (600 MHz, DMSO-d₆) δ ppm 7.44 (br s, 1H), 7.30 (br s, 4H), 7.24 (br s, 1H), 3.45 (br s, 2H), 3.09 (br s, 2H), 2.78 (br s, 2H), 2.48 (br s, 2H), 2.25 (br s, 2H), 1.93 (br s, 2H), 1.53 (br s, 2H); LCMS Rt_{A}=2.16 min, [M+H]⁺=260.2].

### c) tert-Butyl 9-benzyl-5-oxo-1,4,9-triazaspiro[5.5]undecane-1-carboxylate

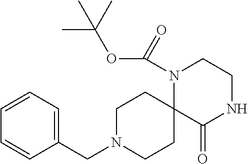

To the solution of 9-benzyl-1,4,9-triazaspiro[5.5]undecan-5-one (371 mg, 1.4 mmol) and DIPEA (0.75 ml, 4.3 mmol) in DCE (4 ml) was added di-tert-butyl dicarbonate (0.4 ml, 1.7 mmol) and the mixture was stirred at rt for 16 h. Di-tert-butyl dicarbonate (1.7 ml, 7.2 mmol) was added again and the mixture stirred for 4 days at rt. The reaction mixture was diluted with ethyl acetate, washed with water. The aqueous layer was extracted ethyl acetate and the organic layers were washed with water and brine. The combined organic layers were dried over anhydrous sodium sulfate, filtered and concentrated under reduced pressure to afford an orange oil. The crude product was purified by flash column chromatography (gradient: 0-5% methanol in DCM) to yield the title compound as a pale beige solid (376 mg, 72%). [LCMS Rt_{A}=3.06 min, [M+H]⁺=360.2].

### d) tert-Butyl 5-oxo-1,4,9-triazaspiro[5.5]undecane-1-carboxylate

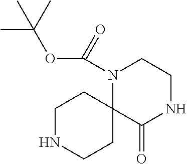

The solution of tert-butyl 9-benzyl-5-oxo-1,4,9-triazaspiro[5.5]undecane-1-carboxylate (376 mg, 1.05 mmol) in methanol/THF (4:1, 20 ml) was hydrogenated in the presence of Pd(OH)₂/C (wet, 20%, 200 mg) in an atmosphere of hydrogen (50 bar) at rt. The mixture was filtered through a pad of celite. The filtrate was concentrated under reduced pressure to afford the title compound as a white powder (280 mg, 99%). [LCMS Rt_{A}=2.67 min, [M+H]⁺=270.2].

### Building Block A2: 4-Oxa-1,9-diazaspiro[5.5]undecan-2-one

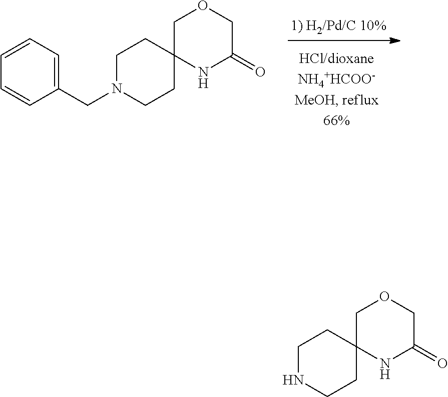

To the solution of 9-benzyl-4-oxa-1,9-diazaspiro[5.5]undecan-2-one (2.43 g, 8.4 mmol) in methanol (40 ml) was added a 4 M solution of hydrogen chloride (gas) in dioxane (0.52 ml, 2.1 mmol), Pd/C (10%, 0.1 g) and ammonium formate (7.9 g, 125 mmol). The mixture was heated at reflux for 3 h. The mixture was filtered through a pad of celite. The filtrate was concentrated under reduced pressure and dissolved in saturated aqueous K₂CO₃ solution and washed with ethyl acetate. The aqueous phase was evaporated to dryness and the solid was triturated with ethanol. The suspension was filtered through a thin layer of silica gel and the remainder was washed with ethanol and 5% aqueous ammonium hydroxide solution. The filtrate was evaporated and the white solid was triturated with DCM/isopropanol (9:1) at 40° C. and filtered. The filtrate was evaporated to afford 1.35 g of a white powder which still contained some salts. This material was used without further purification for derivatization. [¹H NMR (400 MHz, DMSO-d₆) δ ppm 8.30 (s, 1H), 3.94 (s, 2H), 3.61 (s, 2H), 2.89-3.07 (m, 2H), 2.81 (m, 2H), 1.51-1.77 (m, 4H)]

### Building Block A3: 6-Oxo-2,7-diaza-spiro[4.5]decane-2-carboxylic acid tert-butyl ester

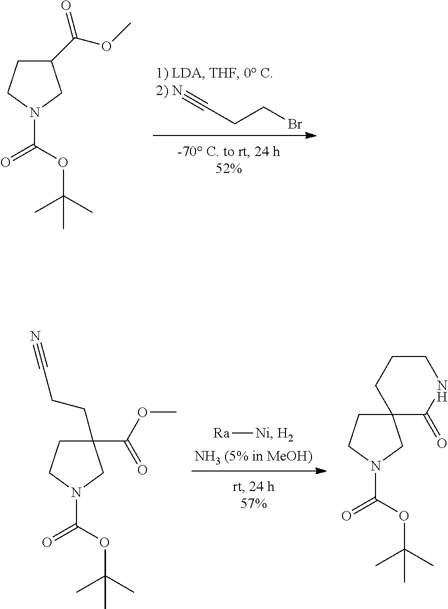

### a) 3-(2-Cyano-ethyl)-pyrrolidine-1,3-dicarboxylic acid 1-tert-butyl ester 3-methyl ester

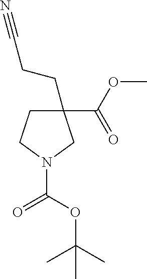

To the solution of diisopropylamine (1.37 ml, 9.6 mmol) in THF (20 ml) at 0° C. under an atmosphere of argon was added buthyl lithium (1.6 M in hexane, 6.0 ml, 9.6 mmol) and the mixture was stirred for 10 min. After cooling to −70° C. the solution of pyrrolidine-1,3-dicarboxylic acid 1-tert-butyl ester 3-methyl ester (2.0 g, 8.7 mmol, in 3 ml THF) was added and the mixture was stirred for 1 h. 3-Bromo-propionitrile (0.79 ml, 9.6 mmol) was added slowly and the reaction mixture was let to warm up over night to rt. The white suspension was poured onto aqueous ammonium chloride and extracted with EtOAc. The organic layer was washed with brine, dried over sodium sulfate, filtered, and the solvents were evaporated under reduced pressure. The crude product was purified by chromatography on silica (flashmaster, hexane to hexane/EtOAc 3/7 in 60 min) to give the product as colorless oil (1.27 g, 52%). [1H-NMR (DMSO, 600 MHz) δ ppm 3.70-3.67 (m, 1H), 3.68 (s, 3H), 3.35-3.31 (m, 1H), 3.22-3.14 (m, 2H), 2.53-2.49 (m, 2H), 2.26-2.20 (m, 1H), 2.00-1.98 (m, 2H), 1.91-1.83 (m, 1H), 1.40 (s, 9H); LCMS Rt_{C}=2.211 min; [M+Na]⁺=305.2]

### b) 6-Oxo-2,7-diaza-spiro[4.5]decane-2-carboxylic acid tert-butyl ester

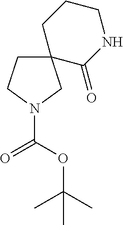

To the solution of 3-(2-cyano-ethyl)-pyrrolidine-1,3-dicarboxylic acid 1-tert-butyl ester 3-methyl ester (1.26 g, 4.24 mmol) in MeOH with 5% NH₃ (15 ml) was added Ra-Ni (Degussa B113W, 0.3 g, followed by two additional portions of 0.5 and 0.2 g during the reduction) and the reaction was stirred under an H₂ atmosphere at rt for 24 h. After filtration the solvent was evaporated under reduced pressure and the product was purified by chromatography on silica (flashmaster, DCM to DCM/MeOH 95/5 over 40 min, followed by DCM/MeOH 95/5 for 10 min) to give a colorless solid (0.61 g, 57%). [1H-NMR (DMSO, 600 MHz) δ ppm 3.51-3.36 (m, 2H), 3.33-3.26 (m, 1H), 3.14-3.09 (m, 3H), 2.20-2.11 (m, 1H), 1.74-1.63 (m, 5H), 1.40 (s, 9H); LCMS Rt_{B}=2.746 min; [M+Na]⁺=277.2]

### Building Block B1: 3-Bromomethyl-1-(toluene-4-sulfonyl)-1H-indazole

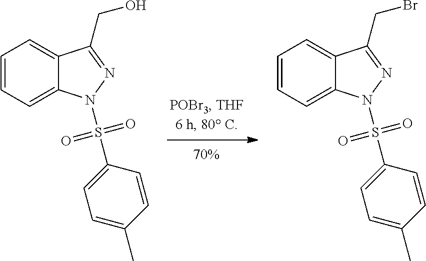

To the solution of [1-(toluene-4-sulfonyl)-1H-indazol-3-yl]-methanol (2.0 g, 5.62 mmol) in THF (15 ml) at 0° C. is added in small portions phosphorous oxybromide (2.63 g, 9.0 mmol). The reaction is let to warm up to rt and is then heated to reflux. After 2 h and 4 h at reflux another portion of phosphorous oxybromide was added (each time 0.6 g, 2.1 mmol) and heating was continued for another 2 h. The reaction was cooled to rt, poured onto saturated sodium bicarbonate solution, and extracted with TBME. The solvents were evaporated under reduced pressure and the product was purified by chromatography on silica (Isolera, hexane to hexane/EtOAc 4/1 in 30 min, then hexane/EtOAc 4/1 for 10 min) to give a colorless solid. [1H-NMR (DMSO, 600 MHz) δ ppm 8.12 (d, 1H), 7.92 (d, 1H), 7.81 (d, 2H), 7.69 (dd, 1H), 7.47 (dd, 1H), 7.38 (d, 2H), 4.97 (s, 2H), 2.31 (s, 3H); LCMS Rt_{C}=3.456 min; [M+H]⁺=365.0/367.0]

### Building Block B2.1: 3-Bromomethyl-1-(toluene-4-sulfonyl)-1H-pyrrolo[2,3-b]pyridine

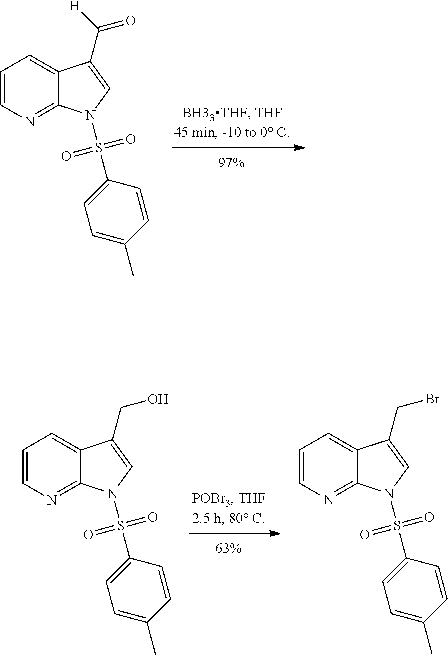

### a) [1-(Toluene-4-sulfonyl)-1H-pyrrolo[2,3-b]pyridin-3-yl]methanol

The solution of 1-(toluene-4-sulfonyl)-1H-pyrrolo[2,3-b]pyridine-3-carbaldehyde ([956716-93-1], 3.63 g, 12.1 mmol) in THF (20 ml) was added slowly to BH₃.THF (1 M in THF, 24.2 ml, 24.2 mmol) at −10° C. After the addition the reaction was stirred at 0° C. for 45 min. After careful addition of brine (5 ml) part of the solvents were evaporated under reduced pressure and the mixture was extracted with EtOAc. The combined organic layers were washed with sodium bicarbonate solution and brine, dried over sodium sulfate, and the solvents were evaporated under reduced pressure. The residue was crystallized from DCM/hexane to give the product as colorless crystals (3.55 g, 97%). [LCMS Rt_{B}=2.968 min; [M+H]⁺=303.0]

### b) 3-Bromomethyl-1-(toluene-4-sulfonyl)-1H-pyrrolo[2,3-b]pyridine

To the solution of [1-(toluene-4-sulfonyl)-1H-pyrrolo[2,3-b]pyridin-3-yl]-methanol (1.0 g, 3.31 mmol) in THF (20 ml) was added phosphorus oxybromide (1.05 g, 3.67 mmol) in small portions, keeping the temperature at 5-10° C. The reaction was then heated at 80° C. for 2.5 h. After cooling to rt the reaction was poured on sodium bicarbonate solution and extracted with TBME. The combined organic layers were dried over sodium sulfate, filtered through a patch of silica, and the solvents were evaporated under reduced pressure. Purification by chromatography on silica (flashmaster, hexan/EtOAc 95/5 to 80/20 in 40 min, then hexan/EtOAc 80/20 for 10 min) gave the product in about 85% purity as colorless solid (0.755 g, 63%). [1H-NMR (CDCl₃, 600 MHz) δ ppm 8.50 (dd, 1H), 8.12 (d, 2H), 8.02 (dd, 1H), 7.82 (s, 1H), 7.32 (d, 2H), 7.28 (dd, 1H), 4.64 (s, 2H), 2.41 (s, 3H); LCMS Rt_{C}=3.221 min; [M+H]⁺=365.0/367.0]

### Building Block B2.2: 3-Bromomethyl-5-fluoro-1-(toluene-4-sulfonyl)-1H-indole

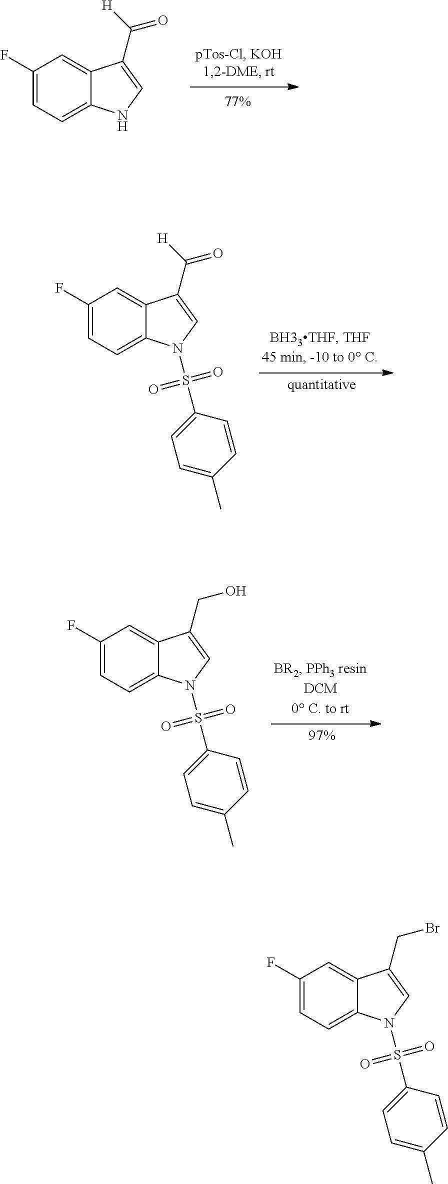

### a) 5-Fluoro-1-tosyl-1H-indole-3-carbaldehyde

To the solution of 5-fluoro-1H-indole-3-carbaldehyde (1.29 g, 7.91 mmol) in DME (13 ml) was added KOH (1.33 g, 23.7 mmol) and the resulting solution stirred at rt for 10 min. Then, toluene-4-sulfonyl chloride (1.66 g, 8.7 mmol) was added and stirring continued at rt. After 2 h, the solvent was removed under reduced pressure, the residue taken up in AcOEt/H₂O and the phases separated. The aqueous phase was extracted two more times with AcOEt, the combined organic phases washed with brine, dried (Na₂SO₄) and the solvent removed under reduced pressure. The resulting solid was used for the next steps without further purification. [1H-NMR (DMSO, 400 MHz) δ ppm 10.03 (s, 1H), 8.93 (s, 1H), 7.93-8.02 (m, 3H), 7.78 (dd, 1H), 7.45 (d, 2H), 7.31 (dt, 1H), 2.33 (s, 3H); LCMS Rt_{E}=2.091 min; [M+H]⁺=318.2]

### b) (5-Fluoro-1-tosyl-1H-indol-3-yl)methanol

To a solution of 5-fluoro-1-tosyl-1H-indole-3-carbaldehyde (500 mg, 1.6 mmol) in THF (12 ml) was added dropwise a 1M solution of BH₃ in THF (1.6 ml, 1.6 mmol) at −10° C. The reaction mixture was stirred at 0° C. for 35 min and then a saturated solution of sodium chloride in water was added dropwise. The mixture was extracted with dichloromethane. The organic phase was washed with water and brine. The combined organic layers were dried over sodium sulfate, filtered and evaporated to give the title compound as an orange oil which was used without further purification in the next step. [LCMS Rt_{B}=3.48 min; [M+Na]⁺=342.0].

### c) 3-(Bromomethyl)-5-fluoro-1-tosyl-1H-indole

To a suspension of polymer-bound triphenylphosphine (790 mg, 2.5 mmol, Aldrich 14664-25G-F) in dry DCM (16 ml) was added dropwise at 0° C. bromine (0.13 ml, 2.5 mmol). The mixture was slowly stirred at 0° C. for 45 min. A solution of (5-fluoro-1-tosyl-1H-indol-3-yl)methanol (500 mg, 1.6 mmol) in dry DCM (6 ml) was added dropwise at 0° C. under argon. The reaction mixture was allowed to reach room temperature and stirring was continued for 2.5 h. The reaction mixture was filtered and the filtrate was evaporated to dryness to afford the not very stable title compound (580 mg, 97%) as a red solid. [¹H NMR (400 MHz, CDCl₃) δ ppm 7.91 (dd, 1H), 7.75 (d, 1H), 7.66 (s, 1H), 7.29 (dd, 1H), 7.24 (s, 1H), 7.08 (td, 1H), 4.57 (s, 2H), 2.36 (s, 3H); LCMS Rt_{C}=3.74 min, [M-Br]⁺=302.0].

The following building blocks were obtained by similar methods starting from the corresponding aldehydes or acids:

| | | | LCMS | |
|---|---|---|---|---|
| | | | Rt [min], | |
| BB | Structure | Name | method | [M + H]⁺ |
| B2.3 | 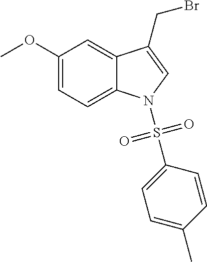 | 3-Bromomethyl-5-methoxy-1- (toluene-4-sulfonyl)-1H-indole | 1.02 (E) | 314.2 ([M − Br]⁺) |
| B2.4 | 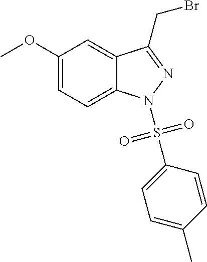 | 3-Bromomethyl-5-methoxy-1- (toluene-4-sulfonyl)-1H- indazole | 3.47 (C) | 395.0/397.0 |

### Building Block B3.1: 4-Bromomethyl-5-(3-methoxymethyl-phenyl)-oxazole

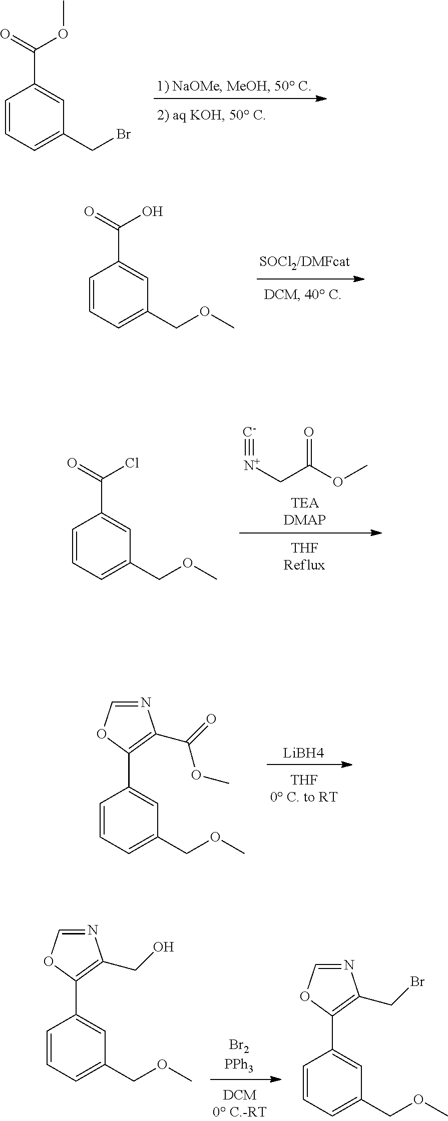

### a) 3-(Methoxymethyl)benzoic acid

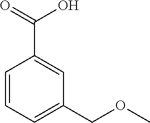

To a solution of methyl 3-(bromomethyl)benzoate, 1.05 g, 4.6 mmol) in dry MeOH (30 ml) under argon at rt was added sodium methanolate (0.74 g, 13.8 mmol). The suspension was stirred at 50° C. for 2 hr and then aqueous KOH (5M) was added and the mixture again stirred at 50° C. for 1.5 hr.

After cooling to rt aqueous HCl was added to adjust the pH to 2-3. The aqueous phase was extracted with ethyl acetate, the organic phase dried over sodium sulfate, and the solvent was evaporated under reduced pressure to afford the title compound as colorless oil (749 mg, 96%). LCMS Rt_{B}=2.47 min; [M+H]⁺=167.0]

### b) 3-(Methoxymethyl)benzoyl chloride

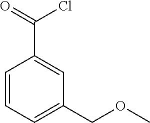

To a solution of 3-(methoxymethyl)benzoic acid (750 mg, 4.4 mmol) in anhydrous DCM (30 ml), DMF (2 drops) and thionyl chloride (0.66 ml, 9.1 mmol) were added. The mixture was stirred at 40° C. under argon for 60 min. More thionyl chloride (0.17 ml, 2.3 mmol) was added and the mixture was stirred for additional 50 min. The solvent was evaporated to give a yellow oil which was used directly in the next step.

### c) Methyl 5-(3-(methoxymethyl)phenyl)oxazole-4-carboxylate

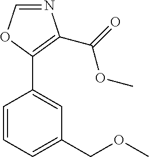

To a solution of methyl isocyanoacetate (0.50 ml, 5.2 mmol) and 3-(methoxymethyl)benzoyl chloride (4.4 mmol) in anhydrous THF, were added triethylamine (3.5 ml, 25 mmol) and DMAP (0.03 g, 0.23 mmol). The resulting brown mixture was stirred at reflux for 21 h. According to LC-MS 1, the reaction was finished. The mixture was filtered, the residue washed with ethyl acetate and the filtrate was washed with water and brine. The organic phase was dried over sodium sulfate, and the solvent was evaporated under reduced pressure. The crude product was purified by flash column chromatography (gradient: 0-3% methanol in DCM) to yield the title compound as a pale brown oil (0.95 g, 87%). [LCMS Rt_{B}=2.94 min, [M+H]⁺=418.0].

### d) (5-(3-(Methoxymethyl)phenyl)oxazol-4-yl)methanol

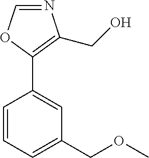

To the solution of methyl 5-(3-(methoxymethyl)phenyl)oxazole-4-carboxylate (0.35 g, 1.4 mmol) in dry THF was added lithium borohydride at 0° C. The mixture was allowed to warm to rt and stirred for 2 hr. The reaction was quenched with acetic acid/water (1:1) and diluted with water. The product was extracted with ethyl acetate. The organic phase was washed with water and brine. The combined organic layers were dried over sodium sulfate, filtered and evaporated to give the title compound (0.31 g, 83%) as an orange oil. [LCMS Rt_{B}=2.55 min, [M+H]⁺=220.0].

### e) 4-(Bromomethyl)-5-(3-(methoxymethyl)Phenyl)oxazole

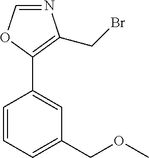

Bromine (0.15 ml, 2.9 mmol) was added dropwise under argon at 0° C. to a solution of triphenylphosphine (0.77 g, 2.9 mmol) in dry DCM (8 ml). After 10 min a solution of (5-(3-(methoxymethyl)phenyl)oxazol-4-yl)methanol in dry DCM (4 ml) was added at 0° C. and the pale yellow solution was allowed to reach rt and stirred for 60 min. The mixture was purified by flash column chromatography (gradient: 0-100% diethyl ether in hexane) to yield the title compound as a pale yellow oil (0.09 g, 27%). [LCMS Rt_{C}=2.52 min, [M+H]⁺=282.0/284.0].

### Building Block B3.2: 4-Bromomethyl-5-(3-methoxymethyl-phenyl)-2-methyl-oxazole

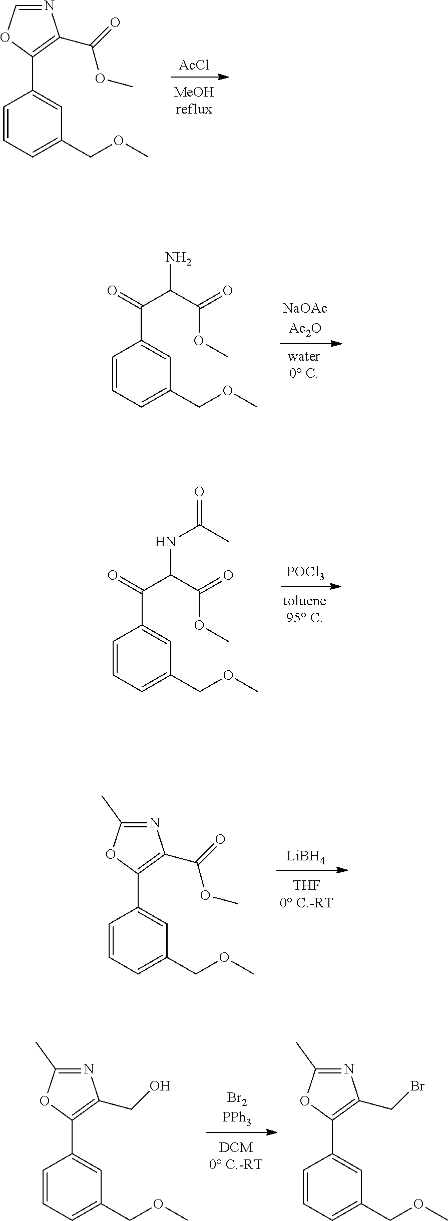

### a) Methyl 2-amino-3-(3-(methoxymethyl)phenyl)-3-oxopropanoate

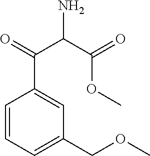

To a solution of methyl 5-(3-(methoxymethyl)phenyl)oxazole-4-carboxylate (building block B3.1c, 25 g, 103 mmol) in MeOH was added dropwise acetyl chloride at 0° C. The mixture was stirred at reflux for 24 h. The solvent was evaporated under reduced pressure, the crude product was triturated with acetone, filtered and the residue washed with acetone to give the title compound as a beige solid as its hydrochloride salt (10.6 g, 38%). [LCMS Rt_{A}=2.53 min, [M+H]⁺=238.2].

### b) Methyl 2-acetamido-3-(3-(methoxymethyl)phenyl)-3-oxopropanoate

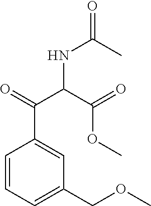

To a solution of methyl 2-amino-3-(3-(methoxymethyl)phenyl)-3-oxopropanoate (10.6 g, 39 mmol) and sodium acetate (3.2 g, 39 mmol) in water (310 ml) was added acetic anhydride (7.7 ml, 81 mmol) at 0° C. and the mixture was stirred at 0° C. for 30 min. The mixture was diluted with water and the product was extracted with DCM. The organic phase was washed with water and brine. The organic layer was dried over sodium sulfate, filtered and evaporated to give 10.5 g (97%) of the title compound as brown oil. [LCMS Rt_{B}=2.57 min, [M+H]⁺=280.2].

### c) Methyl 5-(3-(methoxymethyl)phenyl)oxazole-4-carboxylate

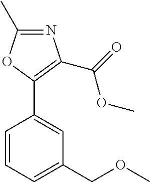

To a solution of methyl 2-acetamido-3-(3-(methoxymethyl)phenyl)-3-oxopropanoate (10.5 g, 37 mmol) in dry toluene (200 ml) was added POCl₃ at room temperature. The solution was stirred at 95° C. for 3 h. After cooling to rt the mixture was poured into water. By addition of an aqueous solution of NaOH (4M) at 0° C. the pH was adjusted to 7. The mixture was extracted with ethyl acetate. The combined organic layers were dried over sodium sulfate, filtered and evaporated to give the title compound as a pale yellow oil (9.2 g, 94%). [LCMS Rt_{B}=3.05 min, [M+H]⁺=262.0].

### d) (5-(3-(Methoxymethyl)phenyl)-2-methyloxazol-4-yl)methanol

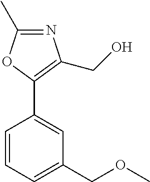

To the solution of methyl 5-(3-(methoxymethyl)phenyl)oxazole-4-carboxylate (9.2 g, 35 mmol) in THF (250 ml) was added at 0° C. LiBH₄ (1.6 g, 70 mmol). The mixture was allowed to reach rt and stirring was continued for 2.5 h. A solution of acetic acid in water (1:1 v/v) was added slowly until the mixture became a clear solution. Stirring was continued for 16 h at rt. The mixture was extracted with DCM, the organic phase was washed with water and brine and the combined organic layers were dried over sodium sulfate, filtered and evaporated to give 6.7 g of a yellow oil which was not further purified.

### e) 4-(Bromomethyl)-5-(3-(methoxymethyl)phenyl)-2-methyloxazole

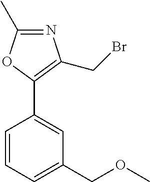

Bromine (3.5 ml, 67 mmol) was added dropwise at 0° C. under argon to a solution of triphenylphosphine (17.7 g, 67 mmol) in dry DCM (250 ml). The pale yellow suspension was stirred at 0° C. for 10 min. The solution of crude (5-(3-(methoxymethyl)phenyl)-2-methyloxazol-4-yl)methanol (6.7 g) in dry DCM (30 ml) was added at 0° C. and the pale yellow solution was allowed to warm to rt and stirring was continued for 1 hr. For completion the reaction mixture was left at 5° C. for 3 days. The reaction mixture was extracted with DCM and the organic phase was washed with water and brine. The combined organic layers were dried over sodium sulfate, filtered and evaporated to give a pale yellow solid. The crude product was purified by flash column chromatography (hexane/diethyl ether 1:1) to yield the title compound as a pale yellow oil (4.0 g, 50%). [LCMS Rt_{C}=2.68 min, [M+H]⁺=296.0/298.0].

The following building blocks were obtained by similar methods starting from the corresponding acid chlorides:

| | | | LCMS | |
|---|---|---|---|---|
| | | | Rt [min], | |
| BB | Structure | Name | method | [M + H]⁺ |
| B3.3 | 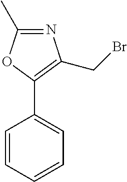 | 4-Bromomethyl- 2-methyl- 5-phenyl-oxazole | 3.46 (B) | 252.0/254.0 |
| B3.3 | 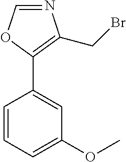 | 4-Bromomethyl- 5-(3-methoxy- phenyl)-oxazole | 2.68 (C) | 268.0/270.0 |
| B3.3 | 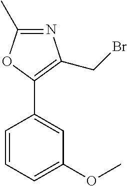 | 4-Bromomethyl- 5-(3-methoxy- phenyl)-2- methyl-oxazole | 2.82 (C) | 282.0/284.0 |

### Building Block B4: 4-Bromomethyl-5-(3-methoxy-phenyl)-2-methyl-thiazole

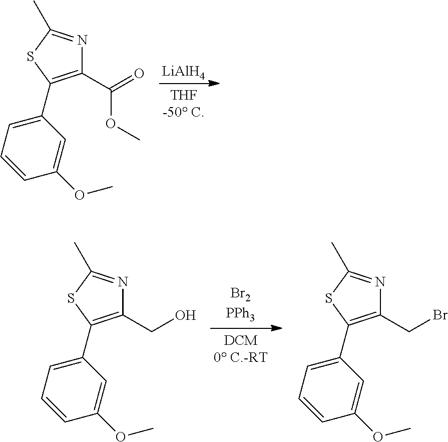

### a) (5-(3-Methoxyphenyl)-2-methylthiazol-4-yl)methanol

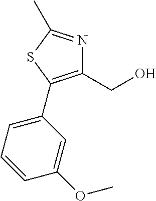

To the solution of methyl 5-(3-methoxyphenyl)-2-methylthiazole-4-carboxylate (WO2010004507, 237 mg, 0.90 mmol) in anhydrous THF was added dropwise a solution of LiAlH₄ (1 M in THF, 3.6 ml, 3.6 mmol) at −50° C. under argon and the mixture was stirred for 130 min at −50° C. An aqueous solution of acetic acid in water (1:1 v/v) was added dropwise at −50° C. until bubbling ceased. The mixture was allowed to warm to 0° C. and then diluted with a saturated solution of NaHCO₃ to adjust to pH 8. The mixture was extracted with ethyl acetate. The organic phase was washed with water and brine. The combined organic layers were dried over sodium sulfate, filtered and evaporated to give the title compound as a yellow solid (196 mg, 92%). [LCMS Rt_{B}=2.75 min, [M+H]⁺=236.0].

### b) 4-(Bromomethyl)-5-(3-methoxyphenyl)-2-methylthiazole

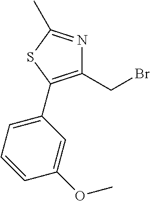

Bromination of (5-(3-Methoxyphenyl)-2-methylthiazol-4-yl)methanol (196 mg, 0.83 mmol) was done analogously to building block B3.2 e) to yield the title compound as a beige solid (182 mg, 68%). [LCMS Rt_{B}=3.66 min, [M+H]⁺=298.0/300.0].

### Building Block B5.1: 4-Bromomethyl-5-(3-methoxy-phenyl)-2-(2-trimethylsilanyl-ethoxymethyl)-2H-[1,2,3]triazole

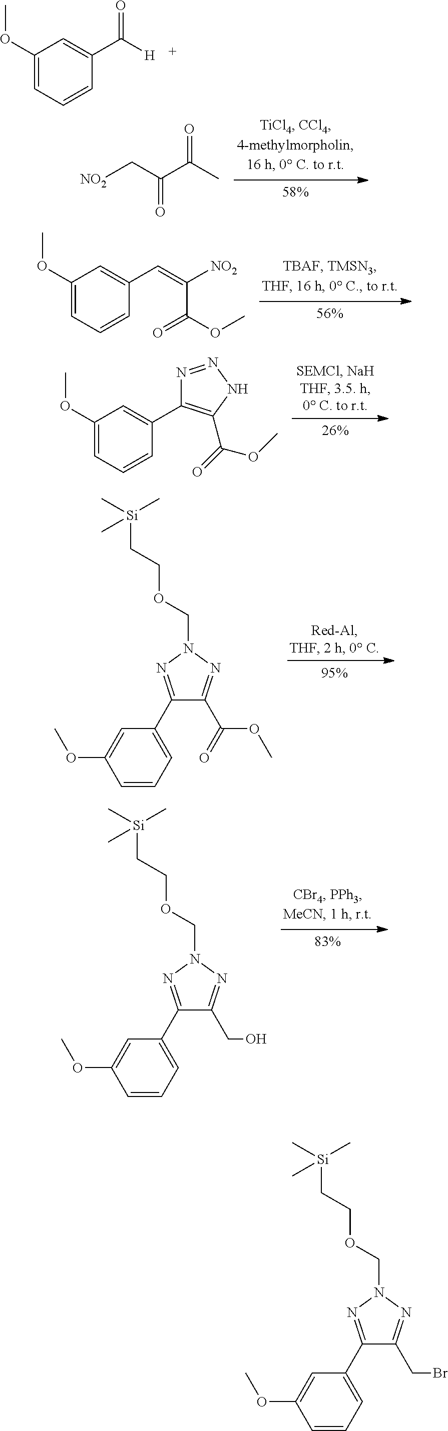

### a) (E/Z)-3-(3-Methoxy-phenyl)-2-nitro-acrylic acid methyl ester

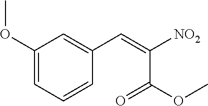

A solution of titan tetrachloride (27.6 g, 142 mmol) in CCl₄ (39 ml) was dropped within 20 min to THF (260 ml) at 0° C. under an atmosphere of argon. Then, m-anisaldehyde (10.0 g, 71.2 mmol) and methyl 2-nitroacetate (8.75 g, 71.2 mmol) were added to the yellow suspension. A solution of N-methylmorpholine (29.4 g, 285 mmol) in THF (47 ml) was added slowly within 110 min at 0° C. to the reaction mixture. The reaction mixture was allowed to warm to rt and stirring was continued for 16 h. The reaction mixture was poured onto water and extracted with TBME. The organic layer was washed with brine, dried over sodium sulfate, filtered, and the solvents were evaporated under reduced pressure. The crude product was purified by chromatography on silica (Biotage Isolera Four, heptane/EtOAc 98/2 for 5 min, then to heptane/EtOAc 80/20 in 50 min, then heptane/EtOAc 80/20 for 10 min) to give the product as mixture of E/Z isomers (10.04 g, 58%). [¹H NMR (400 MHz, DMSO-d₆) δ ppm 7.86/8.38 (s, 1H), 7.02-7.47 (m, 4H), 3.88/3.92 (s, 3H), 3.75/3.77 (s, 3H); LCMS Rt_{D}=1.07/1.08 min; [M+H]⁺=not found]

### b) 5-(3-Methoxy-phenyl)-3H-1,2,3]triazole-4-carboxylic acid methyl ester

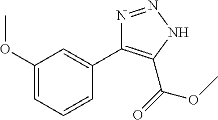

To the solution of (E/Z)-3-(3-methoxy-phenyl)-2-nitro-acrylic acid methyl ester (9.80 g, 40.9 mmol) in THF (100 ml) was added t-butyl ammonium fluoride (4.09 ml, 4.09 mmol, 1M solution in THF) at 0° C. under an atmosphere of argon. After 5 min, trimethylsilyl azide (19.84 g, 164 mmol) was added within 40 min at 0° C. The reaction mixture was allowed to warm to rt and stirring was continued for 16 h. The solvents were evaporated under reduced pressure. The crude product was purified by chromatography on silica (Biotage Isolera Four, heptane/EtOAc 75/25 for 5 min, then to heptane/EtOAc 0/100 in 20 min, then heptane/EtOAc 0/100 for 40 min) to give the title compound (5.43 g, 56%). [¹H NMR (400 MHz, DMSO-d₆) δ ppm 7.28-7.45 (m, 3H), 6.99-7.08 (m, 1H), 3.80 (s, 3H), 3.79 (s, 3H), 3.32 (br s, 1H); LCMS Rt_{D}=0.76 min; [M+H]⁺=234.1]

### c) 5-(3-Methoxy-phenyl)-2-(2-trimethylsilanyl-ethoxymethyl)-2H-1,2,3]triazole-4-carboxylic acid methyl ester

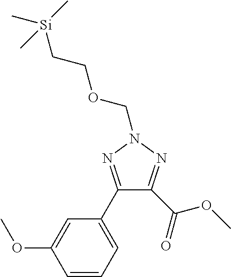

To the solution of 5-(3-methoxy-phenyl)-3H-[1,2,3]triazole-4-carboxylic acid methyl ester (5.20 g, 22.07 mmol) in THF (70 ml) was added sodium hydride (1.324 g, 33.1 mmol, 60% in dispersion oil) at 0° C. under an atmosphere of argon. The mixture was stirred for 30 min at 0° C. Thereafter, SEMCl (4.29 g, 23.2 mmol) was added. The reaction mixture was allowed to warm to rt and stirring was continued for 3.5 h. The reaction mixture was poured onto saturated aqueous NaHCO₃ solution and extracted with TBME (2×). The combined organic layers were washed with brine, dried over sodium sulfate, filtered, and the solvents were evaporated under reduced pressure. The crude product was purified by chromatography on silica (Biotage Isolera Four, heptane/EtOAc 90/10 for 5 min, to heptane/EtOAc 50/50 in 50 min and heptane/EtOAc 50/50 for 20 min) to give the title compound (2.40 g, 26%) and a regioisomer (1.00 g, 12%). [¹H NMR (400 MHz, DMSO-d₆) δ ppm 7.29-7.42 (m, 3H), 7.00-7.06 (m, 1H), 5.77 (s, 2H), 3.82 (s, 3H), 3.78 (s, 3H), 3.68 (t, 2H), 0.86 (t, 2H), −0.10 to −0.01 (m, 9H); LCMS Rt_{D}=1.33 min; [M+H]⁺=364.2, regioisomer: Rt_{D}=1.24 min; [M+H]⁺=364.2]

### d) [543-Methoxy-phenyl)-2-(2-trimethylsilanyl-ethoxymethyl)-2H-[1,2,3]triazol-4-yl]-methanol

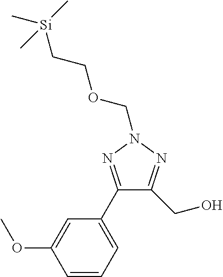

To the solution of 5-(3-methoxy-phenyl)-2-(2-trimethylsilanyl-ethoxymethyl)-2H-[1,2,3]triazole-4-carboxylic acid methyl ester (2.40 g, 5.81 mmol) in THF (24 ml) was added Red-Al (2.094 ml, 5.81 mmol, 55% in toluol) at 0° C. under an atmosphere of argon. The mixture was stirred at 0° C. for 1.5 h. The ice bath was removed, the reaction mixture was poured onto water and stirred for additional 2.5 h. Then brine was added and the mixture was extracted with TBME (2×). The combined organic layers were washed with brine, dried over sodium sulfate, filtered and the solvents were evaporated under reduced pressure. The crude product was purified by chromatography on silica (Biotage Isolera Four, heptane/EtOAc 90/10 for 3 min, then to heptane/EtOAc 50/50 in 25 min, then heptane/EtOAc 50/50 for 5 min) to give the title compound (1.88 g, 95%). [¹H NMR (400 MHz, DMSO-d₆) δ ppm 7.31-7.53 (m, 3H), 6.89-7.04 (m, 1H), 5.65 (s, 2H), 5.48 (t, 1H), 4.62 (d, 2H), 3.79 (s, 3H), 3.65 (t, 2H), 0.85 (t, 2H), −0.06 to −0.04 (m, 9H); LCMS Rt_{D}=1.20 min; [M+H]⁺=336.2]

### e) 4-Bromomethyl-5-(3-methoxy-phenyl)-2-(2-trimethylsilanyl-ethoxymethyl)-2H-[1,2,3]triazole

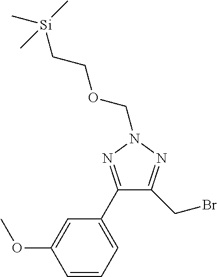

To the solution of [5-(3-methoxy-phenyl)-2-(2-trimethylsilanyl-ethoxymethyl)-2H-[1,2,3]triazol-4-yl]-methanol (1.87 g, 5.52 mmol) and CBr₄ (2.83 g, 8.28 mmol) in acetonitrile (23 ml) was added a solution of PPh₃ (2.28 g, 8.28 mmol) in acetonitrile (22 ml) at room temperature under an atmosphere of argon. The mixture was stirred at room temperature for 1 h. The solvents were evaporated under reduced pressure. The crude product was purified by chromatography on silica (Biotage Isolera Four, heptane/EtOAc 98/2 for 3 min, then to heptane/EtOAc 80/20 in 25 min, then heptane/EtOAc 80/20 for 5 min) to give the title compound (1.85 g, 83%). [¹H NMR (400 MHz, DMSO-d₆) δ ppm 7.44 (t, 1H), 7.36 (d, 1H), 7.27-7.33 (m, 1H), 6.97-7.07 (m, 1H), 5.68 (s, 2H), 4.89 (s, 2H), 3.82 (s, 3H), 3.65 (t, 2H), 0.84 (t, 2H), −0.07 to −0.05 (m, 9H); LCMS Rt_{D}=1.43 min; [M+H]⁺=398.0/400.0]

The following building block was obtained by similar methods starting from the corresponding aldehyde:

| | | | LCMS | |
|---|---|---|---|---|
| | | | Rt [min], | |
| BB | Structure | Name | method | [M + H]⁺ |
| B5.2 | 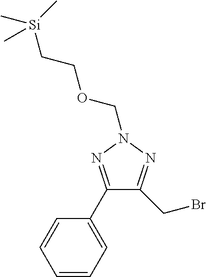 | 4-Bromomethyl-5-phenyl-2-(2- trimethylsilanyl-ethoxymethyl)- 2H-[1,2,3]triazole | 1.45 (D) | 368.3/370.3 |

### Building Block B6: 5-(2-(bromomethyl)phenyl)-3-(methoxymethyl)-1,2,4-oxadiazole

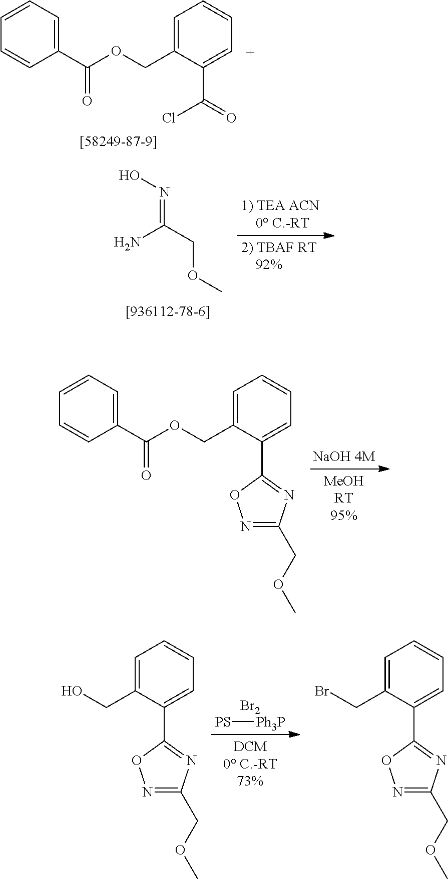

### a) 2-(3-(methoxymethyl)-1,2,4-oxadiazol-5-yl)benzyl benzoate

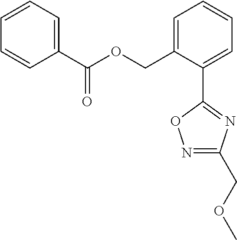

To a solution of 2-(chlorocarbonyl)benzyl benzoate (1.0 g, 3.5 mmol) and triethylamine (1 ml, 7.2 mmol) in dry acetonitrile (36 ml) was added under argon at 0° C. (Z)—N′-hydroxy-2-methoxyacetimidamide (0.44 g, 4.2 mmol). The reaction mixture was stirred at 0° C. for 15 min and at rt for 1.5 h. A 1M solution of tetrabutylammonium fluoride (15 ml, 15 mmol) was added at room temperature and the yellow-orange solution was stirred for 1.5 h.

To the reaction mixture were added DCM and water. The phases were separated. The aqueous layer was extracted with DCM. The organic phases were washed with brine, combined and dried over sodium sulfate, filtered and concentrated under reduced pressure to afford a brown oil. The crude product was purified by flash column chromatography (hexane/ethyl acetate 4:1) to yield the title compound (1.06 g, 92%). [¹H NMR (400 MHz, DMSO-d₆) δ ppm 8.14 (d, 1H), 7.91 (d, 2H), 7.79-7.72 (m, 2H), 7.67-7.60 (m, 2H), 7.51 (t, 2H), 5.76 (s, 2H), 4.57 (s, 2H), 3.30 (s, 3H); LCMS Rt_{J}=2.80 min, [M+H]⁺=325.0].

### b) (2-(3-(methoxymethyl)-1,2,4-oxadiazol-5-yl)phenyl)methanol

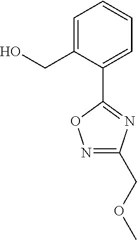

To a solution of 2-(3-(methoxymethyl)-1,2,4-oxadiazol-5-yl)benzyl benzoate (1.06 g, 3.3 mmol) in MeOH (30 ml) was added an aqueous solution of sodium hydroxide (2 ml, 4M). The reaction mixture was stirred at room temperature for 1.5 h. The organic phases were washed with brine, combined and dried over sodium sulfate, filtered and concentrated under reduced pressure to afford a brown oil. The crude product was purified by flash column chromatography (hexane/ethyl acetate 1:1) to yield the title compound (0.68 g, 95%). [¹H NMR (400 MHz, DMSO-d₆) δ ppm 8.03 (d, 1H), 7.80 (d, 1H), 7.68 (t, 1H), 7.48 (t, 1H), 5.36 (t, 1H), 4.90 (d, 2H), 4.62 (s, 2H), 3.38 (s, 3H); LCMS Rt_{B}=2.63 min, [M+H]⁺=221.0].

### c) 5-(2-(bromomethyl)phenyl)-3-(methoxymethyl)-1,2,4-oxadiazole

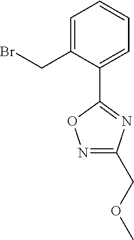

To a suspension of polymer-bound triphenylphosphine (1.4 g, 4.6 mmol, Aldrich 14664-25G-F) in dry DCM (22 ml) was added dropwise at 0° C. bromine (0.24 ml, 4.6 mmol). The mixture was slowly stirred at 0° C. for 1 h. A solution of (2-(3-(methoxymethyl)-1,2,4-oxadiazol-5-yl)phenyl)methanol (0.68 g, 3.1 mmol) in dry DCM (8 ml) was added dropwise at 0° C. The reaction mixture was allowed to reach room temperature and stirred for 19 h. The mixture was filtered and washed with DCM. The solution was evaporated in vacuo to give a yellow oil which was purified by flash column chromatography (hexane/ethyl acetate 4:1) to yield the title compound (0.64 g, 73%). [¹H NMR (400 MHz, DMSO-d₆) δ ppm 8.08 (d, 1H), 7.65-7.77 (m, 2H), 7.55-7.62 (m, 1H), 5.16 (s, 2H), 4.65 (s, 2H), 3.40 (s, 3H); LCMS Rt_{J}=2.05 min, [M+H]⁺=283.0/285.0].

### Building Block B7: 2-(2-(bromomethyl)phenyl)-5-methyloxazole

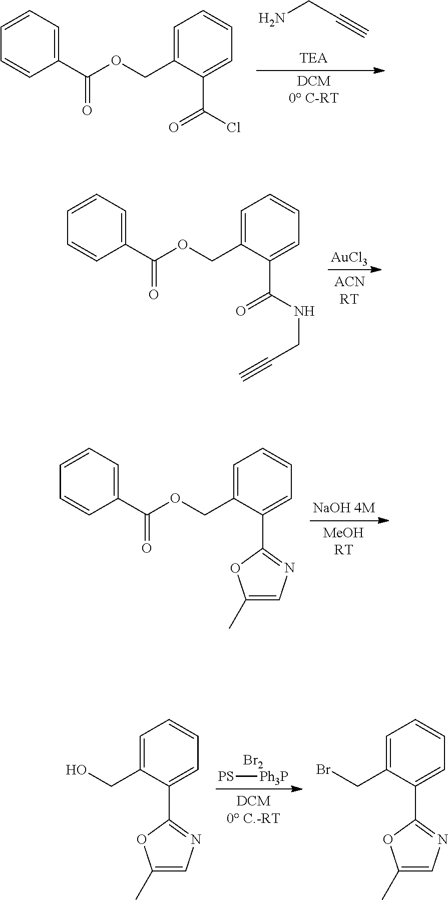

### a) 2-(prop-2-yn-1-ylcarbamoyl)benzyl benzoate

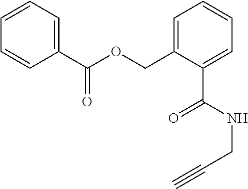

To a solution of 2-(chlorocarbonyl)benzyl benzoate (0.5 g, 1.8 mmol) and triethylamine 0.49 ml, 3.5 mmol) in dry DCM at 0° C. was added propargylamine (0.13 ml, 1.9 mmol). The reaction mixture was stirred at RT for 1 h. To the reaction mixture were added DCM and water. The phases were separated. The aqueous layer was extracted with DCM. The organic phases were washed with brine, combined and dried over sodium sulfate, filtered and concentrated under reduced pressure to afford a beige solid 0.52 g, 99%) which was not further purified. [¹H NMR (400 MHz, DMSO-d₆) δ ppm 8.91 (t, 1H), 7.96 (d, 2H), 7.65 (t, 1H), 7.58-7.39 (m, 6H), 7.51 (t, 2H), 5.48 (s, 2H), 3.96 (dd, 2H); LCMS Rt_{D}=2.44 min, [M+H]⁺=294.0].

### b) 2-(5-methyloxazol-2-yl)benzyl benzoate

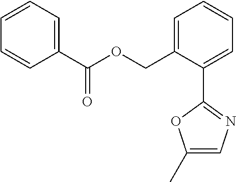

A solution of 2-(prop-2-yn-1-ylcarbamoyl)benzyl benzoate (0.40 g, 1.37 mmol) and AuCl₃ (21 mg, 0.07 mmol) in dry acetonitrile (13 ml) was stirred under argon for 64 h. The solvent was evaporated in vacuo. The residue was purified by flash column chromatography (hexane/ethyl acetate 4:1) to yield the title compound (0.34 g, 84%) as a white solid. LCMS Rt_{F}=2.44 min, [M+H]⁺=294.0].

### c) 2-(2-(bromomethyl)phenyl)-5-methyloxazole

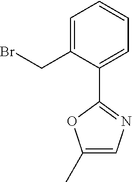

The conversion of 2-(5-methyloxazol-2-yl)benzyl benzoate to 2-(2-(bromomethyl)phenyl)-5-methyloxazole was performed in analogy to the procedure for building block B6.

The title compound was obtained as a beige solid. LCMS Rt_{J}=2.48 min, [M+H]⁺=252.0/254.0].

### Example 1.1

### 4-((1H-indol-3-yl)methyl)-9-(4,6-dimethylpyrimidin-2-yl)-1,4,9-triazaspiro[5.5]undecan-5-one

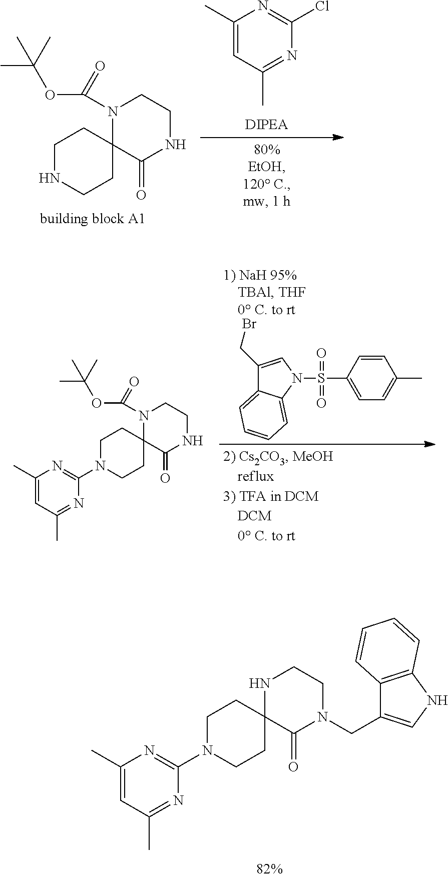

### a) tert-Butyl 9-(4,6-dimethylpyrimidin-2-yl)-5-oxo-1,4,9-triazaspiro[5.5]undecane-1-carbon/late

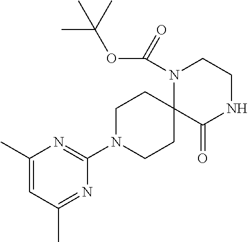

To a solution of tert-butyl 5-oxo-1,4,9-triazaspiro[5.5]undecane-1-carboxylate (building block A1, 267 mg, 0.99 mmol) in ethanol (8 ml) was added 2-chloro-4,6-dimethylpyrimidine (184 mg, 1.3 mmol) and DIPEA (0.69 ml, 4 mmol) in a microwave tube. The tube was sealed and the suspension was heated at 120° C. for 1 h under microwave conditions. The solvent was removed under reduced pressure and the resulting crude product was purified by flash column chromatography (gradient: 0-5% methanol in DCM) to yield the title compound as a white powder (302 mg, 80%). [LCMS Rt_{A}=2.98 min, [M+H]⁺=376.2].

### b) 4-((1H-Indol-3-yl)methyl)-9-(4,6-dimethylpyrimidin-2-yl)-1,4,9-triazaspiro[5.5]undecan-5-one

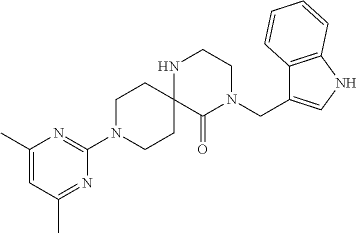

1) To the suspension of tert-butyl 9-(4,6-dimethylpyrimidin-2-yl)-5-oxo-1,4,9-triazaspiro[5.5]undecane-1-carboxylate (282 mg, 0.75 mmol) and TBAI (28 mg, 0.075 mmol) in anhydrous THF (8 ml) was added sodium hydride (95%, 38 mg, 1.5 mmol) at 0° C. After stirring the mixture for 20 min 3-(bromomethyl)-1-tosyl-1H-indole (328 mg, 0.9 mmol) was added and stirring was continued at rt for 2.5 h. Water was added and the aqueous layer was extracted with ethyl acetate. The organic phase was washed with water and brine. The combined organic layers were dried over anhydrous sodium sulfate, filtered and evaporated to give a pale yellow oil. [LCMS Rt_{C}=3.38 min, [M+H]⁺=659.2].

2) To this oil in methanol (5 ml) was added cesium carbonate (2.4 g, 7.5 mmol) and the mixture was refluxed for 40 min. The mixture was diluted with ethyl acetate and water was added. The phases were separated. The aqueous layer was extracted with ethyl acetate. The organic phases were washed with water and brine. The combined organic layers were dried over anhydrous sodium sulfate, filtered and evaporated to give a pale yellow oil. [LCMS Rt_{B}=3.26 min, [M+H]⁺=505.2].

3) The solution of this oil in DCM (3 ml) was treated with TFA (0.58 ml, 7.5 mmol) at rt overnight. Saturated aqueous NaHCO₃ was added and the mixture extracted with ethyl acetate. The organic phase was washed with water and brine. The combined organic layers were dried over anhydrous sodium sulfate, filtered and evaporated to yield an orange oil which was purified by flash column chromatography (gradient: 0-5% methanol in DCM) to yield the title compound as a white powder (250 mg, 82%). [¹H NMR (600 MHz, DMSO-d₆) δ ppm 10.97 (br s, 1H), 7.53 (d, 1H), 7.35 (d, 1H), 7.29 (d, 1H), 7.07 (t, 1H), 6.96 (t, 1H), 6.37 (s, 1H), 4.63 (s, 2H), 4.46 (d, 2H), 3.06-3.19 (m, 4H), 2.77-2.86 (m, 2H), 2.56 (br s, 1H), 2.23 (s, 6H), 1.92 (td, 2H), 1.56 (d, 2H); LCMS Rt_{B}=2.49 min, [M+H]⁺=405.2].

### Example 1.2

### 4-((1H-indol-3-yl)methyl)-9-(4,6-dimethylpyrimidin-2-yl)-1-methyl-1,4,9-triazaspiro[5.5]undecan-5-one

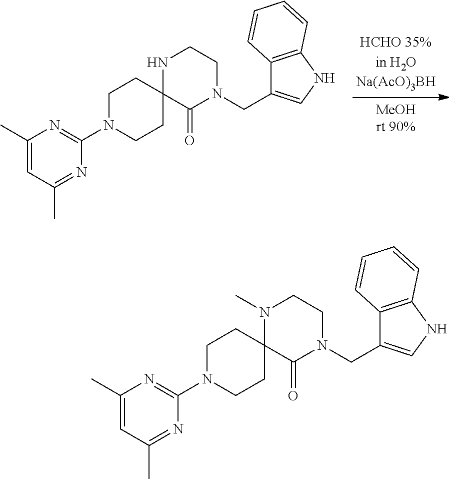

To a solution of 4-((1H-indol-3-yl)methyl)-9-(4,6-dimethylpyrimidin-2-yl)-1,4,9-triazaspiro[5.5]undecan-5-one (Example 1.1, 55 mg, 0.12 mmol) in methanol (1 ml) was added an aqueous solution of formaldehyde (36%, 47 μl, 0.61 mmol), sodium triacetoxy borohydride (182 mg, 0.86 mmol) and acetic acid (49 μl, 0.86 mmol) and the mixture was stirred at rt for 1 h. Water was added and the mixture extracted with ethyl acetate. The organic phase was washed with water and brine and dried over anhydrous sodium sulfate, filtered and evaporated to yield an orange powder which was purified by preparative HPLC (column: Waters Sunfire C18, 5 um, 30×100 mm with guard column 19×10 mm; solvent: Solvent A: water+0.1% TFA; Solvent B: acetonitrile gradient (% B): 10-30% in 16 minutes; flow 50 ml per min) to afford the title compound as a solid (47 mg, (90%). [¹H NMR (600 MHz, DMSO-d₆) δ ppm 10.99 (br s, 1H), 7.54 (d, 1H), 7.35 (d, 1H), 7.31 (d, 1H), 7.07 (t, 1H), 6.97 (t, 1H), 6.38 (s, 1H), 4.65 (s, 2H), 4.37 (m, 2H), 3.24 (t, 2H), 3.12-3.21 (m, 2H), 3.03 (t, 2H), 2.26 (s, 3H), 2.23 (s, 6H), 1.79-1.93 (m, 4H); LCMS Rt_{B}=2.52 min, [M+H]⁺=419.2].

### Example 1.3

### 9-(4,6-Dimethyl-pyrimidin-2-yl)-4-(1-methyl-4-phenyl-1H-pyrazol-3-ylmethyl)-1,4,9-triaza-spiro[5.5]undecan-5-on

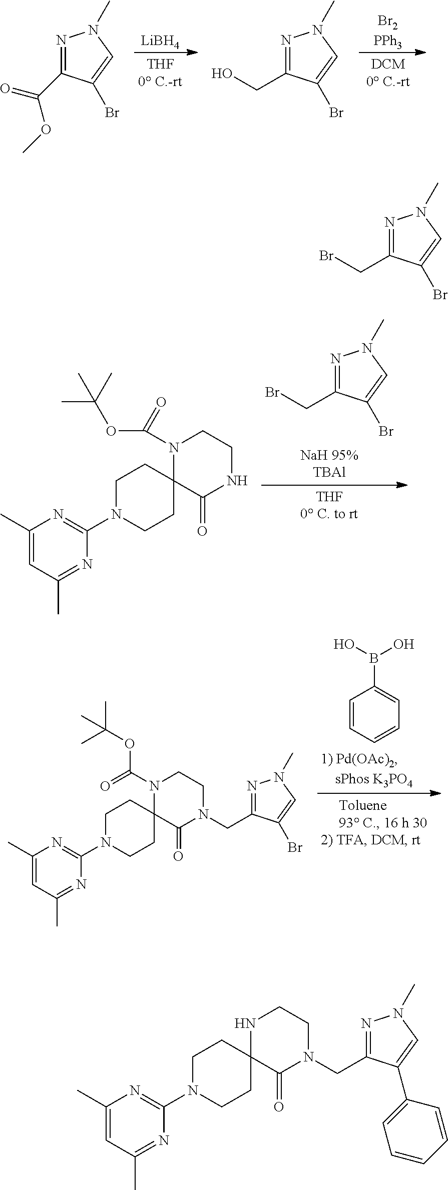

### a) (4-Bromo-1-methyl-1H-pyrazol-3-yl)methanol

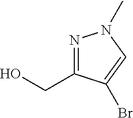

To a solution of methyl 4-bromo-1-methyl-1H-pyrazole-3-carboxylate (3.0 g, 13.7 mmol) in dry THF (90 ml) was added at 0° C. under argon, LiBH₄. The reaction mixture was stirred for 8 h at 0° C. An aqueous solution of acetic acid (1:1 v/v) was added to the reaction mixture at 0° C. until bubbling ceased. The yellow solution was stirred at rt for 16 h. An aqueous solution of NaOH (4M) was added to adjust to pH to 10. The aqueous layer was extracted with DCM and with ethyl acetate. The organic phases were washed with brine, dried over sodium sulfate, filtered and evaporated to give the title compound (2.41 g, 92%) as a white powder. [LCMS Rt_{A}=2.39 min, [M+H]⁺=191.0/193.0].

### b) 4-Bromo-3-(bromomethyl)-1-methyl-1H-pyrazole

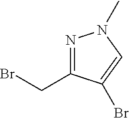

To a solution of PPh₃ (4.7 g, 18 mmol) in dry DCM (70 ml) bromine (0.93 ml, 18 mmol) was added dropwise at 0° C. under argon. The pale yellow suspension was stirred at 0° C. for 10 min. A solution of (4-bromo-1-methyl-1H-pyrazol-3-yl)methanol (2.3 g, 12 mmol) in dry DCM (30 ml) was added at 0° C. and stirring was continued at rt for 45 min. Water was added and the mixture was extracted with DCM. The organic phase was washed with water and brine. The organic layer was dried over sodium sulfate, filtered and evaporated. The resulting oil was purified by flash column chromatography (gradient: diethyl ether in hexane 0-50%) to give the title compound as a solid (2.7 g, 88%). [LCMS Rt_{B}=2.99 min, [M+H]⁺=253.0/254.8/256.8].

### c) tert-Butyl 4-((4-bromo-1-methyl-1H-pyrazol-3-yl)methyl)-9-(4,6-dimethylpyrimidin-2-yl)-5-oxo-1,4,9-triazaspiro[5.5]undecane-1-carboxylate

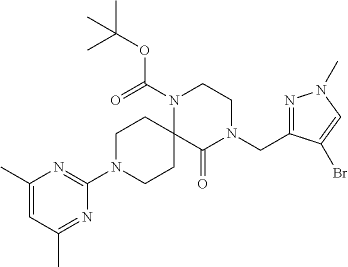

To a white suspension of tert-butyl 9-(4,6-dimethylpyrimidin-2-yl)-5-oxo-1,4,9-triazaspiro[5.5]undecane-1-carboxylate (120 mg, 0.32 mmol) and TBAI (12 mg, 0.032 mmol) in anhydrous THF (3 ml) sodium hydride (16 mg, 0.64 mmol) was added under argon at 0° C. The reaction mixture was stirred for 20 min. 4-Bromo-3-(bromomethyl)-1-methyl-1H-pyrazole (135 mg, 0.43 mmol) was then added at 0° C. The resulting pale yellow suspension was stirred at rt for 3 h. The reaction mixture was cooled again to 0° C. and NaH (16 mg, 0.64 mmol) was added. After 20 min at 0° C. a solution of 4-Bromo-3-(bromomethyl)-1-methyl-1H-pyrazole (81 mg, 0.32 mmol) in dry THF was added. The reaction mixture was stirred for 2.5 h at rt. According to LC-MS 2, the reaction was finished.

A few drops of water was added to the reaction mixture and the resulting clear solution was purified by flash column chromatography (gradient: MeOH in DCM: 0 to 5%) to give the title compound as a colorless oil (174 mg, 99%). [LCMS Rt_{B}=3.13 min, [M+H]⁺=548.2/550.2].

### d) 9-(4,6-dimethylpyrimidin-2-yl)-4-((1-methyl-4-phenyl-1H-pyrazol-3-yl)methyl)-1,4,9-triazaspiro[5.5]undecan-5-one

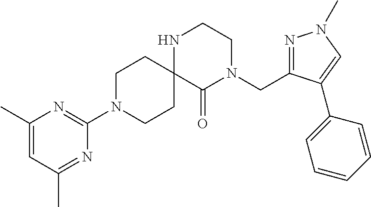

The mixture of tert-butyl 4-((4-bromo-1-methyl-1H-pyrazol-3-yl)methyl)-9-(4,6-dimethylpyrimidin-2-yl)-5-oxo-1,4,9-triazaspiro[5.5]undecane-1-carboxylate (100 mg, 0.16 mmol) in toluene (0.7 ml), phenylboronic acid (26 mg, 0.21 mmol), K₃PO₄ (87 mg, 0.41 mmol), Pd(OAc)₂ (1.8 mg, 0.0082 mmol) and s-Phos (6.7 mg, 16.4 mmol) was heated in a closed vial at 93° C. for 16 h under argon. After cooling to rt water was added and the mixture was extracted with DCM. The organic phase was washed with water and brine, dried over sodium sulfate, filtered and evaporated to yield an orange oil which was dissolved in DCM (3 ml). TFA (1 ml) was added dropwise and the resulting yellow solution was stirred for 90 min at rt. The reaction was quenched by adding a saturated aqueous solution of NaHCO₃ to adjust to pH 10. The mixture was extracted with DCM and the organic phase was washed with water and brine. The combined organic layers were dried over sodium sulfate, filtered and evaporated to afford an orange oil which was purified by preparative HPLC (column: Waters Sunfire C18, 5 um, 30×100 mm with guard column 19×10 mm; solvent: Solvent A: water+0.1% TFA; Solvent B: acetonitrile gradient (% B): 10-30% in 16 minutes; flow 50 ml per min) to afford after removal of TFA a white foam. Crystallization from diethyl ether gave the title compound as white crystals (22 mg, 30%).

[1H NMR (400 MHz, DMSO-d₆) δ ppm 7.92 (s, 1H), 7.26-7.38 (m, 4H), 7.17-7.24 (m, 1H), 6.33 (s, 1H), 4.63 (s, 2H), 4.38 (d, 2H), 3.81 (s, 3H), 3.81 (s, 3H), 3.04-3.16 (m, 4H), 2.77 (t, 2H), 2.20 (s, 6H), 1.78 (td, 2H), 1.44 (d, 2H); LCMS Rt_{B}=2.56 min, [M+H]⁺=446.2].

### Example 1.4

### 9-(4,6-Dimethylpyrimidin-2-yl)-4-((5-fluoro-1H-indol-3-yl)methyl)-1,4,9-triazaspiro[5.5]undecan-5-one

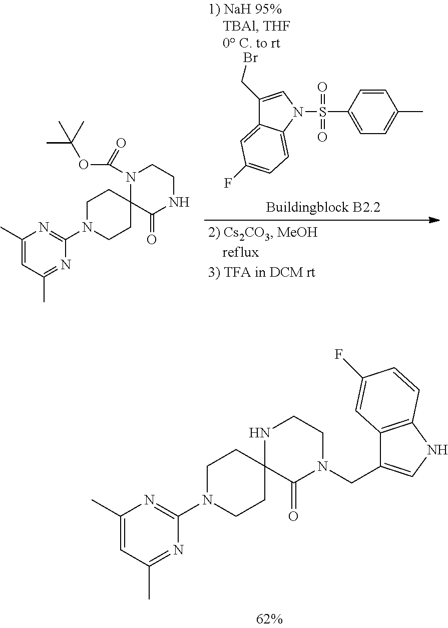

To a suspension of tert-butyl 9-(4,6-dimethylpyrimidin-2-yl)-5-oxo-1,4,9-triazaspiro[5.5]undecane-1-carboxylate (130 mg, 0.35 mmol) and TBAI (13 mg, 0.035 mmol) in anhydrous THF (3 ml) was added sodium hydride (17 mg, 0.69 mmol (95%) at 0° C. and the mixture was stirred for 15 min. 3-(Bromomethyl)-5-fluoro-1-tosyl-1H-indole (146 mg, 0.38 mmol) was then added at 0° C. The mixture was allowed to warm to rt and stirred for 30 min. The reaction mixture was diluted with DCM and water. The organic phase was washed with water and brine. The combined organic layers were dried over sodium sulfate, filtered and evaporated to afford an orange oil which was used in the next step without further purification. [LCMS Rt_{C}=2.58 min, [M+H]⁺=677.2].

To the solution of the above orange oil in MeOH (4 ml) Cs₂O₃ (1.1 g, 3.5 mmol) was added and the mixture refluxed for 30 min. The reaction mixture was diluted with DCM and water was added. The phases were separated and the aqueous layer was extracted with DCM. The organic phase was washed with water and brine. The combined organic layers were dried over sodium sulfate, filtered and evaporated to yield an orange oil which was dissolved in DCM (3 ml) and TFA (2 ml, 26 mmol) was added dropwise. The resulting orange solution was stirred for 15 min at rt. The reaction was quenched by adding a saturated solution of NaHCO₃ to adjust to pH 10. DCM was added and the phases were separated. The aqueous layer was extracted with DCM. The organic phase was washed with water and brine. The combined organic layers were dried over sodium sulfate, filtered and evaporated to yield an orange oil. The oil was purified by flash column chromatography (gradient of MeOH in DCM: 0 to 5%) and subsequent crystallization from diethyl ether/hexane to afford the title compound as colorless crystals (93 mg, 62%). [¹H NMR (400 MHz, DMSO-d₆) δ 11.04 (br s, 1H), 7.36 (d, 1H), 7.32 (dd, 1H), 7.25 (dd, 1H), 6.89 (dt, 1H), 6.35 (s, 1H), 4.56 (s, 2H), 4.44 (d, 2H), 3.05-3.18 (m, 4H), 2.81 (br s, 2H), 2.51-2.64 (m, 1H), 2.20 (s, 6H), 1.90 (dt, 2H), 1.54 (d, 2H); LCMS Rt_{B}=2.56 min, [M+H]⁺=423.2].

The following examples were obtained by analogous methods as described for examples 1.1 to 1.4:

| | | | LCMS | |
|---|---|---|---|---|
| | | | Rt [min], | |
| Ex. | Structure | Name | method | [M + H]⁺ |
| 1.5 | 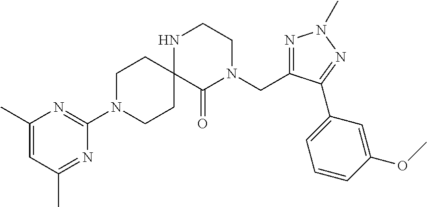 | 9-(4,6- Dimethylpyrimidin-2- yl)-4-((5-(3- methoxyphenyl)-2- methyl-2H-1,2,3- triazol-4-yl)methyl)- 1,4,9- triazaspiro[5.5] undecan-5-one | 2.63 (B) | 477.2 |
| 1.6 | 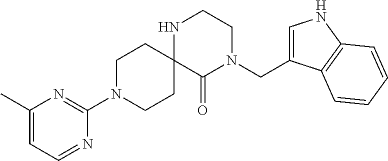 | 4-((1H-Indol-3- yl)methyl)-9-(4- methylpyrimidin-2- yl)-1,4,9- triazaspiro[5.5] undecan-5-one | 2.53 (B) | 391.2 |
| 1.7 | 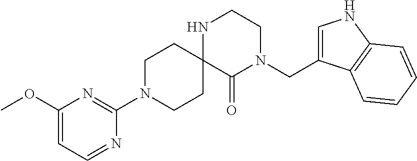 | 4-((1H-Indol-3- yl)methyl)-9-(4- methoxypyrimidin- 2-yl)-1,4,9- triazaspiro[5.5] undecan-5-one | 2.44 (B) | 407.2 |
| 1.8 | 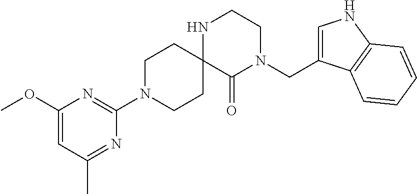 | 4-((1H-Indol-3- yl)methyl)-9-(4- methoxy-6- methylpyrimidin-2- yl)-1,4,9- triazaspiro[5.5] undecan-5-one | 2.49 (B) | 421.2 |
| 1.9 | 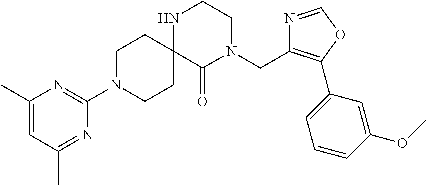 | 9-(4,6- Dimethylpyrimidin- 2-yl)-4-((5-(3- methoxyphenyl) oxazol-4-yl)methyl)- 1,4,9- triazaspiro[5.5] undecan-5-one | 2.66 (B) | 463.2 |
| 1.10 | 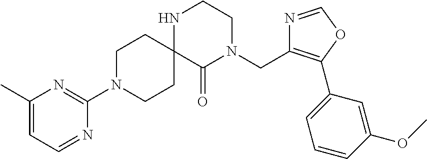 | 4-((5-(3- Methoxyphenyl) oxazol-4-yl)methyl)- 9-(4-methyl- pyrimidin-2-yl)- 1,4,9- triazaspiro[5.5] undecan-5-one | 2.65 (B) | 449.2 |
| 1.11 | 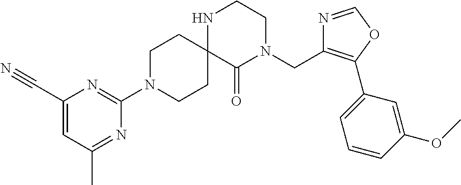 | 2-(4-((5-(3- Methoxyphenyl) oxazol-4-yl)methyl)- 5-oxo-1,4,9- triazaspiro[5.5] undecan-9-yl)-6- methylpyrimidine- 4-carbonitrile | 2.93 (B) | 474.2 |
| 1.12 | 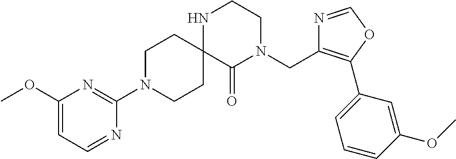 | 4-((5-(3- Methoxyphenyl) oxazol-4-yl)methyl)- 9-(4-methoxy- pyrimidin-2- yl)-1,4,9- triazaspiro[5.5] undecan-5-one | 2.58 (B) | 465.2 |
| 1.13 | 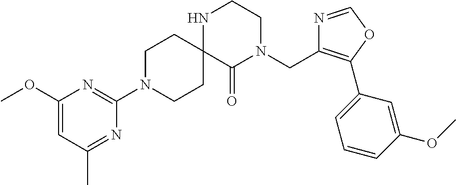 | 9-(4-Methoxy-6- methylpyrimidin- 2-yl)-4-((5-(3- methoxyphenyl) oxazol-4-yl)methyl)- 1,4,9- triazaspiro[5.5] undecan-5-one | 2.63 (B) | 479.2 |
| 1.14 | 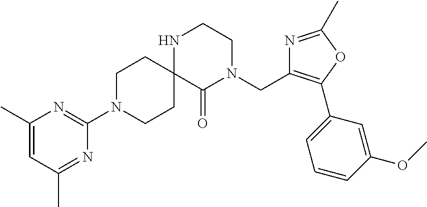 | 9-(4,6- Dimethylpyrimidin- 2-yl)-4-((5-(3- methoxyphenyl)-2- methyloxazol-4- yl)methyl)-1,4,9- triazaspiro[5.5] undecan-5-one | 2.72 (B) | 477.2 |
| 1.15 | 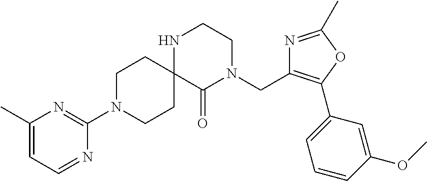 | 4-((5-(3- Methoxyphenyl)-2- methyloxazol-4- yl)methyl)-9-(4- methylpyrimidin- 2-yl)-1,4,9- triazaspiro[5.5] undecan-5-one | 2.73 (B) | 463.2 |
| 1.16 | 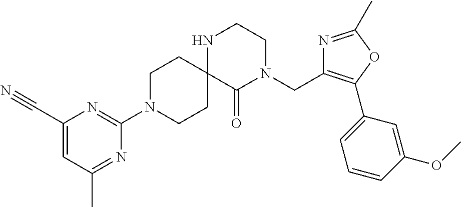 | 2-(4-((5-(3- Methoxyphenyl)-2- methyloxazol-4- yl)methyl)-5-oxo- 1,4,9- triazaspiro[5.5] undecan-9-yl)-6- methylpyrimidine- 4-carbonitrile | 3.00 (B) | 488.2 |
| 1.17 | 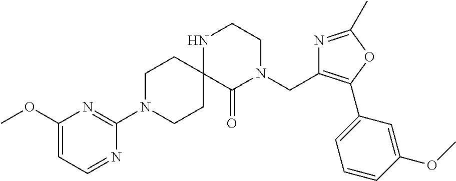 | 4-((5-(3- Methoxyphenyl)-2- methyloxazol-4- yl)methyl)-9-(4- methoxypyrimidin- 2-yl)-1,4,9- triazaspiro[5.5] undecan-5-one | 2.63 (B) | 479.2 |
| 1.18 | 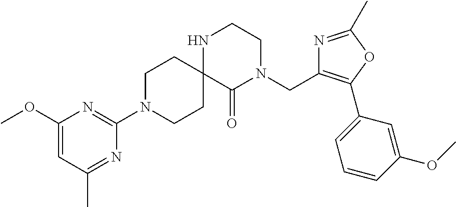 | 9-(4-Methoxy-6- methylpyrimidin- 2-yl)-4-((5-(3- methoxyphenyl)-2- methyloxazol-4- yl)methyl)-1,4,9- triazaspiro[5.5] undecan-5-one | 2.71 (B) | 493.2 |
| 1.19 | 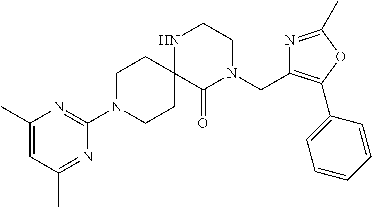 | 9-(4,6-Dimethyl- pyrimidin-2-yl)-4- ((2-methyl-5- phenyloxazol-4- yl)methyl)-1,4,9- triazaspiro[5.5] undecan-5-one | 2.67 (B) | 447.2 |
| 1.20 | 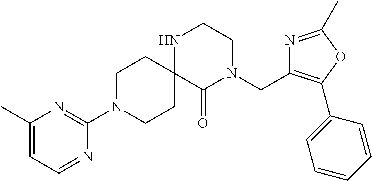 | 9-(4-Methyl- pyrimidin-2-yl)- 4-((2-methyl-5- phenyloxazol-4- yl)methyl)-1,4,9- triazaspiro[5.5] undecan-5-one | 2.687 (B) | 433.2 |
| 1.21 | 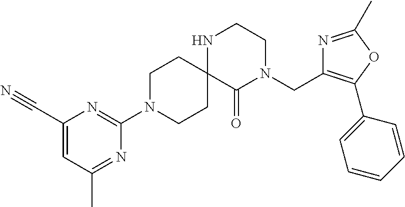 | 6-Methyl-2-(4-((2- methyl-5- phenyloxazol-4- yl)methyl)-5-oxo- 1,4,9- triazaspiro[5.5] undecan-9-yl) pyrimidine-4- carbonitrile | 2.96 (B) | 458.2 |
| 1.22 | 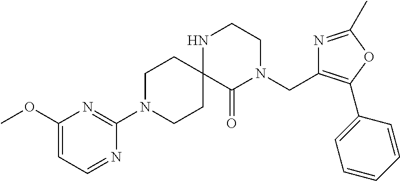 | 9-(4-Methoxy- pyrimidin-2-yl)-4- ((2-methyl-5- phenyloxazol-4- yl)methyl)-1,4,9- triazaspiro[5.5] undecan-5-one | 2.58 (B) | 449.2 |
| 1.23 | 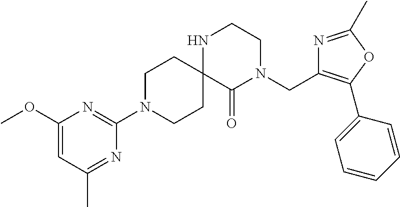 | 9-(4-Methoxy-6- methylpyrimidin- 2-yl)-4-((2-methyl- 5-phenyloxazol-4- yl)methyl)-1,4,9- triazaspiro[5.5] undecan-5-one | 2.63 (B) | 463.2 |
| 1.24 | 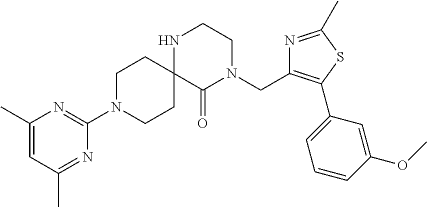 | 9-(4,6-Dimethyl- pyrimidin-2- yl)-4-((5-(3- methoxyphenyl)-2- methylthiazol-4- yl)methyl)-1,4,9- triazaspiro[5.5] undecan-5-one | 2.78 (B) | 493.2 |
| 1.25 | 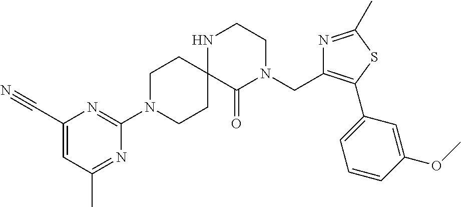 | 2-(4-((5-(3- Methoxyphenyl)-2- methylthiazol-4- yl)methyl)-5-oxo- 1,4,9- triazaspiro[5.5] undecan-9-yl)-6- methylpyrimidine- 4-carbonitrile | 3.07 (B) | 504.2 |
| 1.26 | 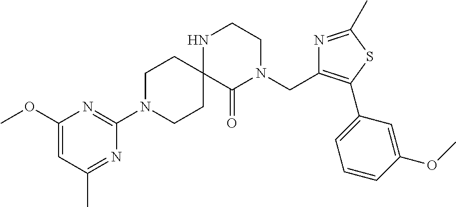 | 9-(4-Methoxy-6- methylpyrimidin-2- yl)-4-((5-(3- methoxyphenyl)-2- methylthiazol-4- yl)methyl)-1,4,9- triazaspiro[5.5] undecan-5-one | 2.73 (B) | 509.2 |
| 1.27 | 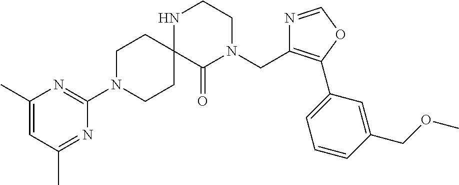 | 9-(4,6-Dimethyl- pyrimidin-2- yl)-4-((5-(3- (methoxymethyl) phenyl)oxazol-4- yl)methyl)-1,4,9- triazaspiro[5.5] undecan-5-one | 2.61 (B) | 477.2 |
| 1.28 | 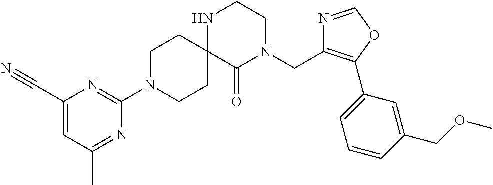 | 2-(4-((5-(3- (Methoxymethyl) phenyl)oxazol-4- yl)methyl)-5-oxo- 1,4,9- triazaspiro[5.5] undecan-9-yl)-6- methylpyrimidine- 4-carbonitrile | 2.88 (B) | 488.2 |
| 1.29 | 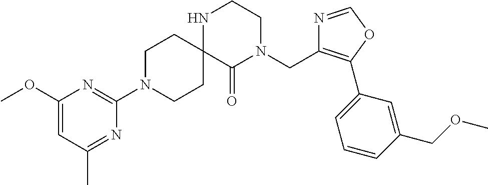 | 9-(4-Methoxy-6- methylpyrimidin- 2-yl)-4-((5-(3- (methoxymethyl) phenyl)oxazol-4- yl)methyl)-1,4,9- triazaspiro[5.5] undecan-5-one | 2.58 (B) | 493.2 |
| 1.30 | 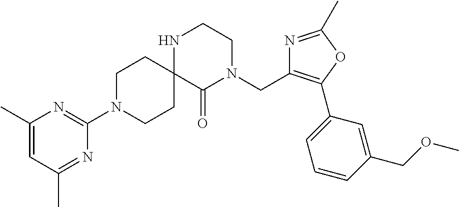 | 9-(4,6-Dimethyl- pyrimidin-2- yl)-4-((5-(3- (methoxymethyl) phenyl)-2-methyl- oxazol-4- yl)methyl)-1,4,9- triazaspiro[5.5] undecan-5-one | 2.67 (B) | 491.2 |
| 1.31 | 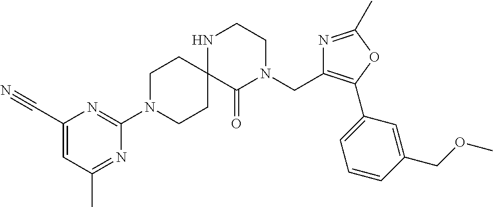 | 2-(4-((5-(3- (Methoxymethyl) phenyl)-2-methyl- oxazol-4- yl)methyl)-5-oxo- 1,4,9- triazaspiro[5.5] undecan-9-yl)-6- methylpyrimidine- 4-carbonitrile | 2.94 (B) | 502.2 |
| 1.32 | 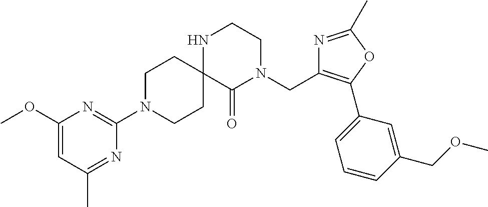 | 9-(4-Methoxy-6- methylpyrimidin- 2-yl)-4-((5-(3- (methoxymethyl) phenyl)-2-methyl- oxazol-4- yl)methyl)-1,4,9- triazaspiro[5.5] undecan-5-one | 2.65 (B) | 507.2 |
| 1.33 | 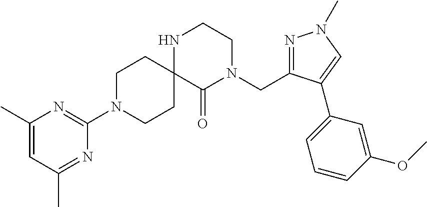 | 9-(4,6-Dimethyl- pyrimidin-2- yl)-4-((4-(3- methoxyphenyl)-1- methyl-1H-pyrazol- 3-yl)methyl)-1,4,9- triazaspiro[5.5] undecan-5-one | 2.61 (B) | 476.2 |
| 1.34 | 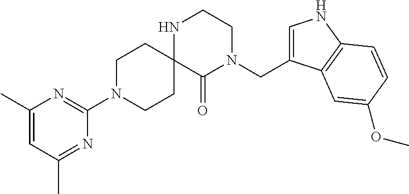 | 9-(4,6-Dimethyl- pyrimidin-2-yl)- 4-((5-methoxy-1H- indol-3-yl)methyl)- 1,4,9- triazaspiro[5.5] undecan-5-one | 2.49 (B) | 435.2 |
| 1.35 | 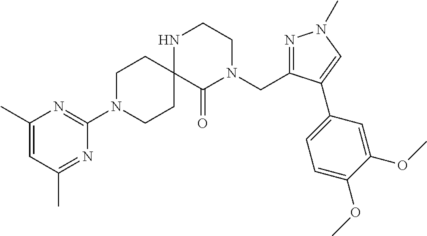 | 4-((4-(3,4- Dimethoxyphenyl)- 1-methyl-1H- pyrazol-3-yl) methyl)-9-(4,6- dimethylpyrimidin- 2-yl)-1,4,9- triazaspiro[5.5] undecan-5-one | 2.51 (B) | 506.2 |
| 1.36 | 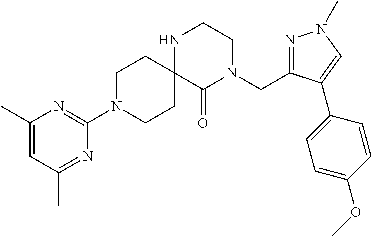 | 9-(4,6-Dimethyl- pyrimidin-2- yl)-4-((4-(4- methoxyphenyl)-1- methyl-1H-pyrazol- 3-yl)methyl)-1,4,9- triazaspiro[5.5] undecan-5-one | 2.59 (B) | 476.2 |
| 1.37 | 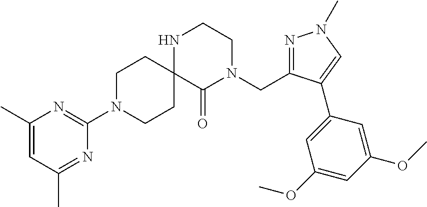 | 4-((4-(3,5- Dimethoxyphenyl)- 1-methyl-1H- pyrazol-3-yl) methyl)-9-(4,6- dimethylpyrimidin- 2-yl)-1,4,9- triazaspiro[5.5] undecan-5-one | 2.64 (B) | 506.2 |
| 1.38 | 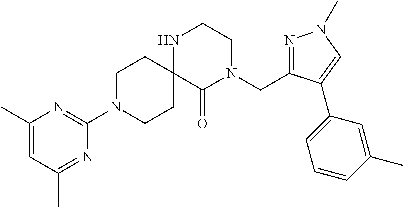 | 9-(4,6-Dimethyl- pyrimidin-2-yl)- 4-((1-methyl-4-(m- tolyl)-1H-pyrazol- 3-yl)methyl)-1,4,9- triazaspiro[5.5] undecan-5-one | 2.70 (B) | 460.2 |
| 1.39 | 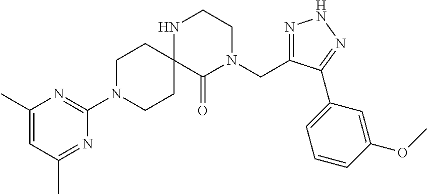 | 9-(4,6-Dimethyl- pyrimidin-2-yl)-4- ((5-(3-methoxy- phenyl)-2H- 1,2,3-triazol-4- yl)methyl)-1,4,9- triazaspiro[5.5] undecan-5-one | 2.49 (B) | 463.2 |
| 1.40 | 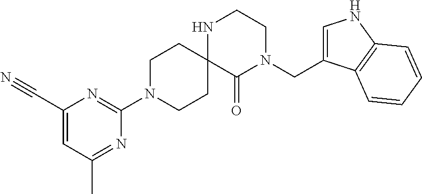 | 2-(4-((1H-Indol-3- yl)methyl)-5-oxo- 1,4,9- triazaspiro[5.5] undecan-9-yl)-6- methylpyrimidine- 4-carbonitrile | 3.00 (B) | 416.2 |
| 1.41 | 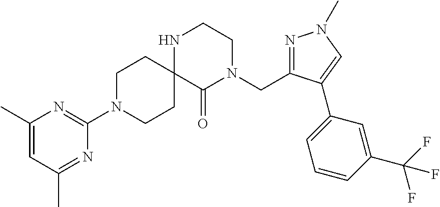 | 9-(4,6-Dimethyl- pyrimidin-2-yl)- 4-((1-methyl-4-(3- (trifluoromethyl) phenyl)-1H- pyrazol-3-yl) methyl)-1,4,9- triazaspiro[5.5] undecan-5-one | 2.83 (B) | 514.2 |
| 1.42 | 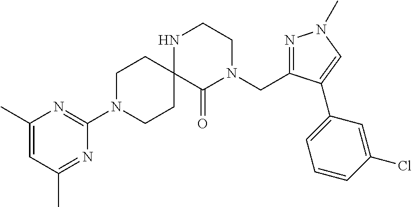 | 4-((4-(3- Chlorophenyl)-1- methyl-1H-pyrazol- 3-yl)methyl)-9- (4,6-dimethyl- pyrimidin-2-yl)- 1,4,9- triazaspiro[5.5] undecan-5-one | 2.73 (B) | 480.2 |
| 1.43 | 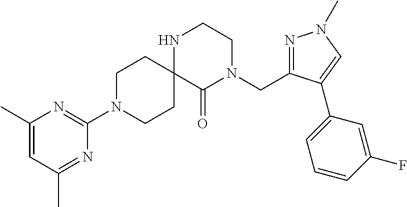 | 9-(4,6-Dimethyl- pyrimidin-2- yl)-4-((4-(3- fluorophenyl)-1- methyl-1H-pyrazol- 3-yl)methyl)- 1,4,9- triazaspiro[5.5] undecan-5-one | 2.62 (B) | 464.2 |
| 1.44 | 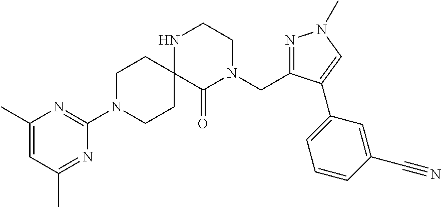 | 3-(3-((9-(4,6- Dimethylpyrimidin- 2-yl)-5-oxo- 1,4,9- triazaspiro[5.5] undecan-4-yl) methyl)-1-methyl- 1H-pyrazol-4- yl)benzonitrile | 2.55 (B) | 471.2 |
| 1.45 | 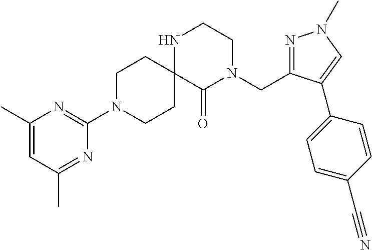 | 4-(3-((9-(4,6- Dimethylpyrimidin- 2-yl)-5-oxo- 1,4,9- triazaspiro[5.5] undecan-4-yl) methyl)-1-methyl- 1H-pyrazol-4- yl)benzonitrile | 2.55 (B) | 471.2 |
| 1.46 | 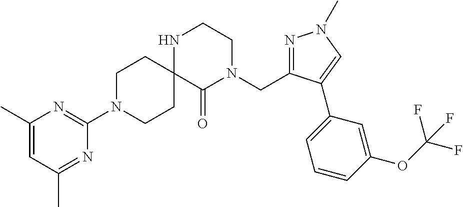 | 9-(4,6-Dimethyl- pyrimidin-2- yl)-4-((1-methyl-4- (3-(trifluoro- methoxy)phenyl)- 1H-pyrazol-3- yl)methyl)-1,4,9- triazaspiro[5.5] undecan-5-one | 2.86 (B) | 530.2 |
| 1.47 | 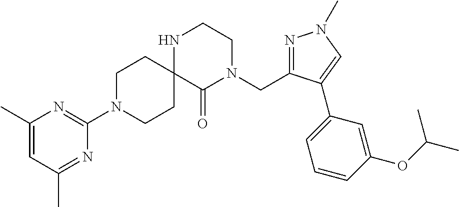 | 9-(4,6-Dimethyl- pyrimidin-2- yl)-4-((4-(3-iso- propoxyphenyl)-1- methyl-1H-pyrazol- 3-yl)methyl)-1,4,9- triazaspiro[5.5] undecan-5-one | 2.80 (B) | 504.2 |
| 1.48 | 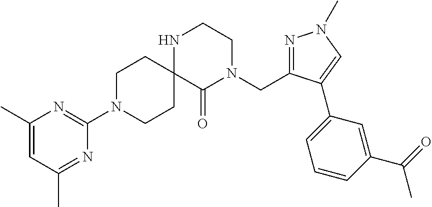 | 4-((4-(3- Acetylphenyl)-1- methyl-1H-pyrazol- 3-yl)methyl)-9- (4,6-dimethyl- pyrimidin-2-yl)- 1,4,9- triazaspiro[5.5] undecan-5-one | 2.51 (B) | 488.2 |
| 1.49 | 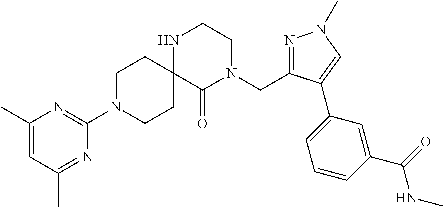 | 3-(3-((9-(4,6- Dimethylpyrimidin- 2-yl)-5-oxo-1,4,9- triazaspiro[5.5] undecan-4-yl) methyl)-1-methyl- 1H-pyrazol-4-yl)- N-methylbenzamide | 2.33 (B) | 503.2 |
| 1.50 | 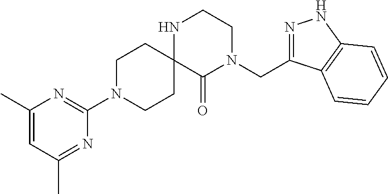 | 4-((1H-Indazol-3- yl)methyl)-9-(4,6- dimethylpyrimidin- 2-yl)-1,4,9- triazaspiro[5.5] undecan-5-one | 2.44 (B) | 406.2 |
| 1.51 | 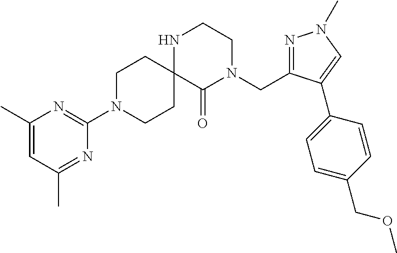 | 9-(4,6-Dimethyl- pyrimidin-2- yl)-4-((4-(4- (methoxymethyl) phenyl)-1-methyl- 1H-pyrazol-3- yl)methyl)-1,4,9- triazaspiro[5.5] undecan-5-one | 2.58 (B) | 490.2 |
| 1.52 | 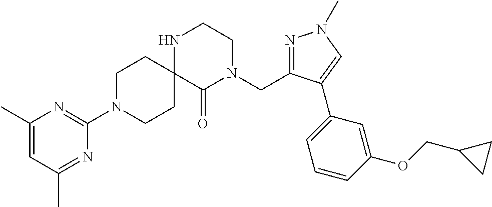 | 4-((4-(3-(Cyclo- propylmethoxy) phenyl)-1-methyl- 1H-pyrazol-3-yl) methyl)-9-(4,6- dimethylpyrimidin- 2-yl)-1,4,9- triazaspiro[5.5] undecan-5-one | 2.86 (B) | 516.2 |
| 1.53 | 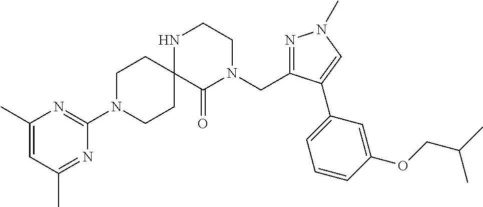 | 9-(4,6-Dimethyl- pyrimidin-2- yl)-4-((4-(3- isobutoxyphenyl)-1- methyl-1H-pyrazol- 3-yl)methyl)-1,4,9- triazaspiro[5.5] undecan-5-one | 3.01 (B) | 518.2 |
| 1.54 | 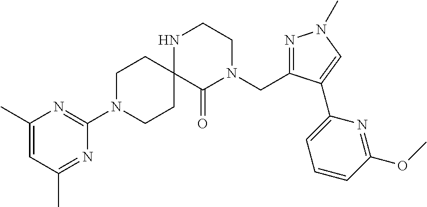 | 9-(4,6-Dimethyl- pyrimidin-2- yl)-4-((4-(6- methoxypyridin-2- yl)-1-methyl-1H- pyrazol-3-yl) methyl)-1,4,9- triazaspiro[5.5] undecan-5-one | 2.57 (B) | 477.2 |
| 1.55 | 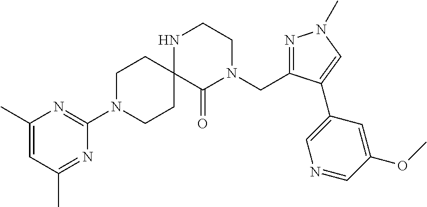 | 9-(4,6-Dimethyl- pyrimidin-2- yl)-4-((4-(5- methoxypyridin-3- yl)-1-methyl-1H- pyrazol-3-yl) methyl)-1,4,9- triazaspiro[5.5] undecan-5-one | 2.41 (A) | 477.2 |
| 1.56 | 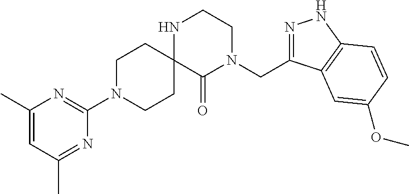 | 9-(4,6-Dimethyl- pyrimidin-2- yl)-4-((5-methoxy- 1H-indazol-3-yl) methyl)-1,4,9- triazaspiro[5.5] undecan-5-one | 2.45 (B) | 436.2 |
| 1.57 | 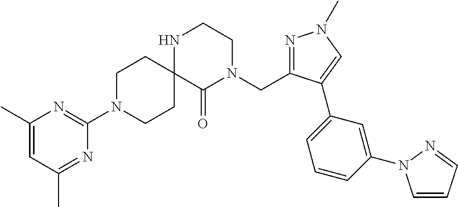 | 4-((4-(3-(1H-Pyrazol- 1-yl)phenyl)-1- methyl-1H-pyrazol- 3-yl)methyl)-9- (4,6-dimethyl- pyrimidin-2-yl)- 1,4,9- triazaspiro[5.5] undecan-5-one | 2.62 (B) | 512.2 |
| 1.58 | 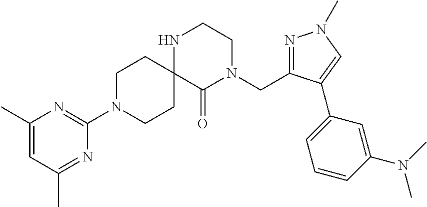 | 4-((4-(3-Dimethyl- amino)phenyl)-1- methyl-1H-pyrazol- 3-yl)methyl)- 9-(4,6-dimethyl- pyrimidin-2- yl)-1,4,9- triazaspiro[5.5] undecan-5-one | 2.27 (B) | 489.2 |
| 1.59 | 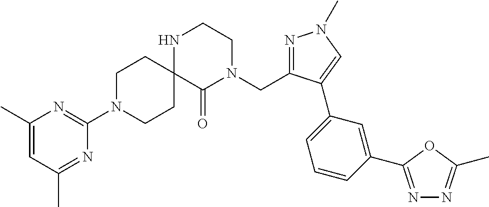 | 9-(4,6-Dimethyl- pyrimidin-2- yl)-4-((1-methyl- 4-(3-(5-methyl- 1,3,4-oxadiazol-2- yl)phenyl)-1H- pyrazol-3-yl) methyl)-1,4,9- triazaspiro[5.5] undecan-5-one | 2.79 (A) | 528.2 |
| 1.60 | 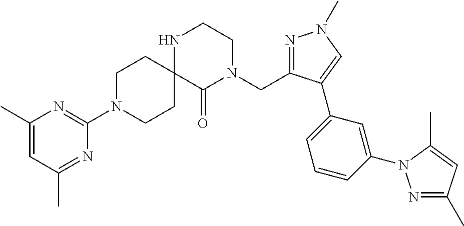 | 4-((4-(3-(3,5- Dimethyl-1H- pyrazol-1-yl) phenyl)-1-methyl- 1H-pyrazol-3- yl)methyl)-9-(4,6- dimethylpyrimidin- 2-yl)-1,4,9- triazaspiro[5.5] undecan-5-one | 2.70 (B) | 540.2 |
| 1.61 | 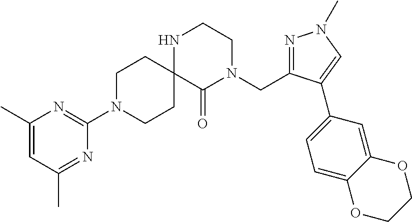 | 4-((4-(2,3-Dihydro- benzo[b][1,4]dioxin- 6-yl)-1-methyl- 1H-pyrazol-3-yl) methyl)-9-(4,6- dimethylpyrimidin- 2-yl)-1,4,9- triazaspiro[5.5] undecan-5-one | 2.57 (B) | 504.2 |
| 1.62 | 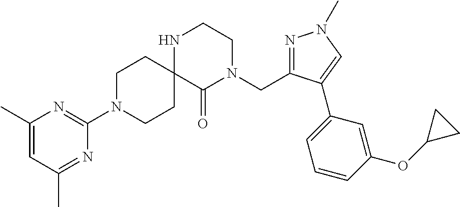 | 4-((4-(3-Cyclo- propoxyphenyl)-1- methyl-1H-pyrazol- 3-yl)methyl)-9- (4,6-dimethyl- pyrimidin-2-yl)- 1,4,9- triazaspiro[5.5] undecan-5-one | 2.76 (B) | 502.2 |
| 1.63 | 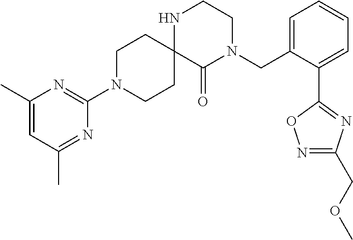 | 9-(4,6-dimethyl- pyrimidin-2- yl)-4-(2-(3- (methoxymethyl)- 1,2,4-oxadiazol-5- yl)benzyl)-1,4,9- triazaspiro[5.5] undecan-5-one | 2.53 (B) | 478.2 |
| 1.64 | 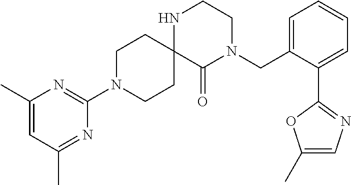 | 9-(4,6-dimethyl- pyrimidin-2- yl)-4-(2-(5- methyloxazol-2- yl)benzyl)-1,4,9- triazaspiro[5.5] undecan-5-one | 2.60 (B) | 447.2 |
| 1.65 | 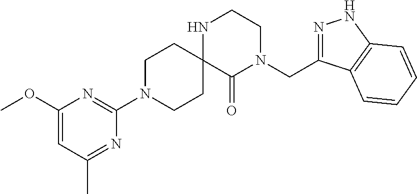 | 4-((1H-indazol-3- yl)methyl)-9-(4- methoxy-6-methyl- pyrimidin-2-yl)- 1,4,9- triazaspiro[5.5] undecan-5-one | 2.69 (A) | 422.2 |
| 1.66 | 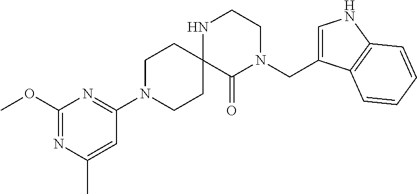 | 4-((1H-indol-3- yl)methyl)-9-(2- methoxy-6-methyl- pyrimidin-4-yl)- 1,4,9- triazaspiro[5.5] undecan-5-one | 2.68 (A) | 421.2 |
| 1.67 | 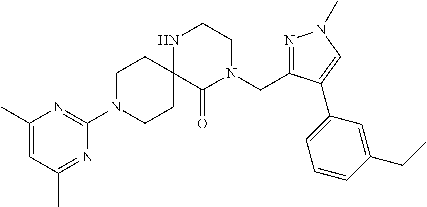 | 9-(4,6-dimethyl- pyrimidin-2-yl)- 4-((4-(3-ethyl- phenyl)-1-methyl- 1H-pyrazol-3-yl) methyl)-1,4,9- triazaspiro[5.5] undecan-5-one | 2.80 (B) | 474.2 |
| 1.68 | 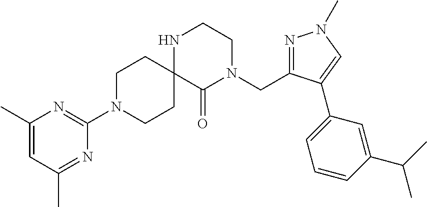 | 9-(4,6-dimethyl- pyrimidin-2-yl)- 4-((4-(3-isopropyl- phenyl)-1-methyl- 1H-pyrazol-3-yl) methyl)-1,4,9- triazaspiro[5.5] undecan-5-one | 2.91 (B) | 488.2 |
| 1.69 | 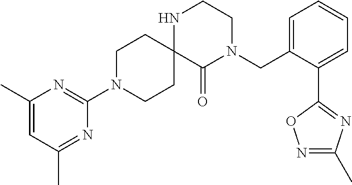 | 9-(4,6-dimethyl- pyrimidin-2-yl)- 4-(2-(3-methyl- 1,2,4-oxadiazol-5- yl)benzyl)-1,4,9- triazaspiro[5.5] undecan-5-one | 2.55 (B) | 448.2 |
| 1.70 | 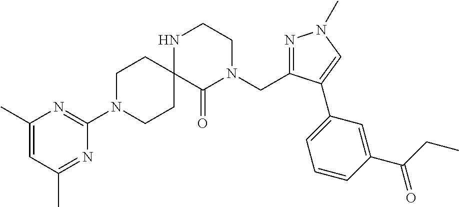 | 9-(4,6-dimethyl- pyrimidin-2-yl)- 4-((1-methyl-4-(3- propionylphenyl)- 1H-pyrazol-3-yl) methyl)-1,4,9- triazaspiro[5.5] undecan-5-one | 2.64 (B) | 502.2 |
| 1.71 | 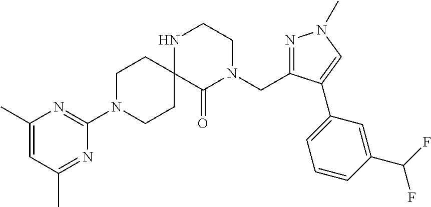 | 4-((4-(3- (difluoromethyl) phenyl)-1-methyl- 1H-pyrazol-3-yl) methyl)-9-(4,6- dimethylpyrimidin- 2-yl)-1,4,9- triazaspiro[5.5] undecan-5-one | 2.68 (B) | 496.2 |
| 1.72 | 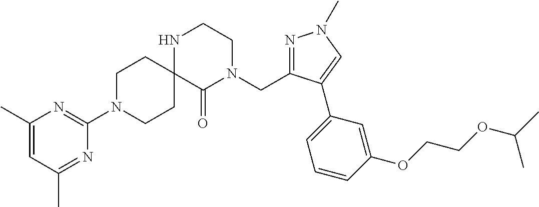 | 9-(4,6-dimethyl- pyrimidin-2-yl)- 4-((4-(3-(2-iso- propoxyethoxy) phenyl)-1-methyl- 1H-pyrazol-3-yl) methyl)-1,4,9- triazaspiro[5.5] undecan-5-one | 2.80 (B) | 548.2 |
| 1.73 | 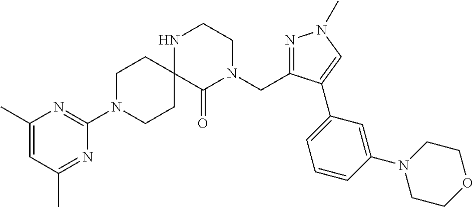 | 9-(4,6-dimethyl- pyrimidin-2-yl)- 4-((1-methyl-4-(3- morpholinophenyl)- 1H-pyrazol-3- yl)methyl)-1,4,9- triazaspiro[5.5] undecan-5-one | 2.76 (A) | 531.2 |
| 1.74 | 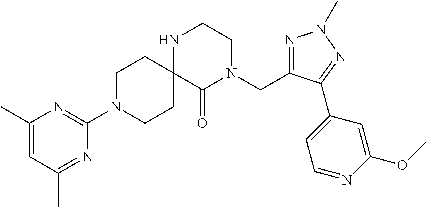 | 9-(4,6-dimethyl- pyrimidin-2-yl)- 4-((5-(2-methoxy- pyridin-4-yl)-2- methyl-2H-1,2,3- triazol-4-yl) methyl)-1,4,9- triazaspiro[5.5] undecan-5-one | 2.42 (B) | 478.2 |
| 1.75 | 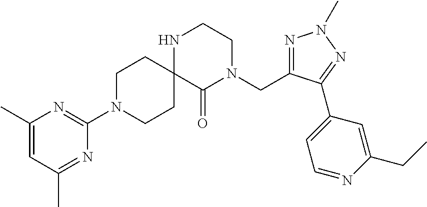 | 9-(4,6-dimethyl- pyrimidin-2-yl)- 4-((5-(2-ethyl- pyridin-4-yl)-2- methyl-2H-1,2,3- triazol-4-yl) methyl)-1,4,9- triazaspiro[5.5] undecan-5-one | 2.48 (A) | 476.2 |
| 1.76 | 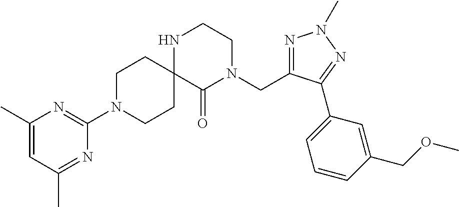 | 9-(4,6-dimethyl- pyrimidin-2-yl)- 4-((5-(3-(methoxy- methyl)phenyl)-2- methyl-2H-1,2,3- triazol-4-yl) methyl)-1,4,9- triazaspiro[5.5] undecan-5-one | 2.54 (B) | 491.2 |
| 1.77 | 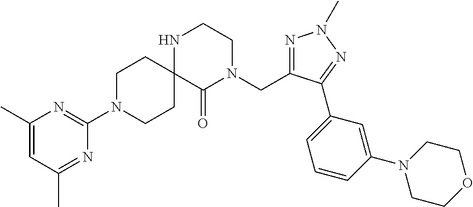 | 9-(4,6-dimethyl- pyrimidin-2-yl)- 4-((2-methyl-5-(3- morpholinophenyl)- 2H-1,2,3-triazol- 4-yl)methyl-1,4,9- triazaspiro[5.5] undecan-5-one | 2.48 (B) | 532.2 |
| 1.78 | 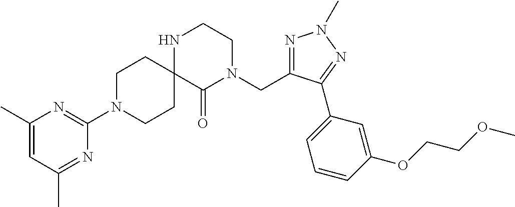 | 9-(4,6-dimethyl- pyrimidin-2-yl)- 4-((5-(3-(2- methoxyethoxy) phenyl)-2-methyl- 2H-1,2,3-triazol-4- yl)methyl)-1,4,9- triazaspiro[5.5] undecan-5-one | 2.54 (B) | 521.2 |
| 1.79 | 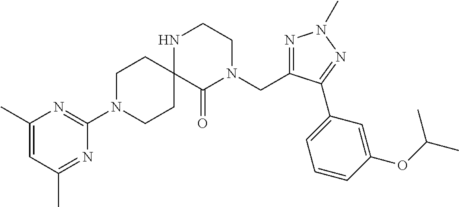 | 9-(4,6-dimethyl- pyrimidin-2-yl)- 4-((5-(3-isopropoxy- phenyl)-2-methyl- 2H-1,2,3-triazol-4- yl)methyl)-1,4,9- triazaspiro[5.5] undecan-5-one | 2.73 (B) | 505.2 |
| 1.80 | 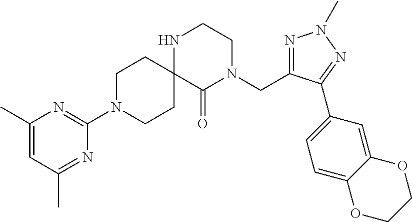 | 4-((5-(2,3-dihydro- benzo[b][1,4]dioxin- 6-yl)-2-methyl-2H- 1,2,3-triazol-4-yl) methyl)-9-(4,6- dimethylpyrimidin- 2-yl)-1,4,9- triazaspiro[5.5] undecan-5-one | 2.81 (A) | 505.2 |
| 1.81 | 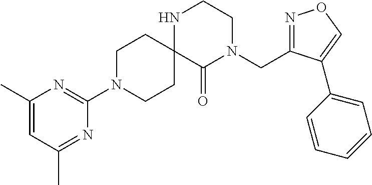 | 9-(4,6-dimethyl- pyrimidin-2-yl)- 4-((4-phenyl- isoxazol-3-yl) methyl)-1,4,9- triazaspiro[5.5] undecan-5-one | 2.57 (B) | 433.2 |
| 1.82 | 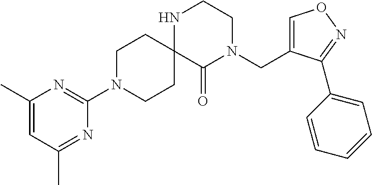 | 9-(4,6-dimethyl- pyrimidin-2-yl)- 4-((3-phenyl- isoxazol-4-yl) methyl)-1,4,9- triazaspiro[5.5] undecan-5-one | 2.51 (B) | 460.2 |
| 1.83 | 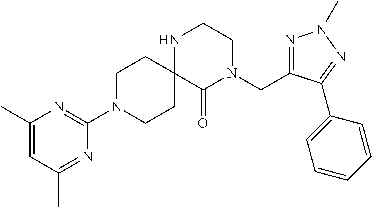 | 9-(4,6-dimethyl- pyrimidin-2-yl)- 4-((2-methyl-5- phenyl-2H-1,2,3- triazol-4-yl) methyl)-1,4,9- triazaspiro[5.5] undecan-5-one | 2.54 (B) | 447.2 |

### Example 2.1

### 4-((1H-indol-3-yl)methyl)-9-(4,6-dimethylpyrimidin-2-yl)-1-oxa-4,9-diazaspiro[5.5]undecan-5-one

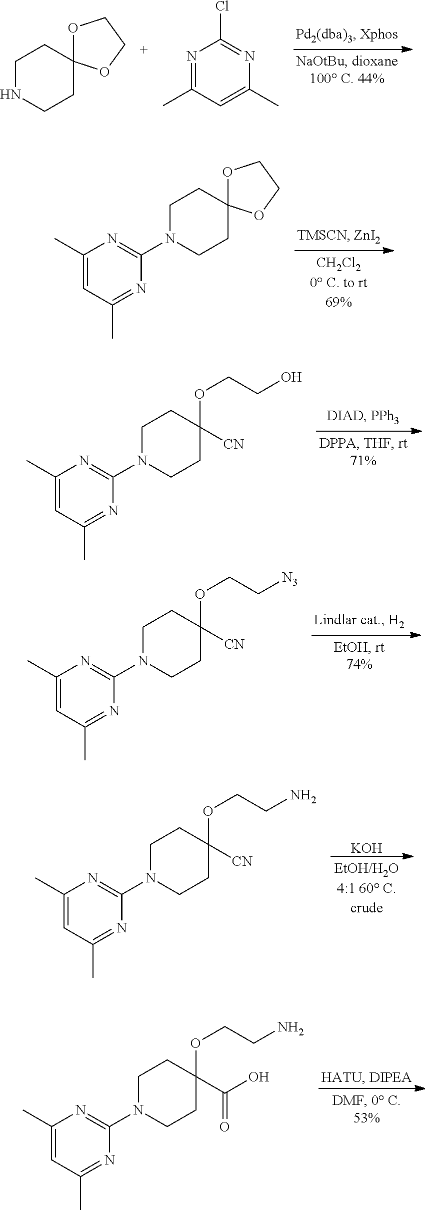

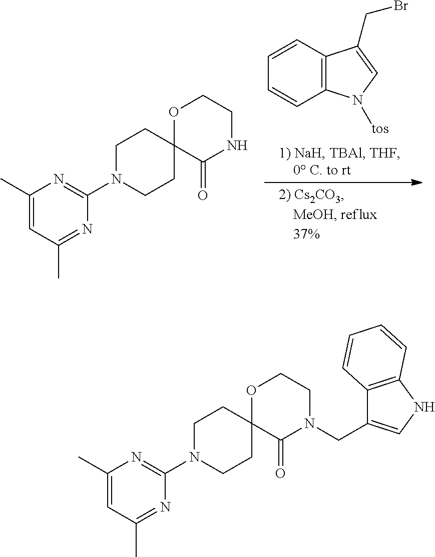

### a) 8-(4,6-Dimethyl-pyrimidin-2-yl)-1,4-dioxa-8-aza-spiro[4.5]decane

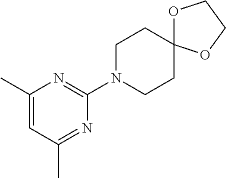

A mixture of 1,4-dioxa-8-azaspiro[4,5]decane (1.12 g, 7.8 mmol), 2-chloro-4,6-dimethylpyrimidine (1.67 g, 11.7 mmol), Pd₂(dba)₃ (286 mg, 0.3 mmol), XPhos (298 mg, 0.62 mmol) and sodium tert-butoxide (1.65 g, 17.2 mmol) in dioxane (8 ml) was placed in a tube. The tube was flushed with nitrogen, sealed and the mixture was heated at 100° C. for 15 h. The mixture was filtered and the residue washed with DCM. The organic phases were washed with brine, dried (Na₂SO₄) and the solvent removed under reduced pressure. The residue was purified by column chromatography (eluent: 20% ethyl acetate in hexane) to yield 0.86 g (44%) of the title compound. [1H NMR (400 MHz, DMSO-d₆) δ ppm 6.37 (s, 1H), 3.82 (s, 4H), 3.63 (br t, 4H), 2.43 (s, 6H), 1.70 (br t, 4H); UPLC-MS Rt_{D}=0.89 min, [M+H]⁺=250.1].

### b) 1-(4,6-Dimethyl-pyrimidin-2-yl)-4-(2-hydroxy-ethoxy)-piperidine-4-carbonitrile

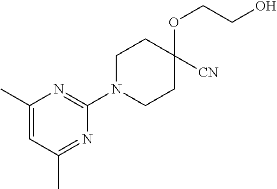

To a solution of 8-(4,6-dimethyl-pyrimidin-2-yl)-1,4-dioxa-8-aza-spiro[4.5]decane (850 mg, 3.4 mmol) in DCM (10 ml) was added ZnI₂ (653 mg, 2.05 mmol) at 0° C. and the solution stirred for 10 min. Then, TMSCN (0.78 ml, 5.8 mmol) was added dropwise and the mixture stirred under warming to room temperature. After 15 h at room temperature, HCl (2N, 2 ml) and MeOH (5 ml) were added and stirring continued for 1 h. Then, the reaction was quenched with 10% Na₂S₂O₃-solution and extracted with DCM. The organics were combined, washed with brine, dried over Na₂SO₄ and the solvents removed under reduced pressure. The residue was purified by column chromatography (10% MeOH in DCM) to yield 650 mg (69%) of the title compound. [1H NMR (400 MHz, DMSO-d₆) δ ppm 6.44 (s, 1H), 4.77 (t, 1H), 4.15-4.04 (br m, 2H), 3.65-3.54 (br m, 6H), 2.22 (s, 6H), 2.15-2.04 (br m, 2H), 1.88-1.77 (br m, 2H); UPLC-MS Rt_{D}=0.82 min, [M+H]⁺=277.1].

### c) 4-(2-Azido-ethoxy)-1-(4,6-dimethyl-pyrimidin-2-yl)-piperidine-4-carbonitrile

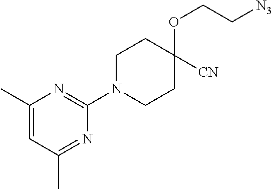

PPh₃ (522 mg, 2.0 mmol) and diisopropyl azodicarboxylate (402 mg, 2.0 mmol) were dissolved in THF (10 ml) at 0° C. After 10 min. a precipitate formed and subsequently 1-(4,6-dimethyl-pyrimidin-2-yl)-4-(2-hydroxy-ethoxy)-piperidine-4-carbonitrile (500 mg, 1.8 mmol) in THF (10 ml) followed by diphenyl phosphoryl azide (548 mg, 2.0 mmol) were added. After removing the ice bath the mixture was stirred for 2 h and then the solvents removed under reduced pressure. The residue was purified by column chromatography (20% EtOAc in hexanes) to yield 420 mg (71%) of the title compound (purity of 92% as determined by UPLC-MS). [1H NMR (400 MHz, DMSO-d₆) δ ppm 6.46 (s, 1H), 4.05-3.95 (br m, 2H), 3.82. (t, 2H), 3.73-3.65 (br m, 2H), 3.51 (t, 2H), 2.24 (s, 6H), 2.18-2.08 (br m, 2H), 1.91-1.83 (br m, 2H); UPLC-MS Rt_{D}=1.15 min, [M+H]⁺=302.1].

### d) 4-(2-Amino-ethoxy)-1-(4,6-dimethyl-pyrimidin-2-yl)-piperidine-4-carbonitrile

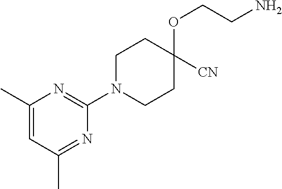

Lindlar catalyst (260 mg, 0.12 mmol) was suspended in EtOH (10 ml) and then 4-(2-azido-ethoxy)-1-(4,6-dimethyl-pyrimidin-2-yl)-piperidine-4-carbonitrile (400 mg, 1.2 mmol) was added and the mixture stirred under H₂ atmosphere for 50 min. Then, the mixture was filtered and the solvent removed under reduced pressure. The residue was purified by column chromatography (15% MeOH in DCM) to yield 250 mg (74%) of the title compound. [1H NMR (400 MHz, DMSO-d₆) δ ppm 6.46 (s, 1H), 4.12-4.05 (br m, 2H), 3.64-3.53 (br m, 4H), 3.71 (br s, 2H), 2.72 (t, 2H), 2.21 (s, 6H), 2.15-2.08 (br m, 2H), 1.88-1.80 (br m, 2H); UPLC-MS Rt_{D}=0.62 min, [M+H]⁺=276.1].

### e) 4-(2-Amino-ethoxy)-1-(4,6-dimethyl-pyrimidin-2-yl)-piperidine-4-carboxylic acid

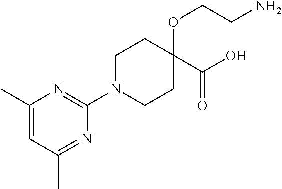

A solution of 4-(2-amino-ethoxy)-1-(4,6-dimethyl-pyrimidin-2-yl)-piperidine-4-carbonitrile (250 mg, 0.91 mmol) in a mixture of EtOH (3 ml) and H₂O (0.75 ml) was treated with KOH (255 mg, 4.5 mmol) and stirred for 17 h at 60° C. After cooling to rt, H₂O was added and the aqueous phase washed with DCM. Then, the aqueous phases were neutralized with 2N HCl and the solvent removed to yield 235 mg (88%) of the title compound containing inorganic impurities. The mixture was used for the next step without purification. [UPLC-MS Rt_{D}=0.45 min, [M+H]⁺=295.1].

### f) 9-(4,6-Dimethyl-pyrimidin-2-yl)-1-oxa-4,9-diaza-spiro[5.5]undecan-5-one

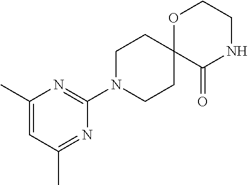

To a stirred solution of 4-(2-amino-ethoxy)-1-(4,6-dimethyl-pyrimidin-2-yl)-piperidine-4-carboxylic acid (235 mg, 0.80 mmol) in DMF (5 ml) were added at 0° C. DIPEA (206 mg, 1.60 mmol) and HATU (334 mg, 0.88 mmol). After stirring for 1 h, brine was added and the aqueous phase extracted with DCM. The combined organic phases were washed with brine, dried over Na₂SO₄, filtered and the solvents removed under reduced pressure. The residue was purified by column chromatography (15% MeOH in CH₂Cl₂) to yield 117 mg (53%) of the title compound. [1H NMR (400 MHz, DMSO-d₆) δ ppm 7.92 (s, 1H), 6.39 (s, 1H), 4.52 (br d, 2H), 3.72 (t, 2H), 3.26-3.20 (br s, 2H), 3.10-3.00 (br m, 2H), 2.23 (s, 6H), 1.81-1.72 (t, 4H); UPLC-MS Rt_{D}=0.66 min, [M+H]⁺=277.1].

### g) 9-(4,6-Dimethyl-pyrimidin-2-yl)-4-(1H-indol-3-ylmethyl)-1-oxa-4,9-diaza-spiro[5.5]undecan-5-one

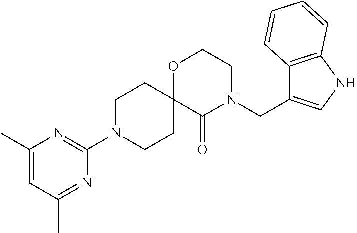

To the solution of NaH (11 mg, 0.43 mmol) in THF (4 ml) was added 9-(4,6-dimethyl-pyrimidin-2-yl)-1-oxa-4,9-diaza-spiro[5.5]undecan-5-one (60 mg, 0.22 mmol) and TBAI (8 mg, 0.02 mmol) and the mixture was stirred for 20 min. Then, 3-bromomethyl-1-(toluene-4-sulfonyl)-1H-indole (116 mg, 0.33 mmol) was added and the ice bath removed. After 16 h, brine was added and the mixture extracted with EtOAc. The combined organic phases were washed with brine, dried over Na₂SO₄, filtered and the solvent removed under reduced pressure. The crude residue (146 mg) was then dissolved in MeOH (3 ml) and Cs₂CO₃ (561 mg, 1.72 mmol) was added. The mixture was stirred at 70° C. for 45 min and then cooled to rt. Brine was added and the aqueous phase extracted with EtOAc, then the combined organic phases were washed with brine, dried over Na₂SO₄, filtered and the solvent removed under reduced pressure. The residue was purified by reverse phase chromatography (LC SunFire C18 OBD column, eluent 10 to 30% CH₃CN/H₂O with 0.1% TFA). Lyophlisation of the product containing fractions, filtration over a SPE cartridge SCX-1 using MeOH, and evaporation of the solvent yielded 25 mg (37%) of the title compound. [1H NMR (400 MHz, DMSO-d₆) δ ppm 11.02 (br s, 1H), 7.51 (d, 1H), 7.42-7.25 (m, 2H), 7.09 (t, 1H), 7.0 (t, 1H), 6.41 (s, 1H), 4.67 (s, 2H), 4.56 (br d, 2H), 3.79 (br t, 2H), 3.22 (t, 2H), 3.21 (br t, 2H), 2.24 (s, 6H), 1.93 (td, 2H), 1.77 (br d, 2H); UPLC-MS Rt_{D}=1.04 min, [M+H]⁺=406.1].

### Example 2.2

### 9-(4,6-Dimethyl-pyrimidin-2-yl)-4-(1H-indazol-3-ylmethyl)-1-oxa-4,9-diaza-spiro[5.5]undecan-5-one

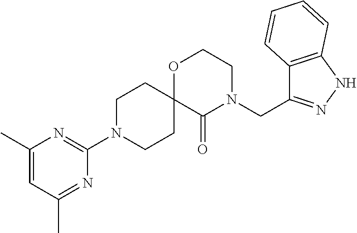

The title compound was obtained in analogy to the method described for example 2.1, using 3-bromomethyl-1-(toluene-4-sulfonyl)-1H-indazole.

[1H-NMR (600 MHz, DMSO-d6) δ ppm 13.0 (s, 1H), 7.68 (d, 1H), 7.51 (d, 1H), 7.36 (t, 1H), 7.11 (t, 1H), 6.41 (s, 1H), 4.87 (s, 2H), 4.56 (d, 2H), 3.83-3.81 (m, 2H), 3.30-3.28 (m, 2H), 3.03 (t, 2H), 2.24 (s, 6H), 1.94-1.88 (m, 2H), 1.79 (d, 2H); LCMS Rt_{E}=0.95 min, [M+H]⁺=407.3]

### Example 2.3

### 9-(4,6-Dimethyl-pyrimidin-2-yl)-4-(2-furan-2-yl-benzyl)-1-oxa-4,9-diaza-spiro[5.5]undecan-5-one

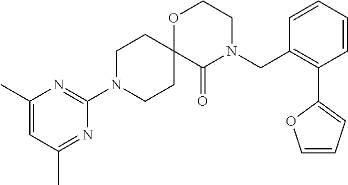

### a) 4-(2-Bromo-benzyl)-9-(4,6-dimethyl-pyrimidin-2-yl)-1-oxa-4,9-diaza-spiro[5.5]undecan-5-one

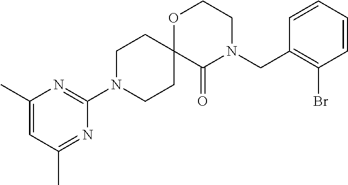

To the cooled solution (ice bath) of NaH (24 mg, 0.61 mmol) in THF (3 ml) was added 9-(4,6-dimethyl-pyrimidin-2-yl)-1-oxa-4,9-diaza-spiro[5.5]undecan-5-one (84 mg, 0.30 mmol) and the mixture was stirred for 20 min. Then, 1-bromo-2-bromomethyl-benzene (76 mg, 0.30 mmol) and TBAI (11 mg, 0.03 mmol) were added. After 15 min the ice bath was removed. After another 2 h, the mixture was cooled with an ice bath, water was added and then the solvent removed under reduced pressure. The crude residue was purified by column chromatography (gradient: 0-20% EtOAc in DCM) to yield 87 mg (64%) of the title compound. [1H NMR (400 MHz, DMSO-d₆) δ ppm 7.63 (d, 1H), 7.39 (t, 1H), 7.23 (t, 1H), 7.16 (d, 1H), 6.37 (s, 1H), 4.50-4.58 (m, 4H), 3.93 (br t, 2H), 3.28-3.32 (m, 2H), 3.05 (dt, 2H), 2.21 (s, 6H), 1.79-1.91 (m, 4H); UPLC-MS Rt_{E}=1.23 min, [M+H]⁺=445.2, 447.2].

### b) 9-(4,6-Dimethyl-pyrimidin-2-yl)-4-(2-furan-2-yl-benzyl)-1-oxa-4,9-diaza-spiro[5.5]undecan-5-one

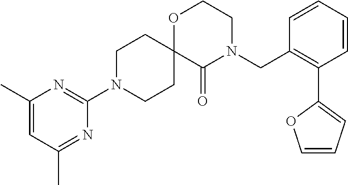

4-(2-Bromo-benzyl)-9-(4,6-dimethyl-pyrimidin-2-yl)-1-oxa-4,9-diaza-spiro[5.5]undecan-5-one (43 mg, 0.1 mmol), 2-(tributylstannyl)furane (69 mg, 0.2 mmol), and PdCl₂(dppf)CH₂Cl₂ (8 mg, 0.01 mmol) were dissolved in dioxane (1 ml) and heated 4 h at 110° C. in a microwave oven. The solvents were evaporated at reduced pressure and the residue purified by chromatography on silica (gradient: 0-20% EtOAc in DCM) to yield the product as colorless oil (41 mg, 98%). [1H-NMR (DMSO, 600 MHz) δ ppm 7.82 (d, 1H), 7.66-7.70 (m, 1H), 7.36-7.41 (m, 2H), 7.16-7.19 (m, 1H), 6.76 (d, 1H), 6.63-6.66 (m, 1H), 6.41 (s, 1H), 4.74 (s, 2H), 4.58 (d, 2H), 3.94 (t, 2H), 3.28 (t, 2H), 3.08 (dt, 2H), 2.24 (s, 6H), 1.84-1.93 (m, 4H); LCMS Rt_{E}=1.27 min, [M+H]⁺=433.3]

### Example 2.4

### 4-((1H-indazol-3-yl)methyl)-9-(4-methoxy-6-methylpyrimidin-2-yl)-1-oxa-4,9-diazaspiro[5.5]undecan-5-one

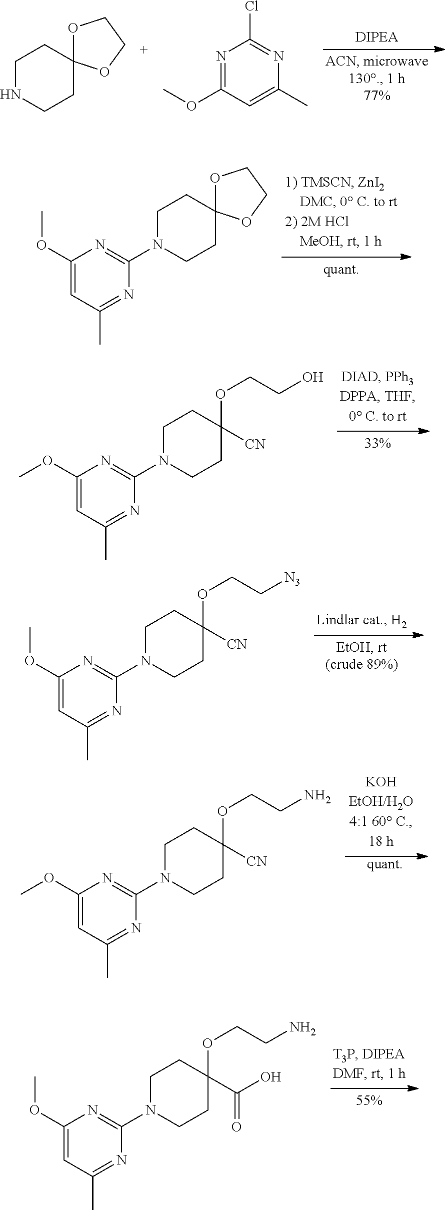

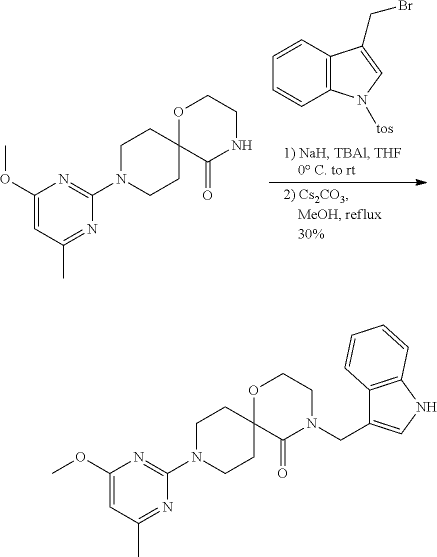

### a) 8-(4-Methoxy-6-methyl-pyrimidin-2-yl)-1,4-dioxa-8-aza-spiro[4.5]decane

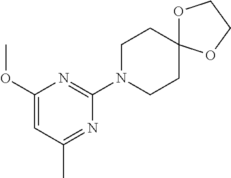

A solution of 2-chloro-4-methoxy-6-methyl-pyrimidine (3.38 g, 21.3 mmol), 1,4-dioxa-azaspiro[4,5]decane (2.61 g, 21.3 mmol) and DIPEA (18.6 ml, 106 mmol) in CAN (40 ml) was evenly distributed into three microwave vials. The vials were sealed and each mixture was heated at 130° C. for 1 h under microwave conditions. All three reaction mixtures were pooled and the solvent removed under reduced pressure. The residue was taken up in DCM, washed with 5% aqueous NaHCO₃ solution and with H₂O. The aqueous layers were extracted two times with DCM and the combined organic phases washed with brine, dried (Na₂SO₄), the solvents evaporated and the residue purified by chromatography on silica (gradient: 0-15% EtOAc in hexanes) to yield the product as colorless oil (4.5 g, 77%). [1H-NMR (DMSO, 400 MHz) δ ppm 5.91 (s, 1H), 3.90 (s, 4H), 3.76-3.82 (m, 7H), 2.16 (s, 3H), 1.56-1.62 (t, 4H); LCMS Rt_{D}=0.76 min, [M+H]⁺=266.3]

### b) 4-(2-Hydroxy-ethoxy)-1-(4-methoxy-6-methyl-pyrimidin-2-yl)-piperidine-4-carbonitrile

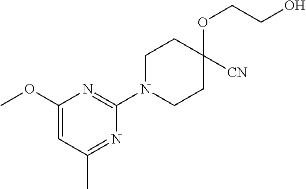

To a solution of 8-(4-methoxy-6-methyl-pyrimidin-2-yl)-1,4-dioxa-8-aza-spiro[4.5]decane (3.4 g, 12.7 mmol) in dry DCM was added at 0° C. ZnI₂ (10.2 g, 31.8 mmol). After 30 min at this temperature, TMSCN solution (4.27 ml, 31.8 mmol) was added slowly to the mixture and then stirring continued under warming to rt. After 18 h, 12.5 mol of 2M HCl solution and 25 ml of MeOH were added and stirring continued for 1 h. Then, 5% aqueous Na₂S₂O₃ solution was added and the mixture extracted three times with DCM. Combined organic layers were washed with brine, dried (Na₂SO₄) and the solvents evaporated. A yellow solid was obtained which was used for the next step without further purification. [LCMS Rt_{D}=0.74 min, [M+H]⁺=293.3]

### c) 4-(2-Azido-ethoxy)-1-(4-methoxy-6-methyl-pyrimidin-2-yl)-piperidine-4-carbonitrile

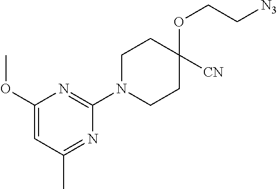

Under nitrogen atmosphere, DIAD (2.77 ml, 13.9 mmol) was added to a solution of PPh3 (3.7 g, 13.9 mmol) if dry THF (50 ml) at 0° C. After 10 min. a solution of 4-(2-hydroxy-ethoxy)-1-(4-methoxy-6-methyl-pyrimidin-2-yl)-piperidine-4-carbonitrile (3.9 g, 12.7 mmol) in dry THF (50 ml) and DPPA 3.2 ml, 13.9 mmol) were added and the mixture was stirred under warming to rt. After 18 h, the solvent was removed under reduced pressure and the residue purified by chromatography on silica (gradient: 0-15% EtOAc in hexanes) to yield the product as yellowish oil (1.4 g, 33%). [1H-NMR (DMSO, 400 MHz) δ ppm 5.98 (s, 1H), 3.91-3.99 (m, 2H), 3.78-3.82 (m, 5H), 3.63-3.72 (m, 2H), 3.48 (t, 2H), 2.18 (s, 3H), 2.10-2.16 (m, 2H), 1.85-1.91 (m, 2H); LCMS Rt_{D}=1.14 min, [M+H]⁺=318.2]

### d) 4-(2-Amino-ethoxy)-1-(4-methoxy-6-methyl-pyrimidin-2-yl)-piperidine-4-carbonitrile

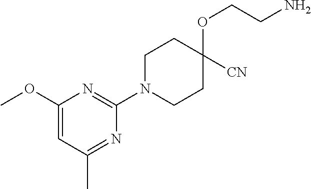

A solution of 4-(2-azido-ethoxy)-1-(4-methoxy-6-methyl-pyrimidin-2-yl)-piperidine-4-carbonitrile and Lindlar Catalyst (0.5 g) were stirred under H2 atmosphere for 16 h. Then, more Lindlar Catalyst (0.2 g) was added and stirring under H2 atmosphere continued. After a total of 38 h, the mixture was filtered and the filtrate concentrated under reduced pressure. This yielded a yellow oil which was used for the next step without purification. [LCMS Rt_{D}=0.58 min, [M+H]⁺=292.3]

### e) 4-(2-Amino-ethoxy)-1-(4-methoxy-6-methyl-pyrimidin-2-yl)-piperidine-4-carboxylic acid

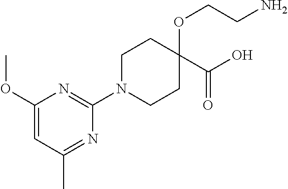

To a solution of 4-(2-amino-ethoxy)-1-(4-methoxy-6-methyl-pyrimidin-2-yl)-piperidine-4-carbonitrile (0.96 g, 2.9 mmol) in EtOH (20 ml) and H₂O (5 ml), KOH (0.81 g, 14.5 mmol) was added and the reaction mixture heated at 60° C. for 18 h. After cooling to rt, HCl (4M, 0.44 ml, 14.5 mmol) was added, the precipitate was filtered off and the filtrate concentrated to yield a light brown solid (1.6 g) containing both the product and inorganic salts. This solid was used for the next step without further purification. [LCMS Rt_{D}=0.428 min, [M+H]⁺=311.2]

### f) 9-(4-Methoxy-6-methyl-pyrimidin-2-yl)-1-oxa-4,9-diaza-spiro[5.5]undecan-5-one

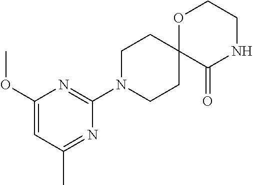

The crude 4-(2-amino-ethoxy)-1-(4-methoxy-6-methyl-pyrimidin-2-yl)-piperidine-4-carboxylic acid was dissolved in DMF (25 ml). Then, DIPEA (1.31 ml, 7.5 mmol) followed by T3P (50% in DMF, 2.2 ml, 3.75 ml) were added dropwise and the mixture stirred for 1 h. Then, the solvent was removed under reduced pressure, the residue was taken up in DCM and washed with 10% NaHCO₃ solution. The aqueous phase was extracted three times with DCM, the combined organic phases dried (Na₂SO₄) and the solvent removed under reduced pressure. The residue was purified by chromatography on silica (gradient: 0-5% MeOH in DCM) to yield the product as yellowish oil (472 mg, 55% yield over 3 steps). [1H-NMR (DMSO, 400 MHz) δ ppm 7.90 (s, 1H), 5.91 (s, 1H), 4.48 (br d, 2H), 3.76-3.81 (m, 2H), 3.78 (s, 3H), 3.22 (br t, 2H), 3.01-3.08 (m, 2H), 2.17 (s, 3H), 1.75-1.80 (m, 4H); LCMS Rt_{D}=0.57 min, [M+H]⁺=293.2]

### g) 4-(1H-Indazol-3-ylmethyl)-9-(4-methoxy-6-methyl-pyrimidin-2-yl)-1-oxa-4,9-diaza-spiro[5.5]undecan-5-one

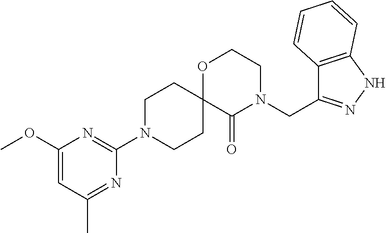

To a solution of 9-(4-methoxy-6-methyl-pyrimidin-2-yl)-1-oxa-4,9-diaza-spiro[5.5]undecan-5-one (210 mg, 0.72 mmol) in THF (6 ml) under N₂ at 0° C. NaH (60%, 58 mg, 1.4 mmol) was added portionwise. After 20 min a solution of 3-bromomethyl-1-(toluene-4-sulfonyl)-1H-indazole (262 mg, 0.72 mmol) in THF (4.5 ml) and TBAI (27 mg, 0.07 mmol) were added. The reaction mixture was stirred at 0° C. for 1 h and then at rt for 18 h. Then, the solvents were removed under reduced pressure, the resulting residue taken up in MeOH (11 ml), treated with Cs₂CO₃ (1.4 g, 4.3 mmol) and the resulting mixture refluxed for 2.5 h. After cooling to rt, the solvent was removed under reduced pressure, the residue dissolved in DCM and washed with H₂O, The aqueous layer was extracted three times with DCM, then the combined organic phases dried (Na₂SO₄) and concentrated. Purification of the residue by chromatography on silica (gradient: 0-5% MeOH in DCM) followed by crystallization from DCM/TBME yielded the product as white solid (95 mg, 30%). [1H-NMR (DMSO, 400 MHz) δ ppm 12.92 (s, 1H), 7.66 (d, 1H), 7.49 (d, 1H), 7.33 (t, 1H), 7.08 (t, 1H), 5.93 (s, 1H), 4.85 (s, 2H), 4.50 (br d, 2H), 3.77-3.82 (m, 2H), 3.80 (s, 3H), 3.77-3.82 (m, 2H), 3.03 (br t, 2H), 2.18 (s, 3H), 1.92 (dt, 2H), 1.77 (br d, 2H); LCMS Rt_{D}=0.83 min, [M+H]⁺=423.4]

| | | | LCMS | |
|---|---|---|---|---|
| | | | Rt [min], | |
| Ex. | Structure | Name | method | [M + H]⁺ |
| 2.5 | 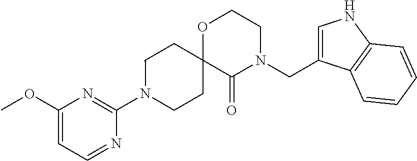 | 4-((1H-Indol-3-yl)methyl)-9- (4-methoxypyrimidin-2-yl)-1- oxa-4,9- diazaspiro[5.5]undecan-5- one | 0.92 (E) | 408.3 |
| 2.6 | 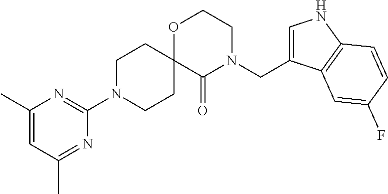 | 9-(4,6-Dimethylpyrimidin-2- yl)-4-((5-fluoro-1H-indol-3- yl)methyl)-1-oxa-4,9- diazaspiro[5.5]undecan-5- one | 1.07 (E) | 424.3 |
| 2.7 | 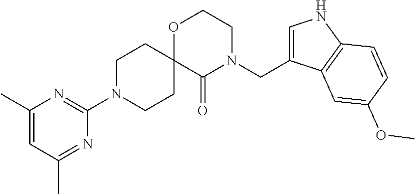 | 9-(4,6-Dimethylpyrimidin-2- yl)-4-((5-methoxy-1H-indol- 3-yl)methyl)-1-oxa-4,9- diazaspiro[5.5]undecan-5- one | 1.01 (E) | 436.3 |
| 2.8 | 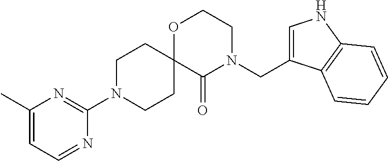 | 4-((1H-Indol-3-yl)methyl)-9- (4-methylpyrimidin-2-yl)-1- oxa-4,9- diazaspiro[5.5]undecan-5- one | 1.00 (E) | 392.3 |
| 2.9 | 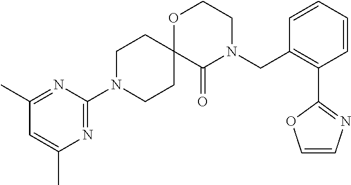 | 9-(4,6-Dimethylpyrimidin-2- yl)-4-(2-(oxazol-2-yl)benzyl)- 1-oxa-4,9- diazaspiro[5.5]undecan-5- one | 1.12 (E) | 434.3 |
| 2.10 | 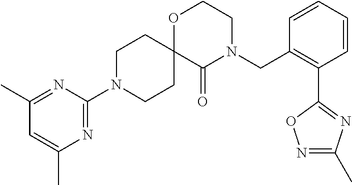 | 9-(4,6-Dimethylpyrimidin-2- yl)-4-(2-(3-methyl-1,2,4- oxadiazol-5-yl)benzyl)-1- oxa-4,9- diazaspiro[5.5]undecan-5- one | 1.14 (E) | 449.3 |
| 2.11 | 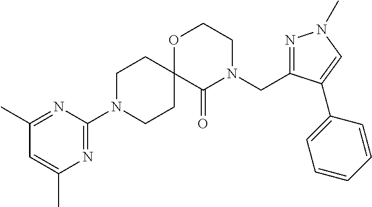 | 9-(4,6-Dimethylpyrimidin-2- yl)-4-((1-methyl-4-phenyl- 1H-pyrazol-3-yl)methyl)-1- oxa-4,9- diazaspiro[5.5]undecan-5- one | 1.08 (E) | 447.3 |
| 2.12 | 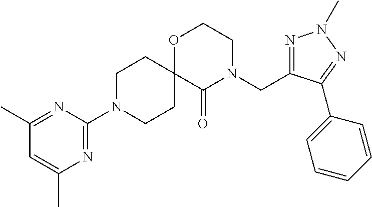 | 9-(4,6-Dimethylpyrimidin-2- yl)-4-((2-methyl-5-phenyl- 2H-1,2,3-triazol-4- yl)methyl)-1-oxa-4,9- diazaspiro[5.5]undecan-5- one | 1.14 (E) | 448.3 |
| 2.13 | 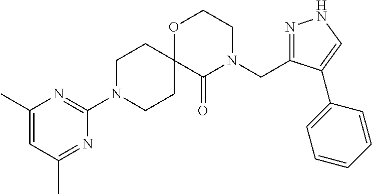 | 9-(4,6-Dimethylpyrimidin-2- yl)-4-((4-phenyl-1H-pyrazol- 3-yl)methyl)-1-oxa-4,9- diazaspiro[5.5]undecan-5- one | 0.99 (E) | 433.3 |
| 2.14 | 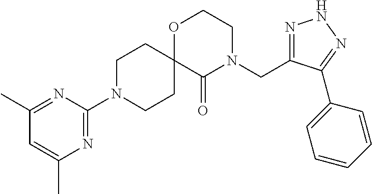 | 9-(4,6-Dimethylpyrimidin-2- yl)-4-((5-phenyl-2H-1,2,3- triazol-4-yl)methyl)-1-oxa- 4,9-diazaspiro[5.5]undecan- 5-one | 0.94 (E) | 434.1 |
| 2.15 | 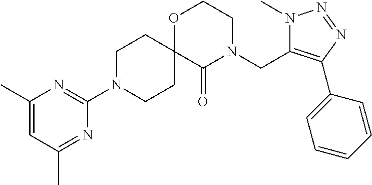 | 9-(4,6-Dimethylpyrimidin-2- yl)-4-((1-methyl-4-phenyl- 1H-1,2,3-triazol-5- yl)methyl)-1-oxa-4,9- diazaspiro[5.5]undecan-5- one | 1.00 (E) | 448.1 |
| 2.16 | 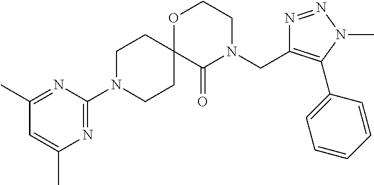 | 9-(4,6-Dimethylpyrimidin-2- yl)-4-((1-methyl-5-phenyl- 1H-1,2,3-triazol-4- yl)methyl)-1-oxa-4,9- diazaspiro[5.5]undecan-5- one | 0.95 (E) | 448.1 |
| 2.17 | 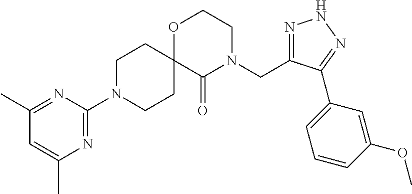 | 9-(4,6-Dimethylpyrimidin-2- yl)-4-((5-(3-methoxyphenyl)- 2H-1,2,3-triazol-4- yl)methyl)-1-oxa-4,9- diazaspiro[5.5]undecan-5- one | 0.96 (E) | 464.3 |
| 2.18 | 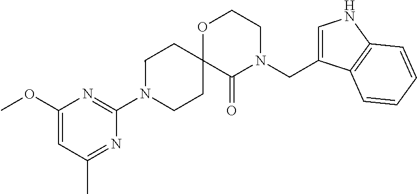 | 4-((1H-Indol-3-yl)methyl)-9- (4-methoxy-6- methylpyrimidin-2-yl)-1-oxa- 4,9-diazaspiro[5.5]undecan- 5-one | 0.99 (E) | 422.3 |
| 2.19 | 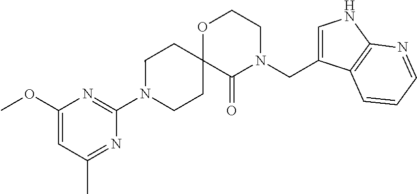 | 4-((1H-Pyrrolo[2,3-b]pyridin- 3-yl)methyl)-9-(4-methoxy- 6-methylpyrimidin-2-yl)-1- oxa-4,9- diazaspiro[5.5]undecan-5- one | 0.76 (E) | 423.3 |
| 2.20 | 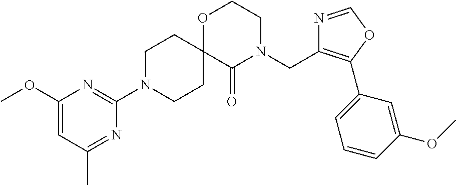 | 9-(4-Methoxy-6- methylpyrimidin-2-yl)-4-((5- (3-methoxyphenyl)oxazol-4- yl)methyl)-1-oxa-4,9- diazaspiro[5.5]undecan-5- one | 1.05 (E) | 480.3 |
| 2.21 | 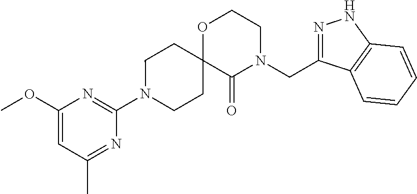 | 4-((1H-indazol-3-yl)methyl)- 9-(4-methoxy-6- methylpyrimidin-2-yl)-1-oxa- 4,9-diazaspiro[5.5]undecan- 5-one | 0.87 (D) | 423.3 |
| 2.22 | 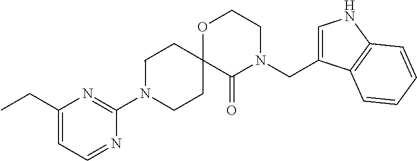 | 4-((1H-indol-3-yl)methyl)-9- (4-ethylpyrimidin-2-yl)-1- oxa-4,9- diazaspiro[5.5]undecan-5- one | 1.08 (D) | 406.5 |
| 2.23 | 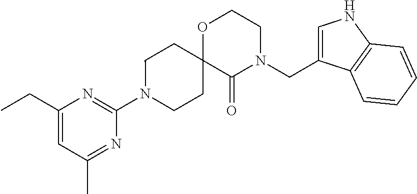 | 4-((1H-indol-3-yl)methyl)-9- (4-ethyl-6-methylpyrimidin- 2-yl)-1-oxa-4,9- diazaspiro[5.5]undecan-5- one | 1.13 (D) | 420.5 |
| 2.24 | 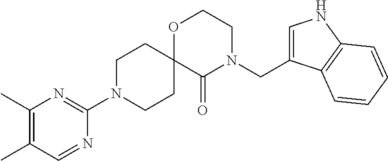 | 4-((1H-indol-3-yl)methyl)-9- (4,5-dimethylpyrimidin-2-yl)- 1-oxa-4,9- diazaspiro[5.5]undecan-5- one | 1.02 (D) | 406.4 |
| 2.25 | 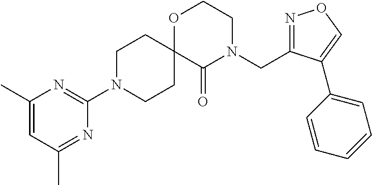 | 9-(4,6-dimethylpyrimidin-2- yl)-4-((4-phenylisoxazol-3- yl)methyl)-1-oxa-4,9- diazaspiro[5.5]undecan-5- one | 2.92 (B) | 434.2 |
| 2.26 | 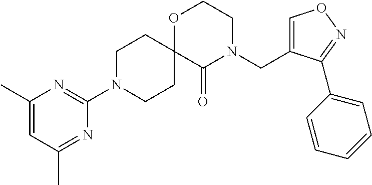 | 9-(4,6-dimethylpyrimidin-2- yl)-4-((3-phenylisoxazol-4- yl)methyl)-1-oxa-4,9- diazaspiro[5.5]undecan-5- one | 2.85 (B) | 434.2 |

### Example 3.1

### 2-((1H-Indol-3-yl)methyl)-8-(4,6-dimethylpyrimidin-2-yl)-2,8-diazaspiro[4.5]decan-1-one

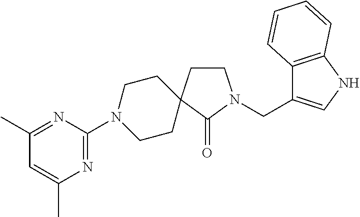

The title compound was obtained in analogy to the method described for example 2.1, starting from tert-butyl 1-oxo-2,8-diazaspiro[4.5]decane-8-carboxylate.

[1H-NMR (600 MHz, DMSO-d6) δ ppm 11.0 (s, 1H), 7.50 (d, 1H), 7.38 (d, 1H), 7.34 (s, 1H), 7.10 (dd, 1H), 6.99 (dd, 1H), 6.39 (s, 1H), 4.52 (s, 2H), 4.51 (m, 2H), 3.11 (t, 2H), 3.01 (dd, 2H), 2.20 (s, 6H), 1.88 (t, 2H), 1.57 (dd, 2H), 1.25 (d, 2H); LCMS Rt_{E}=1.02 min, [M+H]⁺=390.1]

| | | | LCMS | |
|---|---|---|---|---|
| | | | Rt [min], | |
| Ex. | Structure | Name | method | [M + H]⁺ |
| 3.2 | 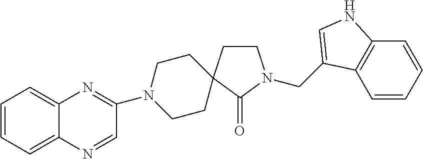 | 2-((1H-Indol-3-yl)methyl)-8- (quinoxalin-2-yl)-2,8- diazaspiro[4.5]decan-1-one | 1.05 (E) | 412.1 |
| 3.3 | 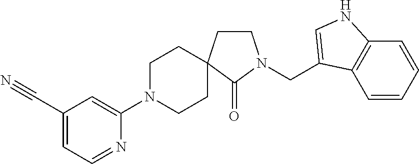 | 2-(2-((1H-Indol-3-yl)methyl)- 1-oxo-2,8- diazaspiro[4.5]decan-8- yl)isonicotinonitrile | 1.05 (E) | 386.0 |
| 3.4 | 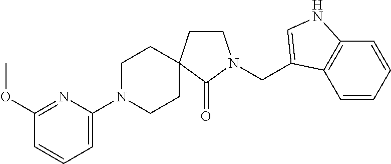 | 2-((1H-Indol-3-yl)methyl)-8- (6-methoxypyridin-2-yl)-2,8- diazaspiro[4.5]decan-1-one | 1.12 (E) | 391.1 |
| 3.5 | 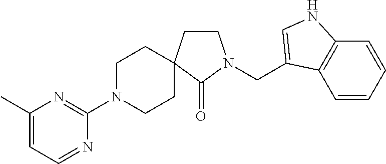 | 2-((1H-Indol-3-yl)methyl)-8- (4-methylpyrimidin-2-yl)-2,8- diazaspiro[4.5]decan-1-one | 0.99 (E) | 376.0 |
| 3.6 | 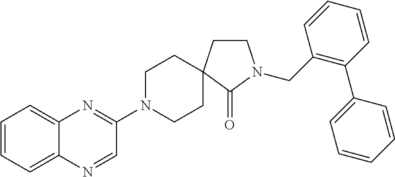 | 2-(biphenyl-2-ylmethyl)-8- (quinoxalin-2-yl)-2,8- diazaspiro[4.5]decan-1-one | 3.653 (E) | 449.2 |
| 3.7 | 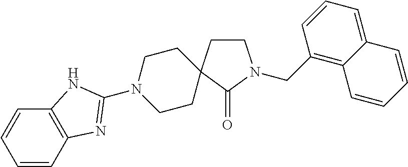 | 8-(1H-Benzo[d]imidazol-2- yl)-2-(naphthalen-1- ylmethyl)-2,8- diazaspiro[4.5]decan-1-one | 3.079 (B) | 411.2 |
| 3.8 | 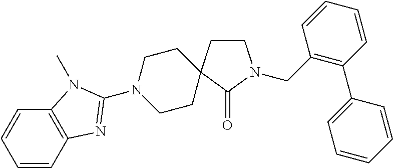 | 2-(Biphenyl-2-ylmethyl)-8- (1-methyl-1H- benzo[d]imidazol-2-yl)-2,8- diazaspiro[4.5]decan-1-one | 3.193 (B) | 451.2 |
| 3.9 | 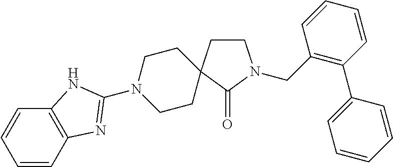 | 8-(1H-Benzo[d]imidazol-2- yl)-2-(biphenyl-2-ylmethyl)- 2,8-diazaspiro[4.5]decan-1- one | 3.155 (B) | 437.2 |
| 3.10 | 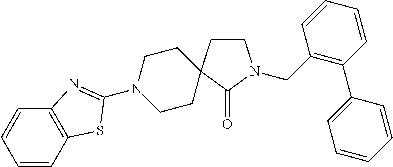 | 8-(Benzo[d]thiazol-2-yl)-2- (biphenyl-2-ylmethyl)-2,8- diazaspiro[4.5]decan-1-one | 3.521 (B) | 454.2 |
| 3.11 | 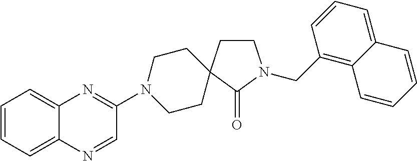 | 2-(Naphthalen-1-ylmethyl)- 8-(quinoxalin-2-yl)-2,8- diazaspiro[4.5]decan-1-one | 3.015 (C) | 423.1 |
| 3.12 | 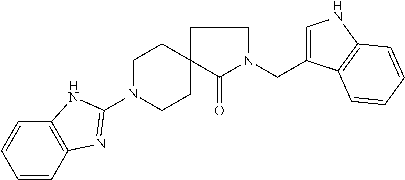 | 8-(1H-Benzoimidazol-2-yl)- 2-(1H-indol-3-ylmethyl)-2,8- diaza-spiro[4.5]decan-1-one | 3.029 (A) | 400.2 |
| 3.13 | 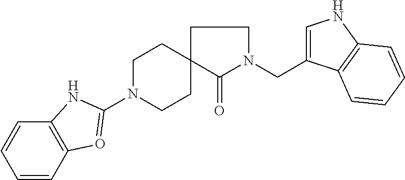 | 8-Benzooxazol-2-yl-2-(1H- indol-3-ylmethyl)-2,8-diaza- spiro[4.5]decan-1-one | 2.073 (G) | 401.2 |
| 3.14 | 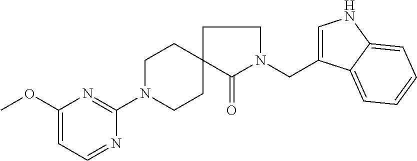 | 2-(1H-Indol-3-ylmethyl)-8- (4-methoxy-pyrimidin-2-yl)- 2,8-diaza-spiro[4.5]decan-1- one | 2.723 (B) | 392.2 |
| 3.15 | 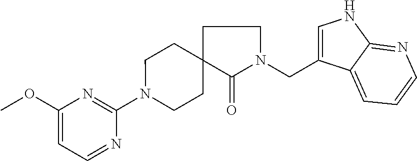 | 8-(4-Methoxy-pyrimidin-2- yl)-2-(1H-pyrrolo[2,3- b]pyridin-3-ylmethyl)-2,8- diaza-spiro[4.5]decan-1-one | 2.159 (B) | 393.2 |
| 3.16 | 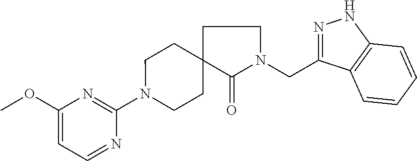 | 2-(1H-Indazol-3-ylmethyl)-8- (4-methoxy-pyrimidin-2-yl)- 2,8-diaza-spiro[4.5]decan-1- one | 2.548 (B) | 393.2 |
| 3.17 | 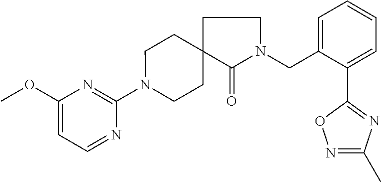 | 8-(4-Methoxy-pyrimidin-2- yl)-2-[2-(3-methyl- [1,2,4]oxadiazol-5-yl)- benzyl]-2,8-diaza- spiro[4.5]decan-1-one | 2.811 (B) | 435.2 |
| 3.18 | 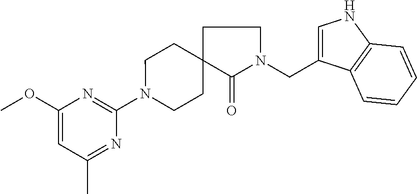 | 2-(1H-Indol-3-ylmethyl)-8- (4-methoxy-6-methyl- pyrimidin-2-yl)-2,8-diaza- spiro[4.5]decan-1-one | 30.68 (A) | 406.2 |
| 3.19 | 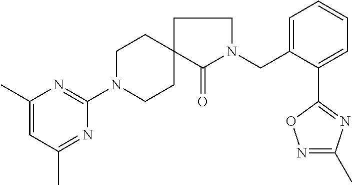 | 8-(4,6-Dimethyl-pyrimidin-2- yl)-2-[2-(3-methyl- [1,2,4]oxadiazol-5-yl)- benzyl]-2,8-diaza- spiro[4.5]decan-1-one | 3.138 (A) | 433.2 |
| 3.20 | 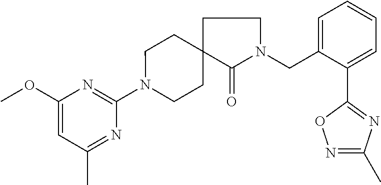 | 8-(4-Methoxy-6-methyl- pyrimidin-2-yl)-2-[2-(3- methyl-[1,2,4]oxadiazol-5- yl)-benzyl]-2,8-diaza- spiro[4.5]decan-1-one | 3.145 (A) | 449.2 |
| 3.21 | 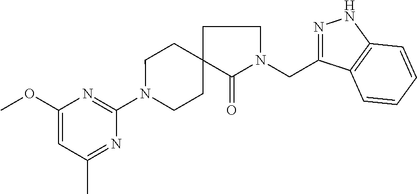 | 2-((1H-indazol-3-yl)methyl)- 8-(4-methoxy-6- methylpyrimidin-2-yl)-2,8- diazaspiro[4.5]decan-1-one | 2.917 (A) | 407.2 |
| 3.22 | 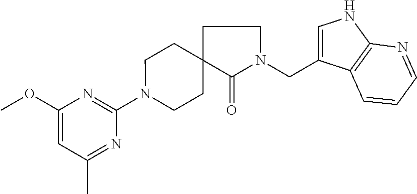 | 2-((1H-pyrrolo[2,3-b]pyridin- 3-yl)methyl)-8-(4-methoxy- 6-methylpyrimidin-2-yl)-2,8- diazaspiro[4.5]decan-1-one | 2.596 (A) | 407.2 |
| 3.23 | 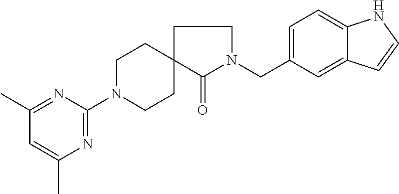 | 2-((1H-indol-5-yl)methyl)-8- (4,6-dimethylpyrimidin-2-yl)- 2,8-diazaspiro[4.5]decan-1- one | 3.004 (A) | 390.2 |
| 3.24 | 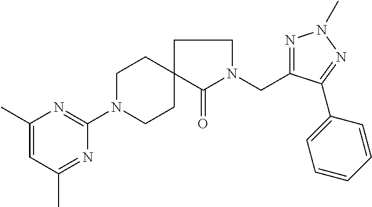 | 8-(4,6-dimethylpyrimidin-2- yl)-2-((2-methyl-5-phenyl- 2H-1,2,3-triazol-4- yl)methyl)-2,8- diazaspiro[4.5]decan-1-one | 3.067 (A) | 432.2 |

### Example 4.1

### 3-((1H-indol-3-yl)methyl)-8-(4,6-dimethylpyrimidin-2-yl)-1,3,8-triazaspiro[4.5]decan-4-one

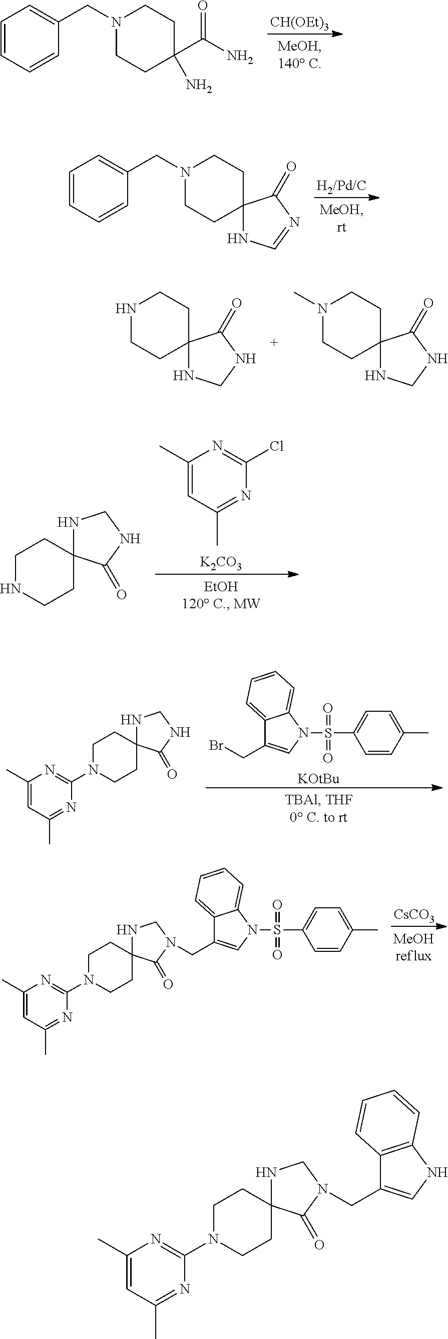

### a) 8-Benzyl-1,3,8-triazaspiro[4.5]dec-2-en-4-one

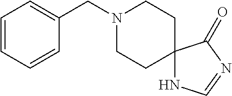

The solution of 4-amino-1-benzylpiperidine-4-carboxamide (1.3 g, 5.7 mmol) in triethoxymethane (50 ml) was heated at 145° C. for 16 h. Triethoxymethane was removed under vacuum (50° C., 18 mbar). The resulting oil was diluted in water and the aqueous layer was extracted 2 times with EtOAc. The organic phases were washed with water and brine. The combined organic layers were dried over sodium sulfate, filtered and evaporated to give of a pale orange oil which still contained some triethoxmethane and was used in the next step without further purification. [LCMS Rt_{A}=2.37 min, [M+H]⁺=244.2].

### b) 1,3,8-Triazaspiro[4.5]decan-4-one

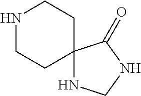

The product 8-benzyl-1,3,8-triazaspiro[4.5]dec-2-en-4-one from the previous step was hydrogenated in methanol at rt in the presence of Pd(OH)₂ at 50 bar for 6 days with occasional addition of fresh catalyst. Due to the presence of triethoxymethane a mixture of the title compound and its N-methyl derivative 8-methyl-1,3,8-triazaspiro[4.5]decan-4-one was formed. The crude product was purified by flash column chromatography (EtOH 95%/aqueous ammonium hydroxide 5%) to afford the title compound. ¹H NMR (600 MHz, DMSO-d₆) δ ppm 7.91 (br. s., 1H), 4.06 (br. s., 2H), 2.74 (m, 2H), 2.57-2.69 (m, 2H), 1.49 (td, 2H), 1.26 (d, 2H).

### c) 8-(4,6-Dimethylpyrimidin-2-yl)-1,3,8-triazaspiro[4.5]decan-4-one

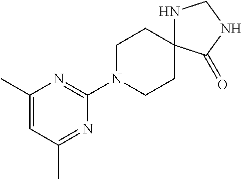

To a solution of 1,3,8-triazaspiro[4.5]decan-4-one (175 mg, 1.1 mmol) in ethanol (11 ml) was added 2-chloro-4,6-dimethylpyrimidine (191 mg, 1.3 mmol) and DIPEA (0.49 ml, 2.8 mmol) in a microwave tube. The tube was sealed and the mixture was heated at 140° C. for 1 h under microwave conditions. The mixture was diluted with phosphate buffer (pH 7) and extracted with DCM. The organic phase was washed with the same buffer and with brine. The organic layer was dried over sodium sulfate, filtered and evaporated. The resulting yellow oil was purified by flash column chromatography (DCM/ethanol 7:3) to afford the title compound as a pale yellow oil (173 mg, 58%). [LCMS Rt_{A}=2.28 min, [M+H]⁺=262.2].

¹H NMR (400 MHz, DMSO-d₆) δ ppm 8.01 (s, 1H), 6.35 (s, 1H), 4.38-4.57 (m, 2H), 4.12 (d, 2H), 3.19-3.28 (m, 1H), 3.04-3.19 (m, 2H), 2.20 (s, 6H), 1.46-1.59 (m, 2H), 1.34-1.46 (m, 2H).

### d) 8-(4,6-Dimethylpyrimidin-2-yl)-3-((1-tosyl-1H-indol-3-yl)methyl)-1,3,8-triazaspiro[4.5]decan-4-one

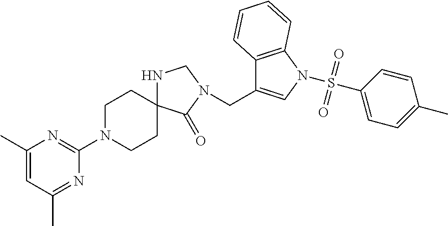

To the solution of 8-(4,6-dimethylpyrimidin-2-yl)-1,3,8-triazaspiro[4.5]decan-4-one (163 mg, 0.62 mmol) and TBAI 23 mg, 0.062 mmol) in anhydrous THF (6 ml) tBuOK (77 mg, 0.69 mmol) was added at 0° C. and the mixture was stirred for 20 min at 0° C. under argon. 3-(Bromomethyl)-1-tosyl-1H-indole (227 mg, 0.62 mmol) was added and stirring was continued at 0° C. for 1 h. Water was added and the aqueous layer was extracted with DCM. The organic phase was washed with water and brine. The combined organic layers were dried over anhydrous sodium sulfate, filtered and evaporated to give the title compound as a white powder (340 mg, 100%). [LCMS Rt_{C}=2.56 min, [M+H]⁺=545.2].

### e) 3-((1H-indol-3-yl)methyl)-8-(4,6-dimethylpyrimidin-2-yl)-1,3,8-triazaspiro[4.5]decan-4-one

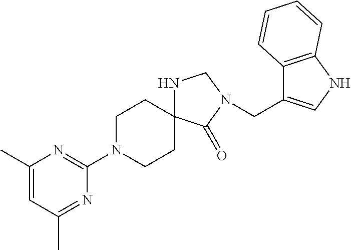

To the suspension of 8-(4,6-dimethylpyrimidin-2-yl)-3-((1-tosyl-1H-indol-3-yl)methyl)-1,3,8-triazaspiro[4.5]decan-4-one (340 mg, 0.62 mmol) in methanol (6 ml) was added cesium carbonate (2.0 g, 6.2 mmol) and the mixture was refluxed for 2 h. The mixture was diluted with ethyl acetate and water was added. The phases were separated. The aqueous layer was extracted with ethyl acetate. The organic phases were washed with a saturated aqueous solution of NHCO₃ and brine. The combined organic layers were dried over anhydrous sodium sulfate, filtered and evaporated to give a pale orange oil. 1H NMR (400 MHz, DMSO-d₆) δ ppm 10.97 (br. s., 1H), 7.49 (d, 1H), 7.34 (d, 1H), 7.31 (s, 1H), 7.07 (t, 1H), 6.96 (t, 1H), 6.36 (s, 1H), 4.41-4.54 (m, 4H), 4.04 (d, 2H), 3.19 (t, 1H), 3.10 (t, 2H), 2.20 (s, 6H), 1.58 (td, 2H), 1.36 (d, 2H). [LCMS Rt_{B}=2.55 min, [M+H]⁺=391.2].

### Example 5.1

### 2-((1H-indol-3-yl)methyl)-7-(4-methoxypyrimidin-2-yl)-2,7-diazaspiro[4.4]nonan-1-one

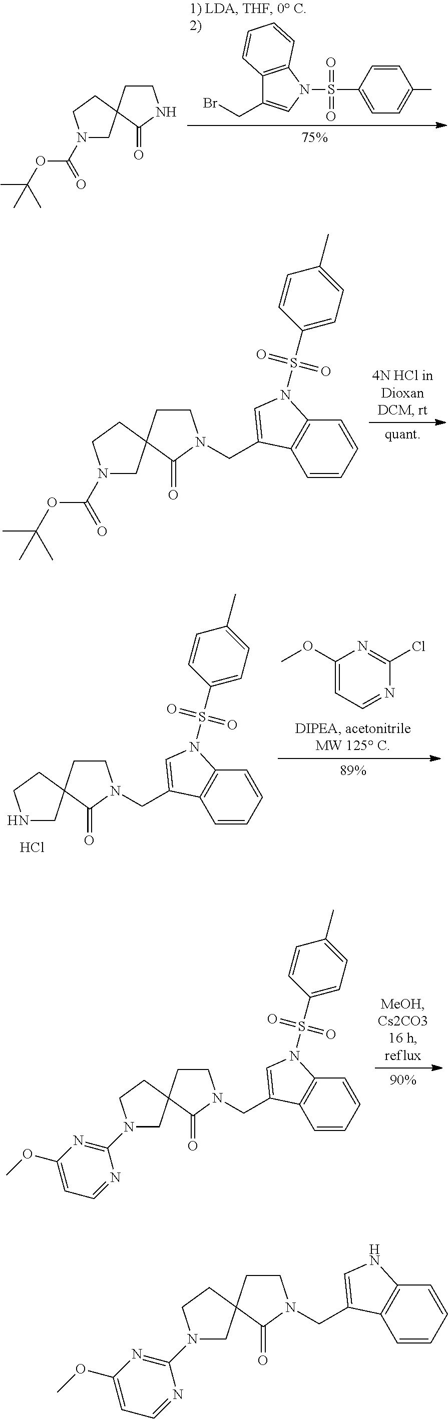

### a) 6-Oxo-7-[1-(toluene-4-sulfonyl)-1H-indol-3-ylmethyl]-2,7-diaza-spiro[4.4]nonane-2-carboxylic acid tert-butyl ester

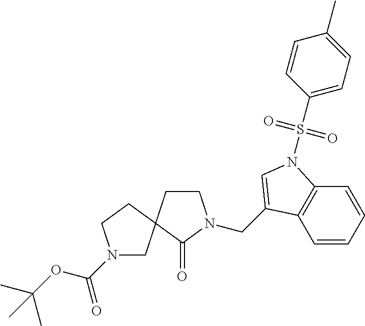

To the solution of diisopropylamine (0.297 ml, 2.08 mmol) in THF (10 ml) at 0° C. was added butyllithium (1.6 M solution in hexan, 1.43 ml, 2.29 mmol), followed by tert-butyl 6-oxo-2,7-diazaspiro[4.4]nonane-2-carboxylate (500 mg, 2.08 mmol) in small portions. After stirring for 30 min at 0° C.-5° C. the solution of 3-(bromomethyl)-1-tosyl-1H-indole (758 mg, 2.08 mmol) in THF (2 ml) was added and stirring was continued for 1 h at 0° C., followed by 20 h at rt. The reaction mixture was poured on ice-water (60 ml) and 10% citric acid (60 ml). The product was extracted with EtOAc, washed with brine, dried over sodium sulfate, and the solvent was evaporated under reduced pressure. The crude product was purified by chromatography on silica (Isolera, hexane to hexane/EtOAc 1/4 in 30 min, 10 min at hexane/EtOAc 1/4 to give the product as colorless solid (811 mg, 74%). [1H-NMR (DMSO, 600 MHz) δ ppm 7.90 (d, 1H), 7.84-7.83 (m, 3H), 7.51 (d, 1H), 7.38 (d, 2H), 7.34 (dd, 1H), 7.24 (dd, 1H), 4.49 (s, 2H), 3.47-3.43 (m, 1H), 3.34-3.18 (m, 3H), 3.12-3.04 (m, 3H), 2.31 (s, 3H), 1.92-1.84 (m, 3H), 1.69-1.64 (m, 1H), 1.39/1.37 (s, 9H); LCMS Rt_{C}=3.631 min; [M+Na]⁺=546.2]

### b) 2-[1-(Toluene-4-sulfonyl)-1H-indol-3-ylmethyl]-2,7-diaza-spiro[4.4]nonan-1-one hydrochloride

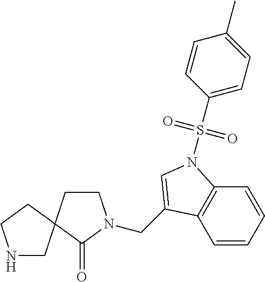

6-Oxo-7-[1-(toluene-4-sulfonyl)-1H-indol-3-ylmethyl]-2,7-diaza-spiro[4.4]nonane-2-carboxylic acid tert-butyl ester (810 mg, 1.55 mmol) was dissolved in DCM (16 m) and 4 N hydrochloric acid in dioxan (4 N in dioxan, 9.7 ml, 39 mmol) and stirred 1 h at rt. The solvents were evaporated under reduced pressure to give the title compound as off-white solid (720 mg, quant.). [1H-NMR (DMSO, 600 MHz) δ ppm 9.16 (s br, 2H), 7.91 (d, 1H), 7.86-7.84 (m, 3H), 7.53 (d, 1H), 7.39 (d, 2H), 7.35 (dd, 1H), 7.25 (dd, 1H), 4.55 (d, 1H), 4.48 (d, 1H), 3.34-3.30 (m, 1H), 3.23-3.08 (m, 5H), 2.31 (s, 3H), 2.03-1.91 (m, 3H), 1.86-1.81 (m, 1H); LCMS Rt_{C}=2.105 min; [M+H]⁺=424.2]

### c) 7-(4-Methoxy-pyrimidin-2-yl)-2-[1-(toluene-4-sulfonyl)-1H-indol-3-ylmethyl]-2,7-diaza-spiro[4.4]nonan-1-one

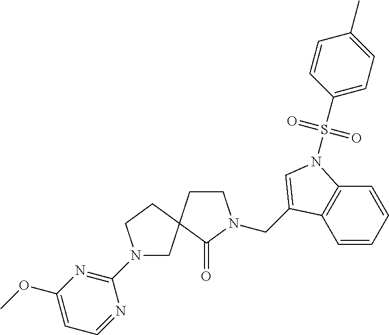

2-[1-(Toluene-4-sulfonyl)-1H-indol-3-ylmethyl]-2,7-diaza-spiro[4.4]nonan-1-one hydrochloride (120 mg, 0.26 mmol), 2-chloro-4-methoxypyrimidine (51 mg, 0.35 mmol), and diisopropylethylamine (0.205 ml, 1.17 mmol) were dissolved in acetonitrile (2 ml) and heated 2 h at 125° C. in a microwave oven. The solvents were evaporated at reduced pressure and the residue purified by chromatography on silica (Isolera, Hex to Hex/EtOAc 1/4 in 20 min, 10 min at Hex/EtOAc 1/4) to yield the product as colorless solid (123 mg, 89%). [1H-NMR (DMSO, 600 MHz) δ ppm 8.06 (d, 1H), 7.93 (d, 1H), 7.87-7.85 (m, 3H), 7.55 (d, 1H), 7.41-7.35 (m, 3H), 7.26 (dd, 1H), 6.06 (d, 1H), 4.54 (s, 2H), 3.85-3.72 (m, 4H), 3.60-3.42 (m, 3H), 3.14 (dd, 2H), 2.32 (s, 3H), 2.09-1.94 (m, 3H), 1.86-1.80 (m, 1H); LCMS Rt_{C}=2.650 min; [M+H]⁺=532.2]

### d) 2-((1H-indol-3-yl)methyl)-7-(4-methoxypyrimidin-2-yl)-2,7-diazaspiro[4.4]nonan-1-one

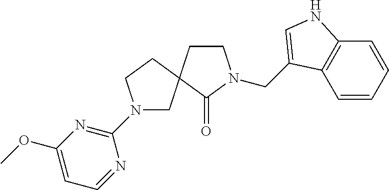

To the solution of 7-(4-methoxy-pyrimidin-2-yl)-2-[1-(toluene-4-sulfonyl)-1H-indol-3-ylmethyl]-2,7-diaza-spiro[4.4]nonan-1-one (122 mg, 0.229 mmol) in MeOH (5 ml) was added caesium carbonate (336 mg, 1.03 mmol) and the mixture was heated at reflux for 19 h. The solvent was evaporated under reduced pressure and the residue purified by chromatography on silica (Isolera, DCM to DCM/MeOH 97.5/2.5 in 20 min, 10 min at DCM/MeOH 97.5/2.5) to give the title compound as colorless solid (78 mg, 90%). [1H-NMR (DMSO, 600 MHz) δ ppm 11.02 (s, 1H), 8.05 (s, 1H), 7.50 (d, 1H), 7.37-7.34 (m, 2H), 7.09 (dd, 1H), 6.97 (dd, 1H), 6.05 (d, 1H), 4.55 (s, 2H), 3.84-3.73 (m, 4H), 3.63-3.42 (m, 3H), 3.14 (dd, 2H), 2.12-2.07 (m, 1H), 1.94-1.92 (m, 2H), 1.85-1.81 (m, 1H); LCMS Rt_{A}=2.949 min; [M+H]⁺=378.2]

### Examples 5.2 and 5.3

### (R)-2-((1H-indol-3-yl)methyl)-7-(4-methoxypyrimidin-2-yl)-2,7-diazaspiro[4.4]nonan-1-one and (S)-2-((1H-indol-3-yl)methyl)-7-(4-methoxypyrimidin-2-yl)-2,7-diazaspiro[4.4]nonan-1-one

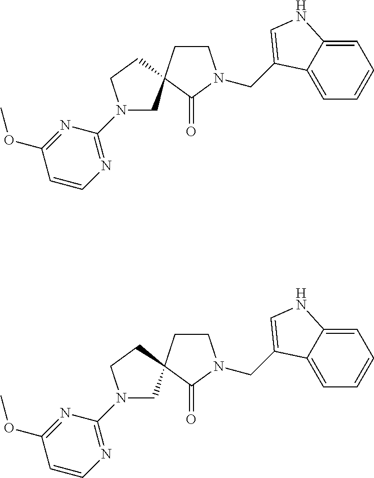

The enantiomers of Example 5.1 have been separated by chiral chromatography (Thar/Waters SFC-100 MS; column Chiralpak AS-H, 30×250 mm; mobile phase scCO2/IPA 6/4 isocratic, 80 ml/min, 120 bar) to give the title compounds as colorless solids.

Example 5.2: Rt_{G}=3.27 min.

Example 5.3: Rt_{G}=6.85 min.

### Example 5.4

### 2-((1H-indol-3-yl)methyl)-7-(4-methoxy-6-methylpyrimidin-2-yl)-2,7-diazaspiro[4.4]nonan-1-one

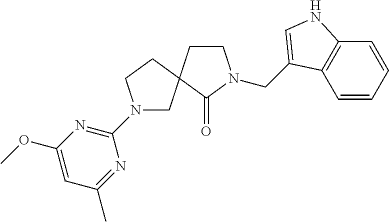

The title compound was obtained in analogy to the method described for example 2.1. [¹H-NMR (600 MHz, DMSO-d6) δ ppm 11.02 (s, 1H), 7.50 (d, 1H), 7.36 (d, 1H), 7.34 (d, 1H), 7.08 (dd, 1H), 6.98 (dd, 1H), 5.92 (s, 1H), 4.55 (s, 2H), 3.81-3.73 (m, 4H), 3.58-3.38 (m, 3H), 3.14 (t, 2H), 2.17 (s, 3H), 2.10-2.05 (m, 1H), 1.92 (t, 2H), 1.83-1-79 (m, 1H); LCMS Rt_{A}=3.021 min, [M+H]⁺=392.2]

### Example 5.17

### 2-((1H-indol-3-yl)methyl)-7-(6-(trifluoromethyl)pyrimidin-4-yl)-2,7-diazaspiro[4.4]nonan-1-on

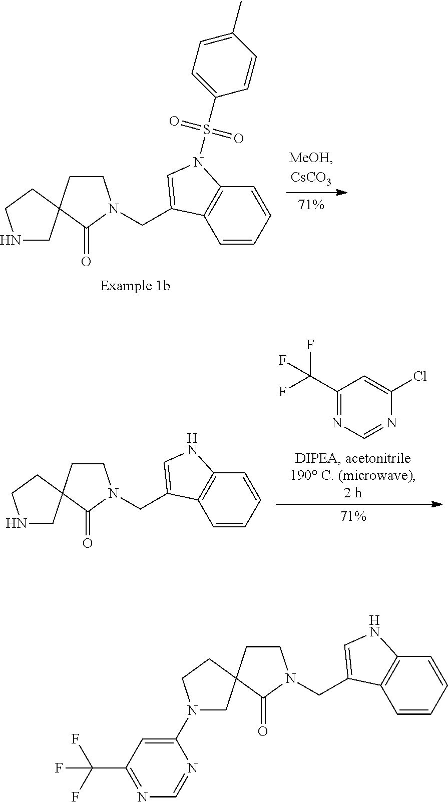

### a) 2-((1H-indol-3-yl)methyl)-2,7-diazaspiro[4.4]nonan-1-one

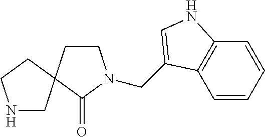

The solution of 2-[1-(toluene-4-sulfonyl)-1H-indol-3-ylmethyl]-2,7-diaza-spiro[4.4]nonan-1-one hydrochloride (see example 1b; 3.4 g, 7.39 mmol) in MeOH (50 ml) was added cesium carbonate (10.84 g, 33.3 mmol) and the mixture was stirred at 80° C. for 20 h. The solvent was removed under reduced pressure to about 10 ml and the residue added to DCM/MeOH/ammonia 90/10/1 (100 ml). After filtration through a glass filter to remove the unsoluble parts the solvents were evaporated and the residue was adsorped on Isolute. Purification by chromatography on silica (Flashmaster, DCM to DCM/MeOH/ammonia 90/10/1 in 30 min, then DCM/MeOH/ammonia 90/10/1 for 60 min, flow rate 35 ml/min) to yield the product as off-white solid (1.42 g, 71%). [¹H-NMR (600 MHz, DMSO-d₆) δ ppm 10.98 (s, 1H), 7.48 (d, 1H), 7.35 (d, 1H), 7.30 (d, 1H), 7.07 (t, 1H), 6.96 (t, 1H), 4.50 (s, 2H), 2.89-2.85 (m, 1H), 2.82-2.78 (m, 2H), 2.59 (d, 1H), 1.93-1.83 (m, 2H), 1.79-1.75 (m, 1H), 1.53-1.49 (m, 1H); LCMS Rt_{B}=2.196 min, [M+H]⁺=270.2]

### b) 2-((1H-Indol-3-yl)methyl)-7-(6-(trifluoromethyl)pyrimidin-4-yl)-2,7-diazaspiro[4.4]nonan-1-one

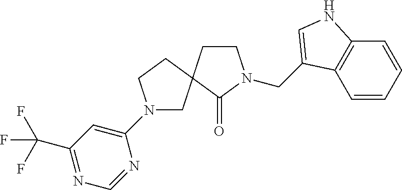

To the solution of 2-((1H-indol-3-yl)methyl)-2,7-diazaspiro[4.4]nonan-1-one (30 mg, 0.111 mmol) in acetonitrile (1 ml) were added DIPEA (0.097 mml, 0.557 mmol) and 4-chloro-6-(trifluoromethyl)pyrimidine (0.111 mmol). The mixture was flushed with argon and then heated in a microwave oven at 190° C. for 2 h. The reaction mixture was diluted with MeOH (1 ml), filtered through a PTFE membrane (0.45 μm) and the resulting filtrate was purified be preparative LC/MS (Waters system with Micromass ZQ MS detection; waters X Bridge C18-ODB 30×150 mm, 5 μm; H₂O with 0.79 g/l ammoniumcarbonate/acetonitrile 95/5 to 25/75, flow 50 ml/min; collection triggered by MS signal). Fractions containing the title compound were collected and freeze dried to receive the product as white powder (33 mg, 72%). [¹H-NMR (400 MHz, DMSO-d₆) δ ppm 11.00 (s br, 1H), 8.61 (d, 1H), 7.51 (t, 1H), 7.39-7.34 (m, 2H), 7.10 (dt, 1H), 6.99 (dt, 1H), 6.90 (d, 1H), 4.56 (s, 2H), 3.88-3.47 (m, 4H), 3.24-3.14 (m, 2H), 2.22-2.07 (m, 1H), 1.99-1.88 (m, 3H); LCMS Rt_{D}=0.97 min, [M+H]⁺=416.2]

| | | | LCMS | |
|---|---|---|---|---|
| | | | Rt [min], | |
| Ex. | Structure | Name | method | [M + H]⁺ |
| 5.5 | 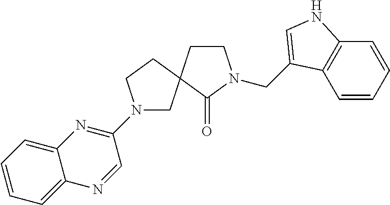 | 2-((1H-Indol-3-yl) methyl)-7- (quinoxalin-2-yl)-2,7- diazaspiro[4.4] nonan-1-one | 0.99 (D) | 398.3 |
| 5.6 | 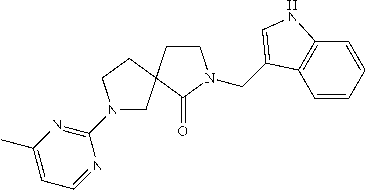 | 2-((1H-Indol-3-yl) methyl)- 7-(4-methylpyrimidin- 2-yl)-2,7-diazaspiro[4.4] nonan-1-one | 2.892 (A) | 362.2 |
| 5.7 | 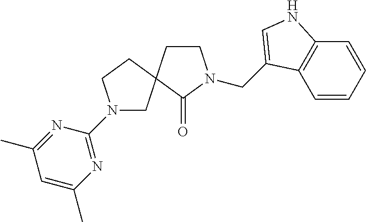 | 2-((1H-Indol-3-yl) methyl)-7- (4,6-dimethyl- pyrimidin-2-yl)- 2,7-diazaspiro[4.4] nonan-1-one | 2.950 (A) | 376.2 |
| 5.8 | 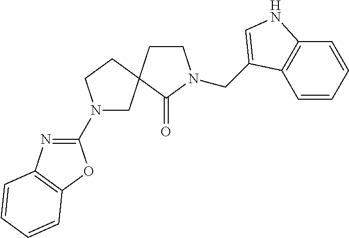 | 2-((1H-Indol-3-yl) methyl)-7- (benzo[d]oxazol- 2-yl)-2,7- diazaspiro[4.4] nonan-1-one | 2.925 (B) | 387.2 |
| 5.9 | 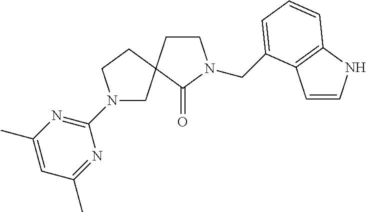 | 2-((1H-Indol-4- yl)methyl)-7- (4,6-dimethyl- pyrimidin-2-yl)- 2,7-diazaspiro[4.4] nonan-1-one | 2.630 (B) | 376.2 |
| 5.10 | 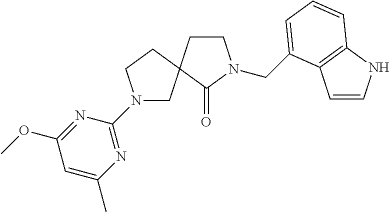 | 2-((1H-Indol-4-yl) methyl)- 7-(4-methoxy-6- methylpyrimidin-2- yl)-2,7-diazaspiro[4.4] nonan-1-one | 2.714 (B) | 392.2 |
| 5.11 | 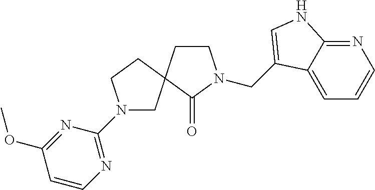 | 2-((1H-Pyrrolo[2,3-b] pyridin-3-yl)methyl)- 7-(4- methoxypyrimidin- 2-yl)-2,7- diazaspiro[4.4] nonan-1-one | 2.506 (A) | 379.2 |
| 5.12 | 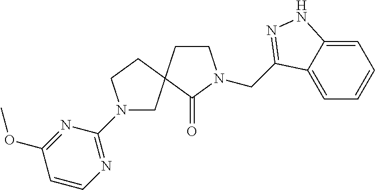 | 2-((1H-Indazol-3-yl) methyl)-7-(4-methoxy- pyrimidin- 2-yl)-2,7-diazaspiro[4.4] nonan-1-one | 2.809 (A) | 379.2 |
| 5.13 | 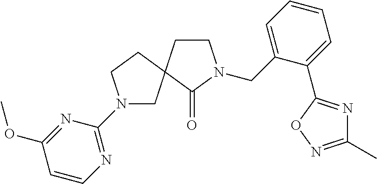 | 7-(4-Methoxypyrimidin- 2-yl)-2- (2-(3-methyl-1,2,4- oxadiazol- 5-yl)benzyl)-2,7- diazaspiro[4.4] nonan-1-one | 3.042 (A) | 421.2 |
| 5.14 | 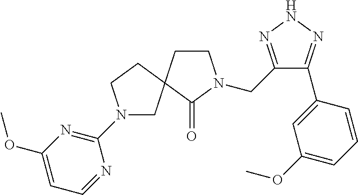 | 2-((5-(3-Methoxy- phenyl)-2H- 1,2,3-triazol-4-yl) methyl)-7-(4- methoxypyrimidin- 2-yl)-2,7- diazaspiro[4.4] nonan-1-one | 2.874 (A) | 436.2 |
| 5.15 | 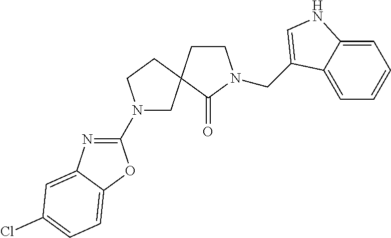 | 2-((1H-Indol-3-yl) methyl)-7-(5- chlorobenzo[d] oxazol-2-yl)- 2,7-diazaspiro[4.4] nonan-1-one | 2.801 (C) | 421.0 |
| 5.16 | 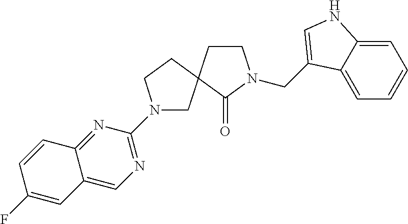 | 2-((1H-Indol-3-yl) methyl)-7-(6- fluoroquinazolin-2- yl)-2,7- diazaspiro[4.4] nonan-1-one | 2.857 (B) | 416.2 |
| 5.18 | 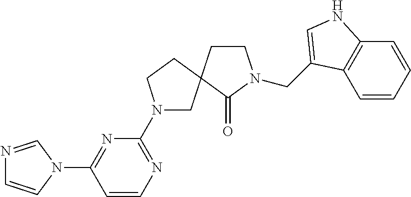 | 7-(4-(1H-imidazol-1- yl)pyrimidin-2-yl)-2- ((1H-indol- 3-yl)methyl)-2,7- diazaspiro[4.4] nonan-1-one | 0.79 (D) | 414.3 |
| 5.19 | 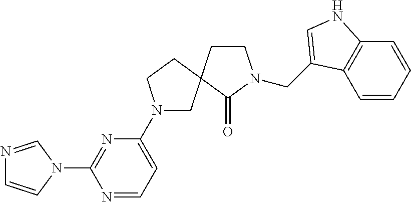 | 7-(2-(1H-imidazol-1- yl)pyrimidin-4-yl)- 2-((1H-indol- 3-yl)methyl)-2,7- diazaspiro[4.4] nonan-1-one | 0.76 (D) | 414.3 |
| 5.20 | 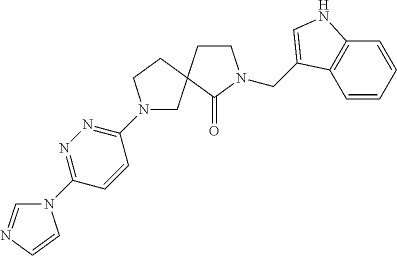 | 7-(6-(1H-imidazol-1- yl)pyridazin-3-yl)- 2-((1H-indol- 3-yl)methyl)-2,7- diazaspiro[4.4] nonan-1-one | 0.66 (D) | 414.3 |
| 5.21 | 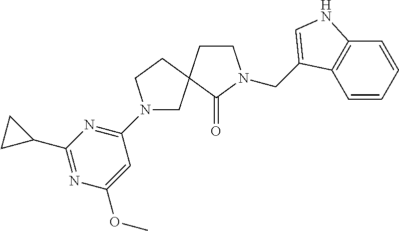 | 2-((1H-indol-3-yl) methyl)-7-(2- cyclopropyl-6- methoxy- pyrimidin-4-yl)-2,7- diazaspiro[4.4] nonan-1-one | 0.94 (D) | 418.3 |
| 5.22 | 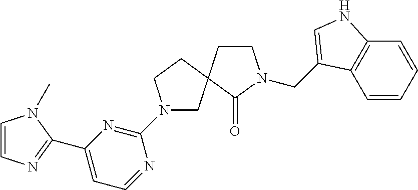 | 2-((1H-indol-3-yl) methyl)-7-(4- (1-methyl-1H- imidazol-2- yl)pyrimidin-2-yl)-2,7- diazaspiro[4.4] nonan-1-one | 0.77 (D) | 428.3 |
| 5.23 | 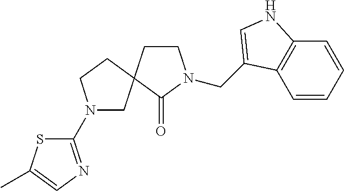 | 2-((1H-indol-3-yl) methyl)-7-(5- methylthiazol- 2-yl)-2,7- diazaspiro[4.4] nonan-1-one | 0.74 (D) | 367.2 |
| 5.24 | 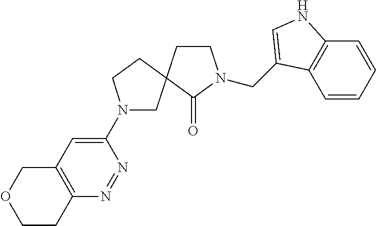 | 2-((1H-indol-3-yl) methyl)-7- (7,8-dihydro-5H- pyrano[4,3- c]pyridazin-3-yl)- 2,7- diazaspiro[4.4] nonan-1-one | 0.65 (D) | 404.2 |
| 5.25 | 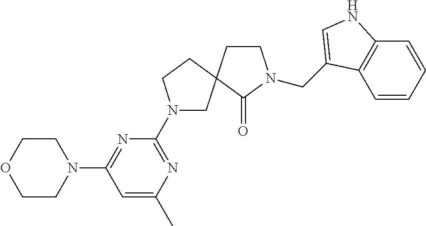 | 2-((1H-indol-3-yl) methyl)-7-(4- methyl-6-morpholino- pyrimidin- 2-yl)-2,7- diazaspiro[4.4] nonan-1-one | 0.73 (D) | 447.3 |
| 5.26 | 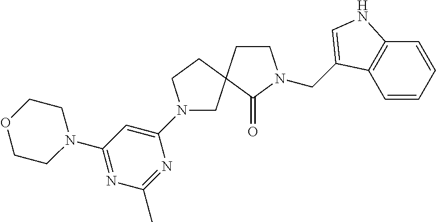 | 2-((1H-indol-3-yl) methyl)-7-(2- methyl-6-morpholino- pyrimidin- 4-yl)-2,7- diazaspiro[4.4] nonan-1-one | 0.71 (D) | 447.3 |
| 5.27 | 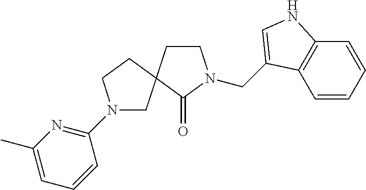 | 2-((1H-indol-3-yl) methyl)-7-(6- methylpyridin- 2-yl)-2,7- diazaspiro[4.4] nonan-1-one | 0.67 (D) | 361.3 |
| 5.28 | 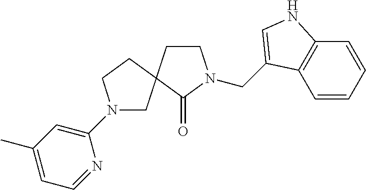 | 2-((1H-indol-3-yl) methyl)-7-(4- methylpyridin- 2-yl)-2,7- diazaspiro[4.4] nonan-1-one | 0.68 (D) | 361.3 |
| 5.29 | 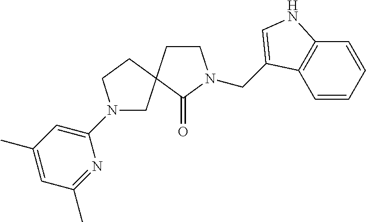 | 2-((1H-indol-3-yl) methyl)-7- (4,6-dimethyl- pyridin-2-yl)-2,7- diazaspiro[4.4] nonan-1-one | 0.72 (D) | 375.3 |
| 5.30 | 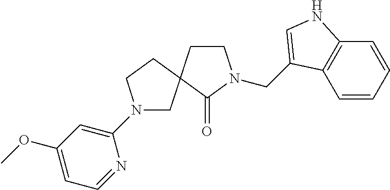 | 2-((1H-indol-3-yl) methyl)-7-(4- methoxypyridin- 2-yl)-2,7- diazaspiro[4.4] nonan-1-one | 0.68 (D) | 377.3 |
| 5.31 | 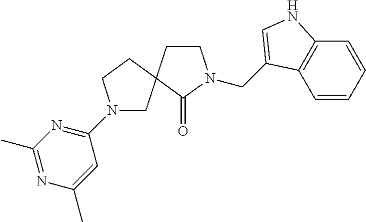 | 2-((1H-indol-3-yl) methyl)-7- (2,6-dimethyl- pyrimidin-4-yl)- 2,7-diazaspiro[4.4] nonan-1-one | 0.66 (D) | 376.3 |
| 5.32 | 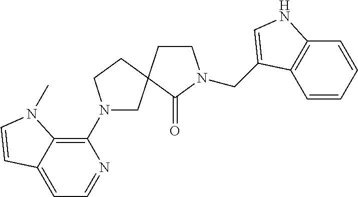 | 2-((1H-indol-3-yl) methyl)-7-(1- methyl-1H-pyrrolo[2,3- c]pyridin-7-yl)-2,7- diazaspiro[4.4] nonan-1-one | 0.74 (D) | 400.3 |
| 5.33 | 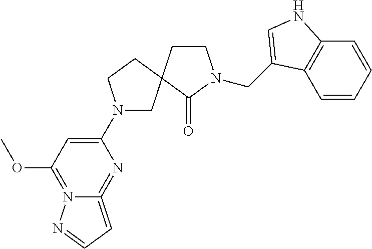 | 2-((1H-indol-3-yl) methyl)-7-(7- methoxypyrazolo[1,5- a]pyrimidin-5-yl)-2,7- diazaspiro[4.4] nonan-1-one | 0.73 (D) | 417.3 |
| 5.34 | 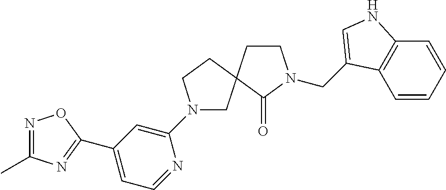 | 2-((1H-indol-3-yl) methyl)-7-(4- (3-methyl-1,2,4- oxadiazol-5- yl)pyridin-2-yl)-2,7- diazaspiro[4.4] nonan-1-one | 0.96 (D) | 429.3 |
| 5.35 | 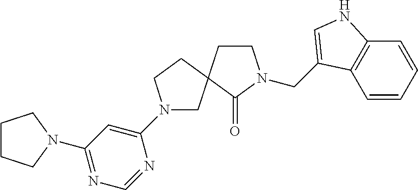 | 2-((1H-indol-3-yl) methyl)-7-(6- (pyrrolidin-1-yl) pyrimidin-4-yl)- 2,7-diazaspiro[4.4] nonan-1-one | 0.75 (D) | 417.3 |
| 5.36 | 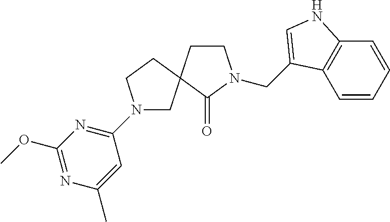 | 2-((1H-indol-3-yl) methyl)-7-(2- methoxy-6-methyl- pyrimidin-4- yl)-2,7-diazaspiro[4.4] nonan-1-one | 0.71 (D) | 392.3 |
| 5.37 | 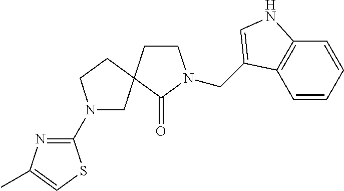 | 2-((1H-indol-3-yl) methyl)-7-(4- methylthiazol- 2-yl)-2,7- diazaspiro[4.4] nonan-1-one | 0.73 (D) | 367.2 |
| 5.38 | 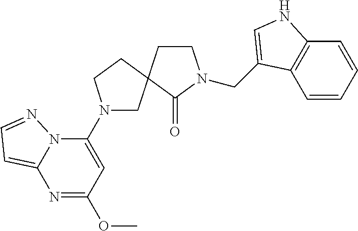 | 2-((1H-indol-3-yl) methyl)-7-(5- methoxypyrazolo[1,5- a]pyrimidin-7-yl)-2,7- diazaspiro[4.4] nonan-1-one | 1.00 (D) | 417.3 |
| 5.39 | 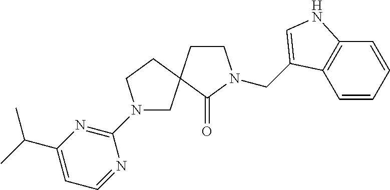 | 2-((1H-indol-3-yl) methyl)-7-(4- isopropylpyrimidin- 2-yl)-2,7- diazaspiro[4.4] nonan-1-one | 1.06 (D) | 390.3 |
| 5.40 | 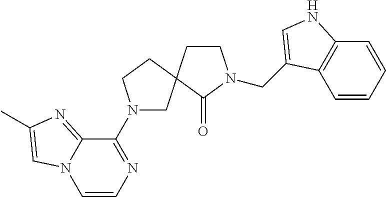 | 2-((1H-indol-3-yl) methyl)-7-(2- methylimidazo [1,2-a]pyrazin- 8-yl)-2,7- diazaspiro[4.4] nonan-1-one | 0.71 (D) | 401.3 |
| 5.41 | 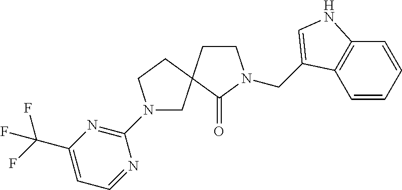 | 2-((1H-indol-3-yl) methyl)-7-(4- (trifluoromethyl) pyrimidin-2- yl)-2,7-diazaspiro [4.4]nonan-1-one | 1.10 (D) | 416.2 |
| 5.42 | 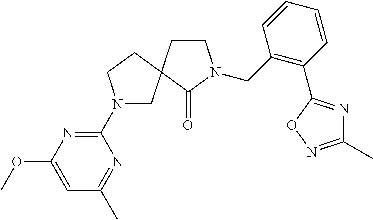 | 7-(4-methoxy-6- methylpyrimidin- 2-yl)-2-(2-(3- methyl-1,2,4-oxadiazol-5- yl)benzyl)-2,7- diazaspiro[4.4] nonan-1-one | 3.075 (A) | 435.2 |
| 5.43 | 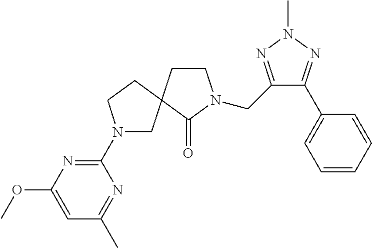 | 7-(4-methoxy-6- methylpyrimidin- 2-yl)-2-((2- methyl-5-phenyl- 2H-1,2,3- triazol-4-yl) methyl)-2,7- diazaspiro[4.4] nonan-1-one | 3.045 (A) | 434.2 |
| 5.44 | 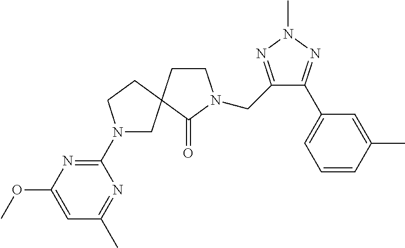 | 7-(4-methoxy-6- methylpyrimidin- 2-yl)-2-((2- methyl-5-(m-tolyl)- 2H-1,2,3- triazol-4-yl) methyl)-2,7- diazaspiro[4.4] nonan-1-one | 2.932 (B) | 448.2 |
| 5.45 | 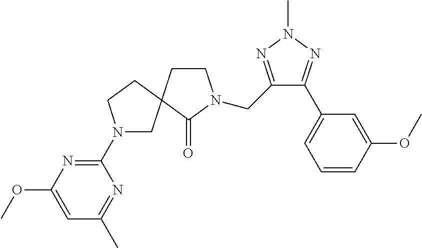 | 7-(4-methoxy-6- methylpyrimidin- 2-yl)-2-((5-(3- methoxyphenyl)- 2-methyl-2H- 1,2,3-triazol-4-yl) methyl)-2,7- diazaspiro[4.4] nonan-1-one | 2.852 (B) | 464.2 |
| 5.46 | 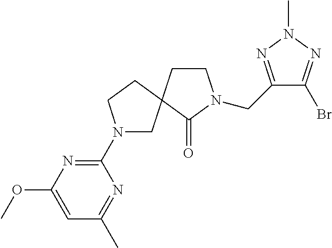 | 2-((5-bromo-2- methyl-2H- 1,2,3-triazol-4-yl) methyl)-7-(4- methoxy-6-methyl- pyrimidin-2- yl)-2,7-diazaspiro [4.4]nonan-1-one | 2.552 (B) | 436.0/438.0 |
| 5.47 | 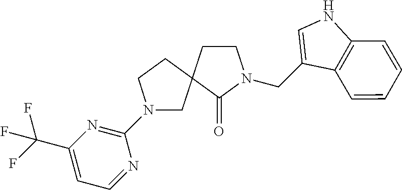 | 2-((1H-indol-3- yl)methyl)-7-(4- (dimethylamino)-6- (trifluoromethyl) pyrimidin-2- yl)-2,7-diazaspiro [4.4]nonan-1-one | 11.92 (I) | 459.85 |

### Example 6.1

### 2-((1H-Indo)-3-yl)methyl)-7-(quinoxalin-2-yl)-2,7-diazaspiro[4.5]decan-1-one

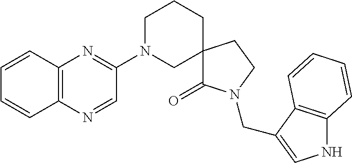

The title compound was obtained in analogy to the method described for example 2.1, starting from tert-butyl 1-oxo-2,7-diazaspiro[4.5]decane-7-carboxylate.

[1H NMR (600 MHz, DMSO-d₆) δ ppm 11.00 (s, 1H), 8.80 (s, 1H), 7.76 (d, 1H), 7.57-7.46 (m, 3H), 7.39-7.30 (m, 3H), 7.09 (t, 1H), 6.99 (t, 1H), 4.65 (d, 1H), 4.48 (d, 1H), 4.43 (d, 1H), 4.35 (d, 1H), 3.22-2.99 (m, 4H), 1.91-1.45 (m, 6H); LCMS Rt_{D}=1.06 min, [M+H]⁺=412.3]

### Example 7.1

### 7-((1H-Indol-3-yl)methyl)-2-(4-methoxy-6-methylpyrimidin-2-yl)-2,7-diazaspiro[4.5]decan-6-one

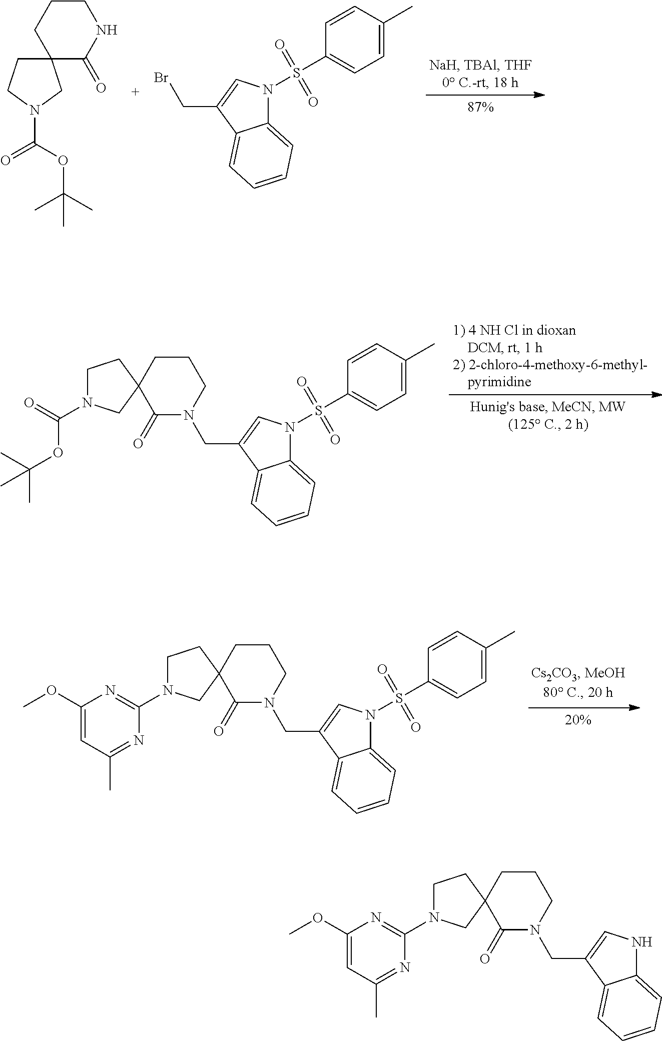

### a) 6-Oxo-7-[1-(toluene-4-sulfonyl)-1H-indol-3-ylmethyl]-2,7-diaza-spiro[4.5]decane-2-carboxylic acid tert-butyl ester

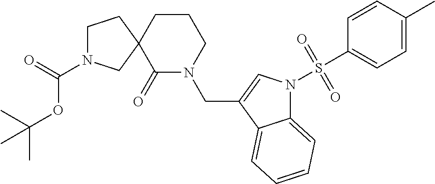

To the solution of 6-oxo-2,7-diaza-spiro[4.5]decane-2-carboxylic acid tert-butyl ester (building block A3, 850 mg, 3.34 mmol) in THF (20 ml) at 0° C. was added sodium hydride (60% in oil, 267 mg, 6.7 mmol) and the reaction was stirred for 20 min. The solution of 3-(bromomethyl)-1-tosyl-1H-indole (1.21 g, 3.34 mmol) in THF (10 ml) and TBAI (123 mg, 0.33 mmol) were added and after stirring for 15 min the ice bath was removed and the reaction was stirred at rt over night. The reaction was cooled in an ice bad and 10 drops of water were added. The mixture was extracted with EtOAc and the combined organic layers were washed with brine, dried over sodium sulfate, and the solvents were evaporated under reduced pressure. The product was purified by chromatography on silica (flashmaster, hexan/EtOAc 7/3 to hexan/EtOAc 3/7 in 40 min, then hexan/EtOAc 3/7 for 10 min) to give a slightly yellow solid. [¹H NMR (600 MHz, DMSO-d₆) δ ppm 7.91 (d, 1H), 7.83 (d, 2H), 7.80 (s, 1H), 7.57 (t, 1H), 7.39-7.33 (m, 3H), 7.24 (t, 1H), 4.68-4.54 (m, 2H), 3.55-3.09 (m, 6H), 2.32 (s, 3H), 2.23-2.11 (m, 1H), 1.75-1.61 (m, 5H), 1.41/1.40 (s, 9H); LCMS Rt_{F}=2.961 min, [M+Na]⁺=560.2].

### b) 2-(4-Methoxy-6-methyl-pyrimidin-2-yl)-7-[1-(toluene-4-sulfonyl)-1H-indol-3-ylmethyl]-2,7-diaza-spiro[4.5]decan-6-one

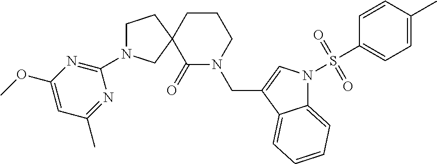

6-Oxo-7-[1-(toluene-4-sulfonyl)-1H-indol-3-ylmethyl]-2,7-diaza-spiro[4.5]decane-2-carboxylic acid tert-butyl ester (1.56 g, 2.90 mmol) was dissolved in DCM (10 ml) and 4 N HCl in dioxane (14.5 ml, 58 mmol). After stirring at rt for 1 h the solvents were evaporated under reduced pressure and the residue dissolved in DCM. The solvent was again evaporated under reduced pressure to give 7-[1-(toluene-4-sulfonyl)-1H-indol-3-ylmethyl]-2,7-diaza-spiro[4.5]decan-6-one hydrochloride as pink solid foam. This intermediate (200 mg, 0.42 mmol) was dissolved in MeCN and after addition of Hunig's base (0.33 ml, 1.90 mmol) and 2-chloro-4-methoxy-6-methyl-pyrimidine (90 mg, 0.57 mmol) the mixture was heated in a microwave apparatus at 125° C. for 2 h. The solvent was evaporated under reduced pressure and the product was purified by chromatography on silica (flashmaster, hexan/EtOAc 7/3 to hexan/EtOAc 1/9 in 40 min, then hexan/EtOAc 1/9 for 10 min) to give a colorless solid. [¹H NMR (600 MHz, DMSO-d₆) δ ppm 7.89 (d, 1H), 7.82-7.78 (m, 3H), 7.56 (d, 1H), 7.37-7.31 (m, 3H), 7.20 (dd, 1H), 5.90 (s, 1H), 4.64-4.58 (m, 2H), 3.82-3.34 (m, 8H), 2.33-2.26 (m, 4H), 2.16 (s, 3H), 1.85-1.65 (m, 5H); LCMS Rt_{C}=2.946 min, [M+H]⁺=560.2].

### c) 7-((1H-indol-3-yl)methyl)-2-(4-methoxy-6-methylpyrimidin-2-yl)-2,7-diazaspiro[4.5]decan-6-one

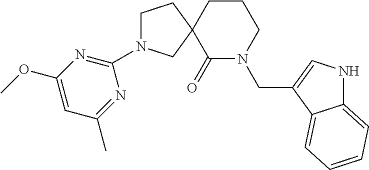

To the solution of 2-(4-methoxy-6-methyl-pyrimidin-2-yl)-7-[1-(toluene-4-sulfonyl)-1H-indol-3-ylmethyl]-2,7-diaza-spiro[4.5]decan-6-one (180 mg, 0.32 mmol) in MeOH (8 ml) was added cesium carbonate (472 mg, 1.45 mmol) and the mixture was stirred at rt for 20 h. The mixture was taken into EtOAc, washed with brine, dried over sodium sulfate, and the solvents were evaporated under reduced pressure. The residue was crystallized from DCM/diethylether/hexane to give the product as colorless crystals (26 mg, 0.064 mmol, 20%). [¹H NMR (600 MHz, DMSO-d₆) δ ppm 10.97 (s, 1H), 7.53 (d, 1H), 7.34 (d, 1H), 7.30 (d, 1H), 7.06 (dd, 1H), 6.93 (dd, 1H), 5.91 (s, 1H), 4.67-4.60 (m, 2H), 3.82-3.76 (m, 4H), 3.73-3.67 (m, 1H), 3.58-3.52 (m, 1H), 3.42-3.34 (m, 1H), 2.37-2.32 (m, 1H), 2.17 (s, 3H), 1.84-1.80 (m, 1H), 1.77-1.63 (m, 4H); LCMS Rt_{B}=2.886 min, [M+H]⁺=406.2].

### Example 7.2

### 7-((1H-Pyrrolo[2,3-b]pyridin-3-yl)methyl)-2-(4-methoxypyrimidin-2-yl)-2,7-diazaspiro[4.5]decan-6-one

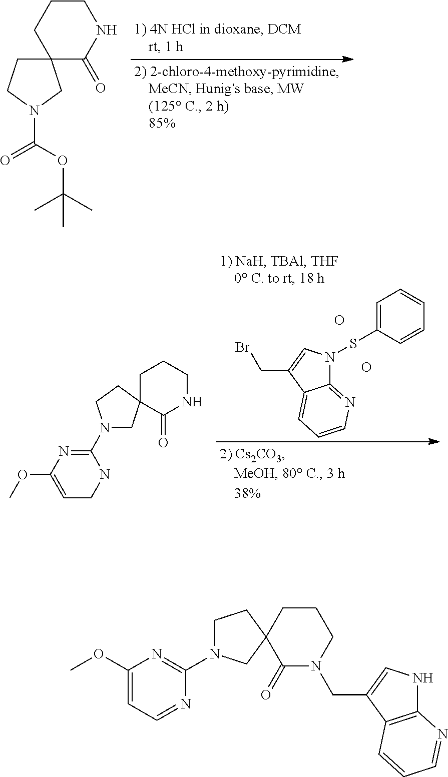

### a) 2-(4-Methoxy-pyrimidin-2-yl)-2,7-diaza-spiro[4.5]decan-6-one

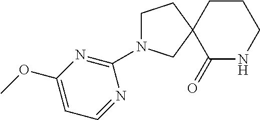

To the solution of 6-oxo-2,7-diaza-spiro[4.5]decane-2-carboxylic acid tert-butyl ester (building block A3, 325 mg, 1.28 mmol) in DCM (3 ml) was added 4N HCl in dioxane (8.0 ml) and the solution was stirred at rt for 2 h. The solvents were evaporated under reduced pressure and the residue was dissolved in MeCN (3 ml). After addition of 2-chloro-4-methoxy-pyrimidine (182 mg, 1.26 mmol) and Hunig's base (1.10 ml, 6.3 mmol) the reaction was heated in a microwave apparatus at 125° C. for 2 h. The solvents were evaporated under reduced pressure and the product was purified by chromatography on silica (flashmaster, DCM to DCM/MeOH 95/5 in 40 min, then DCM/MeOH 95/5 for 10 min), to give a colorless solid (280 mg, 1.07 mmol, 85%). [¹H NMR (600 MHz, DMSO-d₆) δ ppm 8.06 (d, 1H), 7.55 (br s, 1H), 6.04 (d, 1H), 3.84 (s, 3H), 3.74-3.38 (m, 4H), 3.19-3.17 (m, 2H), 2.33-2.28 (m, 1H), 1.88-1.82 (m, 1H), 1.80-1.70 (m, 4H); LCMS Rt_{A}=2.487 min, [M+H]⁺=263.2].

### b) 7-((1H-Pyrrolo[2,3-b]pyridin-3-yl)methyl)-2-(4-methoxypyrimidin-2-yl)-2,7-diazaspiro[4.5]decan-6-one

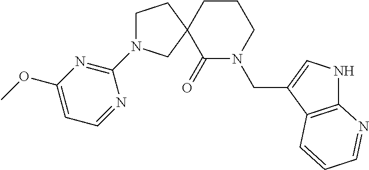

The solution of 2-(4-methoxy-pyrimidin-2-yl)-2,7-diaza-spiro[4.5]decan-6-one (75 mg, 0.286 mmol) in THF (3 ml) was cooled in an ice bath and sodium hydride (60% in oil, 23 mg, 0.57 mmol) was added in portions. After additional stirring for 20 min 1-benzenesulfonyl-3-bromomethyl-1H-pyrrolo[2,3-b]pyridine (100 mg, 0.286 mmol) was added followed by TBAI (10.6 mg, 0.029 mmol) and the reaction was again stirred for 15 min in the ice bath. The reaction was let to warm to rt over night. The solvents were evaporated under reduced pressure and the residue was dissolved in MeOH (3 ml). After addition of cesium carbonate (466 mg, 1.43 mmol) the reaction was heated at 80° C. for 3 h. The mixture was taken into EtOAc, washed with brine, dried over sodium sulfate, and the solvents were evaporated under reduced pressure. The product was purified by preparative HPLC (Waters Sunfire C18, 5 μm, 30×100 mm with guard column 19×10 mm, water (0.1% TFA)/MeCN 99/1 to 4/1 in 19 min, 50 ml/min) and crystallized from DCM/diethylether/hexane to give colorless crystals. [¹H NMR (600 MHz, DMSO-d₆) δ ppm 11.56 (s, 1H), 8.21 (d, 1H), 8.07 (s, 1H), 7.94 (d, 1H), 7.47 (s, 1H), 7.02 (s, 1H), 6.06 (d, 1H), 4.68-4.59 (m, 2H), 3.87-3.77 (m, 4H), 3.77-3.69 (m, 1H), 3.61-3.54 (m, 1H), 3.27-3.23 (m, 2H), 2.40-2.34 (m, 1H), 1.88-1.84 (m, 1H), 1.79-1.68 (m, 4H); LCMS Rt_{B}=2.223 min, [M+H]⁺=393.2].

### Examples 7.9 and 7.10

### (R)-7-((1H-indol-3-yl)methyl)-2-(4-methoxy-6-methylpyrimidin-2-yl)-2,7-diazaspiro[4.5]decan-6-one and (S)-7-((1H-indol-3-yl)methyl)-2-(4-methoxy-6-methylpyrimidin-2-yl)-2,7-diazaspiro[4.5]decan-6-one

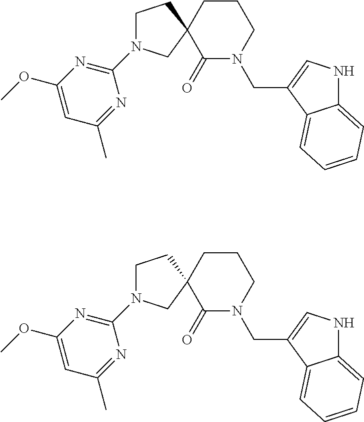

The enantiomers of Example 7.1 have been separated by chiral chromatography (VWR LAPREP P100, loop, P314; column Chiralpak AD 20 μM, 76.5×393 mm; mobile phase heptane/2-propanol 80/20, 80 ml/min) to give the title compounds as colorless solids (absolute stereochemistry arbitrarily assigned).

Example 7.9: Rt_{H}=9.93 min.

Example 7.10: Rt_{H}=14.45 min.

| | | | LCMS | |
|---|---|---|---|---|
| | | | Rt [min], | |
| Ex. | Structure | Name | method | [M + H]⁺ |
| 7.3 | 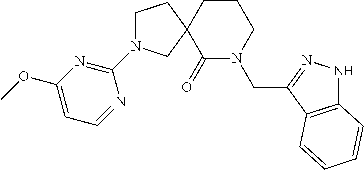 | 7-((1H-Indazol-3-yl)methyl)-2- (4-methoxypyrimidin-2-yl)-2,7- diazaspiro[4.5]decan-6-one | 2.600 (B) | 393.2 |
| 7.4 | 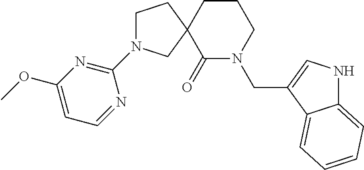 | 7-((1H-Indol-3-yl)methyl)-2-(4- methoxypyrimidin-2-yl)-2,7- diazaspiro[4.5]decan-6-one | 2.806 (B) | 392.2 |
| 7.5 | 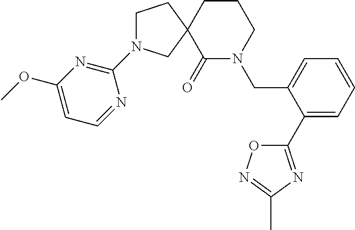 | 2-(4-Methoxypyrimidin-2-yl)-7- (2-(3-methyl-1,2,4-oxadiazol- 5-yl)benzyl)-2,7- diazaspiro[4.5]decan-6-one | 2.898 (B) | 435.2 |
| 7.6 | 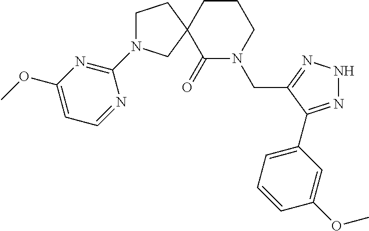 | 7-((5-(3-Methoxyphenyl)-2H- 1,2,3-triazol-4-yl)methyl)-2-(4- methoxypyrimidin-2-yl)-2,7- diazaspiro[4.5]decan-6-one | 2.658 (B) | 450.2 |
| 7.7 | 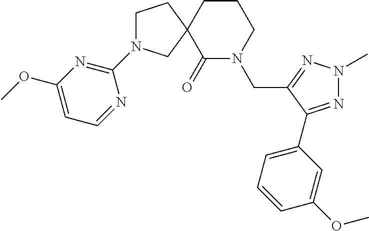 | 7-((5-(3-Methoxyphenyl)-2- methyl-2H-1,2,3-triazol-4- yl)methyl)-2-(4- methoxypyrimidin-2-yl)-2,7- diazaspiro[4.5]decan-6-one | 2.871 (B) | 464.2 |
| 7.8 | 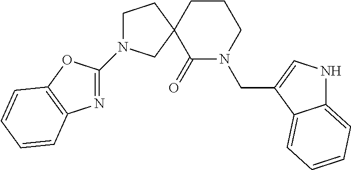 | 7-((1H-Indol-3-yl)methyl)-2- (benzo[d]oxazol-2-yl)-2,7- diazaspiro[4.5]decan-6-one | 2.121 (C) | 401.2 |
| 7.11 | 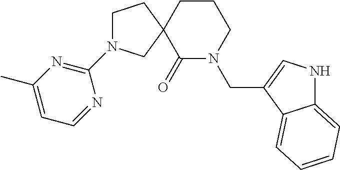 | 7-((1H-indol-3-yl)methyl)-2-(4- methylpyrimidin-2-yl)-2,7- diazaspiro[4.5]decan-6-one | 2.721 (B) | 376.2 |
| 7.12 | 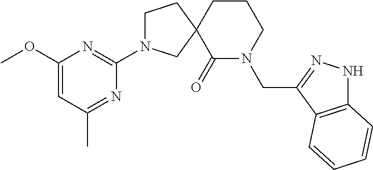 | 7-((1H-indazol-3-yl)methyl)-2- (4-methoxy-6-methylpyrimidin- 2-yl)-2,7- diazaspiro[4.5]decan-6-one | 2.957 (A) | 407.2 |
| 7.13 | 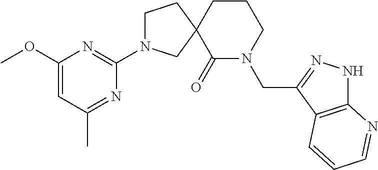 | 7-((1H-pyrrolo[2,3-b]pyridin-3- yl)methyl)-2-(4-methoxy-6- methylpyrimidin-2-yl)-2,7- diazaspiro[4.5]decan-6-one | 2.296 (B) | 407.2 |
| 7.14 | 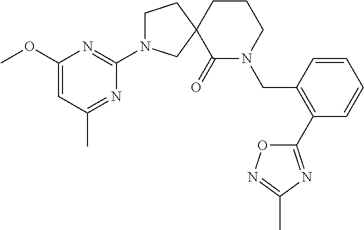 | 2-(4-methoxy-6- methylpyrimidin-2-yl)-7-(2-(3- methyl-1,2,4-oxadiazol-5- yl)benzyl)-2,7- diazaspiro[4.5]decan-6-one | 3.222 (A) | 449.2 |
| 7.15 | 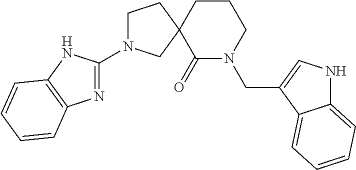 | 7-((1H-indol-3-yl)methyl)-2- (1H-benzo[d]imidazol-2-yl)- 2,7-diazaspiro[4.5]decan-6- one | 2.825 (B) | 400.2 |

### Example 8.1

### 1-((1H-Indo)-4-yl)methyl)-8-(quinoxalin-2-yl)-1,8-diazaspiro[4.5]decan-2-one

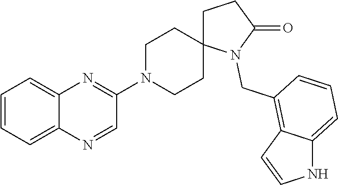

The title compound was obtained in analogy to the method described for example 2.1, starting from tert-butyl 2-oxo-1,8-diazaspiro[4.5]decane-8-carboxylate carboxylate. [¹H NMR (600 MHz, DMSO-d₆) δ ppm 11.06 (br s, 1H), 8.76 (s, 1H), 7.81 (d, 1H), 7.53-7.63 (m, 2H), 7.39 (t, 1H), 7.20 (d, 1H), 7.17 (br s, 1H), 6.83 (t, 1H), 6.75 (d, 1H), 6.43 (br s, 1H), 4.58 (s, 2H), 4.51 (d, 2H), 3.05 (t, 2H), 2.46 (t, 2H), 2.13 (t, 2H), 1.72-1.83 (m, 2H), 1.46 (d, 2H); LCMS Rt_{B}=3.03 min, [M+H]⁺=412.2].

| | | | LCMS | |
|---|---|---|---|---|
| | | | Rt [min], | |
| Ex. | Structure | Name | method | [M + H]⁺ |
| 8.2 | 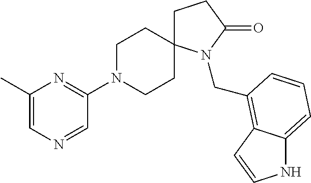 | 1-((1H-Indol-4-yl)methyl)-8-(6- methylpyrazin-2-yl)-1,8- diazaspiro[4.5]decan-2-one | 2.72 (B) | 376.2 |
| 8.3 | 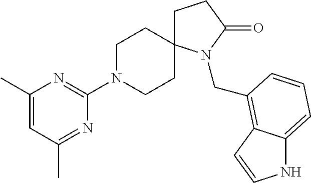 | 1-((1H-Indol-4-yl)methyl)-8- (4,6-dimethylpyrimidin-2-yl)- 1,8-diazaspiro[4.5]decan-2- one | 2.60 (B) | 390.2 |
| 8.4 | 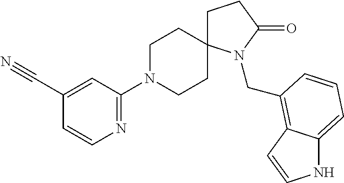 | 2-(1-((1H-Indol-4-yl)methyl)-2- oxo-1,8-diazaspiro[4.5]decan- 8-yl)isonicotinonitrile | 3.26 (A) | 386.2 |
| 8.5 | 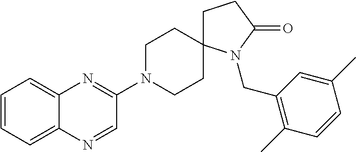 | 1-(2,5-Dimethylbenzyl)-8- (quinoxalin-2-yl)-1,8- diazaspiro[4.5]decan-2-one | 3.42 (B) | 401.2 |
| 8.6 | 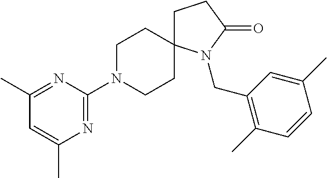 | 1-(2,5-Dimethylbenzyl)-8-(4,6- dimethylpyrimidin-2-yl)-1,8- diazaspiro[4.5]decan-2-one | 2.90 (B) | 379.2 |

### Example 8.7

### 1-(2,5-Dimethylbenzyl)-9-(quinoxalin-2-yl)-4-oxa-1,9-diazaspiro[5.5]undecan-2-one

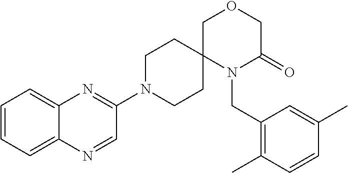

The title compound was obtained in analogy to the method described for example 1.1 starting from building block A2. [¹H NMR (600 MHz, DMSO-d₆) δ ppm 8.79 (s, 1H), 7.80 (d, 1H), 7.53-7.61 (m, 2H), 7.37 (t, 1H), 6.95 (d, 1H), 6.88 (d, 1H), 6.79 (s, 1H), 4.51 (d, 2H), 4.38 (s, 2H), 4.28 (s, 2H), 4.20 (s, 2H), 3.13 (t, 2H), 2.22 (s, 3H), 2.05 (s, 3H), 1.84-1.94 (m, 2H), 1.77 (d, 2H); LCMS Rt_{C}=2.77 min, [M+H]⁺=417.2].

| | | | LCMS | |
|---|---|---|---|---|
| | | | Rt [min], | |
| Ex. | Structure | Name | method | [M + H]⁺ |
| 8.8 | 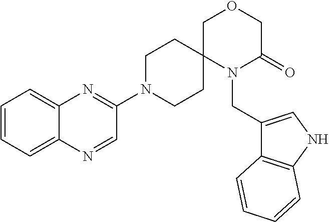 | 1-((1H-Indol-3-yl)methyl)-9- (quinoxalin-2-yl)-4-oxa-1,9- diazaspiro[5.5]undecan-2- one | 3.11 (B) | 428.2 |
| 8.9 | 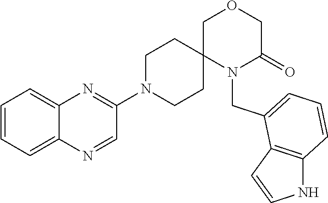 | 1-((1H-Indol-4-yl)methyl)-9- (quinoxalin-2-yl)-4-oxa-1,9- diazaspiro[5.5]undecan-2- one | 3.00 (B) | 428.2 |
| 8.10 | 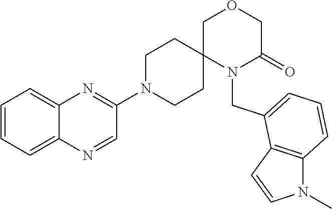 | 1-((1-Methyl-1H-indol-4- yl)methyl)-9-(quinoxalin-2- yl)-4-oxa-1,9- diazaspiro[5.5]undecan-2- one | 3.25 (B) | 442.2 |
| 8.11 | 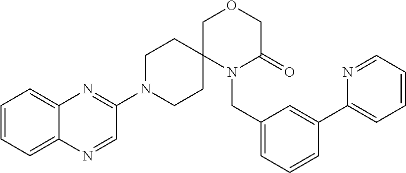 | 1-(3-(Pyridin-2-yl)benzyl)-9- (quinoxalin-2-yl)-4-oxa-1,9- diazaspiro[5.5]undecan-2- one | 2.69 (B) | 466.2 |
| 8.12 | 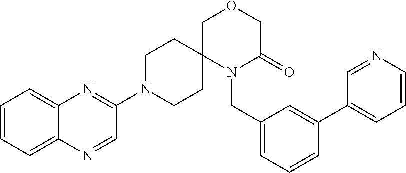 | 1-(3-(Pyridin-3-yl)benzyl)-9- (quinoxalin-2-yl)-4-oxa-1,9- diazaspiro[5.5]undecan-2- one | 2.70 (B) | 466.2 |
| 8.13 | 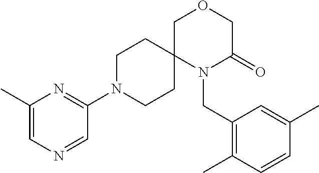 | 1-(2,5-Dimethylbenzyl)-9-(6- methylpyrazin-2-yl)-4-oxa- 1,9-diazaspiro[5.5]undecan- 2-one | 3.35 (A) | 381.2 |
| 8.14 | 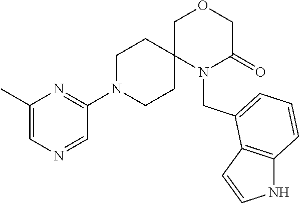 | 1-((1H-Indol-4-yl)methyl)-9- (6-methylpyrazin-2-yl)-4- oxa-1,9- diazaspiro[5.5]undecan-2- one | 2.72 (B) | 392.2 |
| 8.15 | 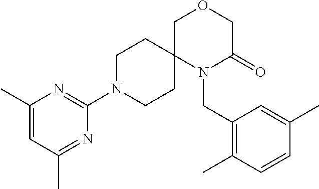 | 1-(2,5-Dimethylbenzyl)-9- (4,6-dimethylpyrimidin-2-yl)- 4-oxa-1,9- diazaspiro[5.5]undecan-2- one | 2.90 (B) | 395.2 |
| 8.16 | 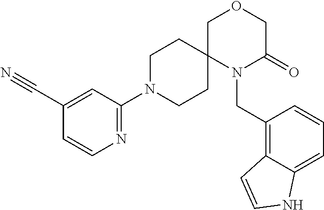 | 2-(1-((1H-Indol-4-yl)methyl)- 2-oxo-4-oxa-1,9- diazaspiro[5.5]undecan-9- yl)isonicotinonitrile | 2.97 (B) | 402.2 |
| 8.17 | 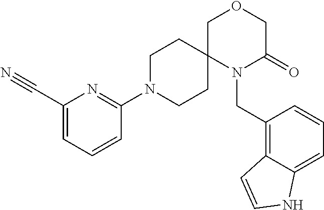 | 6-(1-((1H-Indol-4-yl)methyl)- 2-oxo-4-oxa-1,9- diazaspiro[5.5]undecan-9- yl)picolinonitrile | 3.16 (B) | 402.2 |
| 8.18 | 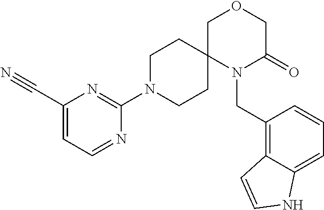 | 2-(1-((1H-Indol-4-yl)methyl)- 2-oxo-4-oxa-1,9- diazaspiro[5.5]undecan-9- yl)pyrimidine-4-carbonitrile | 3.16 (B) | 403.2 |
| 8.19 | 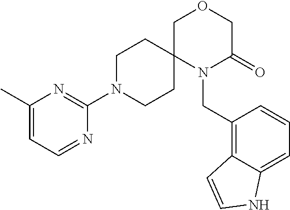 | 1-((1H-Indol-4-yl)methyl)-9- (4-methylpyrimidin-2-yl)-4- oxa-1,9- diazaspiro[5.5]undecan-2- one | 2.87 (A) | 392.2 |

### Example 8.20

### 1-(2,5-Dimethylbenzyl)-8-(quinoxalin-2-yl)-3-oxa-1,8-diazaspiro[4.5]decan-2-one

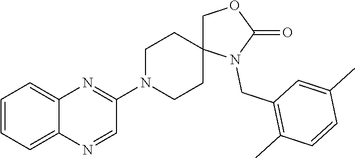

The title compound was obtained in analogy to the method described for example 1.1 starting from 3-oxa-1,8-diazaspiro[4.5]decan-2-one. [¹H NMR (400 MHz, DMSO-d₆) δ ppm 8.79 (s, 1H), 7.79 (d, 1H), 7.53-7.61 (m, 2H), 7.37 (ddd, 1H), 6.94 (d, 1H), 6.92 (s, 1H), 6.86 (d, 1H), 4.57 (d, 2H), 4.45 (s, 2H), 4.23 (s, 2H), 2.99 (t, 2H), 2.11 (s, 3H), 2.06 (s, 3H), 1.63-1.84 (m, 4H); LCMS Rt_{B}=3.56 min, [M+H]⁺=403.2].

### Example 8.21

### 1-((1H-Indo)-3-yl)methyl)-8-(quinoxalin-2-yl)-3-oxa-1,8-diazaspiro[4.5]decan-2-one

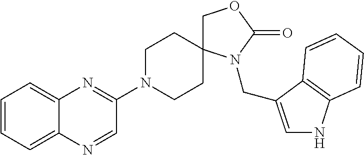

The title compound was obtained in analogy to the method described for example 1.1 starting from 3-oxa-1,8-diazaspiro[4.5]decan-2-one. [¹H NMR (600 MHz, DMSO-d₆) δ ppm 11.10 (br s, 1H), 8.76 (s, 1H), 7.81 (d, 1H), 7.53-7.62 (m, 2H), 7.40 (t, 1H), 7.20-7.26 (m, 1H), 7.19 (br s, 1H), 6.82-6.89 (m, 2H), 6.45 (br s, 1H), 4.57 (s, 2H), 4.51 (d, 2H), 4.39 (s, 2H), 2.95 (t, 2H), 1.72-1.87 (m, 2H), 1.58 (d, 2H)].

| | | | LCMS | |
|---|---|---|---|---|
| | | | Rt [min], | |
| Ex. | Structure | Name | method | [M + H]⁺ |
| 8.22 | 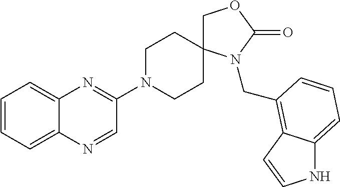 | 1-((1H-Indol-4-yl)methyl)-8- (quinoxalin-2-yl)-3-oxa-1,8- diazaspiro[4.5]decan-2-one | 3.71 (B) | 378.2 |
| 8.23 | 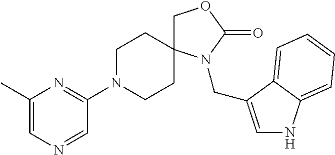 | 1-((1H-Indol-3-yl)methyl)-8- (6-methylpyrazin-2-yl)-3-oxa- 1,8-diazaspiro[4.5]decan-2- one | 1.46 (C) | 378.2 |
| 8.24 | 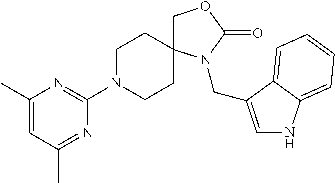 | 1-((1H-Indol-3-yl)methyl)-8- (4,6-dimethylpyrimidin-2-yl)-3- oxa-1,8-diazaspiro[4.5]decan- 2-one | 3.05 (A) | 392.2 |
| 8.25 | 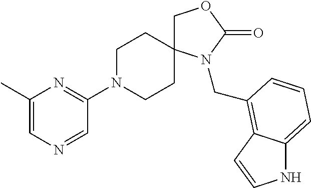 | 1-((1H-Indol-4-yl)methyl)-8- (6-methylpyrazin-2-yl)-3-oxa- 1,8-diazaspiro[4.5]decan-2- one | 3.07 (A) | 378.2 |

### Example 9.1

### 1-((1H-Indo)-4-yl)methyl)-7-(quinoxalin-2-yl)-1,7-diazaspiro[4.4]nonan-2-one

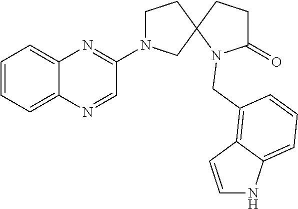

The title compound was obtained in analogy to the method described for example 2.1, starting from tert-butyl 2-oxo-1,7-diazaspiro[4.4]nonane-7-carboxylate. [¹H NMR (600 MHz, DMSO-d₆) δ ppm 11.14 (br s, 1H), 8.33 (br s, 1H), 7.80 (d, 1H), 7.49-7.61 (m, 2H), 7.35 (t, 1H), 7.31 (br s, 1H), 7.26 (d, 1H), 7.00 (t, 1H), 6.83 (d, 1H), 6.54 (br s, 1H), 4.77 (d, 1H), 4.70 (d, 1H), 3.74 (t, 1H), 3.67 (d, 1H), 3.48-3.60 (m, 2H), 2.46-2.64 (m, 3H), 2.04-2.29 (m, 3H); LCMS Rt_{B}=2.39 min, [M+H]⁺=398.2].

Radioligand Binding Assay

For crude cell membrane preparations, cells (CHO, Chinese hamster ovary or HEK, human embryonic kidney) expressing human orexin 1 or human orexin 2 receptors, were washed with HEPES (10 mM, pH 7.5), scraped off the culture plates with the same buffer, and centrifuged at 4° C. for 5 min at 2500×g. The cell pellet was either stored at −80° C. or used directly. Before the experiments, cell membranes were re-suspended in binding assay buffer (10 mM HEPES, 0.5% (w/v) bovine serum albumin, pH 7.5) by homogenisation with a Polytron homogeniser at 50 Hz for 20 s. Cell membranes were also used as made available by commercial providers.

In initial saturation experiments (to calculate Kd and Bmax), cell homogenates (150 μl) were incubated with 0.1 to 15 nM of the radioligand ([³H]-SB649868, 50 μl), 8 concentrations in triplicates in the presence or absence of almorexant (10 μM, 50 μl) to define non specific binding. Bound radioactivity was measured, and data were analysed with the program XLFIT or Graphpad Prism. Protein concentration was determined according to the Bradford/BioRad Protein Assay Kit.

In competition experiments, cell homogenates (150 μl) were incubated in assay buffer (10 mM HEPES, pH 7.5, 0.5% (w/v) bovine serum albumin, 5 mM MgCl₂, 1 mMCaCl₂, and tween 0.05%) for 1 h at room temperature with about 1 nM of the radioligand [³H]-SB649868, 66 Ci/mmole, 50 μl), and with various concentrations of compounds of the invention (50 μl) in triplicates; non-specific binding was determined in the presence of almorexant (10 μM). Reactions were terminated by vacuum filtration, 3 washes of ice cold wash buffer (Tris-HCl pH 7.4/10 mM, with NaCl 154 mM). Competition data is expressed in Table 1 as Kd [μM].

Calcium Accumulation in Cells (FLIPR):

Cells expressing human orexin 1 or human orexin 2 receptors, were seeded at 8,000 cells/well in 384 well black-walled clear bottom, poly-D-lysine coated plates. After 24 h, the medium was removed and cells were washed once with phosphate buffered saline and serum-deprived overnight in assay buffer (130 mM NaCl, 5.4 mM KCl, 1.8 mM CaCl₂, 0.8 mM MgSO₄, 0.9 mM NaH₂PO₄, 25 mM glucose, 20 mM HEPES, pH 7.4) containing bovine serum albumin (1% w/v).

On the day of the experiment, the cells seeded in black plates were treated with assay buffer containing the Ca²⁺ sensitive fluorescent dye Fluo-4-AM (2 μM), and probenecid (0.1 mM). After 1 h plates were washed twice with, and resuspended in, assay buffer containing probenecid (0.1 mM) using a multi plate washer. The plates were placed into a FLIPR II (Fluorometric Imaging Plate Reader, Molecular Devices, Sunnyvale, Calif., USA) and baseline fluorescence (fluorescence light units, FLU) was measured (5 measurements, 2 S each; laser excitation 488 nm at 0.6-1 W, CCD camera exposure 0.4 s) before addition of buffer alone (basal) or containing test compounds (either compound of formula I alone, agonist alone or agonist in the presence of various concentrations of compounds of formula I). Fluorescence measurements were then continued every 1 S for 120 S followed by every 4 S for 240 S.

The measurements were typically made in two sequences:

In the first round, compounds of formula I were tested alone, to confirm that they do not display any significant agonist activity. Compounds of formula I were tested usually in a concentration range from 10⁻⁹ M to 10⁻⁵ M.

In the second round, performed one hour later (to allow for equilibration), Orexin A was tested either in the absence (calibration curves, Orexin A agonist controls) or in the presence of compounds of formula Ito determine antagonism.

Inhibition data is expressed in Table 1 as K_{d} [μM], converted by the Cheng and Prusoff correction (Kd=IC₅₀/1+(L/EC₅₀)), where IC₅₀ is the 50% inhibition value determined in concentration response inhibition curves, EC₅₀ is the half maximal activation concentration determined for orexin A in concentration response curves and L is the concentration of orexin A used in inhibition experiments performed in with a submaximal concentration of orexin A in the presence of up to 8 increasing concentrations of compound of formula I. Inhibition data is also expressed in Table 1 as % inhibition value measured at a concentration of 10 μM of selected compounds of formula I.

TABLE 1

| | FLIPR | FLIPR | Binding | Binding |
|---|---|---|---|---|
| | hOx1R | hOx2R_ | hOx1R | hOx2R |
| Example | Ki [μM] | Ki [μM] | Kd [μM] | Kd [μM] |
| 1.1 | 3.37 | 0.020 | 1.27 | 0.028 |
| 1.2 | 3.65 | 0.449 | n.d. | n.d. |
| 1.3 | 35^{a} | 0.334 | n.d. | n.d. |
| 1.4 | 0.601 | 0.006 | 0.770 | 0.017 |
| 1.5 | 1.49 | 0.007 | 0.928 | 0.031 |
| 1.6 | 26^{a} | 0.222 | n.d. | n.d. |
| 1.7 | 35^{a} | 0.101 | n.d. | n.d. |
| 1.8 | 0.906 | 0.008 | 0.679 | 0.019 |
| 1.9 | 0.230 | 0.004 | 0.397 | 0.025 |
| 1.10 | 2.61 | 0.084 | 3.97 | 0.164 |
| 1.11 | 2.66 | 0.010 | 2.22 | 0.037 |
| 1.12 | 0.621 | 0.065 | 0.686 | 0.230 |
| 1.13 | 0.037 | 0.003 | 0.082 | 0.20 |
| 1.14 | 0.298 | 0.002 | 0.445 | 0.022 |
| 1.15 | 44^{a} | 0.107 | n.d. | n.d. |
| 1.16 | 35^{a} | 0.031 | n.d. | 0.188 |
| 1.17 | 2.09 | 0.092 | 2.346 | 0.251 |
| 1.18 | 0.119 | 0.003 | 0.219 | 0.023 |
| 1.19 | 2.65 | 0.250 | n.d. | n.d. |
| 1.20 | 12^{a} | 1.84 | n.d. | n.d. |
| 1.21 | 29^{a} | 0.337 | n.d. | n.d. |
| 1.22 | 19^{a} | 1.02 | n.d. | n.d. |
| 1.23 | 2.11 | 0.192 | n.d. | n.d. |
| 1.24 | 0.766 | 0.010 | 0.924 | 0.035 |
| 1.25 | 31^{a} | 0.076 | n.d. | 0.394 |
| 1.26 | 0.391 | 0.008 | 0.352 | 0.049 |
| 1.27 | 34^{a} | 0.094 | 5.14 | 0.140 |
| 1.28 | 33^{a} | 0.511 | n.d. | n.d. |
| 1.29 | 1.43 | 0.075 | 1.32 | 0.100 |
| 1.30 | 51^{a} | 0.029 | 2.14 | 0.114 |
| 1.31 | 38^{a} | 0.064 | n.d. | n.d. |
| 1.32 | 2.84 | 0.084 | n.d. | n.d. |
| 1.33 | 1.53 | 0.006 | 1.62 | 0.042 |
| 1.34 | 0.494 | 0.001 | 0.234 | 0.004 |
| 1.35 | 25^{a} | 0.400 | n.d. | n.d. |
| 1.36 | 41^{a} | 0.187 | n.d. | n.d. |
| 1.37 | 40^{a} | 2.333 | n.d. | n.d. |
| 1.38 | 29^{a} | 0.204 | n.d. | n.d. |
| 1.39 | 0.304 | 0.002 | 0.231 | 0.013 |
| 1.40 | 2.81 | 0.020 | 5.30 | 0.079 |
| 1.41 | 1.58 | 0.016 | 3.17 | 0.182 |
| 1.42 | 3.28 | 0.083 | 6.49 | 0.391 |
| 1.43 | 27^{a} | 0.447 | n.d. | n.d. |
| 1.44 | 12^{a} | 1.49 | n.d. | n.d. |
| 1.45 | 21^{a} | 45^{a} | n.d. | n.d. |
| 1.46 | 2.04 | 0.005 | 3.88 | 0.091 |
| 1.47 | 0.135 | 0.001 | 0.187 | 0.007 |
| 1.48 | 2.74 | 0.012 | 2.27 | 0.099 |
| 1.49 | 20^{a} | 38^{a} | n.d. | n.d. |
| 1.50 | 1.18 | 0.086 | n.d. | n.d. |
| 1.51 | <10^{a} | 1.24 | n.d. | n.d. |
| 1.52 | 0.286 | 0.0028 | 0.436 | 0.050 |
| 1.53 | 0.331 | 0.007 | 0.319 | 0.045 |
| 1.54 | 15^{a} | 32^{a} | n.d. | n.d. |
| 1.55 | n.d. | 0.016 | 5.87 | 0.162 |
| 1.56 | 0.594 | 0.0027 | 0.492 | 0.011 |
| 1.57 | 2.67 | 0.175 | n.d. | n.d. |
| 1.58 | 17^{a} | 0.458 | n.d. | n.d. |
| 1.59 | 37^{a} | 0.252 | n.d. | n.d. |
| 1.60 | 13^{a} | 34^{a} | n.d. | n.d. |
| 1.61 | 0.836 | 0.0018 | n.d. | n.d. |
| 1.62 | 0.163 | 0.0010 | 0.142 | 0.008 |
| 1.63 | 4.08 | 0.046 | n.d. | 0.252 |
| 1.64 | 32^{a} | 0.423 | n.d. | n.d. |
| 1.65 | 1.58 | 0.021 | 1.16 | 0.058 |
| 1.66 | 3.92 | 0.632 | n.d. | n.d. |
| 1.67 | 1.14 | 0.005 | 1.77 | 0.050 |
| 1.68 | 0.945 | 0.0040 | 1.44 | 0.013 |
| 1.69 | 13^{a} | 0.235 | n.d. | n.d. |
| 1.70 | 0.856 | 0.002 | 0.465 | 0.021 |
| 1.71 | 2.06 | 0.030 | 1.70 | 0.227 |
| 1.72 | 1.59 | 0.049 | 0.846 | 0.203 |
| 1.73 | 0.862 | 0.020 | 1.38 | 0.163 |
| 1.74 | 4.27 | 0.048 | 2.50 | 0.127 |
| 1.75 | <10^{a} | 35^{a} | n.d. | n.d. |
| 1.76 | 1.636 | 0.086 | n.d. | 0.174 |
| 1.77 | 2.877 | 0.056 | 2.84 | 0.225 |
| 1.78 | 1.869 | 0.009 | 0.709 | 0.028 |
| 1.79 | 0.166 | 0.0037 | 0.535 | 0.022 |
| 1.80 | 0.501 | 0.0043 | 0.860 | 0.027 |
| 1.81 | 33^{a} | 2.19 | n.d. | n.d. |
| 1.82 | 57^{a} | 0.655 | n.d. | n.d. |
| 1.83 | 4.45 | 0.240 | n.d. | n.d. |
| 2.1 | 1.70 | 0.029 | 1.32 | 0.031 |
| 2.2 | 1.80 | 0.055 | 1.84 | 0.071 |
| 2.3 | 1.10 | 0.142 | n.d. | n.d. |
| 2.4 | 0.367 | 0.006 | n.d. | n.d. |
| 2.5 | 1.55 | 0.061 | n.d. | n.d. |
| 2.6 | 1.06 | 0.013 | 1.06 | 0.026 |
| 2.7 | 0.258 | 0.001 | 0.213 | 0.006 |
| 2.8 | 2.46 | 0.105 | 5.61 | 0.078 |
| 2.9 | 42^{a} | 0.659 | n.d. | n.d. |
| 2.10 | 35^{a} | 0.072 | 4.61 | 0.144 |
| 2.11 | 2.32 | 0.075 | 3.18 | 0.229 |
| 2.12 | 16^{a} | 0.364 | n.d. | n.d. |
| 2.13 | 1.50 | 0.038 | 1.56 | 0.143 |
| 2.14 | 2.72 | 0.050 | n.d. | 0.241 |
| 2.15 | 10^{a} | 2.16 | n.d. | n.d. |
| 2.16 | 16^{a} | 27^{a} | n.d. | n.d. |
| 2.17 | 0.220 | 0.001 | 0.363 | 0.016 |
| 2.18 | 0.383 | 0.004 | 0.625 | 0.023 |
| 2.19 | 1.49 | 0.035 | 3.52 | 0.184 |
| 2.20 | 0.043 | 0.002 | 0.057 | 0.007 |
| 2.21 | 3.27 | 0.227 | n.d. | n.d. |
| 2.22 | 2.78 | 0.251 | n.d. | n.d. |
| 2.23 | 0.582 | 0.037 | 1.57 | 0.068 |
| 2.24 | 1.65 | 0.182 | n.d. | n.d. |
| 2.25 | 2.68 | 0.374 | n.d. | n.d. |
| 2.26 | 1.18 | 0.148 | n.d. | n.d. |
| 3.1 | 1.90 | 0.032 | n.d. | 0.228 |
| 3.2 | 1.06 | 0.041 | 0.920 | 0.121 |
| 3.3 | 27^{a} | 0.496 | n.d. | n.d. |
| 3.4 | 3.77 | 0.287 | n.d. | n.d. |
| 3.5 | 25^{a} | 0.296 | n.d. | n.d. |
| 3.6 | 1.63 | 0.784 | n.d. | n.d. |
| 3.7 | n.d. | n.d. | >10^{b} | 1.92^{b} |
| 3.8 | 0.506 | 0.644 | n.d. | n.d. |
| 3.9 | 0.343 | 0.731 | n.d. | n.d. |
| 3.10 | n.d. | 1.08 | n.d. | n.d. |
| 3.11 | 1.07 | 0.195 | 6.36^{b} | 0.414^{b} |
| 3.12 | 4.97 | 0.376 | n.d. | n.d. |
| 3.13 | 0.342 | 0.342 | n.d. | n.d. |
| 3.14 | 2.77 | 0.134 | n.d. | n.d. |
| 3.15 | 26^{a} | 0.637 | n.d. | n.d. |
| 3.16 | 39^{a} | 0.673 | n.d. | n.d. |
| 3.17 | 37^{a} | 0.298 | n.d. | n.d. |
| 3.18 | 1.24 | 0.037 | n.d. | 1.03 |
| 3.19 | 22^{a} | 0.127 | n.d. | n.d. |
| 3.20 | 1.64 | 0.043 | 7.67 | 0.328 |
| 3.21 | 1.01 | 0.117 | n.d. | 0.699 |
| 3.22 | 24^{a} | 0.246 | n.d. | n.d. |
| 3.23 | 10^{a} | 2.39 | n.d. | n.d. |
| 3.24 | 25^{a} | 0.117 | n.d. | n.d. |
| 4.1 | 12^{a} | 2.12 | n.d. | n.d. |
| 5.1 | 2.14 | 0.132 | n.d. | n.d. |
| 5.2 | 49^{a} | 1.26 | n.d. | n.d. |
| 5.3 | 2.64 | 0.103 | n.d. | n.d. |
| 5.4 | 1.05 | 0.119 | n.d. | n.d. |
| 5.5 | 1.23 | 0.430 | n.d. | n.d. |
| 5.6 | 33^{a} | 0.393 | n.d. | n.d. |
| 5.7 | 2.13 | 0.249 | n.d. | n.d. |
| 5.8 | 0.739 | 0.304 | n.d. | n.d. |
| 5.9 | <10^{a} | 1.29 | n.d. | n.d. |
| 5.10 | 1.34 | 0.629 | n.d. | n.d. |
| 5.11 | <10^{a} | 26^{a} | n.d. | n.d. |
| 5.12 | 40^{a} | 0.711 | n.d. | n.d. |
| 5.13 | 4.16 | 0.673 | n.d. | n.d. |
| 5.14 | 0.544 | 0.146 | n.d. | n.d. |
| 5.15 | 29^{a} | 37^{a} | n.d. | n.d. |
| 5.16 | 1.12 | 0.509 | n.d. | n.d. |
| 5.17 | 49^{a} | 1.79 | n.d. | n.d. |
| 5.18 | 1.89 | 1.24 | n.d. | n.d. |
| 5.19 | 1.19 | 0.665 | n.d. | n.d. |
| 5.20 | 34^{a} | 33^{a} | n.d. | n.d. |
| 5.21 | 1.30 | 0.370 | n.d. | n.d. |
| 5.22 | 0.923 | 0.489 | n.d. | n.d. |
| 5.23 | 67^{a} | 0.645 | n.d. | n.d. |
| 5.24 | 25^{a} | 54^{a} | n.d. | n.d. |
| 5.25 | 71^{a} | 66^{a} | n.d. | n.d. |
| 5.26 | 37^{a} | 47^{a} | n.d. | n.d. |
| 5.27 | 2.39 | 0.298 | n.d. | n.d. |
| 5.28 | 34^{a} | 0.182 | n.d. | n.d. |
| 5.29 | 1.49 | 0.114 | n.d. | n.d. |
| 5.30 | 4.35 | 0.193 | n.d. | n.d. |
| 5.31 | <10^{a} | 24^{a} | n.d. | n.d. |
| 5.32 | 4.47 | 2.288 | n.d. | n.d. |
| 5.33 | <10^{a} | <10^{a} | n.d. | n.d. |
| 5.34 | 2.15 | 1.471 | n.d. | n.d. |
| 5.35 | 1.77 | 2.718 | n.d. | n.d. |
| 5.36 | 18^{a} | 0.927 | n.d. | n.d. |
| 5.37 | 3.59 | 1.41 | n.d. | n.d. |
| 5.38 | 2.41 | 1.02 | n.d. | n.d. |
| 5.39 | 1.21 | 0.283 | n.d. | n.d. |
| 5.40 | 1.73 | 0.188 | n.d. | n.d. |
| 5.41 | 2.41 | 0.343 | n.d. | n.d. |
| 5.42 | 1.95 | 0.180 | n.d. | n.d. |
| 5.43 | 0.421 | 0.029 | 0.339 | 0.118 |
| 5.44 | 0.068 | 0.0034 | 0.106 | 0.020 |
| 5.45 | 0.029 | 0.0028 | 0.097 | 0.021 |
| 5.46 | <10^{a} | 21^{a} | n.d. | n.d. |
| 5.47 | 0.193 | 0.097 | 1.03 | 0.518 |
| 6.1 | 1.13 | 0.639 | n.d. | n.d. |
| 7.1 | 0.031 | 0.010 | 0.014 | 0.013 |
| 7.2 | 3.20 | 0.703 | n.d. | n.d. |
| 7.3 | 1.432 | 0.234 | n.d. | n.d. |
| 7.4 | 0.317 | 0.059 | 0.201 | 0.152 |
| 7.5 | 0.981 | 0.221 | n.d. | n.d. |
| 7.6 | 0.189 | 0.065 | 0.187 | 0.170 |
| 7.7 | 0.537 | 0.366 | n.d. | n.d. |
| 7.8 | 0.098 | 0.168 | n.d. | n.d. |
| 7.9 | 0.010 | 0.0019 | 0.012 | 0.012 |
| 7.10 | 0.139 | 0.014 | 0.750 | 0.238 |
| 7.11 | 0.304 | 0.038 | 0.089 | 0.073 |
| 7.12 | 0.309 | 0.048 | 0.173 | 0.138 |
| 7.13 | 0.299 | 0.115 | n.d. | n.d. |
| 7.14 | 0.174 | 0.092 | 0.171 | 0.210 |
| 7.15 | 0.474 | 0.068 | 0.043 | 0.028 |
| 8.1 | 0.078 | 0.0021 | 0.148 | 0.032 |
| 8.2 | 12^{a} | 0.133 | n.d. | n.d. |
| 8.3 | 1.21 | 0.0023 | 2.38 | 0.030 |
| 8.4 | 36^{a} | 0.045 | >10 | 0.348 |
| 8.5 | 0.100 | 0.0076 | 0.081 | 0.077 |
| 8.6 | 2.78 | 0.010 | 4.92 | 0.153 |
| 8.7 | 0.274 | 0.024 | 0.431 | 0.102 |
| 8.8 | 33^{a} | 0.635 | n.d. | n.d. |
| 8.9 | 0.320 | 0.029 | 0.429 | 0.136 |
| 8.10 | 0.265 | 0.202 | n.d. | n.d. |
| 8.11 | 0.053 | 0.783 | 0.053 | 4.54 |
| 8.12 | 0.232 | 30^{a} | n.d. | n.d. |
| 8.13 | <10^{a} | 0.405 | n.d. | n.d. |
| 8.14 | 14^{a} | 0.506 | n.d. | n.d. |
| 8.15 | 15^{a} | 0.028 | >10 | 0.165 |
| 8.16 | <10^{a} | 1.02 | n.d. | n.d. |
| 8.17 | 40^{a} | 0.584 | n.d. | n.d. |
| 8.18 | 25^{a} | 1.01 | n.d. | n.d. |
| 8.19 | 28^{a} | 0.697 | n.d. | n.d. |
| 8.20 | 0.141 | 0.014 | 0.171 | 0.152 |
| 8.21 | 33^{a} | 0.480 | n.d. | n.d. |
| 8.22 | 0.645 | 0.038 | 0.986 | 0.290 |
| 8.23 | <10^{a} | 20^{a} | n.d. | n.d. |
| 8.24 | 21^{a} | 0.448 | n.d. | n.d. |
| 8.25 | <10^{a} | 0.675 | n.d. | n.d. |
| 9.1 | 0.666 | 1.38 | n.d. | n.d. |

n.d. = not determined
^{a}a % inhibition value measured at a concentration of 10 μM of compound of formula I.
^{b}radioligand ([¹²⁵I]orexin A was used instead of [³H]-SB649868 in the binding assay: In competition experiments, cell homogenates (150 μ1) were incubated in assay buffer (10 mM HEPES, pH 7.5, 0.5 % (w/v) bovine serum albumin, 5 mM MgCl₂, 1 mM CaCl₂, and tween 0.05%) for 1 h at room temperature with about 100 pM of the radioligand ([¹²⁵I]orexin A, 2100 Ci/mmole, 50 μl), and with various concentrations of compounds of the invention (50 μl) in triplicates; non-specific binding was determined in the presence of Orexin A (1 μM).
Reactions were terminated by vacuum filtration, 3 washes of ice cold wash buffer (Tris-HCl pH 7.4/10 mM, with NaCI 154 mM). Competition data is expressed in Table 1 as Kd [μM].

The following are further embodiments of the invention:

### Embodiment 1

a compound of the formula I

A-D-C(R₁)₂—B (I),

wherein

each R₁ independently is hydrogen, C₁₋₆alkyl, C₁₋₆halogenalkyl, C₃₋₇cycloalkyl or C₃₋₇cycloalkyl(C₁₋₄alkyl), or two R₁ together with the carbon atom to which they are bound form a C₃₋₄cycloalkyl;

A is a five- to six-membered monocyclic aromatic ring system which may contain from 1 to 4 hetero atoms selected from nitrogen, oxygen and sulfur, and which is substituted once or more than once by R₂;

or A is an eight- to ten-membered fused bicyclic aromatic ring system which may contain from 1 to 4 hetero atoms selected from nitrogen, oxygen and sulfur, and which may be substituted once or more than once by R₂;

each R₂ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; or a three- to seven-membered monocyclic ring system which may be aromatic, saturated or unsaturated non-aromatic, which may contain from 1 to 4 hetero atoms selected from nitrogen, oxygen and sulfur, and which may be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; or two R₂ at adjacent ring atoms form together with said ring atoms a fused five- to seven-membered unsaturated non-aromatic ring system which may contain from 1 to 4 hetero atoms selected from nitrogen, oxygen and sulfur, and which may be substituted once or more than once by R₃;

each R₃ independently is halogen, C₁₋₆alkyl or C₁₋₆alkoxy, or two R₃ at the same ring atom together are oxo;

B is a five- to six-membered monocyclic aromatic ring system which may contain from 1 to 4 hetero atoms selected from nitrogen, oxygen and sulfur, and which is substituted once or more than once by R₄;

or B is a eight- to ten-membered fused bicyclic aromatic ring system which may contain from 1 to 4 hetero atoms selected from nitrogen, oxygen and sulfur, and which may be substituted once or more than once by R₄;

each R₄ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; B1; or two R₄ at adjacent ring atoms form together with said ring atoms a fused five- to seven-membered unsaturated non-aromatic ring system which may contain from 1 to 4 hetero atoms selected from nitrogen, oxygen and sulfur, and which may in turn be substituted once or more than once by R₅;

B1 is a three- to seven-membered monocyclic ring system which may be aromatic, saturated or unsaturated non-aromatic, which may contain from 1 to 4 hetero atoms selected from nitrogen, oxygen and sulfur, and which may in turn be substituted once or more than once by C₁₋₆alkyl, C₁₋₆halogenalkyl, C₁₋₄alkoxy-C₁₋₆alkyl, C₁₋₆alkoxy, C₃₋₇cycloalkoxy, C₁₋₆halogenalkoxy, C₃₋₇cycloalkylC₁₋₄alkoxy, C₁₋₄alkoxy-C₁₋₄alkoxy, C₁₋₄alkylcarbonyl, N—C₁₋₄alkylaminocarbonyl, C₁₋₄alkylamino, di(C₁₋₄alkyl)amino, halogen, cyano, a 6-membered saturated heterocycle containing 1 to 4 hetero atoms selected from nitrogen, oxygen and sulfur, a 5-membered aromatic ring containing 1 to 4 hetero atoms selected from nitrogen, oxygen and sulfur which may be substituted once or more than once by C₁₋₄alkyl;

or two substituents at adjacent ring atoms of B1 form together with said ring atoms a fused five- to seven-membered unsaturated non-aromatic ring system which may contain from 1 to 4 hetero atoms selected from nitrogen, oxygen and sulfur;

each R₅ independently is halogen, C₁₋₆alkyl or C₁₋₆alkoxy, or two R₅ at the same ring atom together are oxo;

D is selected from the group consisting of

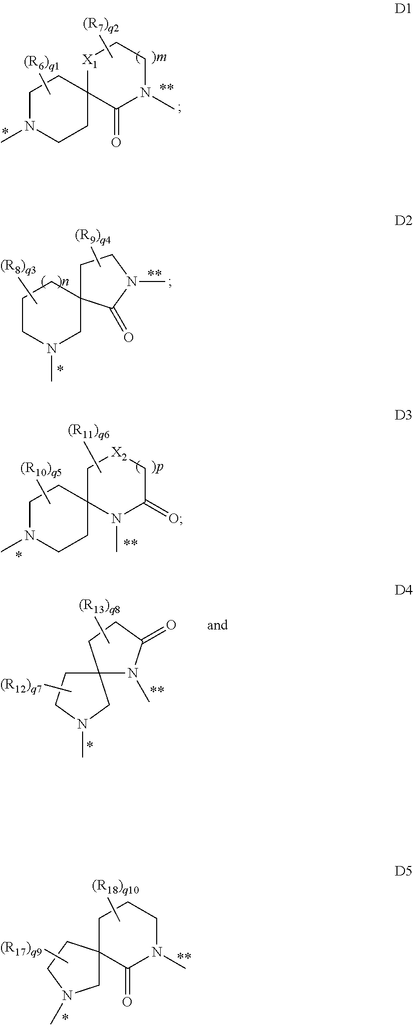

wherein the bond marked with one asterisk is attached to A and the bond marked with two asterisks is attached to C(R₁)₂—B;

wherein when D is D5, B is a five- to six-membered monocyclic aromatic ring system which may contain from 1 to 4 hetero atoms selected from nitrogen, oxygen and sulfur, which is substituted once by B1; or B is a eight- to ten-membered fused bicyclic aromatic ring system which may contain from 1 to 4 hetero atoms selected from nitrogen, oxygen and sulfur, and which may be substituted once or more than once by R₄;

X₁ is —C(R₁₄)₂— or —N(R₁₅)— and m is 0;

or X₁ is —O— or —N(R₁₅)— and m is 1;

each R₆ or R₇ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₆ at the same carbon atom form together with said carbon atom C₃₋₇cycloalkyl, or two R₇ at the same carbon atom form together with said carbon atom C₃₋₇cycloalkyl;

each R₁₄ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

R₁₅ is hydrogen, C₁₋₆alkyl, C₃₋₇cycloalkyl or C₃₋₇cycloalkyl(C₁₋₄alkyl);

q1 is 0, 1, 2, 3, 4, 5 or 6;

q2 is 0, 1, 2, 3 or 4;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6;

q4 is 0, 1, 2, 3 or 4;

X₂ is —C(R₁₆)₂— and p is 0;

or X₂ is —O— and p is 0 or 1;

each R₁₀ or R₁₁ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₀ at the same carbon atom form together with said carbon atom C₃₋₇cycloalkyl, or two R₁₁ at the same carbon atom form together with said carbon atom C₃₋₇cycloalkyl;

each R₁₆ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;

q5 is 0, 1, 2, 3, 4, 5 or 6;

q6 is 0, 1, 2, 3 or 4;

each R₁₂ or R₁₃ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₂ at the same carbon atom form together with said carbon atom C₃₋₇cycloalkyl, or two R₁₃ at the same carbon atom form together with said carbon atom C₃₋₇cycloalkyl;

q7 is 0, 1, 2, 3 or 4; and

q8 is 0, 1, 2, 3 or 4;

each R₁₇ or R₁₈ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₇ at the same carbon atom form together with said carbon atom C₃₋₇cycloalkyl, or two R₁₈ at the same carbon atom form together with said carbon atom C₃₋₇cycloalkyl;

q9 is 0, 1, 2, 3 or 4; and

q10 is 0, 1, 2, 3, 4, 5 or 6;

in free form or in salt form or in pharmaceutically acceptable salt form.

### Embodiment 2

A compound of formula I according to embodiment 1 in free form or in salt form or in pharmaceutically acceptable salt form, wherein D is D1 and each R₁ is hydrogen.

### Embodiment 3

A compound of formula I according to embodiment 2 in free form or in salt form or in pharmaceutically acceptable salt form, wherein or X₁ is —O— or —N(R₁₅)— and m is 1.

### Embodiment 4

A compound of formula I according to any one of embodiments 1 to 3 in free form or in salt form or in pharmaceutically acceptable salt form, wherein A is a five- to six-membered monocyclic aromatic ring system which contains from 1 to 4 hetero atoms selected from nitrogen, oxygen and sulfur, and which is substituted once or more than once by R₂.

### Embodiment 5

A compound of formula I according to any one of embodiments 1 to 3 in free form or in salt form or in pharmaceutically acceptable salt form, A is an eight- to ten-membered fused bicyclic aromatic ring system which may contain from 1 to 4 hetero atoms selected from nitrogen, oxygen and sulfur, and which may be substituted once or more than once by R₂.

### Embodiment 6

A compound of formula I according to any one of embodiments 1 to 5 in free form or in salt form or in pharmaceutically acceptable salt form, wherein B is a nine-membered fused bicyclic aromatic ring system which contains from 1 to 4 hetero atoms selected from nitrogen, oxygen and sulfur, and which may be substituted once or more than once by R₄.

### Embodiment 7

A compound of formula I according to embodiment 1, wherein said compound is selected from the group consisting of
4-((1H-Indo)-3-yl)methyl)-9-(4,6-dimethylpyrimidin-2-yl)-1,4,9-triazaspiro[5.5]undecan-5-one;
4-((1H-Indo)-3-yl)methyl)-9-(4,6-dimethylpyrimidin-2-yl)-1-methyl-1,4,9-triazaspiro[5.5]undecan-5-one;
9-(4,6-Dimethyl-pyrimidin-2-yl)-4-(1-methyl-4-phenyl-1H-pyrazol-3-ylmethyl)-1,4,9-triaza-spiro[5.5]undecan-5-on;
9-(4,6-Dimethylpyrimidin-2-yl)-4-((5-fluoro-1H-indol-3-yl)methyl)-1,4,9-triazaspiro[5.5]undecan-5-one;
9-(4,6-Dimethylpyrimidin-2-yl)-4-((5-(3-methoxyphenyl)-2-methyl-2H-1,2,3-triazol-4-yl)methyl)-1,4,9-triazaspiro[5.5]undecan-5-one;
4-((1H-Indo)-3-yl)methyl)-9-(4-methylpyrimidin-2-yl)-1,4,9-triazaspiro[5.5]undecan-5-one;
4-((1H-Indo)-3-yl)methyl)-9-(4-methoxypyrimidin-2-yl)-1,4,9-triazaspiro[5.5]undecan-5-one;
4-((1H-Indo)-3-yl)methyl)-9-(4-methoxy-6-methylpyrimidin-2-yl)-1,4,9-triazaspiro[5.5]undecan-5-one;
9-(4,6-Dimethylpyrimidin-2-yl)-4-((5-(3-methoxyphenyl)oxazol-4-yl)methyl)-1,4,9-triazaspiro[5.5]undecan-5-one;
4-((5-(3-Methoxyphenyl)oxazol-4-yl)methyl)-9-(4-methylpyrimidin-2-yl)-1,4,9-triazaspiro[5.5]undecan-5-one;
2-(4-((5-(3-Methoxyphenyl)oxazol-4-yl)methyl)-5-oxo-1,4,9-triazaspiro[5.5]undecan-9-yl)-6-methylpyrimidine-4-carbonitrile;
4-((5-(3-Methoxyphenyl)oxazol-4-yl)methyl)-9-(4-methoxypyrimidin-2-yl)-1,4,9-triazaspiro[5.5]undecan-5-one;
9-(4-Methoxy-6-methylpyrimidin-2-yl)-4-((5-(3-methoxyphenyl)oxazol-4-yl)methyl)-1,4,9-triazaspiro[5.5]undecan-5-one;
9-(4,6-Dimethylpyrimidin-2-yl)-4-((5-(3-methoxyphenyl)-2-methyloxazol-4-yl)methyl)-1,4,9-triazaspiro[5.5]undecan-5-one;
4-((5-(3-Methoxyphenyl)-2-methyloxazol-4-yl)methyl)-9-(4-methylpyrimidin-2-yl)-1,4,9-triazaspiro[5.5]undecan-5-one;
2-(4-((5-(3-Methoxyphenyl)-2-methyloxazol-4-yl)methyl)-5-oxo-1,4,9-triazaspiro[5.5]undecan-9-yl)-6-methylpyrimidine-4-carbonitrile;
4-((5-(3-Methoxyphenyl)-2-methyloxazol-4-yl)methyl)-9-(4-methoxypyrimidin-2-yl)-1,4,9-triazaspiro[5.5]undecan-5-one;
9-(4-Methoxy-6-methylpyrimidin-2-yl)-4-((5-(3-methoxyphenyl)-2-methyloxazol-4-yl)methyl)-1,4,9-triazaspiro[5.5]undecan-5-one;
9-(4,6-Dimethylpyrimidin-2-yl)-4-((2-methyl-5-phenyloxazol-4-yl)methyl)-1,4,9-triazaspiro[5.5]undecan-5-one;
9-(4-Methylpyrimidin-2-yl)-4-((2-methyl-5-phenyloxazol-4-yl)methyl)-1,4,9-triazaspiro[5.5]undecan-5-one;
6-Methyl-2-(4-((2-methyl-5-phenyloxazol-4-yl)methyl)-5-oxo-1,4,9-triazaspiro[5.5]undecan-9-yl)pyrimidine-4-carbonitrile;
9-(4-Methoxypyrimidin-2-yl)-4-((2-methyl-5-phenyloxazol-4-yl)methyl)-1,4,9-triazaspiro[5.5]undecan-5-one;
9-(4-Methoxy-6-methylpyrimidin-2-yl)-4-((2-methyl-5-phenyloxazol-4-yl)methyl)-1,4,9-triazaspiro[5.5]undecan-5-one;
9-(4,6-Dimethylpyrimidin-2-yl)-4-((5-(3-methoxyphenyl)-2-methylthiazol-4-yl)methyl)-1,4,9-triazaspiro[5.5]undecan-5-one;
2-(4-((5-(3-Methoxyphenyl)-2-methylthiazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-methoxyphenyl)-2-methylthiazol-4-yl)methyl)-1,4,9-triazaspiro[5.5]undecan-5-one;
9-(4,6-Dimethylpyrimidin-2-yl)-4-((5-(3-(methoxymethyl)phenyl)oxazol-4-yl)methyl)-1,4,9-triazaspiro[5.5]undecan-5-one;
2-(4-((5-(3-(Methoxymethyl)phenyl)oxazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-(methoxymethyl)phenyl)oxazol-4-yl)methyl)-1,4,9-triazaspiro[5.5]undecan-5-one;
9-(4,6-Dimethylpyrimidin-2-yl)-4-((5-(3-(methoxymethyl)phenyl)-2-methyloxazol-4-yl)methyl)-1,4,9-triazaspiro[5.5]undecan-5-one;
2-(4-((5-(3-(Methoxymethyl)phenyl)-2-methyloxazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-(methoxymethyl)phenyl)-2-methyloxazol-4-yl)methyl)-1,4,9-triazaspiro[5.5]undecan-5-one;
9-(4,6-Dimethylpyrimidin-2-yl)-4-((4-(3-methoxyphenyl)-1-methyl-1H-pyrazol-3-yl)methyl)-1,4,9-triazaspiro[5.5]undecan-5-one;
9-(4,6-Dimethylpyrimidin-2-yl)-4-((5-methoxy-1H-indol-3-yl)methyl)-1,4,9-triazaspiro[5.5]undecan-5-one;
4-((4-(3,4-Dimethoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(4-methoxyphenyl)-1-methyl-1H-pyrazol-3-yl)methyl)-1,4,9-triazaspiro[5.5]undecan-5-one;
4-((4-(3,5-Dimethoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((1-methyl-4-(m-tolyl)-1H-pyrazol-3-yl)methyl)-1,4,9-triazaspiro[5.5]undecan-5-one;
9-(4,6-Dimethylpyrimidin-2-yl)-4-((5-(3-methoxyphenyl)-2H-1,2,3-triazol-4-yl)methyl)-1,4,9-triazaspiro[5.5]undecan-5-one;
2-(4-((1H-Indo)-3-yl)methyl)-5-oxo-1,4,9-triazaspiro[5.5]undecan-9-yl)-6-methylpyrimidine-4-carbonitrile;
9-(4,6-Dimethylpyrimidin-2-yl)-4-((1-methyl-4-(3-(trifluoromethyl)phenyl)-1H-pyrazol-3-yl)methyl)-1,4,9-triazaspiro[5.5]undecan-5-one;
4-((4-(3-Chlorophenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(3-fluorophenyl)-1-methyl-1H-pyrazol-3-yl)methyl)-1,4,9-triazaspiro[5.5]undecan-5-one;
3-(3-((9-(4,6-Dimethylpyrimidin-2-yl)-5-oxo-1,4,9-triazaspiro[5.5]undecan-4-yl)methyl)-1-methyl-1H-pyrazol-4-yl)benzonitrile;
4-(3-((9-(4,6-Dimethylpyrimidin-2-yl)-5-oxo-1,4,9-triazaspiro[5.5]undecan-4-yl)methyl)-1-methyl-1H-pyrazol-4-yl)benzonitrile;
9-(4,6-Dimethylpyrimidin-2-yl)-4-((1-methyl-4-(3-(trifluoromethoxy)phenyl)-1H-pyrazol-3-yl)methyl)-1,4,9-triazaspiro[5.5]undecan-5-one;
9-(4,6-Dimethylpyrimidin-2-yl)-4-((4-(3-isopropoxyphenyl)-1-methyl-1H-pyrazol-3-yl)methyl)-1,4,9-triazaspiro[5.5]undecan-5-one;
4-((4-(3-Acetylphenyl)-1-methyl-1H-pyrazol-3-yl)methyl)-9-(4,6-dimethylpyrimidin-2-yl)-1,4,9-triazaspiro[5.5]undecan-5-one;
3-(3-((9-(4,6-Dimethylpyrimidin-2-yl)-5-oxo-1,4,9-triazaspiro[5.5]undecan-4-yl)methyl)-1-methyl-1H-pyrazol-4-yl)-N-methylbenzamide;
4-((1H-Indazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(4-(methoxymethyl)phenyl)-1-methyl-1H-pyrazol-3-yl)methyl)-1,4,9-triazaspiro[5.5]undecan-5-one;
4-((4-(3-(Cyclopropylmethoxy)phenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(3-isobutoxyphenyl)-1-methyl-1H-pyrazol-3-yl)methyl)-1,4,9-triazaspiro[5.5]undecan-5-one;
9-(4,6-Dimethylpyrimidin-2-yl)-4-((4-(6-methoxypyridin-2-yl)-1-methyl-1H-pyrazol-3-yl)methyl)-1,4,9-triazaspiro[5.5]undecan-5-one;
9-(4,6-Dimethylpyrimidin-2-yl)-4-((4-(5-methoxypyridin-3-yl)-1-methyl-1H-pyrazol-3-yl)methyl)-1,4,9-triazaspiro[5.5]undecan-5-one;
9-(4,6-Dimethylpyrimidin-2-yl)-4-((5-methoxy-1H-indazol-3-yl)methyl)-1,4,9-triazaspiro[5.5]undecan-5-one;
4-((4-(3-(1H-Pyrazol-1-yl)phenyl)-1-methyl-1H-pyrazol-3-yl)methyl)-9-(4,6-dimethylpyrimidin-2-yl)-1,4,9-triazaspiro[5.5]undecan-5-one;
4-((4-(3-(Dimethylamino)phenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((1-methyl-4-(3-(5-methyl-1,3,4-oxadiazol-2-yl)phenyl)-1H-pyrazol-3-yl)methyl)-1,4,9-triazaspiro[5.5]undecan-5-one;
4-((4-(3-(3,5-Dimethyl-1H-pyrazol-1-yl)phenyl)-1-methyl-1H-pyrazol-3-yl)methyl)-9-(4,6-dimethylpyrimidin-2-yl)-1,4,9-triazaspiro[5.5]undecan-5-one;
4-((4-(2,3-Dihydrobenzo[b][1,4]dioxin-6-yl)-1-methyl-1H-pyrazol-3-yl)methyl)-9-(4,6-dimethylpyrimidin-2-yl)-1,4,9-triazaspiro[5.5]undecan-5-one;
4-((4-(3-Cyclopropoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-dimethylpyrimidin-2-yl)-4-(2-(3-(methoxymethyl)-1,2,4-oxadiazol-5-yl)benzyl)-1,4,9-triazaspiro[5.5]undecan-5-one;
9-(4,6-dimethylpyrimidin-2-yl)-4-(2-(5-methyloxazol-2-yl)benzyl)-1,4,9-triazaspiro[5.5]undecan-5-one;
4-((1H-indazol-3-yl)methyl)-9-(4-methoxy-6-methylpyrimidin-2-yl)-1,4,9-triazaspiro[5.5]undecan-5-one;
4-((1H-indol-3-yl)methyl)-9-(2-methoxy-6-methylpyrimidin-4-yl)-1,4,9-triazaspiro[5.5]undecan-5-one;
9-(4,6-dimethylpyrimidin-2-yl)-4-((4-(3-ethylphenyl)-1-methyl-1H-pyrazol-3-yl)methyl)-1,4,9-triazaspiro[5.5]undecan-5-one;
9-(4,6-dimethylpyrimidin-2-yl)-4-((4-(3-isopropylphenyl)-1-methyl-1H-pyrazol-3-yl)methyl)-1,4,9-triazaspiro[5.5]undecan-5-one;
9-(4,6-dimethylpyrimidin-2-yl)-4-(2-(3-methyl-1,2,4-oxadiazol-5-yl)benzyl)-1,4,9-triazaspiro[5.5]undecan-5-one;
9-(4,6-dimethylpyrimidin-2-yl)-4-((1-methyl-4-(3-propionylphenyl)-1H-pyrazol-3-yl)methyl)-1,4,9-triazaspiro[5.5]undecan-5-one;
4-((4-(3-(difluoromethyl)phenyl)-1-methyl-1H-pyrazol-3-yl)methyl)-9-(4,6-dimethylpyrimidin-2-yl)-1,4,9-triazaspiro[5.5]undecan-5-one;
9-(4,6-dimethylpyrimidin-2-yl)-4-((4-(3-(2-isopropoxyethoxy)phenyl)-1-methyl-1H-pyrazol-3-yl)methyl)-1,4,9-triazaspiro[5.5]undecan-5-one;
9-(4,6-dimethylpyrimidin-2-yl)-4-((1-methyl-4-(3-morpholinophenyl)-1H-pyrazol-3-yl)methyl)-1,4,9-triazaspiro[5.5]undecan-5-one;
9-(4,6-dimethylpyrimidin-2-yl)-4-((5-(2-methoxypyridin-4-yl)-2-methyl-2H-1,2,3-triazol-4-yl)methyl)-1,4,9-triazaspiro[5.5]undecan-5-one;
9-(4,6-dimethylpyrimidin-2-yl)-4-((5-(2-ethylpyridin-4-yl)-2-methyl-2H-1,2,3-triazol-4-yl)methyl)-1,4,9-triazaspiro[5.5]undecan-5-one;
9-(4,6-dimethylpyrimidin-2-yl)-4-((5-(3-(methoxymethyl)phenyl)-2-methyl-2H-1,2,3-triazol-4-yl)methyl)-1,4,9-triazaspiro[5.5]undecan-5-one;
9-(4,6-dimethylpyrimidin-2-yl)-4-((2-methyl-5-(3-morpholinophenyl)-2H-1,2,3-triazol-4-yl)methyl)-1,4,9-triazaspiro[5.5]undecan-5-one;
9-(4,6-dimethylpyrimidin-2-yl)-4-((5-(3-(2-methoxyethoxy)phenyl)-2-methyl-2H-1,2,3-triazol-4-yl)methyl)-1,4,9-triazaspiro[5.5]undecan-5-one;
9-(4,6-dimethylpyrimidin-2-yl)-4-((5-(3-isopropoxyphenyl)-2-methyl-2H-1,2,3-triazol-4-yl)methyl)-1,4,9-triazaspiro[5.5]undecan-5-one;
4-((5-(2,3-dihydrobenzo[b][1,4]dioxin-6-yl)-2-methyl-2H-1,2,3-triazol-4-yl)methyl)-9-(4,6-dimethylpyrimidin-2-yl)-1,4,9-triazaspiro[5.5]undecan-5-one;
9-(4,6-dimethylpyrimidin-2-yl)-4-((4-phenylisoxazol-3-yl)methyl)-1,4,9-triazaspiro[5.5]undecan-5-one;
9-(4,6-dimethylpyrimidin-2-yl)-4-((3-phenylisoxazol-4-yl)methyl)-1,4,9-triazaspiro[5.5]undecan-5-one;
9-(4,6-dimethylpyrimidin-2-yl)-4-((2-methyl-5-phenyl-2H-1,2,3-triazol-4-yl)methyl)-1,4,9-triazaspiro[5.5]undecan-5-one;
4-((1H-Indo)-3-yl)methyl)-9-(4,6-dimethylpyrimidin-2-yl)-1-oxa-4,9-diazaspiro[5.5]undecan-5-one;
9-(4,6-Dimethyl-pyrimidin-2-yl)-4-(1H-indazol-3-ylmethyl)-1-oxa-4,9-diaza-spiro[5.5]undecan-5-one;
9-(4,6-Dimethyl-pyrimidin-2-yl)-4-(2-furan-2-yl-benzyl)-1-oxa-4,9-diaza-spiro[5.5]undecan-5-one;
4-((1H-indazol-3-yl)methyl)-9-(4-methoxy-6-methylpyrimidin-2-yl)-1-oxa-4,9-diazaspiro[5.5]undecan-5-one;
4-((1H-Indo)-3-yl)methyl)-9-(4-methoxypyrimidin-2-yl)-1-oxa-4,9-diazaspiro[5.5]undecan-5-one;
9-(4,6-Dimethylpyrimidin-2-yl)-4-((5-fluoro-1H-indol-3-yl)methyl)-1-oxa-4,9-diazaspiro[5.5]undecan-5-one;
9-(4,6-Dimethylpyrimidin-2-yl)-4-((5-methoxy-1H-indol-3-yl)methyl)-1-oxa-4,9-diazaspiro[5.5]undecan-5-one;
4-((1H-Indo)-3-yl)methyl)-9-(4-methylpyrimidin-2-yl)-1-oxa-4,9-diazaspiro[5.5]undecan-5-one;
9-(4,6-Dimethylpyrimidin-2-yl)-4-(2-(oxazol-2-yl)benzyl)-1-oxa-4,9-diazaspiro[5.5]undecan-5-one;
9-(4,6-Dimethylpyrimidin-2-yl)-4-(2-(3-methyl-1,2,4-oxadiazol-5-yl)benzyl)-1-oxa-4,9-diazaspiro[5.5]undecan-5-one;
9-(4,6-Dimethylpyrimidin-2-yl)-4-((1-methyl-4-phenyl-1H-pyrazol-3-yl)methyl)-1-oxa-4,9-diazaspiro[5.5]undecan-5-one;
9-(4,6-Dimethylpyrimidin-2-yl)-4-((2-methyl-5-phenyl-2H-1,2,3-triazol-4-yl)methyl)-1-oxa-4,9-diazaspiro[5.5]undecan-5-one;
9-(4,6-Dimethylpyrimidin-2-yl)-4-((4-phenyl-1H-pyrazol-3-yl)methyl)-1-oxa-4,9-diazaspiro[5.5]undecan-5-one;
9-(4,6-Dimethylpyrimidin-2-yl)-4-((5-phenyl-2H-1,2,3-triazol-4-yl)methyl)-1-oxa-4,9-diazaspiro[5.5]undecan-5-one;
9-(4,6-Dimethylpyrimidin-2-yl)-4-((1-methyl-4-phenyl-1H-1,2,3-triazol-5-yl)methyl)-1-oxa-4,9-diazaspiro[5.5]undecan-5-one;
9-(4,6-Dimethylpyrimidin-2-yl)-4-((1-methyl-5-phenyl-1H-1,2,3-triazol-4-yl)methyl)-1-oxa-4,9-diazaspiro[5.5]undecan-5-one;
9-(4,6-Dimethylpyrimidin-2-yl)-4-((5-(3-methoxyphenyl)-2H-1,2,3-triazol-4-yl)methyl)-1-oxa-4,9-diazaspiro[5.5]undecan-5-one;
4-((1H-Indol-3-yl)methyl)-9-(4-methoxy-6-methylpyrimidin-2-yl)-1-oxa-4,9-diazaspiro[5.5]undecan-5-one;
4-((1H-Pyrrolo[2,3-b]pyridin-3-yl)methyl)-9-(4-methoxy-6-methylpyrimidin-2-yl)-1-oxa-4,9-diazaspiro[5.5]undecan-5-one;
9-(4-Methoxy-6-methylpyrimidin-2-yl)-4-((5-(3-methoxyphenyl)oxazol-4-yl)methyl)-1-oxa-4,9-diazaspiro[5.5]undecan-5-one;
4-((1H-Indazol-3-yl)methyl)-9-(4-methylpyrimidin-2-yl)-1-oxa-4,9-diazaspiro[5.5]undecan-5-one;
4-((1H-indol-3-yl)methyl)-9-(4-ethylpyrimidin-2-yl)-1-oxa-4,9-diazaspiro[5.5]undecan-5-one;
4-((1H-indol-3-yl)methyl)-9-(4-ethyl-6-methylpyrimidin-2-yl)-1-oxa-4,9-diazaspiro[5.5]undecan-5-one;
4-((1H-indol-3-yl)methyl)-9-(4,5-dimethylpyrimidin-2-yl)-1-oxa-4,9-diazaspiro[5.5]undecan-5-one;
9-(4,6-dimethylpyrimidin-2-yl)-4-((4-phenylisoxazol-3-yl)methyl)-1-oxa-4,9-diazaspiro[5.5]undecan-5-one;
9-(4,6-dimethylpyrimidin-2-yl)-4-((3-phenylisoxazol-4-yl)methyl)-1-oxa-4,9-diazaspiro[5.5]undecan-5-one;
2-((1H-Indo)-3-yl)methyl)-8-(4,6-dimethylpyrimidin-2-yl)-2,8-diazaspiro[4.5]decan-1-one;
2-((1H-Indo)-3-yl)methyl)-8-(quinoxalin-2-yl)-2,8-diazaspiro[4.5]decan-1-one;
2-(2-((1H-Indol-3-yl)methyl)-1-oxo-2,8-diazaspiro[4.5]decan-8-yl)isonicotinonitrile;
2-((1H-Indo)-3-yl)methyl)-8-(6-methoxypyridin-2-yl)-2,8-diazaspiro[4.5]decan-1-one;
2-((1H-Indo)-3-yl)methyl)-8-(4-methylpyrimidin-2-yl)-2,8-diazaspiro[4.5]decan-1-one;
2-(Biphenyl-2-ylmethyl)-8-(quinoxalin-2-yl)-2,8-diazaspiro[4.5]decan-1-one;
8-(1H-Benzo[d]imidazol-2-yl)-2-(naphthalen-1-ylmethyl)-2,8-diazaspiro[4.5]decan-1-one;
2-(Biphenyl-2-ylmethyl)-8-(1-methyl-1H-benzo[d]imidazol-2-yl)-2,8-diazaspiro[4.5]decan-1-one;
8-(1H-Benzo[d]imidazol-2-yl)-2-(biphenyl-2-ylmethyl)-2,8-diazaspiro[4.5]decan-1-one;
8-(Benzo[d]thiazol-2-yl)-2-(biphenyl-2-ylmethyl)-2,8-diazaspiro[4.5]decan-1-one;
2-(Naphthalen-1-ylmethyl)-8-(quinoxalin-2-yl)-2,8-diazaspiro[4.5]decan-1-one;
8-(1H-Benzoimidazol-2-yl)-2-(1H-indol-3-ylmethyl)-2,8-diaza-spiro[4.5]decan-1-one;
8-Benzooxazol-2-yl-2-(1H-indol-3-ylmethyl)-2,8-diaza-spiro[4.5]decan-1-one;
2-(1H-Indo)-3-ylmethyl)-8-(4-methoxy-pyrimidin-2-yl)-2,8-diaza-spiro[4.5]decan-1-one;
8-(4-Methoxy-pyrimidin-2-yl)-2-(1H-pyrrolo[2,3-b]pyridin-3-ylmethyl)-2,8-diaza-spiro[4.5]decan-1-one;
2-(1H-Indazol-3-ylmethyl)-8-(4-methoxy-pyrimidin-2-yl)-2,8-diaza-spiro[4.5]decan-1-one;
8-(4-Methoxy-pyrimidin-2-yl)-2-[2-(3-methyl-[1,2,4]oxadiazol-5-yl)-benzyl]-2,8-diaza-spiro[4.5]decan-1-one;
2-(1H-Indo)-3-ylmethyl)-8-(4-methoxy-6-methyl-pyrimidin-2-yl)-2,8-diaza-spiro[4.5]decan-1-one;
8-(4,6-Dimethyl-pyrimidin-2-yl)-2-[2-(3-methyl-[1,2,4]oxadiazol-5-yl)-benzyl]-2,8-diaza-spiro[4.5]decan-1-one;
8-(4-Methoxy-6-methyl-pyrimidin-2-yl)-2-[2-(3-methyl-[1,2,4]oxadiazol-5-yl)-benzyl]-2,8-diaza-spiro[4.5]decan-1-one;
2-((1H-indazol-3-yl)methyl)-8-(4-methoxy-6-methylpyrimidin-2-yl)-2,8-diazaspiro[4.5]decan-1-one;
2-((1H-pyrrolo[2,3-b]pyridin-3-yl)methyl)-8-(4-methoxy-6-methylpyrimidin-2-yl)-2,8-diazaspiro[4.5]decan-1-one;
2-((1H-indol-5-yl)methyl)-8-(4,6-dimethylpyrimidin-2-yl)-2,8-diazaspiro[4.5]decan-1-one;
8-(4,6-dimethylpyrimidin-2-yl)-2-((2-methyl-5-phenyl-2H-1,2,3-triazol-4-yl)methyl)-2,8-diazaspiro[4.5]decan-1-one;
3-((1H-indol-3-yl)methyl)-8-(4,6-dimethylpyrimidin-2-yl)-1,3,8-triazaspiro[4.5]decan-4-one;
2-((1H-Indo)-3-yl)methyl)-7-(4-methoxypyrimidin-2-yl)-2,7-diazaspiro[4.4]nonan-1-one;
2-((1H-indol-3-yl)methyl)-7-(4-methoxypyrimidin-2-yl)-2,7-diazaspiro[4.4]nonan-1-one;
2-((1H-Indol-3-yl)methyl)-7-(4-methoxy-6-methylpyrimidin-2-yl)-2,7-diazaspiro[4.4]nonan-1-one;
2-((1H-Indo)-3-yl)methyl)-7-(quinoxalin-2-yl)-2,7-diazaspiro[4.4]nonan-1-one;
2-((1H-Indo)-3-yl)methyl)-7-(4-methylpyrimidin-2-yl)-2,7-diazaspiro[4.4]nonan-1-one;
2-((1H-Indo)-3-yl)methyl)-7-(4,6-dimethylpyrimidin-2-yl)-2,7-diazaspiro[4.4]nonan-1-one;
2-((1H-Indol-3-yl)methyl)-7-(benzo[d]oxazol-2-yl)-2,7-diazaspiro[4.4]nonan-1-one;
2-((1H-Indo)-4-yl)methyl)-7-(4,6-dimethylpyrimidin-2-yl)-2,7-diazaspiro[4.4]nonan-1-one;
2-((1H-Indol-4-yl)methyl)-7-(4-methoxy-6-methylpyrimidin-2-yl)-2,7-diazaspiro[4.4]nonan-1-one;
2-((1H-Pyrrolo[2,3-b]pyridin-3-yl)methyl)-7-(4-methoxypyrimidin-2-yl)-2,7-diazaspiro[4.4]nonan-1-one;
2-((1H-Indazol-3-yl)methyl)-7-(4-methoxypyrimidin-2-yl)-2,7-diazaspiro[4.4]nonan-1-one;
7-(4-Methoxypyrimidin-2-yl)-2-(2-(3-methyl-1,2,4-oxadiazol-5-yl)benzyl)-2,7-diazaspiro[4.4]nonan-1-one;
2-((5-(3-Methoxyphenyl)-2H-1,2,3-triazol-4-yl)methyl)-7-(4-methoxypyrimidin-2-yl)-2,7-diazaspiro[4.4]nonan-1-one;
2-((1H-Indo)-3-yl)methyl)-7-(5-chlorobenzo[d]oxazol-2-yl)-2,7-diazaspiro[4.4]nonan-1-one;
2-((1H-Indo)-3-yl)methyl)-7-(6-fluoroquinazolin-2-yl)-2,7-diazaspiro[4.4]nonan-1-one;
2-((1H-indol-3-yl)methyl)-7-(6-(trifluoromethyl)pyrimidin-4-yl)-2,7-diazaspiro[4.4]nonan-1-one;
7-(4-(1H-imidazol-1-yl)pyrimidin-2-yl)-2-((1H-indol-3-yl)methyl)-2,7-diazaspiro[4.4]nonan-1-one;
7-(2-(1H-imidazol-1-yl)pyrimidin-4-yl)-2-((1H-indol-3-yl)methyl)-2,7-diazaspiro[4.4]nonan-1-one;
7-(6-(1H-imidazol-1-yl)pyridazin-3-yl)-2-((1H-indol-3-yl)methyl)-2,7-diazaspiro[4.4]nonan-1-one;
2-((1H-indol-3-yl)methyl)-7-(2-cyclopropyl-6-methoxypyrimidin-4-yl)-2,7-diazaspiro[4.4]nonan-1-one;
2-((1H-indol-3-yl)methyl)-7-(4-(1-methyl-1H-imidazol-2-yl)pyrimidin-2-yl)-2,7-diazaspiro[4.4]nonan-1-one;
2-((1H-indol-3-yl)methyl)-7-(5-methylthiazol-2-yl)-2,7-diazaspiro[4.4]nonan-1-one;
2-((1H-indol-3-yl)methyl)-7-(7,8-dihydro-5H-pyrano[4,3-c]pyridazin-3-yl)-2,7-diazaspiro[4.4]nonan-1-one;
2-((1H-indol-3-yl)methyl)-7-(4-methyl-6-morpholinopyrimidin-2-yl)-2,7-diazaspiro[4.4]nonan-1-one;
2-((1H-indol-3-yl)methyl)-7-(2-methyl-6-morpholinopyrimidin-4-yl)-2,7-diazaspiro[4.4]nonan-1-one;
2-((1H-indol-3-yl)methyl)-7-(6-methylpyridin-2-yl)-2,7-diazaspiro[4.4]nonan-1-one;
2-((1H-indol-3-yl)methyl)-7-(4-methylpyridin-2-yl)-2,7-diazaspiro[4.4]nonan-1-one;
2-((1H-indol-3-yl)methyl)-7-(4,6-dimethylpyridin-2-yl)-2,7-diazaspiro[4.4]nonan-1-one;
2-((1H-indol-3-yl)methyl)-7-(4-methoxypyridin-2-yl)-2,7-diazaspiro[4.4]nonan-1-one;
2-((1H-indol-3-yl)methyl)-7-(2,6-dimethylpyrimidin-4-yl)-2,7-diazaspiro[4.4]nonan-1-one;
2-((1H-indol-3-yl)methyl)-7-(1-methyl-1H-pyrrolo[2,3-c]pyridin-7-yl)-2,7-diazaspiro[4.4]nonan-1-one;
2-((1H-indol-3-yl)methyl)-7-(7-methoxypyrazolo[1,5-a]pyrimidin-5-yl)-2,7-diazaspiro[4.4]nonan-1-one;
2-((1H-indol-3-yl)methyl)-7-(4-(3-methyl-1,2,4-oxadiazol-5-yl)pyridin-2-yl)-2,7-diazaspiro[4.4]nonan-1-one;
2-((1H-indol-3-yl)methyl)-7-(6-(pyrrolidin-1-yl)pyrimidin-4-yl)-2,7-diazaspiro[4.4]nonan-1-one;
2-((1H-indol-3-yl)methyl)-7-(2-methoxy-6-methylpyrimidin-4-yl)-2,7-diazaspiro[4.4]nonan-1-one;
2-((1H-indol-3-yl)methyl)-7-(4-methylthiazol-2-yl)-2,7-diazaspiro[4.4]nonan-1-one;
2-((1H-indol-3-yl)methyl)-7-(5-methoxypyrazolo[1,5-a]pyrimidin-7-yl)-2,7-diazaspiro[4.4]nonan-1-one;
2-((1H-indol-3-yl)methyl)-7-(4-isopropylpyrimidin-2-yl)-2,7-diazaspiro[4.4]nonan-1-one;
2-((1H-indol-3-yl)methyl)-7-(2-methylimidazo[1,2-a]pyrazin-8-yl)-2,7-diazaspiro[4.4]nonan-1-one;
2-((1H-indol-3-yl)methyl)-7-(4-(trifluoromethyl)pyrimidin-2-yl)-2,7-diazaspiro[4.4]nonan-1-one;
7-(4-methoxy-6-methylpyrimidin-2-yl)-2-(2-(3-methyl-1,2,4-oxadiazol-5-yl)benzyl)-2,7-diazaspiro[4.4]nonan-1-one;
7-(4-methoxy-6-methylpyrimidin-2-yl)-2-((2-methyl-5-phenyl-2H-1,2,3-triazol-4-yl)methyl)-2,7-diazaspiro[4.4]nonan-1-one;
7-(4-methoxy-6-methylpyrimidin-2-yl)-2-((2-methyl-5-(m-tolyl)-2H-1,2,3-triazol-4-yl)methyl)-2,7-diazaspiro[4.4]nonan-1-one;
7-(4-methoxy-6-methylpyrimidin-2-yl)-2-((5-(3-methoxyphenyl)-2-methyl-2H-1,2,3-triazol-4-yl)methyl)-2,7-diazaspiro[4.4]nonan-1-one;
2-((5-bromo-2-methyl-2H-1,2,3-triazol-4-yl)methyl)-7-(4-methoxy-6-methylpyrimidin-2-yl)-2,7-diazaspiro[4.4]nonan-1-one;
2-((1H-indol-3-yl)methyl)-7-(4-(dimethylamino)-6-(trifluoromethyl)pyrimidin-2-yl)-2,7-diazaspiro[4.4]nonan-1-one;
2-((1H-Indo)-3-yl)methyl)-7-(quinoxalin-2-yl)-2,7-diazaspiro[4.5]decan-1-one;
7-((1H-Indol-3-yl)methyl)-2-(4-methoxy-6-methylpyrimidin-2-yl)-2,7-diazaspiro[4.5]decan-6-one;
7-((1H-Pyrrolo[2,3-b]pyridin-3-yl)methyl)-2-(4-methoxypyrimidin-2-yl)-2,7-diazaspiro[4.5]decan-6-one;
7-((1H-Indazol-3-yl)methyl)-2-(4-methoxypyrimidin-2-yl)-2,7-diazaspiro[4.5]decan-6-one;
7-((1H-Indo)-3-yl)methyl)-2-(4-methoxypyrimidin-2-yl)-2,7-diazaspiro[4.5]decan-6-one;
2-(4-Methoxypyrimidin-2-yl)-7-(2-(3-methyl-1,2,4-oxadiazol-5-yl)benzyl)-2,7-diazaspiro[4.5]decan-6-one;
7-((5-(3-Methoxyphenyl)-2H-1,2,3-triazol-4-yl)methyl)-2-(4-methoxypyrimidin-2-yl)-2,7-diazaspiro[4.5]decan-6-one;
7-((5-(3-Methoxyphenyl)-2-methyl-2H-1,2,3-triazol-4-yl)methyl)-2-(4-methoxypyrimidin-2-yl)-2,7-diazaspiro[4.5]decan-6-one;
7-((1H-Indol-3-yl)methyl)-2-(benzo[d]oxazol-2-yl)-2,7-diazaspiro[4.5]decan-6-one;
7-((1H-indol-3-yl)methyl)-2-(4-methylpyrimidin-2-yl)-2,7-diazaspiro[4.5]decan-6-one;
7-((1H-indazol-3-yl)methyl)-2-(4-methoxy-6-methylpyrimidin-2-yl)-2,7-diazaspiro[4.5]decan-6-one;
7-((1H-pyrrolo[2,3-b]pyridin-3-yl)methyl)-2-(4-methoxy-6-methylpyrimidin-2-yl)-2,7-diazaspiro[4.5]decan-6-one;
2-(4-methoxy-6-methylpyrimidin-2-yl)-7-(2-(3-methyl-1,2,4-oxadiazol-5-yl)benzyl)-2,7-diazaspiro[4.5]decan-6-one;
7-((1H-indol-3-yl)methyl)-2-(1H-benzo[d]imidazol-2-yl)-2,7-diazaspiro[4.5]decan-6-one;
1-((1H-Indo)-4-yl)methyl)-8-(quinoxalin-2-yl)-1,8-diazaspiro[4.5]decan-2-one;
1-((1H-Indo)-4-yl)methyl)-8-(6-methylpyrazin-2-yl)-1,8-diazaspiro[4.5]decan-2-one;
1-((1H-Indo)-4-yl)methyl)-8-(4,6-dimethylpyrimidin-2-yl)-1,8-diazaspiro[4.5]decan-2-one;
2-(1-((1H-Indol-4-yl)methyl)-2-oxo-1,8-diazaspiro[4.5]decan-8-yl)isonicotinonitrile;
1-(2,5-Dimethylbenzyl)-8-(quinoxalin-2-yl)-1,8-diazaspiro[4.5]decan-2-one;
1-(2,5-Dimethylbenzyl)-8-(4,6-dimethylpyrimidin-2-yl)-1,8-diazaspiro[4.5]decan-2-one;
1-(2,5-Dimethylbenzyl)-9-(quinoxalin-2-yl)-4-oxa-1,9-diazaspiro[5.5]undecan-2-one;
1-((1H-Indo)-3-yl)methyl)-9-(quinoxalin-2-yl)-4-oxa-1,9-diazaspiro[5.5]undecan-2-one;
1-((1H-Indo)-4-yl)methyl)-9-(quinoxalin-2-yl)-4-oxa-1,9-diazaspiro[5.5]undecan-2-one;
1-((1-Methyl-1H-indol-4-yl)methyl)-9-(quinoxalin-2-yl)-4-oxa-1,9-diazaspiro[5.5]undecan-2-one;
1-(3-(Pyridin-2-yl)benzyl)-9-(quinoxalin-2-yl)-4-oxa-1,9-diazaspiro[5.5]undecan-2-one;
1-(3-(Pyridin-3-yl)benzyl)-9-(quinoxalin-2-yl)-4-oxa-1,9-diazaspiro[5.5]undecan-2-one;
1-(2,5-Dimethylbenzyl)-9-(6-methylpyrazin-2-yl)-4-oxa-1,9-diazaspiro[5.5]undecan-2-one;
1-((1H-Indo)-4-yl)methyl)-9-(6-methylpyrazin-2-yl)-4-oxa-1,9-diazaspiro[5.5]undecan-2-one;
1-(2,5-Dimethylbenzyl)-9-(4,6-dimethylpyrimidin-2-yl)-4-oxa-1,9-diazaspiro[5.5]undecan-2-one;
2-(1-((1H-Indol-4-yl)methyl)-2-oxo-4-oxa-1,9-diazaspiro[5.5]undecan-9-yl)isonicotinonitrile;
6-(1-((1H-Indol-4-yl)methyl)-2-oxo-4-oxa-1,9-diazaspiro[5.5]undecan-9-yl)picolinonitrile;
2-(1-((1H-Indo)-4-yl)methyl)-2-oxo-4-oxa-1,9-diazaspiro[5.5]undecan-9-yl)pyrimidine-4-carbonitrile;
1-((1H-Indo)-4-yl)methyl)-9-(4-methylpyrimidin-2-yl)-4-oxa-1,9-diazaspiro[5.5]undecan-2-one;
1-(2,5-Dimethylbenzyl)-8-(quinoxalin-2-yl)-3-oxa-1,8-diazaspiro[4.5]decan-2-one;
1-((1H-Indo)-3-yl)methyl)-8-(quinoxalin-2-yl)-3-oxa-1,8-diazaspiro[4.5]decan-2-one;
1-((1H-Indo)-4-yl)methyl)-8-(quinoxalin-2-yl)-3-oxa-1,8-diazaspiro[4.5]decan-2-one;
1-((1H-Indo)-3-yl)methyl)-8-(6-methylpyrazin-2-yl)-3-oxa-1,8-diazaspiro[4.5]decan-2-one;
1-((1H-Indo)-3-yl)methyl)-8-(4,6-dimethylpyrimidin-2-yl)-3-oxa-1,8-diazaspiro[4.5]decan-2-one;
1-((1H-Indo)-4-yl)methyl)-8-(6-methylpyrazin-2-yl)-3-oxa-1,8-diazaspiro[4.5]decan-2-one, and
1-((1H-Indo)-4-yl)methyl)-7-(quinoxalin-2-yl)-1,7-diazaspiro[4.4]nonan-2-one;

and wherein said compound is in free form or in salt form or in pharmaceutically acceptable salt form.

### Embodiment 8

A pharmaceutical composition comprising a therapeutically effective amount of a compound according to any one of embodiments 1 to 7 in free form or in salt form or in pharmaceutically acceptable salt form and one or more pharmaceutically acceptable carriers.

### Embodiment 9

A combination comprising a therapeutically effective amount of the compound according to any one of embodiments 1 to 7 in free form or in salt form or in pharmaceutically acceptable salt form and one or more therapeutically active agents.

### Embodiment 10

A method of inhibiting orexin receptor activity in a subject, wherein the method comprises administering to the subject a therapeutically effective amount of the compound according to any one of embodiments 1 to 7 in free form or in salt form or in pharmaceutically acceptable salt form.

### Embodiment 11

A method of treating a disorder or a disease in a subject mediated by orexin receptors, wherein the method comprises administering to the subject a therapeutically effective amount of the compound according to any one of embodiments 1 to 7 in free form or in salt form or in pharmaceutically acceptable salt form.

### Embodiment 12

A compound according to any one of embodiments 1 to 7 in free form or in salt form or in pharmaceutically acceptable salt form, for use as a medicament.

### Embodiment 13

Use of a compound according to any one of embodiments 1 to 7 in free form or in salt form or in pharmaceutically acceptable salt form, for the treatment of a disorder or disease in a subject mediated by orexin receptors.

### Embodiment 14

Use of a compound according to any one of embodiments 1 to 7 in free form or in salt form or in pharmaceutically acceptable salt form, for the treatment of a disorder or disease in a subject characterized by an abnormal activity of orexin receptors.

### Embodiment 15

A compound of formula I according to embodiment 1 in free form or in salt form or in pharmaceutically acceptable salt form, wherein

each R₁ is hydrogen;

A is a five- to six-membered aromatic ring system which contains from 1 to 4 hetero atoms selected from nitrogen, oxygen and sulfur, and which is substituted once or more than once by R₂;

B is an eight- to ten-membered fused bicyclic aromatic ring system which may contain from 1 to 4 hetero atoms selected from nitrogen, oxygen and sulfur, and which may be substituted once or more than once by R₄;

each R₂ independently is halogen, cyano, C₁₋₆alkyl, C₁₋₆halogenalkyl, C₁₋₄alkoxy-C₁₋₆alkyl or C₁₋₆alkoxy; and

each R₄ independently is halogen, cyano, C₁₋₆alkyl, C₁₋₆halogenalkyl, C₁₋₄alkoxy-C₁₋₆alkyl or C₁₋₆alkoxy.

### Embodiment 16

A compound of formula I according to embodiment 15 in free form or in salt form or in pharmaceutically acceptable salt form, wherein D is D1; X₁ is —O—; m is 1; and q1 and q2 are both 0.

### Embodiment 17

A compound of formula I according to embodiment 15 in free form or in salt form or in pharmaceutically acceptable salt form, wherein D is D1; X₁ is —N(R₁₅)—; m is 1; R₁₅ is hydrogen; and q1 and q2 are both 0.

### Embodiment 18

A compound of formula I according to embodiment 15 in free form or in salt form or in pharmaceutically acceptable salt form, wherein D is D1; X₁ is —N(R₁₅)—; m is 0; R₁₅ is hydrogen; and q1 and q2 are both 0.

### Embodiment 19

A compound of formula I according to embodiment 15 in free form or in salt form or in pharmaceutically acceptable salt form, wherein D is D2a

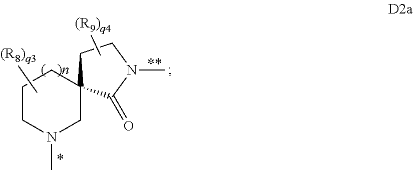

wherein the bond marked with one asterisk is attached to A and the bond marked with two asterisks is attached to C(R₁)₂—B;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6; and

q4 is 0, 1, 2, 3 or 4.

### Embodiment 20

A compound of formula I according to embodiment 19 in free form or in salt form or in pharmaceutically acceptable salt form, wherein n, q3 and q4 are all 0.

### Embodiment 21

A compound of formula I according to embodiment 19 in free form or in salt form or in pharmaceutically acceptable salt form, wherein n is 1 and q3 and q4 are both 0.

### Embodiment 22

A compound of formula I according to embodiment 15 in free form or in salt form or in pharmaceutically acceptable salt form, wherein D is D2b

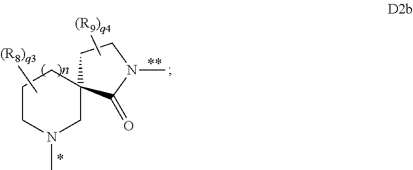

wherein the bond marked with one asterisk is attached to A and the bond marked with two asterisks is attached to C(R₁)₂—B;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6; and

q4 is 0, 1, 2, 3 or 4.

### Embodiment 23

A compound of formula I according to embodiment 22 in free form or in salt form or in pharmaceutically acceptable salt form, wherein n, q3 and q4 are all 0.

### Embodiment 24

A compound of formula I according to embodiment 22 in free form or in salt form or in pharmaceutically acceptable salt form, wherein n is 1 and q3 and q4 are both 0.

### Embodiment 25

A compound of formula I according to embodiment 1 in free form or in salt form or in pharmaceutically acceptable salt form, wherein

each R₁ is hydrogen;

A is a five- to six-membered aromatic ring system which contains from 1 to 4 hetero atoms selected from nitrogen, oxygen and sulfur, and which is substituted once or more than once by R₂;

each R₂ independently is halogen, cyano, C₁₋₆alkyl, C₁₋₆halogenalkyl, C₁₋₄alkoxy-C₁₋₆alkyl or C₁₋₆alkoxy;

B is a six-membered monocyclic aromatic ring system which may contain 1 to 2 nitrogen atoms, and which is substituted once by R₄ₐ, and which may be further substituted once or more than once by R_{4b};

R₄ₐ is a five-membered monocyclic aromatic ring system, which contains from 1 to 4 hetero atoms selected from nitrogen, oxygen and sulfur, and which may in turn be substituted once or more than once by C₁₋₆alkyl, C₁₋₆halogenalkyl, C₁₋₆alkoxy, C₁₋₄alkoxy-C₁₋₆alkyl, C₁₋₆halogenalkoxy, halogen or cyano; and

each R_{4b} independently is halogen, cyano, C₁₋₆alkyl, C₁₋₆halogenalkyl, C₁₋₄alkoxy-C₁₋₆alkyl or C₁₋₆alkoxy.

### Embodiment 26

A compound of formula I according to embodiment 25 in free form or in salt form or in pharmaceutically acceptable salt form, wherein D is D1; X₁ is —O—; m is 1; and q1 and q2 are both 0.

### Embodiment 27

A compound of formula I according to embodiment 25 in free form or in salt form or in pharmaceutically acceptable salt form, wherein D is D1; X₁ is —N(R₁₅)—; m is 1; R₁₅ is hydrogen; and q1 and q2 are both 0.

### Embodiment 28

A compound of formula I according to embodiment 25 in free form or in salt form or in pharmaceutically acceptable salt form, wherein D is D2a

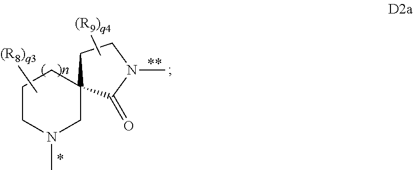

wherein the bond marked with one asterisk is attached to A and the bond marked with two asterisks is attached to C(R₁)₂—B;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6; and

q4 is 0, 1, 2, 3 or 4.

### Embodiment 29

A compound of formula I according to embodiment 28 in free form or in salt form or in pharmaceutically acceptable salt form, wherein n, q3 and q4 are all 0.

### Embodiment 30

A compound of formula I according to embodiment 28 in free form or in salt form or in pharmaceutically acceptable salt form, wherein n is 1 and q3 and q4 are both 0.

### Embodiment 31

A compound of formula I according to embodiment 25 in free form or in salt form or in pharmaceutically acceptable salt form, wherein D is D2b

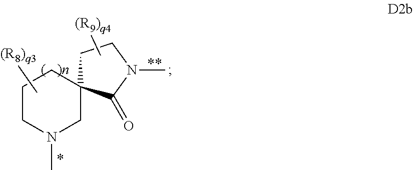

wherein the bond marked with one asterisk is attached to A and the bond marked with two asterisks is attached to C(R₁)₂—B;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6; and

q4 is 0, 1, 2, 3 or 4.

### Embodiment 32

A compound of formula I according to embodiment 31 in free form or in salt form or in pharmaceutically acceptable salt form, wherein n, q3 and q4 are all 0.

### Embodiment 33

A compound of formula I according to embodiment 31 in free form or in salt form or in pharmaceutically acceptable salt form, wherein n is 1 and q3 and q4 are both 0.

### Embodiment 34

A compound of formula I according to embodiment 1 in free form or in salt form or in pharmaceutically acceptable salt form, wherein each R₁ is hydrogen;

A is a five- to six-membered aromatic ring system which contains from 1 to 4 hetero atoms selected from nitrogen, oxygen and sulfur, and which is substituted once or more than once by R₂;

each R₂ independently is halogen, cyano, C₁₋₆alkyl, C₁₋₆halogenalkyl, C₁₋₄alkoxy-C₁₋₆alkyl or C₁₋₆alkoxy;

B is a five-membered monocyclic aromatic ring system which contains from 1 to 4 hetero atoms selected from nitrogen, oxygen and sulfur, and which is substituted once by R₄ₐ, and which may be further substituted once or more than once by R_{4b};

R₄ₐ is a six-membered monocyclic aromatic ring system, which may contain from 1 to 2 nitrogen atoms, and which may in turn be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; and each R_{4b} independently is halogen, cyano, C₁₋₆alkyl, C₁₋₆halogenalkyl, C₁₋₄alkoxy-C₁₋₆alkyl or C₁₋₆alkoxy.

### Embodiment 35

A compound of formula I according to embodiment 34 in free form or in salt form or in pharmaceutically acceptable salt form, wherein D is D1; X₁ is —O—; m is 1; and q1 and q2 are both 0.

### Embodiment 36

A compound of formula I according to embodiment 34 in free form or in salt form or in pharmaceutically acceptable salt form, wherein D is D1; X₁ is —N(R₁₅)—; m is 1; R₁₅ is hydrogen; and q1 and q2 are both 0.

### Embodiment 37

A compound of formula I according to embodiment 34 in free form or in salt form or in pharmaceutically acceptable salt form, wherein D is D2a

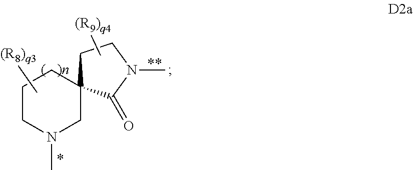

wherein the bond marked with one asterisk is attached to A and the bond marked with two asterisks is attached to C(R₁)₂—B;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6; and

q4 is 0, 1, 2, 3 or 4.

### Embodiment 38

A compound of formula I according to embodiment 37 in free form or in salt form or in pharmaceutically acceptable salt form, wherein n, q3 and q4 are all 0.

### Embodiment 39

A compound of formula I according to embodiment 37 in free form or in salt form or in pharmaceutically acceptable salt form, wherein n is 1 and q3 and q4 are both 0.

### Embodiment 40

A compound of formula I according to embodiment 34 in free form or in salt form or in pharmaceutically acceptable salt form, wherein D is D2b

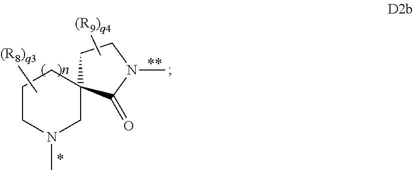

wherein the bond marked with one asterisk is attached to A and the bond marked with two asterisks is attached to C(R₁)₂—B;

n is 0 or 1;

each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;

q3 is 0, 1, 2, 3, 4, 5 or 6; and

q4 is 0, 1, 2, 3 or 4.

### Embodiment 41

A compound of formula I according to embodiment 40 in free form or in salt form or in pharmaceutically acceptable salt form, wherein n, q3 and q4 are all 0.

### Embodiment 42

A compound of formula I according to embodiment 40 in free form or in salt form or in pharmaceutically acceptable salt form, wherein n is 1 and q3 and q4 are both 0.

### Embodiment 43

A compound of formula I according to any one of embodiments 1 to 5 in free form or in salt form or in pharmaceutically acceptable salt form, wherein B is a five- to six-membered monocyclic aromatic ring system which may contain from 1 to 4 hetero atoms selected from nitrogen, oxygen and sulfur, and which is substituted once or more than once by R₄.

### Embodiment 44

A compound of formula I according to embodiment 43 in free form or in salt form or in pharmaceutically acceptable salt form, wherein B is a six-membered monocyclic aromatic ring system which may contain 1 to 2 nitrogen atoms, and which is substituted once by R₄ₐ, and which may be further substituted once or more than once by R_{4b};

R₄ₐ is a five-membered monocyclic aromatic ring system, which contains from 1 to 4 hetero atoms selected from nitrogen, oxygen and sulfur, and which may in turn be substituted once or more than once by C₁₋₆alkyl, C₁₋₆halogenalkyl, C₁₋₆alkoxy, C₁₋₄alkoxy-C₁₋₆alkyl, C₁₋₆halogenalkoxy, halogen or cyano; and

each R_{4b} independently is halogen, cyano, C₁₋₆alkyl, C₁₋₆halogenalkyl, C₁₋₄alkoxy-C₁₋₆alkyl or Cl_{—}6alkoxy.

### Embodiment 45

A compound of formula I according to embodiment 43 in free form or in salt form or in pharmaceutically acceptable salt form, wherein B is a five-membered monocyclic aromatic ring system which contains from 1 to 4 hetero atoms selected from nitrogen, oxygen and sulfur, and which is substituted once by R₄ₐ, and which may be further substituted once or more than once by R_{4b};

R₄ₐ is a six-membered monocyclic aromatic ring system, which may contain from 1 to 2 nitrogen atoms, and which may in turn be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; and

each R_{4b} independently is halogen, cyano, C₁₋₆alkyl, C₁₋₆halogenalkyl, C₁₋₄alkoxy-C₁₋₆alkyl or Cl_{—}6alkoxy.

1. A compound of the formula I
A-D-C(R₁)₂—B (I),
wherein
each R₁ independently is hydrogen, C₁₋₆alkyl, C₁₋₆halogenalkyl, C₃₋₇cycloalkyl or C₃₋₇cycloalkyl(C₁₋₄alkyl), or two R₁ together with the carbon atom to which they are bound form a C₃₋₄cycloalkyl;
A is a five- to six-membered monocyclic aromatic ring system which may contain from 1 to 4 hetero atoms selected from nitrogen, oxygen and sulfur, and which is substituted once or more than once by R₂;
or A is an eight- to ten-membered fused bicyclic aromatic ring system which may contain from 1 to 4 hetero atoms selected from nitrogen, oxygen and sulfur, and which may be substituted once or more than once by R₂;
each R₂ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; or a three- to seven-membered monocyclic ring system which may be aromatic, saturated or unsaturated non-aromatic, which may contain from 1 to 4 hetero atoms selected from nitrogen, oxygen and sulfur, and which may be substituted once or more than once by C₁₋₆alkyl, C₁₋₆halogenalkyl, C₁₋₄alkoxy-C₁₋₆alkyl, C₁₋₆alkoxy, C₁₋₆halogenalkoxy, halogen or cyano; or two R₂ at adjacent ring atoms form together with said ring atoms a fused five- to seven-membered unsaturated non-aromatic ring system which may contain from 1 to 4 hetero atoms selected from nitrogen, oxygen and sulfur, and which may be substituted once or more than once by R₃;
each R₃ independently is halogen, C₁₋₆alkyl or C₁₋₆alkoxy, or two R₃ at the same ring atom together are oxo;
B is a five- to six-membered monocyclic aromatic ring system which may contain from 1 to 4 hetero atoms selected from nitrogen, oxygen and sulfur, and which is substituted once or more than once by R₄;
or B is a eight- to ten-membered fused bicyclic aromatic ring system which may contain from 1 to 4 hetero atoms selected from nitrogen, oxygen and sulfur, and which may be substituted once or more than once by R₄;
each R₄ independently is halogen; cyano; hydroxy; amino; C₁₋₆alkyl; C₁₋₆halogenalkyl; C₁₋₆hydroxyalkyl; C₁₋₄alkoxy-C₁₋₆alkyl; C₁₋₆aminoalkyl; C₁₋₄alkylamino-C₁₋₆alkyl; di(C₁₋₄alkyl)amino-C₁₋₆alkyl; C₂₋₆alkenyl; C₂₋₆halogenalkenyl; C₂₋₆alkynyl; C₂₋₆halogenalkynyl; C₁₋₆alkoxy; C₁₋₆halogenalkoxy; C₁₋₄alkoxy-C₁₋₆alkoxy; C₁₋₆alkylamino; di(C₁₋₆alkyl)amino; B1; or two R₄ at adjacent ring atoms form together with said ring atoms a fused five- to seven-membered unsaturated non-aromatic ring system which may contain from 1 to 4 hetero atoms selected from nitrogen, oxygen and sulfur, and which may in turn be substituted once or more than once by R₅;
B1 is a three- to seven-membered monocyclic ring system which may be aromatic, saturated or unsaturated non-aromatic, which may contain from 1 to 4 hetero atoms selected from nitrogen, oxygen and sulfur, and which may in turn be substituted once or more than once by C₁₋₆alkyl, C₁₋₆halogenalkyl, C₁₋₄alkoxy-C₁₋₆alkyl, C₁₋₆alkoxy, C₃₋₇cycloalkoxy, C₁₋₆halogenalkoxy, C₃₋₇cycloalkylC₁₋₄alkoxy, C₁₋₄alkoxy-C₁₋₄alkoxy, C₁₋₄alkylcarbonyl, N—C₁₋₄alkylaminocarbonyl, C₁₋₄alkylamino, di(C₁₋₄alkyl)amino, halogen, cyano, a 6-membered saturated heterocycle containing 1 to 4 hetero atoms selected from nitrogen, oxygen and sulfur, a 5-membered aromatic ring containing 1 to 4 hetero atoms selected from nitrogen, oxygen and sulfur which may be substituted once or more than once by C₁₋₄alkyl;
or two substituents at adjacent ring atoms of B1 form together with said ring atoms a fused five- to seven-membered unsaturated non-aromatic ring system which may contain from 1 to 4 hetero atoms selected from nitrogen, oxygen and sulfur;
each R₅ independently is halogen, C₁₋₆alkyl or C₁₋₆alkoxy, or two R₅ at the same ring atom together are oxo;
D is selected from the group consisting of 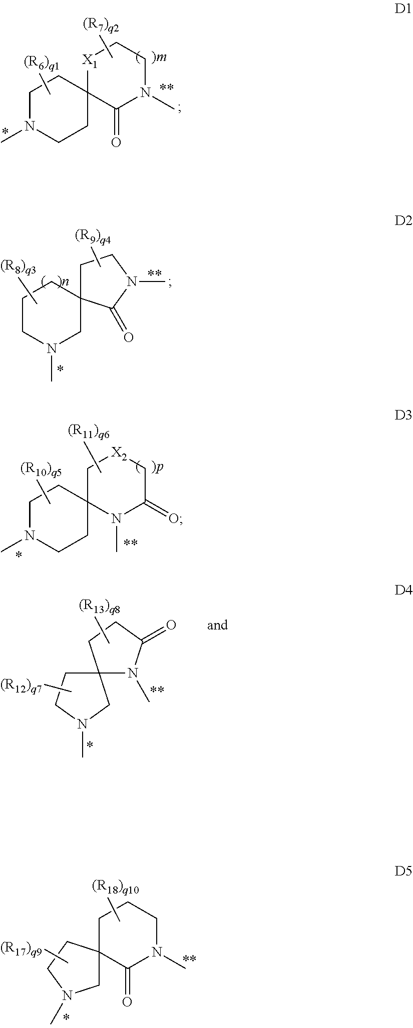
wherein the bond marked with one asterisk is attached to A and the bond marked with two asterisks is attached to C(R₁)₂—B;
wherein when D is D5, B is a five- to six-membered monocyclic aromatic ring system which may contain from 1 to 4 hetero atoms selected from nitrogen, oxygen and sulfur, which is substituted once by B1; or B is a eight- to ten-membered fused bicyclic aromatic ring system which may contain from 1 to 4 hetero atoms selected from nitrogen, oxygen and sulfur, and which may be substituted once or more than once by R₄;
X₁ is —C(R₁₄)₂— or —N(R₁₅)— and m is 0;
or X₁ is —O— or —N(R₁₅)— and m is 1;
each R₆ or R₇ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₆ at the same carbon atom form together with said carbon atom C₃₋₇cycloalkyl, or two R₇ at the same carbon atom form together with said carbon atom C₃₋₇cycloalkyl;
each R₁₄ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;
R₁₅ is hydrogen, C₁₋₆alkyl, C₃₋₇cycloalkyl or C₃₋₇cycloalkyl(C₁₋₄alkyl);
q1 is 0, 1, 2, 3, 4, 5 or 6;
q2 is 0, 1, 2, 3 or 4;
n is 0 or 1;
each R₈ or R₉ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₈ at the same carbon atom form together with said carbon atom C₃₋₇cycloalkyl, or two R₉ at the same carbon atom form together with said carbon atom C₃₋₇cycloalkyl;
q3 is 0, 1, 2, 3, 4, 5 or 6;
q4 is 0, 1, 2, 3 or 4;
X₂ is —C(R₁₆)₂— and p is 0;
or X₂ is —O— and p is 0 or 1;
each R₁₀ or R₁₁ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₀ at the same carbon atom form together with said carbon atom C₃₋₇cycloalkyl, or two R₁₁ at the same carbon atom form together with said carbon atom C₃₋₇cycloalkyl;
each R₁₆ independently is hydrogen, halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy;
q5 is 0, 1, 2, 3, 4, 5 or 6;
q6 is 0, 1, 2, 3 or 4;
each R₁₂ or R₁₃ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₂ at the same carbon atom form together with said carbon atom C₃₋₇cycloalkyl, or two R₁₃ at the same carbon atom form together with said carbon atom C₃₋₇cycloalkyl;
q7 is 0, 1, 2, 3 or 4; and
q8 is 0, 1, 2, 3 or 4;
each R₁₇ or R₁₈ independently is halogen, C₁₋₆alkyl, C₁₋₆halogenalkyl, C₃₋₇cycloalkyl, C₃₋₇cycloalkyl(C₁₋₄alkyl), C₁₋₆alkoxy, or C₁₋₆halogenalkoxy, or two R₁₇ at the same carbon atom form together with said carbon atom C₃₋₇cycloalkyl, or two R₁₈ at the same carbon atom form together with said carbon atom C₃₋₇cycloalkyl;
q9 is 0, 1, 2, 3 or 4; and
q10 is 0, 1, 2, 3, 4, 5 or 6; in free form or in salt form or in pharmaceutically acceptable salt form.

2. The compound of claim 1 in free form or in salt form or in pharmaceutically acceptable salt form, wherein D is D1 and each R₁ is hydrogen.

3. The compound of claim 2 in free form or in salt form or in pharmaceutically acceptable salt form, wherein or X₁ is —O— or —N(R₁₅)— and m is 1.

4. The compound of claim 1 in free form or in salt form or in pharmaceutically acceptable salt form, wherein A is a five- to six-membered monocyclic aromatic ring system which contains from 1 to 4 hetero atoms selected from nitrogen, oxygen and sulfur, and which is substituted once or more than once by R₂.

5. The compound of claim 1 in free form or in salt form or in pharmaceutically acceptable salt form, wherein A is an eight- to ten-membered fused bicyclic aromatic ring system which may contain from 1 to 4 hetero atoms selected from nitrogen, oxygen and sulfur, and which may be substituted once or more than once by R₂.

6. The compound of claim 1 in free form or in salt form or in pharmaceutically acceptable salt form, wherein B is a nine-membered fused bicyclic aromatic ring system which contains from 1 to 4 hetero atoms selected from nitrogen, oxygen and sulfur, and which may be substituted once or more than once by R₄.

7. The compound of claim 1 selected from:
4-((1H-Indo)-3-yl)methyl)-9-(4,6-dimethylpyrimidin-2-yl)-1,4,9-triazaspiro[5.5]undecan-5-one;
4-((1H-Indo)-3-yl)methyl)-9-(4,6-dimethylpyrimidin-2-yl)-1-methyl-1,4,9-triazaspiro[5.5]undecan-5-one;
9-(4,6-Dimethyl-pyrimidin-2-yl)-4-(1-methyl-4-phenyl-1H-pyrazol-3-ylmethyl)-1,4,9-triaza-spiro[5.5]undecan-5-on;
9-(4,6-Dimethylpyrimidin-2-yl)-4-((5-fluoro-1H-indol-3-yl)methyl)-1,4,9-triazaspiro[5.5]undecan-5-one;
9-(4,6-Dimethylpyrimidin-2-yl)-4-((5-(3-methoxyphenyl)-2-methyl-2H-1,2,3-triazol-4-yl)methyl)-1,4,9-triazaspiro[5.5]undecan-5-one;
4-((1H-Indo)-3-yl)methyl)-9-(4-methylpyrimidin-2-yl)-1,4,9-triazaspiro[5.5]undecan-5-one;
4-((1H-Indo)-3-yl)methyl)-9-(4-methoxypyrimidin-2-yl)-1,4,9-triazaspiro[5.5]undecan-5-one;
4-((1H-Indol-3-yl)methyl)-9-(4-methoxy-6-methylpyrimidin-2-yl)-1,4,9-triazaspiro[5.5]undecan-5-one;
9-(4,6-Dimethylpyrimidin-2-yl)-4-((5-(3-methoxyphenyl)oxazol-4-yl)methyl)-1,4,9-triazaspiro[5.5]undecan-5-one;
4-((5-(3-Methoxyphenyl)oxazol-4-yl)methyl)-9-(4-methylpyrimidin-2-yl)-1,4,9-triazaspiro[5.5]undecan-5-one;
2-(4-((5-(3-Methoxyphenyl)oxazol-4-yl)methyl)-5-oxo-1,4,9-triazaspiro[5.5]undecan-9-yl)-6-methylpyrimidine-4-carbonitrile;
4-((5-(3-Methoxyphenyl)oxazol-4-yl)methyl)-9-(4-methoxypyrimidin-2-yl)-1,4,9-triazaspiro[5.5]undecan-5-one;
9-(4-Methoxy-6-methylpyrimidin-2-yl)-4-((5-(3-methoxyphenyl)oxazol-4-yl)methyl)-1,4,9-triazaspiro[5.5]undecan-5-one;
9-(4,6-Dimethylpyrimidin-2-yl)-4-((5-(3-methoxyphenyl)-2-methyloxazol-4-yl)methyl)-1,4,9-triazaspiro[5.5]undecan-5-one;
4-((5-(3-Methoxyphenyl)-2-methyloxazol-4-yl)methyl)-9-(4-methylpyrimidin-2-yl)-1,4,9-triazaspiro[5.5]undecan-5-one;
2-(4-((5-(3-Methoxyphenyl)-2-methyloxazol-4-yl)methyl)-5-oxo-1,4,9-triazaspiro[5.5]undecan-9-yl)-6-methylpyrimidine-4-carbonitrile;
4-((5-(3-Methoxyphenyl)-2-methyloxazol-4-yl)methyl)-9-(4-methoxypyrimidin-2-yl)-1,4,9-triazaspiro[5.5]undecan-5-one;
9-(4-Methoxy-6-methylpyrimidin-2-yl)-4-((5-(3-methoxyphenyl)-2-methyloxazol-4-yl)methyl)-1,4,9-triazaspiro[5.5]undecan-5-one;
9-(4,6-Dimethylpyrimidin-2-yl)-4-((2-methyl-5-phenyloxazol-4-yl)methyl)-1,4,9-triazaspiro[5.5]undecan-5-one;
9-(4-Methylpyrimidin-2-yl)-4-((2-methyl-5-phenyloxazol-4-yl)methyl)-1,4,9-triazaspiro[5.5]undecan-5-one;
6-Methyl-2-(4-((2-methyl-5-phenyloxazol-4-yl)methyl)-5-oxo-1,4,9-triazaspiro[5.5]undecan-9-yl)pyrimidine-4-carbonitrile;
9-(4-Methoxypyrimidin-2-yl)-4-((2-methyl-5-phenyloxazol-4-yl)methyl)-1,4,9-triazaspiro[5.5]undecan-5-one;
9-(4-Methoxy-6-methylpyrimidin-2-yl)-4-((2-methyl-5-phenyloxazol-4-yl)methyl)-1,4,9-triazaspiro[5.5]undecan-5-one;
9-(4,6-Dimethylpyrimidin-2-yl)-4-((5-(3-methoxyphenyl)-2-methylthiazol-4-yl)methyl)-1,4,9-triazaspiro[5.5]undecan-5-one;
2-(4-((5-(3-Methoxyphenyl)-2-methylthiazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-methoxyphenyl)-2-methylthiazol-4-yl)methyl)-1,4,9-triazaspiro[5.5]undecan-5-one;
9-(4,6-Dimethylpyrimidin-2-yl)-4-((5-(3-(methoxymethyl)phenyl)oxazol-4-yl)methyl)-1,4,9-triazaspiro[5.5]undecan-5-one;
2-(4-((5-(3-(Methoxymethyl)phenyl)oxazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-(methoxymethyl)phenyl)oxazol-4-yl)methyl)-1,4,9-triazaspiro[5.5]undecan-5-one;
9-(4,6-Dimethylpyrimidin-2-yl)-4-((5-(3-(methoxymethyl)phenyl)-2-methyloxazol-4-yl)methyl)-1,4,9-triazaspiro[5.5]undecan-5-one;
2-(4-((5-(3-(Methoxymethyl)phenyl)-2-methyloxazol-4-yl)methyl)-5-oxo-1,4,9-triazaspiro[5.5]undecan-9-yl)-6-methylpyrimidine-4-carbonitrile;
9-(4-Methoxy-6-methylpyrimidin-2-yl)-4-((5-(3-(methoxymethyl)phenyl)-2-methyloxazol-4-yl)methyl)-1,4,9-triazaspiro[5.5]undecan-5-one;
9-(4,6-Dimethylpyrimidin-2-yl)-4-((4-(3-methoxyphenyl)-1-methyl-1H-pyrazol-3-yl)methyl)-1,4,9-triazaspiro[5.5]undecan-5-one;
9-(4,6-Dimethylpyrimidin-2-yl)-4-((5-methoxy-1H-indol-3-yl)methyl)-1,4,9-triazaspiro[5.5]undecan-5-one;
4-((4-(3,4-Dimethoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(4-methoxyphenyl)-1-methyl-1H-pyrazol-3-yl)methyl)-1,4,9-triazaspiro[5.5]undecan-5-one;
4-((4-(3,5-Dimethoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((1-methyl-4-(m-tolyl)-1H-pyrazol-3-yl)methyl)-1,4,9-triazaspiro[5.5]undecan-5-one;
9-(4,6-Dimethylpyrimidin-2-yl)-4-((5-(3-methoxyphenyl)-2H-1,2,3-triazol-4-yl)methyl)-1,4,9-triazaspiro[5.5]undecan-5-one;
2-(4-((1H-Indo)-3-yl)methyl)-5-oxo-1,4,9-triazaspiro[5.5]undecan-9-yl)-6-methylpyrimidine-4-carbonitrile;
9-(4,6-Dimethylpyrimidin-2-yl)-4-((1-methyl-4-(3-(trifluoromethyl)phenyl)-1H-pyrazol-3-yl)methyl)-1,4,9-triazaspiro[5.5]undecan-5-one;
4-((4-(3-Chlorophenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(3-fluorophenyl)-1-methyl-1H-pyrazol-3-yl)methyl)-1,4,9-triazaspiro[5.5]undecan-5-one;
3-(3-((9-(4,6-Dimethylpyrimidin-2-yl)-5-oxo-1,4,9-triazaspiro[5.5]undecan-4-yl)methyl)-1-methyl-1H-pyrazol-4-yl)benzonitrile;
4-(3-((9-(4,6-Dimethylpyrimidin-2-yl)-5-oxo-1,4,9-triazaspiro[5.5]undecan-4-yl)methyl)-1-methyl-1H-pyrazol-4-yl)benzonitrile;
9-(4,6-Dimethylpyrimidin-2-yl)-4-((1-methyl-4-(3-(trifluoromethoxy)phenyl)-1H-pyrazol-3-yl)methyl)-1,4,9-triazaspiro[5.5]undecan-5-one;
9-(4,6-Dimethylpyrimidin-2-yl)-4-((4-(3-isopropoxyphenyl)-1-methyl-1H-pyrazol-3-yl)methyl)-1,4,9-triazaspiro[5.5]undecan-5-one;
4-((4-(3-Acetylphenyl)-1-methyl-1H-pyrazol-3-yl)methyl)-9-(4,6-dimethylpyrimidin-2-yl)-1,4,9-triazaspiro[5.5]undecan-5-one;
3-(3-((9-(4,6-Dimethylpyrimidin-2-yl)-5-oxo-1,4,9-triazaspiro[5.5]undecan-4-yl)methyl)-1-methyl-1H-pyrazol-4-yl)-N-methylbenzamide;
4-((1H-Indazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(4-(methoxymethyl)phenyl)-1-methyl-1H-pyrazol-3-yl)methyl)-1,4,9-triazaspiro[5.5]undecan-5-one;
4-((4-(3-(Cyclopropylmethoxy)phenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((4-(3-isobutoxyphenyl)-1-methyl-1H-pyrazol-3-yl)methyl)-1,4,9-triazaspiro[5.5]undecan-5-one;
9-(4,6-Dimethylpyrimidin-2-yl)-4-((4-(6-methoxypyridin-2-yl)-1-methyl-1H-pyrazol-3-yl)methyl)-1,4,9-triazaspiro[5.5]undecan-5-one;
9-(4,6-Dimethylpyrimidin-2-yl)-4-((4-(5-methoxypyridin-3-yl)-1-methyl-1H-pyrazol-3-yl)methyl)-1,4,9-triazaspiro[5.5]undecan-5-one;
9-(4,6-Dimethylpyrimidin-2-yl)-4-((5-methoxy-1H-indazol-3-yl)methyl)-1,4,9-triazaspiro[5.5]undecan-5-one;
4-((4-(3-(1H-Pyrazol-1-yl)phenyl)-1-methyl-1H-pyrazol-3-yl)methyl)-9-(4,6-dimethylpyrimidin-2-yl)-1,4,9-triazaspiro[5.5]undecan-5-one;
4-((4-(3-(Dimethylamino)phenyl)-1-methyl-1H-pyrazol-3-yl)methyl)-9-(4,6-dimethylpyrimidin-2-yl)-1,4,9-triazaspiro[5.5]undecan-5-one;
9-(4,6-Dimethylpyrimidin-2-yl)-4-((1-methyl-4-(3-(5-methyl-1,3,4-oxadiazol-2-yl)phenyl)-1H-pyrazol-3-yl)methyl)-1,4,9-triazaspiro[5.5]undecan-5-one;
4-((4-(3-(3,5-Dimethyl-1H-pyrazol-1-yl)phenyl)-1-methyl-1H-pyrazol-3-yl)methyl)-9-(4,6-dimethylpyrimidin-2-yl)-1,4,9-triazaspiro[5.5]undecan-5-one;
4-((4-(2,3-Dihydrobenzo[b][1,4]dioxin-6-yl)-1-methyl-1H-pyrazol-3-yl)methyl)-9-(4,6-dimethylpyrimidin-2-yl)-1,4,9-triazaspiro[5.5]undecan-5-one;
4-((4-(3-Cyclopropoxyphenyl)-1-methyl-1H-pyrazol-3-yl)methyl)-9-(4,6-dimethylpyrimidin-2-yl)-1,4,9-triazaspiro[5.5]undecan-5-one;
9-(4,6-dimethylpyrimidin-2-yl)-4-(2-(3-(methoxymethyl)-1,2,4-oxadiazol-5-yl)benzyl)-1,4,9-triazaspiro[5.5]undecan-5-one;
9-(4,6-dimethylpyrimidin-2-yl)-4-(2-(5-methyloxazol-2-yl)benzyl)-1,4,9-triazaspiro[5.5]undecan-5-one;
4-((1H-indazol-3-yl)methyl)-9-(4-methoxy-6-methylpyrimidin-2-yl)-1,4,9-triazaspiro[5.5]undecan-5-one;
4-((1H-indol-3-yl)methyl)-9-(2-methoxy-6-methylpyrimidin-4-yl)-1,4,9-triazaspiro[5.5]undecan-5-one;
9-(4,6-dimethylpyrimidin-2-yl)-4-((4-(3-ethylphenyl)-1-methyl-1H-pyrazol-3-yl)methyl)-1,4,9-triazaspiro[5.5]undecan-5-one;
9-(4,6-dimethylpyrimidin-2-yl)-4-((4-(3-isopropylphenyl)-1-methyl-1H-pyrazol-3-yl)methyl)-1,4,9-triazaspiro[5.5]undecan-5-one;
9-(4,6-dimethylpyrimidin-2-yl)-4-(2-(3-methyl-1,2,4-oxadiazol-5-yl)benzyl)-1,4,9-triazaspiro[5.5]undecan-5-one;
9-(4,6-dimethylpyrimidin-2-yl)-4-((1-methyl-4-(3-propionylphenyl)-1H-pyrazol-3-yl)methyl)-1,4,9-triazaspiro[5.5]undecan-5-one;
4-((4-(3-(difluoromethyl)phenyl)-1-methyl-1H-pyrazol-3-yl)methyl)-9-(4,6-dimethylpyrimidin-2-yl)-1,4,9-triazaspiro[5.5]undecan-5-one;
9-(4,6-dimethylpyrimidin-2-yl)-4-((4-(3-(2-isopropoxyethoxy)phenyl)-1-methyl-1H-pyrazol-3-yl)methyl)-1,4,9-triazaspiro[5.5]undecan-5-one;
9-(4,6-dimethylpyrimidin-2-yl)-4-((1-methyl-4-(3-morpholinophenyl)-1H-pyrazol-3-yl)methyl)-1,4,9-triazaspiro[5.5]undecan-5-one;
9-(4,6-dimethylpyrimidin-2-yl)-4-((5-(2-methoxypyridin-4-yl)-2-methyl-2H-1,2,3-triazol-4-yl)methyl)-1,4,9-triazaspiro[5.5]undecan-5-one;
9-(4,6-dimethylpyrimidin-2-yl)-4-((5-(2-ethylpyridin-4-yl)-2-methyl-2H-1,2,3-triazol-4-yl)methyl)-1,4,9-triazaspiro[5.5]undecan-5-one;
9-(4,6-dimethylpyrimidin-2-yl)-4-((5-(3-(methoxymethyl)phenyl)-2-methyl-2H-1,2,3-triazol-4-yl)methyl)-1,4,9-triazaspiro[5.5]undecan-5-one;
9-(4,6-dimethylpyrimidin-2-yl)-4-((2-methyl-5-(3-morpholinophenyl)-2H-1,2,3-triazol-4-yl)methyl)-1,4,9-triazaspiro[5.5]undecan-5-one;
9-(4,6-dimethylpyrimidin-2-yl)-4-((5-(3-(2-methoxyethoxy)phenyl)-2-methyl-2H-1,2,3-triazol-4-yl)methyl)-1,4,9-triazaspiro[5.5]undecan-5-one;
9-(4,6-dimethylpyrimidin-2-yl)-4-((5-(3-isopropoxyphenyl)-2-methyl-2H-1,2,3-triazol-4-yl)methyl)-1,4,9-triazaspiro[5.5]undecan-5-one;
4-((5-(2,3-dihydrobenzo[b][1,4]dioxin-6-yl)-2-methyl-2H-1,2,3-triazol-4-yl)methyl)-9-(4,6-dimethylpyrimidin-2-yl)-1,4,9-triazaspiro[5.5]undecan-5-one;
9-(4,6-dimethylpyrimidin-2-yl)-4-((4-phenylisoxazol-3-yl)methyl)-1,4,9-triazaspiro[5.5]undecan-5-one;
9-(4,6-dimethylpyrimidin-2-yl)-4-((3-phenylisoxazol-4-yl)methyl)-1,4,9-triazaspiro[5.5]undecan-5-one;
9-(4,6-dimethylpyrimidin-2-yl)-4-((2-methyl-5-phenyl-2H-1,2,3-triazol-4-yl)methyl)-1,4,9-triazaspiro[5.5]undecan-5-one;
4-((1H-Indo)-3-yl)methyl)-9-(4,6-dimethylpyrimidin-2-yl)-1-oxa-4,9-diazaspiro[5.5]undecan-5-one;
9-(4,6-Dimethyl-pyrimidin-2-yl)-4-(1H-indazol-3-ylmethyl)-1-oxa-4,9-diaza-spiro[5.5]undecan-5-one;
9-(4,6-Dimethyl-pyrimidin-2-yl)-4-(2-furan-2-yl-benzyl)-1-oxa-4,9-diaza-spiro[5.5]undecan-5-one;
4-((1H-indazol-3-yl)methyl)-9-(4-methoxy-6-methylpyrimidin-2-yl)-1-oxa-4,9-diazaspiro[5.5]undecan-5-one;
4-((1H-Indol-3-yl)methyl)-9-(4-methoxypyrimidin-2-yl)-1-oxa-4,9-diazaspiro[5.5]undecan-5-one;
9-(4,6-Dimethylpyrimidin-2-yl)-4-((5-fluoro-1H-indol-3-yl)methyl)-1-oxa-4,9-diazaspiro[5.5]undecan-5-one;
9-(4,6-Dimethylpyrimidin-2-yl)-4-((5-methoxy-1H-indol-3-yl)methyl)-1-oxa-4,9-diazaspiro[5.5]undecan-5-one;
4-((1H-Indo)-3-yl)methyl)-9-(4-methylpyrimidin-2-yl)-1-oxa-4,9-diazaspiro[5.5]undecan-5-one;
9-(4,6-Dimethylpyrimidin-2-yl)-4-(2-(oxazol-2-yl)benzyl)-1-oxa-4,9-diazaspiro[5.5]undecan-5-one;
9-(4,6-Dimethylpyrimidin-2-yl)-4-(2-(3-methyl-1,2,4-oxadiazol-5-yl)benzyl)-1-oxa-4,9-diazaspiro[5.5]undecan-5-one;
9-(4,6-Dimethylpyrimidin-2-yl)-4-((1-methyl-4-phenyl-1H-pyrazol-3-yl)methyl)-1-oxa-4,9-diazaspiro[5.5]undecan-5-one;
9-(4,6-Dimethylpyrimidin-2-yl)-4-((2-methyl-5-phenyl-2H-1,2,3-triazol-4-yl)methyl)-1-oxa-4,9-diazaspiro[5.5]undecan-5-one;
9-(4,6-Dimethylpyrimidin-2-yl)-4-((4-phenyl-1H-pyrazol-3-yl)methyl)-1-oxa-4,9-diazaspiro[5.5]undecan-5-one;
9-(4,6-Dimethylpyrimidin-2-yl)-4-((5-phenyl-2H-1,2,3-triazol-4-yl)methyl)-1-oxa-4,9-diazaspiro[5.5]undecan-5-one;
9-(4,6-Dimethylpyrimidin-2-yl)-4-((1-methyl-4-phenyl-1H-1,2,3-triazol-5-yl)methyl)-1-oxa-4,9-diazaspiro[5.5]undecan-5-one;
9-(4,6-Dimethylpyrimidin-2-yl)-4-((1-methyl-5-phenyl-1H-1,2,3-triazol-4-yl)methyl)-1-oxa-4,9-diazaspiro[5.5]undecan-5-one;
9-(4,6-Dimethylpyrimidin-2-yl)-4-((5-(3-methoxyphenyl)-2H-1,2,3-triazol-4-yl)methyl)-1-oxa-4,9-diazaspiro[5.5]undecan-5-one;
4-((1H-Indol-3-yl)methyl)-9-(4-methoxy-6-methylpyrimidin-2-yl)-1-oxa-4,9-diazaspiro[5.5]undecan-5-one;
4-((1H-Pyrrolo[2,3-b]pyridin-3-yl)methyl)-9-(4-methoxy-6-methylpyrimidin-2-yl)-1-oxa-4,9-diazaspiro[5.5]undecan-5-one;
9-(4-Methoxy-6-methylpyrimidin-2-yl)-4-((5-(3-methoxyphenyl)oxazol-4-yl)methyl)-1-oxa-4,9-diazaspiro[5.5]undecan-5-one;
4-((1H-Indazol-3-yl)methyl)-9-(4-methylpyrimidin-2-yl)-1-oxa-4,9-diazaspiro[5.5]undecan-5-one;
4-((1H-indol-3-yl)methyl)-9-(4-ethylpyrimidin-2-yl)-1-oxa-4,9-diazaspiro[5.5]undecan-5-one;
4-((1H-indol-3-yl)methyl)-9-(4-ethyl-6-methylpyrimidin-2-yl)-1-oxa-4,9-diazaspiro[5.5]undecan-5-one;
4-((1H-indol-3-yl)methyl)-9-(4,5-dimethylpyrimidin-2-yl)-1-oxa-4,9-diazaspiro[5.5]undecan-5-one;
9-(4,6-dimethylpyrimidin-2-yl)-4-((4-phenylisoxazol-3-yl)methyl)-1-oxa-4,9-diazaspiro[5.5]undecan-5-one;
9-(4,6-dimethylpyrimidin-2-yl)-4-((3-phenylisoxazol-4-yl)methyl)-1-oxa-4,9-diazaspiro[5.5]undecan-5-one;
2-((1H-Indo)-3-yl)methyl)-8-(4,6-dimethylpyrimidin-2-yl)-2,8-diazaspiro[4.5]decan-1-one;
2-((1H-Indo)-3-yl)methyl)-8-(quinoxalin-2-yl)-2,8-diazaspiro[4.5]decan-1-one;
2-(2-((1H-Indol-3-yl)methyl)-1-oxo-2,8-diazaspiro[4.5]decan-8-yl)isonicotinonitrile;
2-((1H-Indo)-3-yl)methyl)-8-(6-methoxypyridin-2-yl)-2,8-diazaspiro[4.5]decan-1-one;
2-((1H-Indo)-3-yl)methyl)-8-(4-methylpyrimidin-2-yl)-2,8-diazaspiro[4.5]decan-1-one;
2-(Biphenyl-2-ylmethyl)-8-(quinoxalin-2-yl)-2,8-diazaspiro[4.5]decan-1-one;
8-(1H-Benzo[d]imidazol-2-yl)-2-(naphthalen-1-ylmethyl)-2,8-diazaspiro[4.5]decan-1-one;
2-(Biphenyl-2-ylmethyl)-8-(1-methyl-1H-benzo[d]imidazol-2-yl)-2,8-diazaspiro[4.5]decan-1-one;
8-(1H-Benzo[d]imidazol-2-yl)-2-(biphenyl-2-ylmethyl)-2,8-diazaspiro[4.5]decan-1-one;
8-(Benzo[d]thiazol-2-yl)-2-(biphenyl-2-ylmethyl)-2,8-diazaspiro[4.5]decan-1-one;
2-(Naphthalen-1-ylmethyl)-8-(quinoxalin-2-yl)-2,8-diazaspiro[4.5]decan-1-one;
8-(1H-Benzoimidazol-2-yl)-2-(1H-indol-3-ylmethyl)-2,8-diaza-spiro[4.5]decan-1-one;
8-Benzooxazol-2-yl-2-(1H-indol-3-ylmethyl)-2,8-diaza-spiro[4.5]decan-1-one;
2-(1H-Indo)-3-ylmethyl)-8-(4-methoxy-pyrimidin-2-yl)-2,8-diaza-spiro[4.5]decan-1-one;
8-(4-Methoxy-pyrimidin-2-yl)-2-(1H-pyrrolo[2,3-b]pyridin-3-ylmethyl)-2,8-diaza-spiro[4.5]decan-1-one;
2-(1H-Indazol-3-ylmethyl)-8-(4-methoxy-pyrimidin-2-yl)-2,8-diaza-spiro[4.5]decan-1-one;
8-(4-Methoxy-pyrimidin-2-yl)-2-[2-(3-methyl-[1,2,4]oxadiazol-5-yl)-benzyl]-2,8-diaza-spiro[4.5]decan-1-one;
2-(1H-Indo)-3-ylmethyl)-8-(4-methoxy-6-methyl-pyrimidin-2-yl)-2,8-diaza-spiro[4.5]decan-1-one;
8-(4,6-Dimethyl-pyrimidin-2-yl)-2-[2-(3-methyl-[1,2,4]oxadiazol-5-yl)-benzyl]-2,8-diaza-spiro[4.5]decan-1-one;
8-(4-Methoxy-6-methyl-pyrimidin-2-yl)-2-[2-(3-methyl-[1,2,4]oxadiazol-5-yl)-benzyl]-2,8-diaza-spiro[4.5]decan-1-one;
2-((1H-indazol-3-yl)methyl)-8-(4-methoxy-6-methylpyrimidin-2-yl)-2,8-diazaspiro[4.5]decan-1-one;
2-((1H-pyrrolo[2,3-b]pyridin-3-yl)methyl)-8-(4-methoxy-6-methylpyrimidin-2-yl)-2,8-diazaspiro[4.5]decan-1-one;
2-((1H-indol-5-yl)methyl)-8-(4,6-dimethylpyrimidin-2-yl)-2,8-diazaspiro[4.5]decan-1-one;
8-(4,6-dimethylpyrimidin-2-yl)-2-((2-methyl-5-phenyl-2H-1,2,3-triazol-4-yl)methyl)-2,8-diazaspiro[4.5]decan-1-one;
3-((1H-indol-3-yl)methyl)-8-(4,6-dimethylpyrimidin-2-yl)-1,3,8-triazaspiro[4.5]decan-4-one;
2-((1H-Indo)-3-yl)methyl)-7-(4-methoxypyrimidin-2-yl)-2,7-diazaspiro[4.4]nonan-1-one;
2-((1H-indol-3-yl)methyl)-7-(4-methoxypyrimidin-2-yl)-2,7-diazaspiro[4.4]nonan-1-one;
2-((1H-Indol-3-yl)methyl)-7-(4-methoxy-6-methylpyrimidin-2-yl)-2,7-diazaspiro[4.4]nonan-1-one;
2-((1H-Indo)-3-yl)methyl)-7-(quinoxalin-2-yl)-2,7-diazaspiro[4.4]nonan-1-one;
2-((1H-Indo)-3-yl)methyl)-7-(4-methylpyrimidin-2-yl)-2,7-diazaspiro[4.4]nonan-1-one;
2-((1H-Indo)-3-yl)methyl)-7-(4,6-dimethylpyrimidin-2-yl)-2,7-diazaspiro[4.4]nonan-1-one;
2-((1H-Indol-3-yl)methyl)-7-(benzo[d]oxazol-2-yl)-2,7-diazaspiro[4.4]nonan-1-one;
2-((1H-Indo)-4-yl)methyl)-7-(4,6-dimethylpyrimidin-2-yl)-2,7-diazaspiro[4.4]nonan-1-one;
2-((1H-Indol-4-yl)methyl)-7-(4-methoxy-6-methylpyrimidin-2-yl)-2,7-diazaspiro[4.4]nonan-1-one;
2-((1H-Pyrrolo[2,3-b]pyridin-3-yl)methyl)-7-(4-methoxypyrimidin-2-yl)-2,7-diazaspiro[4.4]nonan-1-one;
2-((1H-Indazol-3-yl)methyl)-7-(4-methoxypyrimidin-2-yl)-2,7-diazaspiro[4.4]nonan-1-one;
7-(4-Methoxypyrimidin-2-yl)-2-(2-(3-methyl-1,2,4-oxadiazol-5-yl)benzyl)-2,7-diazaspiro[4.4]nonan-1-one;
2-((5-(3-Methoxyphenyl)-2H-1,2,3-triazol-4-yl)methyl)-7-(4-methoxypyrimidin-2-yl)-2,7-diazaspiro[4.4]nonan-1-one;
2-((1H-Indo)-3-yl)methyl)-7-(5-chlorobenzo[d]oxazol-2-yl)-2,7-diazaspiro[4.4]nonan-1-one;
2-((1H-Indo)-3-yl)methyl)-7-(6-fluoroquinazolin-2-yl)-2,7-diazaspiro[4.4]nonan-1-one;
2-((1H-indol-3-yl)methyl)-7-(6-(trifluoromethyl)pyrimidin-4-yl)-2,7-diazaspiro[4.4]nonan-1-one;
7-(4-(1H-imidazol-1-yl)pyrimidin-2-yl)-2-((1H-indol-3-yl)methyl)-2,7-diazaspiro[4.4]nonan-1-one;
7-(2-(1H-imidazol-1-yl)pyrimidin-4-yl)-2-((1H-indol-3-yl)methyl)-2,7-diazaspiro[4.4]nonan-1-one;
7-(6-(1H-imidazol-1-yl)pyridazin-3-yl)-2-((1H-indol-3-yl)methyl)-2,7-diazaspiro[4.4]nonan-1-one;
2-((1H-indol-3-yl)methyl)-7-(2-cyclopropyl-6-methoxypyrimidin-4-yl)-2,7-diazaspiro[4.4]nonan-1-one;
2-((1H-indol-3-yl)methyl)-7-(4-(1-methyl-1H-imidazol-2-yl)pyrimidin-2-yl)-2,7-diazaspiro[4.4]nonan-1-one;
2-((1H-indol-3-yl)methyl)-7-(5-methylthiazol-2-yl)-2,7-diazaspiro[4.4]nonan-1-one;
2-((1H-indol-3-yl)methyl)-7-(7,8-dihydro-5H-pyrano[4,3-c]pyridazin-3-yl)-2,7-diazaspiro[4.4]nonan-1-one;
2-((1H-indol-3-yl)methyl)-7-(4-methyl-6-morpholinopyrimidin-2-yl)-2,7-diazaspiro[4.4]nonan-1-one;
2-((1H-indol-3-yl)methyl)-7-(2-methyl-6-morpholinopyrimidin-4-yl)-2,7-diazaspiro[4.4]nonan-1-one;
2-((1H-indol-3-yl)methyl)-7-(6-methylpyridin-2-yl)-2,7-diazaspiro[4.4]nonan-1-one;
2-((1H-indol-3-yl)methyl)-7-(4-methylpyridin-2-yl)-2,7-diazaspiro[4.4]nonan-1-one;
2-((1H-indol-3-yl)methyl)-7-(4,6-dimethylpyridin-2-yl)-2,7-diazaspiro[4.4]nonan-1-one;
2-((1H-indol-3-yl)methyl)-7-(4-methoxypyridin-2-yl)-2,7-diazaspiro[4.4]nonan-1-one;
2-((1H-indol-3-yl)methyl)-7-(2,6-dimethylpyrimidin-4-yl)-2,7-diazaspiro[4.4]nonan-1-one;
2-((1H-indol-3-yl)methyl)-7-(1-methyl-1H-pyrrolo[2,3-c]pyridin-7-yl)-2,7-diazaspiro[4.4]nonan-1-one;
2-((1H-indol-3-yl)methyl)-7-(7-methoxypyrazolo[1,5-a]pyrimidin-5-yl)-2,7-diazaspiro[4.4]nonan-1-one;
2-((1H-indol-3-yl)methyl)-7-(4-(3-methyl-1,2,4-oxadiazol-5-yl)pyridin-2-yl)-2,7-diazaspiro[4.4]nonan-1-one;
2-((1H-indol-3-yl)methyl)-7-(6-(pyrrolidin-1-yl)pyrimidin-4-yl)-2,7-diazaspiro[4.4]nonan-1-one;
2-((1H-indol-3-yl)methyl)-7-(2-methoxy-6-methylpyrimidin-4-yl)-2,7-diazaspiro[4.4]nonan-1-one;
2-((1H-indol-3-yl)methyl)-7-(4-methylthiazol-2-yl)-2,7-diazaspiro[4.4]nonan-1-one;
2-((1H-indol-3-yl)methyl)-7-(5-methoxypyrazolo[1,5-a]pyrimidin-7-yl)-2,7-diazaspiro[4.4]nonan-1-one;
2-((1H-indol-3-yl)methyl)-7-(4-isopropylpyrimidin-2-yl)-2,7-diazaspiro[4.4]nonan-1-one;
2-((1H-indol-3-yl)methyl)-7-(2-methylimidazo[1,2-a]pyrazin-8-yl)-2,7-diazaspiro[4.4]nonan-1-one;
2-((1H-indol-3-yl)methyl)-7-(4-(trifluoromethyl)pyrimidin-2-yl)-2,7-diazaspiro[4.4]nonan-1-one;
7-(4-methoxy-6-methylpyrimidin-2-yl)-2-(2-(3-methyl-1,2,4-oxadiazol-5-yl)benzyl)-2,7-diazaspiro[4.4]nonan-1-one;
7-(4-methoxy-6-methylpyrimidin-2-yl)-2-((2-methyl-5-phenyl-2H-1,2,3-triazol-4-yl)methyl)-2,7-diazaspiro[4.4]nonan-1-one;
7-(4-methoxy-6-methylpyrimidin-2-yl)-2-((2-methyl-5-(m-tolyl)-2H-1,2,3-triazol-4-yl)methyl)-2,7-diazaspiro[4.4]nonan-1-one;
7-(4-methoxy-6-methylpyrimidin-2-yl)-2-((5-(3-methoxyphenyl)-2-methyl-2H-1,2,3-triazol-4-yl)methyl)-2,7-diazaspiro[4.4]nonan-1-one;
2-((5-bromo-2-methyl-2H-1,2,3-triazol-4-yl)methyl)-7-(4-methoxy-6-methylpyrimidin-2-yl)-2,7-diazaspiro[4.4]nonan-1-one;
2-((1H-indol-3-yl)methyl)-7-(4-(dimethylamino)-6-(trifluoromethyl)pyrimidin-2-yl)-2,7-diazaspiro[4.4]nonan-1-one;
2-((1H-Indo)-3-yl)methyl)-7-(quinoxalin-2-yl)-2,7-diazaspiro[4.5]decan-1-one;
7-((1H-Indol-3-yl)methyl)-2-(4-methoxy-6-methylpyrimidin-2-yl)-2,7-diazaspiro[4.5]decan-6-one;
7-((1H-Pyrrolo[2,3-b]pyridin-3-yl)methyl)-2-(4-methoxypyrimidin-2-yl)-2,7-diazaspiro[4.5]decan-6-one;
7-((1H-Indazol-3-yl)methyl)-2-(4-methoxypyrimidin-2-yl)-2,7-diazaspiro[4.5]decan-6-one;
7-((1H-Indo)-3-yl)methyl)-2-(4-methoxypyrimidin-2-yl)-2,7-diazaspiro[4.5]decan-6-one;
2-(4-Methoxypyrimidin-2-yl)-7-(2-(3-methyl-1,2,4-oxadiazol-5-yl)benzyl)-2,7-diazaspiro[4.5]decan-6-one;
7-((5-(3-Methoxyphenyl)-2H-1,2,3-triazol-4-yl)methyl)-2-(4-methoxypyrimidin-2-yl)-2,7-diazaspiro[4.5]decan-6-one;
7-((5-(3-Methoxyphenyl)-2-methyl-2H-1,2,3-triazol-4-yl)methyl)-2-(4-methoxypyrimidin-2-yl)-2,7-diazaspiro[4.5]decan-6-one;
7-((1H-Indol-3-yl)methyl)-2-(benzo[d]oxazol-2-yl)-2,7-diazaspiro[4.5]decan-6-one;
7-((1H-indol-3-yl)methyl)-2-(4-methylpyrimidin-2-yl)-2,7-diazaspiro[4.5]decan-6-one;
7-((1H-indazol-3-yl)methyl)-2-(4-methoxy-6-methylpyrimidin-2-yl)-2,7-diazaspiro[4.5]decan-6-one;
7-((1H-pyrrolo[2,3-b]pyridin-3-yl)methyl)-2-(4-methoxy-6-methylpyrimidin-2-yl)-2,7-diazaspiro[4.5]decan-6-one;
2-(4-methoxy-6-methylpyrimidin-2-yl)-7-(2-(3-methyl-1,2,4-oxadiazol-5-yl)benzyl)-2,7-diazaspiro[4.5]decan-6-one;
7-((1H-indol-3-yl)methyl)-2-(1H-benzo[d]imidazol-2-yl)-2,7-diazaspiro[4.5]decan-6-one;
1-((1H-Indo)-4-yl)methyl)-8-(quinoxalin-2-yl)-1,8-diazaspiro[4.5]decan-2-one;
1-((1H-Indo)-4-yl)methyl)-8-(6-methylpyrazin-2-yl)-1,8-diazaspiro[4.5]decan-2-one;
1-((1H-Indo)-4-yl)methyl)-8-(4,6-dimethylpyrimidin-2-yl)-1,8-diazaspiro[4.5]decan-2-one;
2-(1-((1H-Indol-4-yl)methyl)-2-oxo-1,8-diazaspiro[4.5]decan-8-yl)isonicotinonitrile;
1-(2,5-Dimethylbenzyl)-8-(quinoxalin-2-yl)-1,8-diazaspiro[4.5]decan-2-one;
1-(2,5-Dimethylbenzyl)-8-(4,6-dimethylpyrimidin-2-yl)-1,8-diazaspiro[4.5]decan-2-one;
1-(2,5-Dimethylbenzyl)-9-(quinoxalin-2-yl)-4-oxa-1,9-diazaspiro[5.5]undecan-2-one;
1-((1H-Indo)-3-yl)methyl)-9-(quinoxalin-2-yl)-4-oxa-1,9-diazaspiro[5.5]undecan-2-one;
1-((1H-Indo)-4-yl)methyl)-9-(quinoxalin-2-yl)-4-oxa-1,9-diazaspiro[5.5]undecan-2-one;
1-((1-Methyl-1H-indol-4-yl)methyl)-9-(quinoxalin-2-yl)-4-oxa-1,9-diazaspiro[5.5]undecan-2-one;
1-(3-(Pyridin-2-yl)benzyl)-9-(quinoxalin-2-yl)-4-oxa-1,9-diazaspiro[5.5]undecan-2-one;
1-(3-(Pyridin-3-yl)benzyl)-9-(quinoxalin-2-yl)-4-oxa-1,9-diazaspiro[5.5]undecan-2-one;
1-(2,5-Dimethylbenzyl)-9-(6-methylpyrazin-2-yl)-4-oxa-1,9-diazaspiro[5.5]undecan-2-one;
1-((1H-Indo)-4-yl)methyl)-9-(6-methylpyrazin-2-yl)-4-oxa-1,9-diazaspiro[5.5]undecan-2-one;
1-(2,5-Dimethylbenzyl)-9-(4,6-dimethylpyrimidin-2-yl)-4-oxa-1,9-diazaspiro[5.5]undecan-2-one;
2-(1-((1H-Indol-4-yl)methyl)-2-oxo-4-oxa-1,9-diazaspiro[5.5]undecan-9-yl)isonicotinonitrile;
6-(1-((1H-Indol-4-yl)methyl)-2-oxo-4-oxa-1,9-diazaspiro[5.5]undecan-9-yl)picolinonitrile;
2-(1-((1H-Indol-4-yl)methyl)-2-oxo-4-oxa-1,9-diazaspiro[5.5]undecan-9-yl)pyrimidine-4-carbonitrile;
1-((1H-Indo)-4-yl)methyl)-9-(4-methylpyrimidin-2-yl)-4-oxa-1,9-diazaspiro[5.5]undecan-2-one;
1-(2,5-Dimethylbenzyl)-8-(quinoxalin-2-yl)-3-oxa-1,8-diazaspiro[4.5]decan-2-one;
1-((1H-Indo)-3-yl)methyl)-8-(quinoxalin-2-yl)-3-oxa-1,8-diazaspiro[4.5]decan-2-one;
1-((1H-Indo)-4-yl)methyl)-8-(quinoxalin-2-yl)-3-oxa-1,8-diazaspiro[4.5]decan-2-one;
1-((1H-Indo)-3-yl)methyl)-8-(6-methylpyrazin-2-yl)-3-oxa-1,8-diazaspiro[4.5]decan-2-one;
1-((1H-Indo)-3-yl)methyl)-8-(4,6-dimethylpyrimidin-2-yl)-3-oxa-1,8-diazaspiro[4.5]decan-2-one;
1-((1H-Indo)-4-yl)methyl)-8-(6-methylpyrazin-2-yl)-3-oxa-1,8-diazaspiro[4.5]decan-2-one, and
1-((1H-Indo)-4-yl)methyl)-7-(quinoxalin-2-yl)-1,7-diazaspiro[4.4]nonan-2-one;
and wherein said compound is in free form or in salt form or in pharmaceutically acceptable salt form.

8. A pharmaceutical composition comprising a therapeutically effective amount of a compound of claim 1 in free form or in salt form or in pharmaceutically acceptable salt form and one or more pharmaceutically acceptable carriers.

9. A combination comprising a therapeutically effective amount of the compound of claim 1 in free form or in salt form or in pharmaceutically acceptable salt form and one or more therapeutically active agents.

10. A method of inhibiting orexin receptor activity in a subject, wherein the method comprises administering to the subject a therapeutically effective amount of the compound of claim 1 in free form or in salt form or in pharmaceutically acceptable salt form.

11. A method of treating a disorder or a disease in a subject mediated by orexin receptors, wherein the method comprises administering to the subject a therapeutically effective amount of the compound of claim 1 in free form or in salt form or in pharmaceutically acceptable salt form.

12. The method of claim 11 wherein the disease is Alzheimer's.
